SEQUENCE LISTING (96 sequences)

SEQ ID NO: 1 - protein, 116 aa, synthetic construct
  VARIANT 34: note = X is Ne-(tert-butoxycarbonyl) lysine or N6-(2-Oxoazetidine-1-carbonyl)-L-lysine
MGSSHHHHHHSSGTENLYFQGVSSVPTKLEVVAXTPTSLLISWDAPAVTVFFYVITYGETGHGVGAFQAFKVPGSKSTATISGLKPGVDYTITVYARGYSKQGPYKPSPISINKRT

SEQ ID NO: 2 - protein, 116 aa, synthetic construct
  VARIANT 34: note = X is Ne-(tert-butoxycarbonyl) lysine or N6-(2-Oxoazetidine-1-carbonyl)-L-lysine
MGSSHHHHHHSSGTENLYFQGVSSVPTKLKVVAXTPTSLLISWDAPAVTVFFYVITYGETGHGVGAFKAFKVPGSKSTATISGLKPGVDYTITVYARGYSKKGPYKPSPISINKRT

SEQ ID NO: 3 - protein, 116 aa, synthetic construct
  VARIANT 34: note = X is Ne-(tert-butoxycarbonyl) lysine or N6-(2-Oxoazetidine-1-carbonyl)-L-lysine
MGSSHHHHHHSSGTENLYFQGVSKVPTKLEVVAXTPTSLLIKWDAPAVTVKFYVITYGEKGHGVGAFQAFKVPGSKRTATIKGLKPGVDYTITVYARGYSKQGPYKPSPISINKRT

SEQ ID NO: 4 - protein, 116 aa, synthetic construct
MGSSHHHHHHSSGTENLYFQGCVSSVPTKLEVVAATPTSLLISWDAPAVTVDYYVITYGETGSGGYAWQEFEVPGSKSTATISGLKPGVDYTITVYAGYYGYPTYYSSPISINKRT

SEQ ID NO: 5 - protein, 116 aa, synthetic construct
  MOD_RES 35: note = X is Ne-(tert-butoxycarbonyl) lysine
MGSSHHHHHHSSGTENLYFQGCVSSVPTKLEVVAXTPTSLLISWDAPAVTVDYYVITYGETGSGGYAWQEFEVPGSKSTATISGLKPGVDYTITVYAGYYGYPTYYSSPISINYRT

SEQ ID NO: 6 - protein, 115 aa, synthetic construct
  MOD_RES 34: note = X is N6-(2-Oxoazetidine-1-carbonyl)-L-lysine
MGSSHHHHHHSSGTENLYFQGVSSKPTKLRVVRXTPTSLKISWDAPAVTVDYYVITYGEKGSGGYAWQEFEVPGSKRTATISGLKPGVDYTITVYAGYKGYPTYYSSPISINYRT

SEQ ID NO: 7 - protein, 115 aa, synthetic construct
  MOD_RES 34: note = X is N6-(2-Oxoazetidine-1-carbonyl)-L-lysine
MGSSHHHHHHSSGTENLYFQGVSSKPTKLRVVRXTPTSLKISWDAPAVTVDYYVITYGEKGSGGYAWQEFEVPGSKRTATISGLKPGVDYTITVYAGYKGYPTYYSSPISINKRT

SEQ ID NO: 8 - protein, 115 aa, synthetic construct; identical to SEQ ID NO: 7
  MOD_RES 34: note = X is Ne-(tert-butoxycarbonyl) lysine

SEQ ID NO: 9 - protein, 116 aa, synthetic construct
  MOD_RES 35: note = X is N6-(2-Oxoazetidine-1-carbonyl)-L-lysine
MGSSHHHHHHSSGTENLYFQGCVSSKPTKLRVVRXTPTSLKISWDAPAVTVDYYVITYGEKGSGGYAWQEFEVPGSKRTATISGLKPGVDYTITVYAGYKGYPTYYSSPISINKRT

SEQ ID NO: 10 - protein, 116 aa, synthetic construct; identical to SEQ ID NO: 9
  MOD_RES 35: note = X is Ne-(tert-butoxycarbonyl) lysine

SEQ ID NO: 11 - protein, 115 aa, synthetic construct
  MOD_RES 34: note = X is N6-(2-Oxoazetidine-1-carbonyl)-L-lysine
MGSSHHHHHHSSGTENLYFQGVSSKPTKLRVVRXTPTSLKIKWDAPAVTVDYYVITYGEKGRGGYAWQEFEVPGSKRTATISGLKPGVDYTITVYAGYKGYPTYYSSPISINYRT

SEQ ID NO: 12 - protein, 116 aa, synthetic construct
  MOD_RES 35: note = X is N6-(2-Oxoazetidine-1-carbonyl)-L-lysine
MGSSHHHHHHSSGTENLYFQGCVSSKPTKLRVVRXTPTSLKIKWDAPAVTVDYYVITYGEKGRGGYAWQEFEVPGSKRTATISGLKPGVDYTITVYAGYKGYPTYYSSPISINKRT

SEQ ID NO: 13 - protein, 116 aa, synthetic construct; identical to SEQ ID NO: 12
  MOD_RES 35: note = X is Ne-(tert-butoxycarbonyl) lysine

SEQ ID NO: 14 - protein, 115 aa, synthetic construct
  MOD_RES 34: note = X is N6-(2-Oxoazetidine-1-carbonyl)-L-lysine
MGSSHHHHHHSSGTENLYFQGVSSKPTKLRVVRXTPTSLKIKWDAPAKTVDYYVITYGETGRGGYAWQRFEVPGSKRTATIKGLKPGVDYTITVYAGYKGYPTYYSSPISINYRT

SEQ ID NO: 15 - protein, 116 aa, synthetic construct
  MOD_RES 35: note = X is N6-(2-Oxoazetidine-1-carbonyl)-L-lysine
MGSSHHHHHHSSGTENLYFQGCVSSKPTKLRVVRXTPTSLKIKWDAPAKTVDYYVITYGETGRGGYAWQRFEVPGSKRTATIKGLKPGVDYTITVYAGYKGYPTYYSSPISINYRT

SEQ ID NO: 16 - protein, 116 aa, synthetic construct
  MOD_RES 35: note = X is N6-(2-Oxoazetidine-1-carbonyl)-L-lysine
MGSSHHHHHHSSGTENLYFQGCVSSKPTKLRVVRXTPTSLKIKWDAPAKTVDYYVITYGETGRGGYAWQRFEVPGSKRTATIKGLKPGVDYTITVYAGYKGYPTYYSSPISINKRT

SEQ ID NO: 17 - protein, 116 aa, synthetic construct; identical to SEQ ID NO: 16
  MOD_RES 35: note = X is Ne-(tert-butoxycarbonyl) lysine

SEQ ID NO: 18 - protein, 115 aa, synthetic construct
  MOD_RES 34: note = X is N6-(2-Oxoazetidine-1-carbonyl)-L-lysine
MGSSHHHHHHSSGTENLYFQGVKSKPTKLRVVRXTPTSLKISWKAPKKTVDYYVITYGKTGSGGYAWQRFRVPGSKRTAKIKGLKPGVDYTITVYAGYKGYPTYYSSPISINYRT

SEQ ID NO: 19 - protein, 115 aa, synthetic construct
  MOD_RES 34: note = X is N6-(2-Oxoazetidine-1-carbonyl)-L-lysine
MGSSHHHHHHSSGTENLYFQGVKSKPTKLRVVRXTPTSLKISWKAPKKTVDYYVITYGKTGSGGYAWQRFRVPGSKRTAKIKGLKPGVDYTITVYAGYKGYPTYYSSPISINKRT

SEQ ID NO: 20 - protein, 116 aa, synthetic construct
  MOD_RES 35: note = X is N6-(2-Oxoazetidine-1-carbonyl)-L-lysine
MGSSHHHHHHSSGTENLYFQGCVKSKPTKLRVVRXTPTSLKISWKAPKKTVDYYVITYGKTGSGGYAWQRFRVPGSKRTAKIKGLKPGVDYTITVYAGYKGYPTYYSSPISINKRT

SEQ ID NO: 21 - protein, 116 aa, synthetic construct; identical to SEQ ID NO: 20
  MOD_RES 35: note = X is Ne-(tert-butoxycarbonyl) lysine

SEQ ID NO: 22 - protein, 116 aa, synthetic construct
  MOD_RES 34: note = X is N6-(2-Oxoazetidine-1-carbonyl)-L-lysine
MGSSHHHHHHSSGTENLYFQGVSSKPTKLRVVRXTPTSLKIKWDAPAKTVDYYVITYGETGRGGYAWQRFEVPGSKRTATIKGLKPGVDYTITVYAGYKGYPTYYSSPISINYRTC

SEQ ID NO: 23 - protein, 116 aa, synthetic construct; identical to SEQ ID NO: 15
  MOD_RES 35: note = X is N6-(2-Oxoazetidine-1-carbonyl)-L-lysine

SEQ ID NO: 24 - protein, 454 aa, synthetic construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 25 - protein, 454 aa, synthetic construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLVNYARKLDRALPDPIKIFEIGPCYRKESDGKEHLEEFTMLNFFQMGSGCTRENLESIITDFLNHLGIDFKIVGDSCMVFGDTLDVMHGDLELSSAVVGPIPLDREWGIDKPWIGAGFGLERLLKVKHDFKNIKRAARSESYYNGISTNL

SEQ ID NO: 26 - DNA, 37 bp, synthetic construct
  source 1..37: mol_type = other DNA
aacaaaaagtttgaattgggtttgtagtttcccaatc

SEQ ID NO: 27 - DNA, 39 bp, synthetic construct
  source 1..39: mol_type = other DNA
aatataataaggaagattgggaaactacaaacccaattc

SEQ ID NO: 28 - DNA, 26 bp, synthetic construct
  source 1..26: mol_type = other DNA
agagcgtgcatatatttcaatgcttg

SEQ ID NO: 29 - DNA, 18 bp, synthetic construct
  source 1..18: mol_type = other DNA
tttggacaaccacccaac

SEQ ID NO: 30 - DNA, 18 bp, synthetic construct
  source 1..18: mol_type = other DNA
tttggacaaccacccaac

SEQ ID NO: 31 - DNA, 18 bp, synthetic construct
  source 1..18: mol_type = other DNA
tttggacaaccacccaac

SEQ ID NO: 32 - DNA, 26 bp, synthetic construct
  source 1..26: mol_type = other DNA
agagcgtgcacatatttcaatgcttg

SEQ ID NO: 33 - DNA, 18 bp, synthetic construct
  source 1..18: mol_type = other DNA
tttggacaaccacccaac

SEQ ID NO: 34 - DNA, 21 bp, synthetic construct
  source 1..21: mol_type = other DNA
gagcgtgcagcgatttcaatg

SEQ ID NO: 35 - DNA, 19 bp, synthetic construct
  source 1..19: mol_type = other DNA
ttttggacaaccacccaac

SEQ ID NO: 36 - DNA, 29 bp, synthetic construct
  source 1..29: mol_type = other DNA
agagcgtgcatctatttcaatgcttgaag

SEQ ID NO: 37 - DNA, 18 bp, synthetic construct
  source 1..18: mol_type = other DNA
tttggacaaccacccaac

SEQ ID NO: 38 - DNA, 26 bp, synthetic construct
  source 1..26: mol_type = other DNA
agagcgtgcagatatttcaatgcttg

SEQ ID NO: 39 - DNA, 26 bp, synthetic construct
  source 1..26: mol_type = other DNA
agagcgtgcagatatttcaatgcttg

SEQ ID NO: 40 - DNA, 26 bp, synthetic construct
  source 1..26: mol_type = other DNA
agagcgtgcagatatttcaatgcttg

SEQ ID NO: 41 - DNA, 18 bp, synthetic construct
  source 1..18: mol_type = other DNA
tttggacaaccacccaac

SEQ ID NO: 42 - DNA, 26 bp, synthetic construct
  source 1..26: mol_type = other DNA
agagcgtgcaatgatttcaatgcttg

SEQ ID NO: 43 - DNA, 18 bp, synthetic construct
  source 1..18: mol_type = other DNA
tttggacaaccacccaac

SEQ ID NO: 44 - DNA, 32 bp, synthetic construct
  source 1..32: mol_type = other DNA
tttatgtcatgcaacatatttaaatggtgatc

SEQ ID NO: 45 - DNA, 20 bp, synthetic construct
  source 1..20: mol_type = other DNA
cgatcttcgaacattttcag

SEQ ID NO: 46 - DNA, 24 bp, synthetic construct
  source 1..24: mol_type = other DNA
tagcattaattttcgcacctaagg

SEQ ID NO: 47 - DNA, 18 bp, synthetic construct
  source 1..18: mol_type = other DNA
atcgggctactataatag

SEQ ID NO: 48 - DNA, 20 bp, synthetic construct
  source 1..20: mol_type = other DNA
gccctggaaatacaggtttt

SEQ ID NO: 49 - protein, 115 aa, synthetic construct
MGSSHHHHHHSSGTENLYFQGVSSVPTKLEVVAATPTSLLISWDAPAVTVDYYVITYGETGSGGYAWQEFEVPGSKSTATISGLKPGVDYTITVYAGYYGYPTYYSSPISINYRT

SEQ ID NO: 50 - protein, 115 aa, synthetic construct; identical to SEQ ID NO: 14
  VARIANT 34: note = X is 1/2-carboxy-4-aryltriazole lysine or Ne-(tert-butoxycarbonyl) lysine

SEQ ID NO: 51 - protein, 125 aa, synthetic construct
  VARIANT 4: note = X is 1/2-carboxy-4-aryltriazole lysine or Ne-(tert-butoxycarbonyl) lysine
MAQXQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSLEHHHHHH

SEQ ID NO: 52 - DNA, 22 bp, synthetic construct
  source 1..22: mol_type = other DNA
cgcacagatgggatcgggatgt

SEQ ID NO: 53 - DNA, 24 bp, synthetic construct
  source 1..24: mol_type = other DNA
aacgccagcatggtaaactcttcg

SEQ ID NO: 54 - DNA, 23 bp, synthetic construct
  source 1..23: mol_type = other DNA
taccatgctggtgttcgcacaga

SEQ ID NO: 55 - DNA, 22 bp, synthetic construct
  source 1..22: mol_type = other DNA
aactcttcgaggtgttctttgc

SEQ ID NO: 56 - DNA, 24 bp, synthetic construct
  source 1..24: mol_type = other DNA
gctggcgttcctgcagatgggatc

SEQ ID NO: 57 - DNA, 18 bp, synthetic construct
  source 1..18: mol_type = other DNA
atggtaaactcttcgagg

SEQ ID NO: 58 - DNA, 22 bp, synthetic construct
  source 1..22: mol_type = other DNA
cctgcagatgggatcgggatgt

SEQ ID NO: 59 - DNA, 24 bp, synthetic construct
  source 1..24: mol_type = other DNA
aagaccagcatggtaaactcttcg

SEQ ID NO: 60 - DNA, 37 bp, synthetic construct; identical to SEQ ID NO: 26
  source 1..37: mol_type = other DNA

SEQ ID NO: 61 - DNA, 39 bp, synthetic construct; identical to SEQ ID NO: 27
  source 1..39: mol_type = other DNA

SEQ ID NO: 62 - DNA, 26 bp, synthetic construct
  source 1..26: mol_type = other DNA
agagcgtgcatatatttcaatgcttg

SEQ ID NO: 63 - DNA, 18 bp, synthetic construct
  source 1..18: mol_type = other DNA
tttggacaaccacccaac

SEQ ID NO: 64 - DNA, 26 bp, synthetic construct
  source 1..26: mol_type = other DNA
agagcgtgcatgcatttcaatgcttg

SEQ ID NO: 65 - DNA, 18 bp, synthetic construct
  source 1..18: mol_type = other DNA
tttggacaaccacccaac

SEQ ID NO: 66 - DNA, 26 bp, synthetic construct
  source 1..26: mol_type = other DNA
agagcgtgcacatatttcaatgcttg

SEQ ID NO: 67 - DNA, 18 bp, synthetic construct
  source 1..18: mol_type = other DNA
tttggacaaccacccaac

SEQ ID NO: 68 - DNA, 29 bp, synthetic construct
  source 1..29: mol_type = other DNA
agagcgtgcatctatttcaatgcttgaag

SEQ ID NO: 69 - DNA, 18 bp, synthetic construct
  source 1..18: mol_type = other DNA
tttggacaaccacccaac

SEQ ID NO: 70 - DNA, 26 bp, synthetic construct
  source 1..26: mol_type = other DNA
agagcgtgcagatatttcaatgcttg

SEQ ID NO: 71 - DNA, 18 bp, synthetic construct
  source 1..18: mol_type = other DNA
tttggacaaccacccaac

SEQ ID NO: 72 - DNA, 26 bp, synthetic construct
  source 1..26: mol_type = other DNA
agagcgtgcaaccatttcaatgcttg

SEQ ID NO: 73 - DNA, 18 bp, synthetic construct
  source 1..18: mol_type = other DNA
tttggacaaccacccaac

SEQ ID NO: 74 - DNA, 21 bp, synthetic construct
  source 1..21: mol_type = other DNA
tgcgtgagcagcgtgccgacc

SEQ ID NO: 75 - DNA, 27 bp, synthetic construct
  source 1..27: mol_type = other DNA
gccctgaaaatacagattttcggtacc

SEQ ID NO: 76 - DNA, 21 bp, synthetic construct
  source 1..21: mol_type = other DNA
tgcgttagtagtaaaccgacc

SEQ ID NO: 77 - DNA, 21 bp, synthetic construct
  source 1..21: mol_type = other DNA
accctggaaatacaggttttc

SEQ ID NO: 78 - DNA, 21 bp, synthetic construct
  source 1..21: mol_type = other DNA
tgcgttagtagtaaaccgacc

SEQ ID NO: 79 - DNA, 21 bp, synthetic construct
  source 1..21: mol_type = other DNA
accctggaaatacaggttttc

SEQ ID NO: 80 - DNA, 21 bp, synthetic construct
  source 1..21: mol_type = other DNA
tgcgtgagcagtaaaccgacc

SEQ ID NO: 81 - DNA, 20 bp, synthetic construct
  source 1..20: mol_type = other DNA
gccctggaaatacaggtttt

SEQ ID NO: 82 - DNA, 21 bp, synthetic construct
  source 1..21: mol_type = other DNA
tgcgtgaaaagcaaaccgacc

SEQ ID NO: 83 - DNA, 21 bp, synthetic construct
  source 1..21: mol_type = other DNA
accctggaaatacaggttttc

SEQ ID NO: 84 - DNA, 21 bp, synthetic construct
  source 1..21: mol_type = other DNA
tagtattaaacgtacctaagg

SEQ ID NO: 85 - DNA, 18 bp, synthetic construct
  source 1..18: mol_type = other DNA
atcgggctgctataatag

SEQ ID NO: 86 - DNA, 24 bp, synthetic construct
  source 1..24: mol_type = other DNA
tagtattaataaacgtacctaagg

SEQ ID NO: 87 - DNA, 18 bp, synthetic construct
  source 1..18: mol_type = other DNA
atcgggctgctataatag

SEQ ID NO: 88 - DNA, 23 bp, synthetic construct
  source 1..23: mol_type = other DNA
tagcattaataaacgcacctaag

SEQ ID NO: 89 - DNA, 18 bp, synthetic construct
  source 1..18: mol_type = other DNA
atcggactgctataatag

SEQ ID NO: 90 - DNA, 22 bp, synthetic construct
  source 1..22: mol_type = other DNA
cgcacagatgggatcgggatgt

SEQ ID NO: 91 - DNA, 24 bp, synthetic construct
  source 1..24: mol_type = other DNA
aacgccagcatggtaaactcttcg

SEQ ID NO: 92 - protein, 21 aa, synthetic construct
MGSSHHHHHHSSGTENLYFQG

SEQ ID NO: 93 - protein, 7 aa, synthetic construct
XTPTSLK

SEQ ID NO: 94 - protein, 15 aa, synthetic construct
GYPTYYSSPISINYR

SEQ ID NO: 95 - protein, 8 aa, synthetic construct
IKWDAPAK

SEQ ID NO: 96 - protein, 11 aa, synthetic construct
XTPTSLKISWK

(19) United States
(12) Patent Application Publication
(10) Pub. No.: US 2026/0117274 A1
(43) Pub. Date: Apr. 30, 2026

(54) ORTHOGONALLY CROSSLINKED PROTEINS, METHODS OF MAKING, AND USES THEREOF

(71) Applicant: The Research Foundation for The State University of New York, Amherst, NY (US)

(72) Inventor: Qing LIN, Getzville, NY (US)

(21) Appl. No.: 18/847,417

(22) PCT Filed: Mar. 14, 2023
(86) PCT No.: PCT/US2023/064341

§ 371 Date: Sep. 16, 2024

Related U.S. Application Data
(60) Provisional application No. 63/319,576, filed on Mar. 14, 2022.
(60) Provisional application No. 63/448,121, filed on Feb. 24, 2023.

(51) Int. Cl. C12P 21/00; C07K 14/00; C12N 9/00

(52) U.S. Cl. CPC C12P 21/00; C07K 14/00; C12N 9/93; C12Y 601/01026

(57) ABSTRACT

Compounds, proteins, crosslinked proteins, compositions thereof, and methods of making and uses thereof. A compound, which may be an alpha-amino acid, comprises one or more beta-lactam group(s), one or more triazole groups, substituted analogs thereof, or any combination thereof. A protein comprises one or more amino acid residue(s), each residue comprising a beta-lactam group, a triazole group, or a substituted analog thereof. A protein can be made by a recombinant method using one or more compound(s). A cross-linked protein comprises one or more intramolecular crosslink(s) and/or one or more intermolecular crosslink(s). In various examples, a crosslink is formed, e.g., in solution or in vivo, by a proximity enabled beta-lactam ring opening reaction or an acyl transfer reaction between a beta-lactam group or a triazole group and a nucleophilic side-chain group, where both groups are on a single polypeptide or on different polypeptide chains. Crosslinked protein(s) can be used in methods of treatment.

### CROSS-REFERENCE TO RELATED APPLICATIONS

This application claims the benefit of U.S. Provisional Patent Application Nos. 63/319,576, filed Mar. 14, 2022, and 63/448,121, filed Feb. 24, 2023, the contents of the above-identified applications are hereby fully incorporated herein by reference in their entirety.

### STATEMENT REGARDING FEDERALLY SPONSORED RESEARCH

This invention was made with government support under Grant Number GM130307 awarded by the National Institutes of Health and Grant Number CHE-1904558 awarded by the National Science Foundation. The government has certain rights in the invention.

### SEQUENCE LISTING

This application contains a sequence listing filed in electronic form as an xml file entitled RFSUNY-0110WP_ST26.xml, created on Mar. 14, 2023, and having size of 97,789 bytes. The content of the sequence listing is incorporated herein in its entirety.

### BACKGROUND OF THE DISCLOSURE

The disulfide bond has been the principle natural crosslink in protein structure, offering a redox-active covalent crosslink for regulating protein stability and function. For engineering purposes, the exogenous disulfide bonds have been engineered into proteins to enhance protein stability. However, this approach has two major limitations: 1) recombinant expression of the cysteine-rich proteins in bacteria frequently leads to misfolding and formation of the inclusion bodies, requiring a lengthy refolding process to obtain native protein structure; 2) the disulfide bond is labile in the reducing environment of mammalian cytosol, rendering it unsuitable for intracellular applications.

Since their seminal discovery by Kohler and Milstein in 1975, monoclonal antibodies have profoundly transformed biomedical science. Coupled with powerful molecular evolution techniques such as phage display, monoclonal antibodies that bind to virtually any extracellular targets with high affinity and specificity can be rapidly developed. However, monoclonal antibodies are generally not cell-permeable, precluding their use in targeting intracellular proteins. On the other hand, small antibody or antibody-like structures, e.g., heavy chain-only nanobodies found in camels and sharks and synthetic antibody mimetics derived from the fibronectin type III domain (FN3) called monobodies, provide attractive scaffolds for targeting intracellular proteins, owing to their small size (10-15 kDa), robust immunoglobin fold, and versatile binding. Therefore, strategies to make small-format antibodies cell-permeable are invaluable and expected to impact biologics' development significantly.

A proven strategy to endow cell permeability to small-format antibodies is through supercharging. To this end, two approaches have been reported: 1) chemical supercharging in which a cell-penetrating peptide such as cyclic dodeca-arginine is conjugated to nanobodies; and 2) genetic supercharging in which a large number of solvent-exposed surface residues are mutated to lysines or arginines. Compared to chemical supercharging, genetic modification has several advantages: 1) the expression and purification are facile; 2) there is no significant increase in mass; and 3) the charged residues can be judiciously placed throughout small-format antibody surface to maximize cytosolic uptake without compromising its function. However, the disadvantage of the genetic approach is that extensive mutagenesis often destabilizes the immunoglobin fold, leading to its potential entrapment in the endosomes.

### SUMMARY OF THE DISCLOSURE

The present disclosure provides, inter alia, compounds, which can be used to make proteins, crosslinked proteins, compositions thereof. The present disclosure also provides uses of the compounds, proteins, and crosslinked proteins.

In various examples, a compound comprises (or consists of) the following structure:

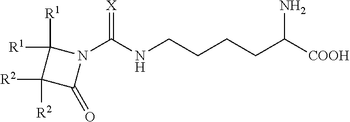

or a structural analog thereof, or a pharmaceutically acceptable salt, a salt, a partial salt, a solvate, a polymorph thereof, or a stereoisomer or a mixture of stereoisomers, an isotopic variant, or a tautomer thereof, where X is O or S or the like, R¹ and R² are independently at each occurrence chosen from hydrogen group, halide groups, alkyl groups, cycloalkyl groups, alkoxy groups, alkylamino groups, alkylthiol groups, and structural analogs thereof, and optionally, a R¹ and a R² form a hydrocarbon ring, a heterocyclic ring, and structural analogs thereof. In various examples, a compound comprises (or consists of) the following structure:

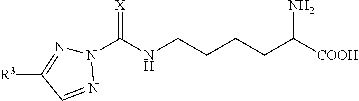

or a structural analog thereof, or a pharmaceutically acceptable salt, a salt, a partial salt, a solvate, a polymorph, or a stereoisomer or a mixture of stereoisomers, an isotopic variant, or a tautomer thereof, where X is O or S or the like, and R³ is chosen from hydrogen group, alkyl groups, cycloalkyl groups, aromatic groups, heteroaromatic groups, and structural analogs thereof. In various examples, the R³ group comprises (or consists of) the following structure:

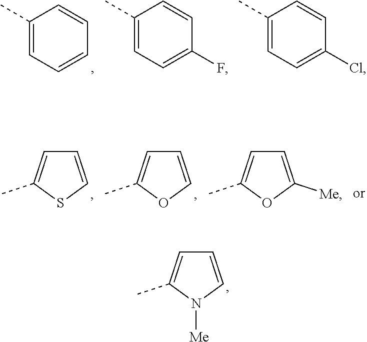

where Me is a methyl group, or a structural analog thereof. In various examples, the compound comprises the following structure:

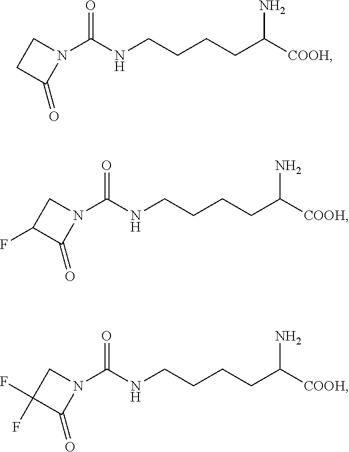 [SMILES: NC(CCCCNC(=O)N1CCC1=O)C(=O)O ; NC(CCCCNC(=O)N1CC(F)C1=O)C(=O)O ; NC(CCCCNC(=O)N1CC(F)(F)C1=O)C(=O)O]

or a structural analog thereof. In various examples, a composition comprises one or more of the compound(s). In various examples, a cell comprises one or more of the compound(s).

In various examples, a protein comprises (or consists of) one or more first amino acid residue(s) comprising a side-chain reactive site, the first amino acid residue(s) comprising the following structure:

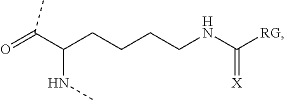

where RG is a reactive group independently at each occurrence comprising (or consisting of) the following structure:

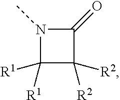

where X is O or S, R¹ and R² are independently at each occurrence chosen from hydrogen group, halide groups, alkyl groups, cycloalkyl groups, alkoxy groups, alkylamino groups, alkylthiol groups, and structural analogs thereof, and optionally, a R¹ and a R² form a hydrocarbon ring or a heterocyclic ring, or

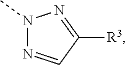

Where R³ is chosen from hydrogen group, alkyl groups, cycloalkyl groups, aromatic groups, heteroaromatic groups, and structural analogs thereof. In various examples, the RG independently at each occurrence comprises the following structure:

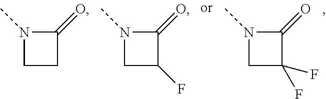

or a structural analog thereof. In various examples, the R³ group independently at each occurrence comprises:

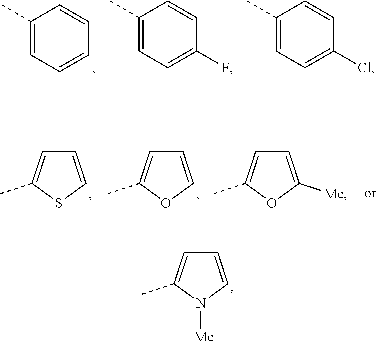

where Me is a methyl group, or a structural analog thereof. In various examples, the protein further comprising one or more second amino acid residue(s), comprising a nucleophilic side-chain reactive site, wherein one or more or all of the first amino acid residue(s) is/are each in proximity to a second amino acid residue, such that the side-chain reactive site of each of the one or more or all first amino acid residue(s) is capable of reacting with the side-chain reactive site of a second amino acid residue in proximity thereto to form one or more intramolecular crosslink(s) and/or one or more intermolecular crosslink(s). In various examples, the nucleophilic side-chain reactive site is a side-chain terminal group chosen from a hydroxyl group, a thiol group, a primary amine group, and imidazole groups. In various examples, the second amino acid residue(s) is/are independently at each occurrence chosen from lysine, tyrosine, histidine, cysteine, serine, and threonine. In various examples, the protein further comprises one or more cysteine disulfide bond(s). In various examples, the protein is capable of forming the one or more intramolecular and/or one or more intermolecular crosslink(s) without interfering with one or more cysteine disulfide bond(s) and/or one or more other cysteine residue(s) which are not second amino acid residue(s). In various examples, the protein is a single protein capable of forming one or more inter-strand intramolecular crosslink(s) and/or one or more intra-strand intramolecular crosslink(s). In various examples, the protein is a complex of a plurality of single proteins, wherein each single protein of the plurality is capable of forming one or more intramolecular crosslink(s) and/or one or more intermolecular crosslink(s) with one or more other single protein(s) of the plurality of single proteins. In various examples, the protein is capable of forming the one or more intramolecular crosslink(s) and/or one or more intermolecular crosslink(s) under neutral or basic pH conditions (e.g., about pH 7.0 or higher). In various examples, the protein is supercharged. In various examples, the protein comprises an overall net surface charge of from about +1 to about +20. In various examples, the protein is an engineered protein. In various examples, the protein comprises (or is) an antibody or the like or a portion thereof. In various examples, the antibody comprises (or is) a monoclonal antibody, an antibody fragment, a single-chain variable fragment, a fusion protein, a monobody, a nanobody, an affibody, an aptamer, an affilin, an affimer, an affitin, an alphabody, an anticalin, an avimer, a knottin, an armadillo repeat protein, designed ankyrin repeat proteins (DARPins), fynomers, gastrobodies, clostridal antibody mimetic proteins (nanoCLAMPs), optimers, repebodies, recombinant fibronectins, a centyrin, obody, or the like, or a portion thereof. In various examples, the protein further comprises one or more therapeutic modalit(ies), one or more diagnostic modalit(ies), or the like, or any combination thereof. In various examples, the protein is formed by a DNA-based recombinant method, and wherein the first amino acid residue(s) is/are independently at each occurrence site-specifically incorporated into the protein via a wild-type or mutant pyrrolysyl-tRNA synthetase/tRNA^{Pyl} pair. In various examples, a protein comprises two or more or any combination of the aforementioned features.

In various examples, a crosslinked protein comprises (or consists of) one or more intramolecular crosslink(s) and/or one or more intermolecular crosslink(s), the intramolecular crosslink(s) and/or the intermolecular crosslink(s) independently at each occurrence comprising the following structure:

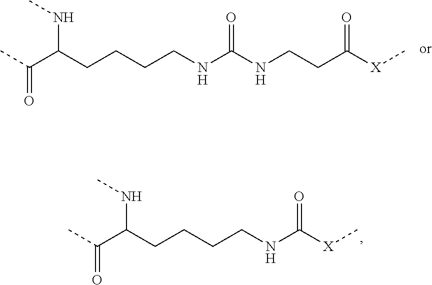

wherein X is independently at each occurrence an O atom, S atom, N atom, or NH group. In various examples, the crosslinked protein comprises intramolecular crosslink(s) and/or one or more intermolecular crosslink(s) formed by reaction of one or more first amino acid residue(s) comprising a side-chain reactive site, the first amino acid residue(s) comprising the following structure:

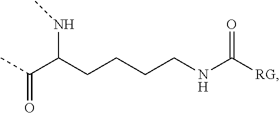

where RG is a reactive group independently at each occurrence comprising the following structure:

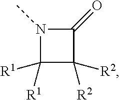

where R¹ and R² are independently at each occurrence chosen from hydrogen group, halide groups, alkyl groups, cycloalkyl groups, alkoxy groups, alkylamino groups, alkylthiol groups, and structural analogs thereof, and optionally, a R¹ and a R² form a hydrocarbon ring or a heterocyclic ring, or

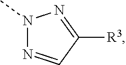

where R³ is chosen from hydrogen group, alkyl groups, cycloalkyl groups, aromatic groups, heteroaromatic groups, and structural analogs thereof, and one or more second amino acid residue(s) comprising a nucleophilic side-chain reactive site, wherein one or more or all of the first amino acid residue(s) is/are each in proximity to a second amino acid residue, such that the one or more intramolecular crosslink(s) and/or the one or more intermolecular crosslink(s) are formed by the reaction of the side-chain reactive site of each of the one or more or all first amino acid residue(s) with the side-chain reactive site of a second amino acid residue in proximity thereto. In various examples, a first protein comprises the first amino acid residue(s) and a second protein comprises the second amino acid residue(s). In various examples, the first protein and the second protein are comprised within a single protein and wherein the crosslink(s) is/are intramolecular crosslink(s). In various examples, the first protein and the second protein are comprised within separate proteins and wherein the crosslinks(s) is/are intermolecular crosslink(s). In various examples, the one or more intramolecular and/or one or more intermolecular crosslink(s) is/are formed under neutral pH conditions (e.g., about pH 7.0 or intracellular conditions) or the like. In various examples, the crosslinked protein is supercharged or the like. In various examples, the crosslinked protein comprises an overall net surface charge of from about +1 to about +20, including all integer values and ranges therebetween. In various examples, the crosslinked protein is a crosslinked engineered protein. In various examples, the crosslinked protein comprises (or is) a protein chosen from antibodies, monoclonal antibodies, antibody fragments, single-chain variable fragments, fusion proteins, monobodies, nanobodies, affibodies, aptamers, affilins, affimers, affitins, alphabodies, anticalins, avimers, knottins, armadillo repeat proteins, designed ankyrin repeat proteins (DARPins), fynomers, gastrobodies, clostridal antibody mimetic proteins (nanoCLAMPs), optimers, repebodies, recombinant fibronectins, centyrins, obodies, and the like, and any portion thereof. In various examples, the crosslinked protein further comprises one or more therapeutic modalit(ies), one or more diagnostic modalit(ies), or any combination thereof. In various examples, the crosslinked protein further comprises one or more biological activit(ies). In various examples, a crosslinked protein comprises two or more or any combination of the aforementioned features.

In various examples, a composition comprises one or more of the crosslinked protein(s). In various examples, the composition comprises one or more pharmaceutically acceptable excipient(s) or the like. In various examples, a cell comprises one or more of the crosslinked protein(s). In various examples, the second amino acid residue(s) are present in a protein disposed on a surface of the cell. In various examples, the cell is chosen from a bacterial cell, a fungal cell, a plant cell, an archaeal cell, an animal cell, and the like. In various examples, the animal cell is a human cell or the like.

In various examples, a method of forming the crosslinked protein comprises contacting a first protein with a second protein, where the first protein comprises one or more first amino acid residue(s) comprising a side-chain reactive site, the first amino acid residue(s) comprising the following structure:

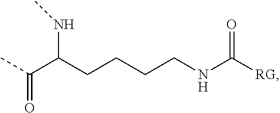

where RG is a reactive group independently at each occurrence comprising the following structure:

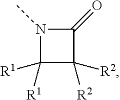

where R¹ and R² are independently at each occurrence chosen from hydrogen group, halide groups, alkyl groups, cycloalkyl groups, alkoxy groups, alkylamino groups, alkylthiol groups, and structural analogs thereof, and optionally, a R¹ and a R² form a hydrocarbon ring, a heterocyclic ring or the like, or

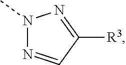

where R³ is chosen from hydrogen group, alkyl groups, cycloalkyl groups, aromatic groups, heteroaromatic groups, and structural analogs thereof, and where the second protein comprises one or more second amino acid residue(s) comprising a nucleophilic side-chain reactive site, wherein one or more or all of the first amino acid residue(s) is/are each in proximity to a second amino acid residue, such that the side-chain reactive site of each of the one or more or all first amino acid residue(s) is capable of reacting with the side-chain reactive site of a second amino acid residue in proximity thereto to form one or more intramolecular crosslink(s) and/or one or more intermolecular crosslink(s), thereby forming the crosslinked protein. In various examples, the first protein and the second protein are comprised within a single protein and the crosslink(s) is/are intramolecular crosslink(s). In various examples, first protein and the second protein are comprised within separate proteins and the crosslinks(s) is/are intermolecular crosslink(s). In various examples, the contacting is performed inside a cell or at the surface of a cell, or the like. In various examples, the contacting is performed in solution. In various examples, the contacting is performed in vitro or in vivo. In various examples, the one or more intramolecular and/or one or more intermolecular crosslink(s) is/are formed under neutral pH conditions or intracellular conditions.

In various examples, a method of covalent binding a protein to a target on a cell comprises contacting the cell with one or more of the protein(s), where the protein(s) is/are independently capable of specifically binding to the target on the surface of the cell, whereby the protein forms one or more intermolecular crosslink(s) with the target. In various examples, the intermolecular crosslink(s) is/are formed through a beta-lactam ring opening reaction or an acyl transfer reaction. In various examples, intermolecular crosslink(s) is/are formed through a proximity-enabled beta-lactam ring opening or acyl transfer reaction. In various examples, the intermolecular crosslink(s) independently comprise the following structure:

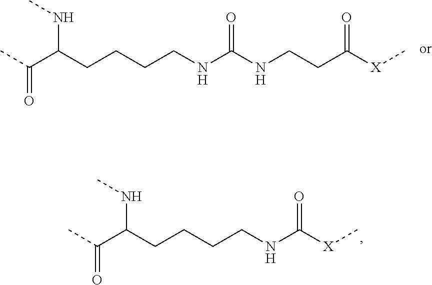

wherein X is independently at each occurrence an O atom, S atom, N atom, or NH group. In various examples, the protein(s) comprise or is/are antibod(ies), antibody fragment(s), single-chain variable fragment(s), fusion protein(s), monobodies (which may also be referred to as Adnectins), nanobod(ies), affibody(ies), aptamer(s), affilin(s), affimer(s), affitin(s), alphabod(ies), anticalin(s), avimer(s), knottin(s), armadillo repeat protein(s), designed ankyrin repeat protein(s) (DARPin(s)), fynomer(s), gastrobod(ies), clostridal antibody mimetic protein(s) (nanoCLAMP(s)), optimer(s), repebod(ies), recombinant fibronectin(s), centyrin(s), obod(ies), or the like. In various examples, the target is an intracellular protein or the like. In various examples, the protein(s) is/are capable of binding to a target on a surface of a cell or the like. In various examples, the target on the surface of the cell is a receptor or the like. In various examples, the receptor is a membrane receptor, a hormone receptor, or the like. In various examples, the target is a receptor chosen from an acetylcholine receptor, an adenosine receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor (EGFR), a formyl peptide receptor, a free fatty acid receptor, a galanin receptor, a ghrelin receptor, a glycoprotein hormone receptor, a gonadotrophin-releasing hormone receptor, a G protein-coupled estrogen receptor, a histamine receptor, a hydroxycarboxylic acid receptor, human epidermal growth factor receptor 2 (HER2), a kisspeptin receptor, a leukotriene receptor, a lysophospholipid receptor, a lysophospholipid SiP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, a vasopressin receptor, or the like. In various examples, a method of cellular delivery comprises contacting one or more of the crosslinked of the protein(s) with a cell or a population of cells, where the crosslinked protein(s) are delivered into the cell or the population of cells. In various examples, the crosslinked protein is or comprises a therapeutic compound for a present condition, disease, or disease state, or any combination thereof, and wherein the contacting step occurs in an individual in need of treatment for the present condition, disease, or disease state, or any combination thereof, and/or the crosslinked protein comprises or is a prophylactic compound for a potential condition, disease, disease state, or any combination thereof, and wherein the contacting step occurs in an individual in need of prophylaxis for the potential condition, disease, disease state, or any combination thereof; and/or the crosslinked protein is or comprises a diagnostic compound for a present or potential condition, disease, disease state, or any combination thereof, and wherein the contacting step occurs in an individual in need of diagnosis for the present or potential condition, disease, disease state, or any combination thereof. In various examples, the condition, disease, or disease state is chosen from a cancer, an auto-immune disease, a metabolic disease, an infectious disease, or the like, or any combination thereof, and where the individual has or is at risk of developing the condition, disease, disease state, or any combination thereof.

In various examples, an engineered pyrrolysyl-tRNA synthetase comprising one or more amino acid mutation(s) within a substrate-binding site as compared to a wild-type pyrrolysyl-tRNA synthetase, wherein the substrate-binding site comprises amino acid 306, amino acid 309, amino acid 348 of SEQ ID NO: 24 or in corresponding positions thereto in a variant thereof. In various examples, the one or more amino acid mutation(s) comprise a Y306V, a L309A, a C348F, a Y384F, or any combination thereof. In various examples, the engineered pyrrolysyl-tRNA synthetase comprises 80% up to, but excluding, 100% homology with the wild-type pyrrolysyl-tRNA synthetase (SEQ ID NO: 24). In various examples, the engineered pyrrolysyl-tRNA synthetase comprises a polypeptide comprising (or consisting of) a sequence according to SEQ ID NO: 1. In various examples, a polynucleotide comprises encoding the engineered pyrrolysyl-tRNA synthetase. In various examples, a vector comprises the polynucleotide, where the polynucleotide is optionally operatively coupled to one or more regulatory element(s) or the like. In various examples, a cell comprises the engineered pyrrolysyl-tRNA synthetase, the polynucleotide, the vector, or any combination thereof. In various examples, the cell is a bacterial cell, a fungal cell, a plant cell, an archaeal cell, an animal cell, or the like. In various examples, the polynucleotide is integrated into the genome of the cell. In various examples, a complex comprises the engineered pyrrolysyl-tRNA synthetase and the compound. In various examples, a cytoplasmic extract obtained from the cell.

In various examples, a method of producing the protein comprises contacting a nucleic acid with the engineered pyrrolysyl-tRNA synthetase a tRNA^{Pyl}, and a compound, where the nucleic acid encodes a protein, and the nucleic acid comprises at least one codon recognized by a tRNA^{Pyl}, thereby producing the protein. In various examples, the contacting is in vitro or in vivo. In various examples, the contacting is in a cell or the like. In various examples, the cell is a bacterial cell, a fungal cell, a plant cell, an archaeal cell, an animal cell or the like.

### BRIEF DESCRIPTION OF THE FIGURES

For a fuller understanding of the nature and objects of the disclosure, reference should be made to the following detailed description taken in conjunction with the accompanying figures.

FIG. 1A-1B shows orthogonal protein crosslinking via a proximity-driven acyl transfer reaction. a) Reaction scheme showing orthogonal crosslinking mediated by a genetically encoded amino acid. LG=leaving group. b) Structures of noncanonical electrophilic amino acids of the present disclosure.

FIG. 2A-2C—shows identification of CATKRS and validation of its activity. (a) Crystal structure of MmPylRS in complex with Pyl-AMP (PDB code: 2ZIM) with five contact residues shown in green tube model and Pyl-AMP shown in yellow tube model. (b) Fluorescence-based detection of CATK incorporation into sfGFP in BL21(DE3) cells expressing CATKRS. (c) Deconvoluted intact mass of the sfGFP-204CATK-1 mutant analyzed by QTOF-LC/MS.

FIG. 3A-3B shows assessment of the CATK crosslinking reactivity in SjGST dimers. (a) Scheme for intermolecular covalent crosslinking of the GST-CATK dimer. The crosslinking bonds were marked as red lines between the two monomers. The glutathione S-transferase structure (PDB code: 1Y6E) was rendered using PyMOL. The four free cysteines in one monomer were shown in a CPK model. (b) Coomassie blue-stained SDS-PAGE gel of the CATK and FPheK-encoded GST proteins showing the covalent GST dimer formation.

FIG. 4A-4C shows assessment of CATK-mediated intermolecular crosslinking specificity. (a) A close-up view of residues from the opposing GST monomer (colored in gray) surrounding CATK-1. PDB code: 1Y6E. (b) SDS-PAGE analysis of CATK-1-encoded GST mutants lacking certain adjacent nucleophilic residues. (c) Examining crosslinking specificity of GST-E52CATK-1 mutants containing potential nucleophilic residues at position-92 by western blot. The covalent GST dimer was probed using anti-His6 antibody. The crosslinking yields were listed underneath each lane.

FIG. 5A-5C shows inter-strand crosslinking of nanobody NB1 and monobody NSa1 mediated by CATK-1. (a) Nanobody NB1 structure (PDB: 3ogo, left) and wild-type NSa1 structure (PDB: 4je4, right) showing the crosslinking sites. Cys-24 and Cys-98 were rendered in blue CPK model. (b) Coomassie blue stained SDS-PAGE gels of NB1-V4BocK and NB1-V4CATK-1 (left), and NSa1, NSa1(+10)-A13BocK and NSa1(+10)-A13CATK-1 (right). Asterisk indicates the impurity derived from Ni-NTA affinity purification. (c) Deconvoluted mass spectra of NB1-V4CATK-1 (left) and NSa1(+10)-A13CATK-1 (right). The non-crosslinked starting materials [M−Met+H⁺] (calcd 12990.43 Da) and potential GSH adduct (calcd 13152.60 Da) were not observed for NSa1(+10)-A13CATK-1.

FIG. 6A-6D shows assessment of effect of CATK-1-mediated inter-strand crosslinking on monobody cellular uptake and endosomal stability. (a) SDS-PAGE analysis of the AF488-labeled NSa1(+10) monobodies encoding either CATK-1 or BocK. In-gel fluorescence image was shown on the top and silver staining image was shown at the bottom. The design of the NSa1 expression construct was shown on the right. (b) Scatter plots of HeLa cells without or with NSa1(+10) treatment. A total of 10,000 events were recorded in each measurement. (c) Plot of mean fluorescence intensity of HeLa cells after treatment with the NSa1(+10) mutants. The error bars represent the standard deviations from three independent measurements. (d) Stability of the supercharged NSa1 mutants against cathepsin B. The total ion counts of the intact proteins were used in quantification. Data at each time point represent mean±SEM of three independent experiments. The data were fitted to one-phase decay equation using GraphPad Prism 9.2.

FIG. 7 shows an example of site-specific incorporation of an electrophilic CATK amino acid into a protein, method of crosslinking through proximity-driven acyl transfer reaction, and structure of an orthogonal crosslinked protein.

FIG. 8 shows a crystal structure of a protected thiophenyl-triazole-lysine (S3-4a). Thermal ellipsoids are drawn at 50% probability level. Hydrogen atoms are omitted for clarity with the exception of H4 and H5.

FIG. 9 shows fluorescence-based assessment of CATK incorporation into sfGFP-Q204TAG by CATKRS. The bacterial lysates overexpressing sfGFP-Q204CATK proteins were used directly in the fluorescence measurement.

FIG. 10A-10B shows purification and characterization of sfGFP-Q204CATK mutants. (a) Scheme depicting site-specific incorporation of CATK into sfGFP via genetic code expansion. (b) Coomassie blue stained SDS-PAGE gel of sfGFP-Q204CATK mutants. The expression yields are shown at the bottom.

FIG. 11A-11C shows QTOF-LC/MS spectra of recombinant sfGFP mutants encoding (a) CATK-1, (b) CATK-2, and (c) CATK-7. The charge ladders are shown on the first panel, whereas the corresponding deconvoluted intact masses are shown on the second panel.

FIG. 12A-12B shows QTOF-ESI/MS spectrum of GST-E52BocK-E92K showing (a) charge ladder; and (b) deconvoluted intact mass. Calcd for [M−Met+H⁺] 26,588.67 Da, found 26,587.94 Da; calcd for [M−Met+GSH−2H+H⁺] 26,893.98 Da, found 26,893.26 Da; The small mass peaks 26,619.63 Da and 26,924.90 Da correspond to [M+H⁺] 26,619.71 Da and [M+GSH−2H+H⁺]26,925.02 Da of GST-E52Q/E92K, respectively, a product of near-cognate suppression. The expression yield of GST-E52BocK-E92K was calculated to be 35 mg L⁻¹.

FIG. 13A-13B shows characterization of CATK-1-encoded GST proteins purified using Ni-NTA resin or glutathione-agarose beads (a). Protein yield=7.5 mg L⁻¹ for Ni-NTA resin and 2.9 mg L⁻¹ for glutathione-agarose beads. (b) After protein expression, cells were lysed with lysis buffer with pH 8.0 or 7.4, and directly probed with anti-His antibody to detect GST dimer formation. The Coomassie Blue (CBB) stained image of the same samples are shown on the right.

FIG. 14A-14B shows intact masses of GST-E52CATK-1-E92K dimers. (a) Cartoon showing possible GST dimer structures. The possible dimer species, M1 and M4, are shown in boxes. (b) Deconvoluted masses and the zoom-in spectrum show mass assignment. The crosslinked heterodimer M4 is formed between GST-E52CATK-1-E92K and GST-E52W-E92K (a product of near-cognate suppression with Trp).

FIG. 15A-15B shows characterization of FPheK-encoded SjGST mutants. (a) SDS-PAGE (left) and western blot (right) analyses of GST mutants after purification from the cell lysates in DPBS, pH 7.4. (b) SDS-PAGE (first panel) and western blot (second panel) analyses of GST mutants after buffer exchange into HEPES buffer (50 mM HEPES, pH 8.5) and an extended incubation at 37° C. for 12 h. The SDS-PAGE gels were stained with Coomassie blue, and the western blots were probed with anti-His₆ antibody. The crosslinking yields were determined using ImageJ. Two forms of GST dimers were detected.

FIG. 16A-16C shows expression and characterization of sfGFP-Q204FSY. (a) Fluorescence of the lysates of Acella cells transformed with the pET-sfGFP-Q204TAG and pEVOL-FSYRS plasmids and grown in the absence and presence of 1 mM FSY. (b) SDS-PAGE gel and western blot of the purified sfGFP-Q204FSY. (c) Charge ladder and deconvoluted mass of the purified sfGFP-Q204FSY: [M−Met+H⁺] calcd 27,827.85 Da, found 27,826.77 Da; [M+H⁺] calcd 27,959.11 Da, found 27,959.14 Da; [M−Met−F⁻] calcd 27,807.91 Da, found 27,806.66 Da; [M−F⁻] calcd 27,939.11 Da, found 27,939.85 Da. The smaller mass peak at 27,710.29 Da corresponds to sfGFP-Q204 (calcd 27,710.82 Da), a product of near-cognate suppression.

FIG. 17A-17B expression and characterization of FSY-encoded SjGST mutants: (a) SDS-PAGE and (b) western blot of three GST mutants after Ni-NTA affinity purification. The crosslinking yields were determined using ImageJ.

FIG. 18 shows characterization of NB1 encoding BocK and CATK-1 by mass spectrometry.

FIG. 19A-19D shows characterization of NSa1 mutants by mass spectrometry. Charge ladder and deconvoluted mass of (a) wild-type NSa1; (b) NSa1(+10)-A13BocK; and (c) NSa1(+10)-A13CATK-1. (d) Expression and MS analysis of NSa1(+10)-A13CATK-1/Y92F. The crosslinking efficiency dropped from 100% to 9.5% based on ions counts. The minor peaks of 13136.02 Da and 13177.57 Da can be assigned to the GSH adducts owing to the high reactivity of CATK-1 in the absence of a proximal nucleophile.

FIG. 20A-20B shows expression and characterization of NSa1(+10)-A13FSY. (a) Coomassie blue staining. (b) mass spectrometry analysis. The red circled peaks 12935.54 and 12916.14 were assigned to intact NSa1(+10)-A13FSY (non-cross-linked starting materials, calcd 12936.33 Da) and intramolecular cross-linked NSa1 (calcd 12916.33 Da), respectively. Other peaks were impurities from Ni-NTA resin purification. The intramolecular cross-linking yield between FSY and Tyr92 was determined to be 27.5% based on the ion counts.

FIG. 21 (SEQ ID NO: 93-94) shows LC-MS analysis of NSa1(+10)-A13CATK-1 protein sample after trypsin digestion. The protein in elution buffer was directly digested with TPCK-treated immobilized trypsin at 4° C. overnight before mass spectrometry analysis. The LC-MS data were searched against NSa1 sequences using Agilent BioConfirm 10.0 software. Protein identification indicated the sequence of NSa1 with 40.9% coverage. The MS for the possible crosslink fragment between Y92 and the CATK-1 at site 13 in NSa1 protein was searched and ion extracted from the same mass chromatography data using Agilent Qualitative Analysis 10.0.

FIG. 22A-22B shows mass spectrometry characterization of the NSa1(+10) mutant proteins encoding either (a) CATK-1 or (b) BocK after labeling with AF488-NHS.

FIG. 23 shows cell viability assay results. HEK293T cells were incubated with various concentrations of CATK-1 and CATK-2 overnight. Error bars represent s.e.m; n=3.

FIG. 24A-24C shows site-specific incorporation of CATK-1 into mCherry-TAG-EGFP in HEK293T cells. (a) Construct design of the mCherry-TAG-EGFP-HA reporter. (b) Bright field and fluorescence micrographs of HEK293T cells transfected with the plasmids encoding mCherry-TAG-EGFP and CATKRS-tRNAPylcuA and cultured in DMEM supplemented with 10% FBS in the absence or presence of 0.5 mM CATK-1. (c) Western blot analysis of the HEK293T cell lysates probed with anti-HA tag antibody.

FIG. 25A-25B shows (a) scheme for BeLaK-mediated orthogonal crosslinking in protein structure. The structures of BeLaK and BocK (used as a negative cotrol) were shown at the bottom. (b) Site-specific incorporation of BeLaK into sfGFP-204TAG analyzed by fluorescence measurement.

FIG. 26A-26C shows recombinant expression of an orthogonally crosslinked monobody 12VC1 via site-specific incorporation of BeLaK. (a) A structural model of the orthogonally crosslinked monobody 12VC1 based on PDB code: 7L0G. The two β-strands are covalently linked through the orthogonal crosslinker colored in orange. The crosslinking pair BeLaK13-K93 was depicted in stick models. (b) SDS-PAGE gel showing successful expression of 12VC1 mutants encoding either BocK or BeLaK. UAA=unnatural amino acid. (c) Deconvoluted mass of 12VC1-BeLaK13-K93 after incubating the monobody with 2 mM β-mercaptoethanol at 37° C. for 24 hours. The recombinant 12VC1 contains the His-tag and TEV cleavage site at its N-terminus: MGSSHHHHHHHSSGTENLYFQ/G, (SEQ ID NO: 92) which adds a mass of 2387.49 Da to the monobody. The TEV sequence can be removed quantitatively through treatment with TEV protease.

FIG. 27A-27B shows purification and characterization of sfGFP-Q204BeLaK. a) Scheme showing site-specific incorporation of BeLaK into sfGFP via genetic code expansion. b) Coomassie blue stained SDS-PAGE gel (4-12%) of sfGFP encoding BeLaK. Expression yield=28.8 mg/L.

FIG. 28 shows QTOF-LC/MS spectra of recombinantly expressed sfGFP-Q204BeLaK proteins. The charge ladder is shown on the top, whereas the corresponding deconvoluted intact mass spectra is shown on the bottom.

FIG. 29A-29G shows QTOF-LC/MS spectra of recombinantly expressed GST-E52BeLaK-E92 mutants. The charge ladders are shown on the left, whereas the corresponding deconvoluted intact mass spectra are shown on the right. (a) Lysine mutant, (b) Tyrosine mutant, (c) Cysteine mutant, (d) Serine mutant, (e) Histidine mutant, (f) Threonine mutant, and (g) Aspartic acid mutant. * Denotes unassigned peaks

FIG. 30 shows SDS-PAGE analysis of the purified monobodies using 16% Tris-Tricine gels and Coomassie Blue staining.

FIG. 31 shows genetic supercharging of an orthogonally crosslinked NSa1monobody (PDB code: 4JE4) using a genetically encoded electrophilic amino acid BeLaK. The binding regions are colored in orange on ribbon models. The positive-charged residues are rendered in blue tube model. The crosslink is rendered in purple tube model with its chemical structure shown on the right.

FIG. 32A-32C shows design of β-lactam amino acids and their site-specific incorporation into sfGFP. (a) Structures of three β-lactam amino acids synthesized and tested, along with crystal structure of BeLaK protected with the p-nitrobenzyloxycarbonyl group (omitted for clarity). (b) Site-specific incorporation of BeLaK into sfGFP as assessed by fluorescence of the cell lysates. (c) Coomassie-blue stained SDS-PAGE gel of BeLaK-encoded sfGFP. (d) Deconvoluted intact mass of sfGFP-Q204BeLaK.

FIG. 33A-33B shows the assessment of inter-molecular crosslinking reactivity of BeLaK in GST dimers. (a) Selection of appropriate crosslinking sites at the GST dimer interface (PDB code: 1Y6E). A close-up view is shown on the right. (b) Determination of the crosslinking yields by western blot using anti-His₆ antibody.

FIG. 34A-34D (SEQ ID NO: 93, 95) shows BeLaK-mediated orthogonal crosslinking of NSa1 monobodies. (a) A model of +11 charged NSa1 monobody in complex with N—SH2 domain of SHP2 (PDB: 4JE4) showing genetic supercharging in blue tube model and BeLaK in magenta tube model. (b) Coomassie blue stained SDS-PAGE gel of NSa1 mutants encoding either BocK or BeLaK. (c) The interpolated charge surface (top) and the deconvoluted intact mass (bottom) of the supercharged NSa1 mutants. The calculated masses are for [M−Met+H⁺]. (d) Mass of a crosslinked fragment in NSa1(+11)-BeLaK.

FIG. 35A-35B shows (a) measurement of thermostability of supercharged NSa1 mutants encoding either BocK or BeLaK, and (b) comparison of thermostability of supercharged NSa1 mutants at 75° C.

FIG. 36A-36D shows examination of cellular uptake of supercharged monobody mutants. (a) Flow cytometry of HeLa cells treated with AF488-modified supercharged monobodies. (b) Histogram of mean fluorescence intensity. The error bars represent the standard deviations from three independent measurements. (c) Confocal microscopy of HeLa cells after 18-hour incubation with AF488-modified+11 charged monobodies encoding either BocK or BeLaK. Scale bar=5 μm. (d) Line profiles showing intracellular distribution of +11-charged monobodies with the red lines marked on the overlay images in c.

FIG. 37A-37M shows fluorescence-based assessment of BeLaF-1/2 incorporation into sfGFP-Q204TAG by MmPylRS variants: (a) AcrKRS, (b) CATKRS, (c) CpKRS, (d) FPheKRS, (e) FSYRS, (f) mPyTKRS, (g) PhTKRS, (h) WT, (i) TCOKRS, (j) PylRS-N346A-C348A, (k) PylRS-N346V-C348L, (1) PylRS-N346V-C348A, or (m) PylRS-N346V-C348L. The bacterial cell lysates were used directly in fluorescence measurement.

FIG. 38 shows crystal structure of a para-nitrobenzyloxycarbonyl protected 3-lactam-lysine. Thermal ellipsoids are drawn at 50% probability level.

FIG. 39 shows characterization of sfGFP-Q204BeLaK by QTOF-LC/MS: deconvoluted intact mass.

FIG. 40A-40C shows characterization of BeLaK-encoded GST mutant proteins. (a) Coomassie blue stained SDS-PAGE analysis of GST mutants encoding BeLaK. (b) Western blot analysis of GST mutants encoding BeLaK. (c) Characterization table of GST mutants encoding BeLaK. a The expression yield was determined using Pierce™ BCA protein assay kit (Thermo Fisher Scientific). ^{b}The extent of dimer formation was calculated by comparing the GST-dimer band intensity to the monomer band intensity on western blot.

FIG. 41A-41B shows characterization of NSa1 mutants. (a) Coomassie blue stained SDS-PAGE gel of NSa1 mutants encoding either BeLaK or BocK. (b) Summary of expression and MS characterization of NSa1 mutants encoding either BeLaK or BocK.

FIG. 42A-42B—(SEQ ID NO: 93, 95-96) shows QTOF-LC/MS analysis of NSa1-A13BeLaK fragments following trypsin digestion. The purified proteins in Ni-NTA elution buffer were digested with TPCK-treated immobilized trypsin at 37° C. for 6 hours before analysis. The data were searched against Nsa1 sequences using Agilent BioConfirm 10.0 software, which revealed sequence coverage of 33% and 63% for (a) Nsa1(+11) and (b) Nsa1(+18), respectively. The MS for all possible crosslinked fragments between the surrounding lysines and BeLaK at position-13 were searched and ion-extracted using Agilent Qualitative Analysis 10.0 software.

FIG. 43A-43B shows characterization of Nsa1-C1 mutants. (a) Coomassie blue stained SDS-PAGE gel of Nsa1-C1 mutants encoding either BocK or BeLaK. (b) Characterization table for expression and MS analysis of Nsa1-C1 mutants encoding either BocK or BeLaK.

FIG. 44 shows cytotoxicity assay of Nsa1 mutants encoding either BocK or BeLaK toward HeLa cells. Ca ionophore=calcium ionophore. Nsa1 protein variants were serially diluted two-fold from a stock solution in Dulbecco's modified eagle medium (DMEM, Life Technologies) supplemented with 10% (v/v) fetal bovine serum (FBS, Life Technologies) in 12.5 microliter (μL) volumes into a 384-plate (Corning). HeLa cells were added at 10,000 cells/well in a 12.5 μL volume. The plate was briefly mixed manually and then incubated for 18 hours at 37° C. in 5% CO₂. The CytoTox-Glo™ Cytotoxicity Assay Reagent (Promega) was prepared, and then 12.5 μL was added to each well. After another brief mix, the 384-plate was incubated at room temperature for 15 minutes and the luminescence signal was measured using a Synergy H1 microplate reader (BioTek).

FIG. 45A-45B shows site-specific incorporation of BeLaK into mCherry-TAG-EGFP in HEK293T cells. (a) Structure of mCherry-TAG-EGFP-HA reporter. (b) Bright field and fluorescence micrographs of HEK293T cells transfected with the plasmids encoding mCherry-TAG-EGFP and wtPylRS-tRNAPyl CUA and cultured in DMEM supplemented with 10% FBS in the absence or presence of 0.25 mM BeLaK.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also, unless otherwise stated, include individual values (e.g., about 1%, about 2%, about 3%, about 4%, etc.) and the sub-ranges (e.g., about 0.5% to about 1.1%, about 0.5% to about 2.4%, about 0.5% to about 3.2%, about 0.5% to about 4.4%, and other possible sub-ranges) within the indicated range. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise stated, “about” or “the like”, when used in connection with a measurable variable (such as, for example, a parameter, an amount, a temporal duration, or the like) or a list of alternatives, is meant to encompass variations of and from the specified value including those within experimental error (which can be determined by e.g. given data set, art accepted standard, and/or with e.g. a given confidence interval (e.g., 90%, 95%, or more confidence interval from the mean), such as variations of +/−10% or less, +/−5% or less, +/−1% or less, and +/−0.1% or less of and from the specified value, insofar such variations and variations in the alternatives are appropriate to perform in the instant disclosure. As used herein, unless otherwise stated, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

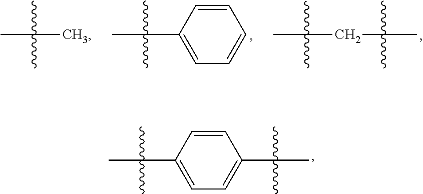

and the like.

As used herein, unless otherwise stated, the term “alkyl group” refers to branched or unbranched saturated hydrocarbon groups. Examples of alkyl groups include, but are not limited to, methyl groups, ethyl groups, propyl groups, butyl groups, isopropyl groups, tert-butyl groups, and the like. In various examples, an alkyl group is C₁ to C₂₀, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). An alkyl group may be unsubstituted or substituted with one or more substituent(s). Examples of substituents include, but are not limited to, halide groups (—F, —Cl, —Br, and —I), aryl groups, halogenated aryl groups, alkoxide groups, amine groups, nitro groups, carboxylate groups, carboxylic acids, ether groups, silyl ether groups, alcohol groups, alkyne groups (e.g., acetylenyl groups and the like), and the like, and any combination thereof.

As used herein, unless otherwise expressly stated, “cycloalkyl group” refers to a cyclic compound comprising a ring in which all of the atoms forming the ring are carbon atoms. The carbocyclic group is a saturated group. In various examples, a cycloalkyl group is a C₃ to C₆ cycloalkyl group, including all integer numbers of carbons and ranges of numbers of carbons therebetween (e.g., C₃, C₄, C₅, and C₆). A cycloalkyl group may be unsubstituted or substituted with one or more substituent(s). Examples of substituents include, but are not limited to, halide groups (—F, —Cl, —Br, and —I), aryl groups, halogenated aryl groups, alkoxide groups, amine groups, nitro groups, carboxylate groups, carboxylic acids, ether groups, silyl ether groups, alcohol groups, alkyne groups (e.g., acetylenyl groups and the like), and the like, and any combination thereof.

As used herein, unless otherwise stated, the term “aromatic group” refers to C₅ to C₃₀ aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). Aromatic groups include groups such as, for example, fused ring, biaryl groups, or a combination thereof. In various examples, an aromatic group is multicyclic (e.g., bicyclic, tricyclic, or the like). An aromatic group may be unsubstituted or substituted with one or more substituent(s). Examples of substituents include, but are not limited to, halide groups (—F, —Cl, —Br, and —I), alkyl groups, halogenated alkyl groups (e.g., trifluoromethyl group and the like), alkoxide groups, amine groups, nitro groups, carboxylate groups, carboxylic acids, ether groups, silyl ether groups, alcohol groups, a alkyne groups (e.g., acetylenyl groups and the like), and the like, and any combination thereof. Aromatic groups may include one or more heteroatom(s) in the ring(s) of an aryl group, such as, for example, oxygen (e.g., furanyl groups and the like), nitrogen (e.g., pyrrolyl groups and the like), sulfur (e.g., thiophenyl groups and the like), and the like. Such groups may be referred to as heteroaromatic groups. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like.

As used herein, unless otherwise stated, the term “alpha(a)-amino acid” or simply “amino acid” refers to a molecule containing both an amino group and a carboxyl group bound to a carbon which is designated as the α-carbon. Suitable amino acids include, but are not limited to, both the D- and L-isomers of the amino acids and amino acids prepared by organic synthesis or other metabolic routes. Unless the context specifically indicates otherwise, the term amino acid, as used herein, unless otherwise stated, is intended to include amino acid analogs. Non-limiting examples of suitable amino acids include, “naturally occurring amino acids” or “canonical amino acids”, which refers to any one of the twenty amino acids commonly found in proteins synthesized in nature (Alanine=Ala or A, Cysteine=Cys or C, Aspartic acid=Asp or D, Glutamic acid=Glu or E, Phenylalanine=Phe or F, Glycine=Gly or G, Histidine=His or H, Isoleucine=Ile or I, Lysine=Lys or K, Leucine=Leu or L, Methionine=Met or M, Asparagine=Asn or N, Proline=Pro or P, Glutamine=Gln or Q, Arginine=Arg or R, Serine=Ser or S, Threonine=Thr or T, Valine=Val or V, Tryptophan=Trp or W, and Tyrosine=Tyr or Y).

As used herein, unless otherwise stated, “non-canonical amino acid,” “synthetic amino acid,” “amino acid analog,” “amino acid derivative”, “non-standard amino acid,” “non-natural amino acid,” “unnatural amino acid,” and the like may all be used interchangeably, and is meant to include all amino acid-like compounds that are similar in structure and/or overall shape to one or more of the twenty L-amino acids commonly found in naturally occurring proteins. Amino acid analogs can also be natural amino acids with modified side chains or backbones.

As used herein, unless otherwise stated, “protein engineering” refers to the modification of the structural, catalytic and/or binding properties of natural proteins and the de novo design of artificial proteins. Protein engineering relies on an efficient recognition mechanism for incorporating mutant amino acids in the desired protein sequences. Though this process has been very useful for designing new macromolecules with precise control of composition and architecture, a major limitation is that the mutagenesis is restricted to the 20 naturally occurring amino acids. However, the incorporation of non-canonical amino acids (ncAAs) can extend the scope and impact of protein engineering methods.

As used herein, unless otherwise stated, the term “protein” or “polypeptide”, refers to one polypeptide chain or collectively two or more polypeptide chains, where the individual polypeptide chains each has greater than 50 amino acid residues, which can be obtained, for example, from either chemical synthesis or DNA-based recombinant methods.

As used herein, unless otherwise stated, the term “amino acid residue” refers to an amino acid that is part of a protein. The residues are amino acids connected to other amino acid residues through a peptide bond or bonds to form proteins (also referred to herein as polypeptides). Unless the context specifically indicates otherwise, the term amino acid is intended to include amino acid resides.

As used herein, unless otherwise stated, the term “crosslink” as used herein, unless otherwise stated, refers to the intramolecular or intermolecular connection of two amino acid residues.

As used herein, unless otherwise stated, the term “enzymatic stability” as used herein, unless otherwise stated, refers to the ability of the proteins to stay intact in the presence of an enzyme comprising proteolytic activity such as, for example, pepsin, trypsin, chymotrypsin, endosomal cathepsin, or the like, or any combination thereof in biological buffers or a mixture of proteolytic enzymes present in simulated or native gastric fluid or simulated intestine fluid or human serum. In various examples, the proteolytic stability of a crosslinked protein is measured by liquid chromatography-mass spectrometry (LC-MS), or the like.

As used herein, unless otherwise stated, the term “structural analog” refers to any group that can be envisioned to arise from an original group, compound, protein, or crosslinked protein if one atom or group of atoms, functional group(s), substructure(s), or the like thereof is replaced with another atom or group of atoms, functional group(s), substructure(s), or the like. In various examples, the term “structural analog” refers to any group that is derived from an original group, compound, original group, compound, protein, or crosslinked protein by a chemical reaction, where the any group, original group, compound, protein, or crosslinked protein is modified or partially substituted such that at least one structural feature of the original group, original group, compound, protein, or crosslinked protein is retained.

In an aspect, the present disclosure provides compounds. In various examples, a compound comprises a beta-lactam group, a triazole group (such as, for example, a 1,2,3-triazole group, or the like) or the like. In various examples, a compound is a lysine derivative or the like. In various examples, a compound is a non-natural amino acid. In various examples, a compound is made by a method of the present disclosure. In various examples, one or more compound(s) is/are used in a method of the present disclosure. Non-limiting examples of compounds are disclosed herein.

In various examples, a compound comprises one or more beta-lactam group(s), one or more triazole group(s) (such as, for example, 1,2,3-triazole group or the like), or the like, or any combination thereof. In various examples, beta-lactam group(s), triazole group(s), or the like, or any combination thereof is are, independently, a group (e.g., a terminal group) of a side-chain of an amino acid (such as, for example, an alpha-amino acid or the like). In various examples, the beta-lactam group, the triazole group (e.g., the 1,2,3-triazole group or the like) is covalently linked to the amino-acid side chain via a linking group. Non-limiting examples of linking groups include an amide group, a thioamide group, or the like. In various examples, a compound comprises (or consists of) the following structure:

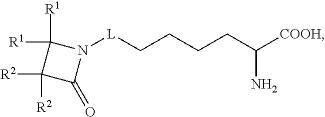

or a structural analog thereof, or a pharmaceutically acceptable salt, a salt, a partial salt, a solvate, a polymorph thereof, or a stereoisomer or a mixture of stereoisomers, an isotopic variant, a tautomer thereof, where L is a linking group, R¹ and R² are independently at each occurrence chosen from hydrogen group (such as, for example, a deuterium group, a tritium group or the like), halide groups, alkyl groups (such as, for example, C₁, C₂, C₃, C₄, C₅, and C₆ alkyl groups (e.g., methyl group, ethyl group, propyl groups, butyl groups, and the like)), cycloalkyl groups (such as, for example, C₃, C₄, C₅, and C₆ cyclolkyl groups (e.g., cyclopropyl groups, cyclobutyl groups, and the like)), alkoxy groups (such as, for example, C₁, C₂, C₃, C₄ alkoxy groups (e.g., methoxy group, ethoxy group, and the like)), alkylamino groups (such as, for example, C₁, C₂, C₃, C₄, C₅, and C₆ alkylamino groups (e.g., methylamino group, ethylamino group, and the like)), alkylthiol groups (such as, for example, C₁, C₂, C₃, C₄, C₅, and C₆ alkylthio groups (e.g., methylthiol group, ethylthiol group, and the like)), and the like. In various examples, a R¹ and a R² taken together form a hydrocarbon ring, a heterocyclic ring, or the like. In various examples, a compound comprises (or consists of) the following structure:

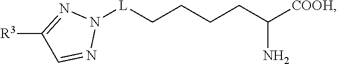

or a structural analog thereof, or a pharmaceutically acceptable salt, a salt, a partial salt, a solvate, a polymorph, a prodrug thereof, or a stereoisomer or a mixture of stereoisomers, an isotopic variant, a tautomer thereof, where L is a linking group and R³ is chosen from hydrogen group (such as, for example, a deuterium group, a tritium group or the like), halide groups, alkyl groups (such as, for example, methyl group, ethyl group, propyl groups, butyl groups, and the like), cycloalkyl groups (such as, for example, cyclopropyl groups and cyclobutyl groups, and the like), aromatic groups (such as, for example, phenyl groups and the like), heteroaromatic groups (such as, for example, pyrrolyl groups, furanyl groups, thiophenyl groups, and the like). In various examples, an R³ group comprises (or consists of) the following structure:

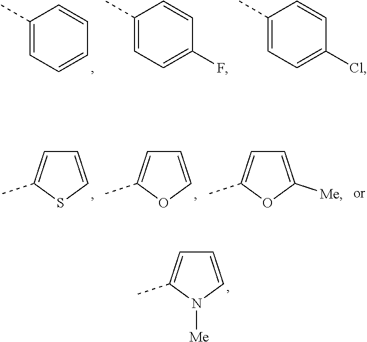

wherein Me is a methyl group, or a structural analog thereof. In various examples, a compound comprises (or consists of) the following structure:

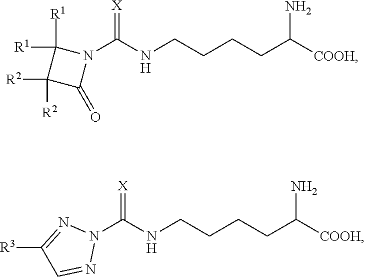

Where X is O, S, or the like, or the like.

In various examples, a R¹ and a R² taken together form a hydrocarbon ring group, a heterocyclic ring group, or the like. In various examples, a hydrocarbon ring group comprises a ring in which all of the atoms forming the ring are carbon atoms. In various examples, the hydrocarbon ring group is a saturated group. In various examples, a hydrocarbon group is a C₃ to C₆ (e.g., C₃, C₄, C₅, and C₆) cycloalkyl group. A hydrocarbon group may be unsubstituted or substituted with one or more substituent(s). Examples of substituents include, but are not limited to, halide groups (—F, —Cl, —Br, and —I), aryl groups, halogenated aryl groups, alkoxide groups, amine groups, nitro groups, carboxylate groups, carboxylic acids, ether groups, silyl ether groups, alcohol groups, alkyne groups (e.g., acetylenyl groups and the like), and the like, and any combination thereof. In various examples, a heterocyclic ring group comprises a ring comprising carbon atoms and one or more heteroatom(s) (such as, for example, oxygen, nitrogen, sulfur, and the like. In various examples, the heterocyclic ring group is a saturated group. In various examples, a heterocyclic ring group is a C₃ to C₆ (e.g., C₃, C₄, C₅, and C₆) cycloalkyl group. A hydrocarbon group may be unsubstituted or substituted with one or more substituent(s). Examples of substituents include, but are not limited to, halide groups (—F, —Cl, —Br, and —I), aryl groups, halogenated aryl groups, alkoxide groups, amine groups, nitro groups, carboxylate groups, carboxylic acids, ether groups, silyl ether groups, alcohol groups, alkyne groups (e.g., acetylenyl groups and the like), and the like, and any combination thereof.

In various examples, a compound is monofluorinated, difluorinated, or the like. In various examples, one or both R¹ groups are fluorinated. In various examples, a compound comprises the following structure:

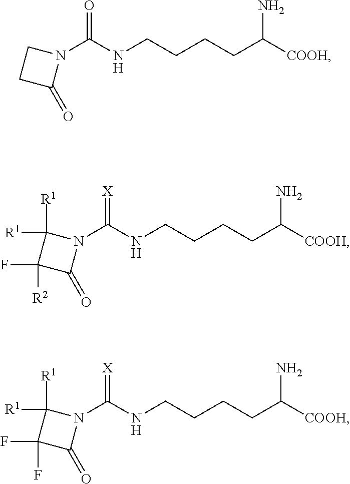

or the like, or a structural analog thereof, or a pharmaceutically acceptable salt, a salt, a partial salt, a solvate, a polymorph thereof, or a stereoisomer or a mixture of stereoisomers, an isotopic variant, a tautomer thereof. In various examples, the remaining R¹ and/or R² groups are hydrogen groups, where X is O, S, or the like.

In an aspect, the present disclosure provides compositions. In various examples, a composition comprises one or more compound(s) of the present disclosure. Non-limiting examples of compositions are disclosed herein.

In an aspect, the present disclosure provides proteins. In various examples, these proteins are not crosslinked. In various examples, a protein is an engineered protein. In various examples, a protein comprises (or consists of) a sequence of any crosslinked protein of the present disclosure, where the protein is not crosslinked. In various examples, a protein is made by a method of the present disclosure. Non-limiting examples of non-crosslinked proteins are disclosed herein.

In various examples, a protein (which may be a first polypeptide chain) comprises one or more first amino acid residue(s) and one or more second amino acid residue(s). In various examples, each of the first amino acid residue(s) (which may be one or more first lysine derivative residue(s), or the like, or any combination thereof) comprise(s) a reactive site (which may be a terminal group on the side chain of each first amino acid residue). A protein can comprise various first amino acid residue(s). In various examples, the first reactive site of a first amino acid is a leaving group. In various examples, a first amino acid residue(s) comprise(s) the following structure:

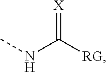

where RG is a reactive group and X is O, S, or the like. In various examples, a first amino acid residue(s) comprise(s) the following structure:

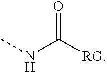

In various examples, a first amino acid residue(s) comprise(s) the following structure:

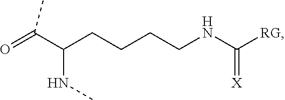

where X is O, S, or the like. In various examples, RG independently at each occurrence comprises (or consists of) the following structure:

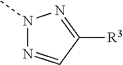

(which may be referred to as leaving group),

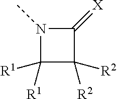

(such as, for example,

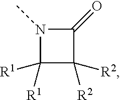

or the like, where X, R¹, R², and R³ are as described with respect to the compounds of the present disclosure. In various examples, RG independently at each occurrence comprises (or consists of) the following structure:

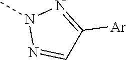

(which may be referred to as leaving group),

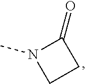

or the like, where Ar is an aromatic group, or a substituted analog. In various examples, Ar independently at each occurrence is or comprises a phenyl group, a substituted phenyl group, a thiophenyl group, a substituted thiophenyl group, a furanyl group, a substituted furanyl group, a pyrrolyl group (which may be a N-alkyl pyrrolyl group (e.g., a N-methyl pyrrolyl group or the like), or a substituted pyrrolyl group (which may be a substituted N-alkyl pyrrolyl group, (e.g., a substituted N-methyl pyrrolyl group or the like) (e.g., comprises (or consists of) the follow structure

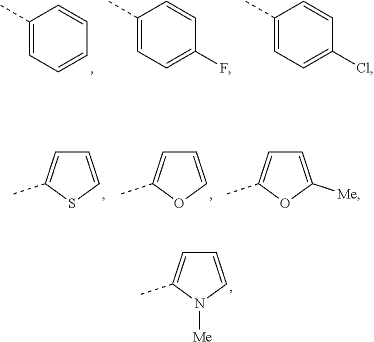

where Me is a methyl group, or a substituted analog thereof.

A protein can comprise various second amino acid residue(s). A second amino acid group may a nucleophilic amino acid residue (e.g., formed from a nucleophilic amino acid or the like). In various examples, a second amino acid residue(s) comprise(s) a nucleophilic reactive site (which may be a nucleophilic terminal group (e.g., a hydroxyl group, a thiol group, a primary amine group, a secondary amine group, or the like) on the side chain of each second amino acid residue). In various examples, a second amino acid residue is independently at each occurrence chosen from lysine, tyrosine, histidine, cysteine, serine, threonine, and the like. In various examples, the second amino acid residue is present in a second polypeptide chain of a protein. In various examples, the first amino acid residue and the second amino acid residue are present in the same polypeptide chain of a protein. In various examples, the first amino acid reside and the second amino acid residue are present in the different polypeptide chains of a protein (e.g., a homodimer where the polypeptide chains have the same structure or a heterodimer where the polypeptide chains have the different structure).

A protein can be capable of various modes of crosslinking. In various examples, a protein is capable of proximity-driven crosslinking. In various examples, proximity-driven crosslinking occurs spontaneously after formation of a protein. In various examples, one or more or all first amino acid residue(s) is/are each in proximity to a second amino acid residue, such that a reactive site of each first amino acid residue is capable of reacting (e.g., spontaneously reacting or the like) with a reactive site of a second amino acid residue in proximity thereto to form one or more intramolecular crosslink(s) and/or one or more intermolecular crosslink(s). In various examples, a protein is capable of forming one or more intramolecular crosslink(s) and/or one or more intermolecular crosslink(s) under neutral or basic pH conditions (e.g., about pH 7.0 or higher).

In various examples, a protein is capable of orthogonal crosslinking (e.g., where a first reactive group and a second reactive group specifically (e.g., exclusively) crosslinks with one another). In various examples, a protein is capable of forming one or more intramolecular and/or intermolecular crosslink(s) without interfering with (e.g., without reacting with) one or more cysteine disulfide bond(s) and/or one or more other cysteine residue(s) which are not second amino acid residue(s). In various examples, a protein further comprises one or more cysteine disulfide bond(s). In various examples, one or more cysteine disulfide bond(s) form prior to, simultaneously with, or after formation of one or more orthogonal crosslink(s) between first reactive group(s) (e.g., of a first amino acid residue or the like) and second reactive group(s) (e.g., of a second amino acid residue or the like).

A protein can be capable of forming various intramolecular and/or intermolecular crosslinks. In various examples, a protein is a single protein capable of forming one or more inter-strand intramolecular crosslink(s) and/or intra-strand intramolecular crosslink(s). In various examples, a protein is a complex of a plurality of single proteins (such as, for example, a dimer complex of two single proteins or the like), wherein each single protein of the plurality is capable of forming one or more inter-strand intramolecular crosslink(s) and/or one or more intra-strand intramolecular crosslink(s), and/or one or more intermolecular crosslink(s) with one or more other single protein(s) of the plurality of single proteins. In various examples, the plurality of single proteins are the same proteins (e.g., forming a homodimer or the like). In various examples, the plurality of single proteins comprises two different proteins (e.g., forming a heterodimer or the like).

A protein can have various number of and distribution of positively charged protein surface groups. In various examples, a protein is supercharged (e.g., comprises one or more surface exposed positively charged amino acid residues or the like), In various examples, a protein comprises an overall net surface charge of from about +1 to about +20, including all integer values and ranges therebetween (e.g., about +1, about +2, about +3, about +4, about +5, about +6, about +7, about +8, about +9, about +10, about +11, about +12, about +13, about +14, about +15, about +16, about +17, about +18, about +19, or about +20) (e.g., at least about +5 or greater, at least about +6 or greater, at least about +7 or greater, at least about +8 or greater, at least about +9 or greater, at least about +10 or greater, at least about +11 or greater, at least about +12 or greater, at least about +13 or greater, at least about +14 or greater, or at least about +15 or greater).

In various examples, a protein is an engineered protein. In various examples, an engineered protein comprises an engineered protein chosen from antibodies (such as, for example, monoclonal antibodies and the like), antibody fragments (such as, for example, antigen-binding antibody fragments and the like), single-chain variable fragments, fusion proteins, monobodies (which may also be referred to as Adnectins), nanobodies, affibodies, aptamers, affilins, affimers, affitins, alphabodies, anticalins, avimers, knottins, armadillo repeat proteins, designed ankyrin repeat proteins (DARPins), fynomers, gastrobodies, clostridal antibody mimetic proteins (nanoCLAMPs), optimers, repebodies, recombinant fibronectins (e.g., Pronectin™ and the like), centyrins, and obodies, and the like, and any portion thereof. In various examples, a protein further comprises one or more therapeutic compound(s), one or more diagnostic compound(s), or the like or any combination thereof. In various examples, a crosslinked protein further comprises one or more biological activit(ies) (e.g., anticancer activit(ies) or the like). In various examples, an engineered protein is an antibody mimic or the like. In various examples, an engineered protein a single-domain antibody (such as, for example, a nanobody, a synthetic antibody mimic (e.g., a monobody or the like) or the like.

In various examples, a protein (or a crosslinked protein thereof) comprises at least a portion of or all (or consists of) of a protein of described herein. In various examples, a protein is a 12VC1 mutant (or a crosslinked protein thereof) or the like. In various examples, a protein (or a crosslinked protein thereof) comprises at least a portion of or all (or consists of) of a protein comprising the following sequence:

12VC1-WT
[SEQ. ID. NO: 1]
MGSSHHHHHHSSGTENLYFQGVS SVPTKLEV VA*TPTSLLI
SWDAPAVTVF FYVITYGETG HGVGAFQAFK VPGSKSTATI
SGLKPGVDYT ITVYARGYSK QGPYKPSPIS INKRT
(* = incorporation site for a first amino acid
(e.g., BocK, BeLaK, or the like);
12VC1(+8)
[SEQ. ID. NO: 2]
MGSSHHHHHHSSGTENLYFQGVS SVPTKLKV VA*TPTSLLI
SWDAPAVTVF FYVITYGETG HGVGAFKAFK VPGSKSTATI
SGLKPGVDYT ITVYARGYSK KGPYKPSPIS INKRT
(* = incorporation site for a first amino acid
(e.g., BocK, BeLaK, or the like);
or
12VC1(+10)
[SEQ. ID. NO: 3]
MGSSHHHHHHSSGTENLYFQGVSKVPTKLEV VA*TPTSLLI
KWDAPAVTVK FYVITYGEKG HGVGAFQAFK VPGSKRTATI
KGLKPGVDYT ITVYARGYSK QGPYKPSPIS INKRT
(* = incorporation site for a first amino acid
(e.g., BocK, BeLaK, or the like)).

In various examples, a protein is a Nsa1 mutant or the like. In various examples, a protein comprises at least a portion of or all (or consists of) of a protein comprising the following sequence:

NSa1-Y92K-C1
[SEQ. ID. NO: 4]
MGSSHHHHHHSSGTENLYFQGC VSSVPTKLEV VAATPTSLLI
SWDAPAVTVD YYVITYGETG SGGYAWQEFE VPGSKSTATI SGLKPGVDYT
ITVYAGYYGY PTYYSSPISI NKRT;
NSa1-A13BocK-C1
[SEQ. ID. NO: 5]
MGSSHHHHHHSSGTENLYFQGC VSSVPTKLEV VA*TPTSLLI
SWDAPAVTVD YYVITYGETG SGGYAWQEFE VPGSKSTATI SGLKPGVDYT
ITVYAGYYGY PTYYSSPISI NYRT (* = BocK);
NSa1(+5)-A13BeLaK
[SEQ. ID. NO: 6]
MGSSHHHHHHSSGTENLYFQG VSSKPTKLRV VR*TPTSLKI
SWDAPAVTVD YYVITYGEKG SGGYAWQEFE VPGSKRTATI SGLKPGVDYT
ITVYAGYKGY PTYYSSPISI NYRT (* = BeLaK);
NSa1(+5)-A13BeLaK-Y92K
[SEQ. ID. NO: 7]
MGSSHHHHHHSSGTENLYFQG VSSKPTKLRV VR*TPTSLKI
SWDAPAVTVD YYVITYGEKG SGGYAWQEFE VPGSKRTATI SGLKPGVDYT
ITVYAGYKGY PTYYSSPISI NKRT (* = BeLaK);
NSa1(+5)-A13BocK-Y92K
[SEQ. ID. NO: 8]
MGSSHHHHHHSSGTENLYFQG VSSKPTKLRV VR*TPTSLKI
SWDAPAVTVD YYVITYGEKG SGGYAWQEFE VPGSKRTATI SGLKPGVDYT
ITVYAGYKGY PTYYSSPISI NKRT (* = BocK);
NSa1(+5)-A13BeLaK-Y92K-C1
[SEQ. ID. NO: 9]
MGSSHHHHHHSSGTENLYFQGC VSSKPTKLRV VR*TPTSLKI
SWDAPAVTVD YYVITYGEKG SGGYAWQEFE VPGSKRTATI SGLKPGVDYT
ITVYAGYKGY PTYYSSPISI NKRT (* = BeLaK);
NSa1(+5)-A13BocK-Y92K-C1
[SEQ. ID. NO: 10]
MGSSHHHHHHSSGTENLYFQGC VSSKPTKLRV VR*TPTSLKI
SWDAPAVTVD YYVITYGEKG SGGYAWQEFE VPGSKRTATI SGLKPGVDYT
ITVYAGYKGY PTYYSSPISI NKRT (* = BocK);
NSa1(+7)-A13BeLaK
[SEQ. ID. NO: 11]
MGSSHHHHHHSSGTENLYFQG VSSKPTKLRV VR*TPTSLKI
KWDAPAVTVD YYVITYGEKG RGGYAWQEFE VPGSKRTATI SGLKPGVDYT
ITVYAGYKGY PTYYSSPISI NYRT (* = BeLaK);
NSa1(+7)-A13BeLaK-Y92K-C1
[SEQ. ID. NO: 12]
MGSSHHHHHHSSGTENLYFQGC VSSKPTKLRV VR*TPTSLKI
KWDAPAVTVD YYVITYGEKG RGGYAWQEFE VPGSKRTATI SGLKPGVDYT
ITVYAGYKGY PTYYSSPISI NKRT (* = BeLaK);
NSa1(+7)-A13BocK-Y92K-C1
[SEQ. ID. NO: 13]
MGSSHHHHHHSSGTENLYFQGC VSSKPTKLRV VR*TPTSLKI
KWDAPAVTVD YYVITYGEKG RGGYAWQEFE VPGSKRTATI SGLKPGVDYT
ITVYAGYKGY PTYYSSPISI NKRT (* = BocK);
NSa1(+10)-A13BeLaK
[SEQ. ID. NO: 14]
MGSSHHHHHHSSGTENLYFQG VSSKPTKLRV VR*TPTSLKI
KWDAPAKTVD YYVITYGETG RGGYAWQRFE VPGSKRTATI KGLKPGVDYT
ITVYAGYKGY PTYYSSPISI NYRT (* = BeLaK);
NSa1(+10)-A13BeLaK-C1
[SEQ. ID. NO: 15]
MGSSHHHHHHSSGTENLYFQGC VSSKPTKLRV VR*TPTSLKI
KWDAPAKTVD YYVITYGETG RGGYAWQRFE VPGSKRTATI KGLKPGVDYT
ITVYAGYKGY PTYYSSPISI NYRT (* = BeLaK);
NSa1(+10)-A13BeLaK-Y92K-C1
[SEQ. ID. NO: 16]
MGSSHHHHHHSSGTENLYFQGC VSSKPTKLRV VR*TPTSLKI
KWDAPAKTVD YYVITYGETG RGGYAWQRFE VPGSKRTATI KGLKPGVDYT
ITVYAGYKGY PTYYSSPISI NKRT (* = BeLaK);
NSa1(+10)-A13BocK-Y92K-C1
[SEQ. ID. NO: 17]
MGSSHHHHHHSSGTENLYFQGC VSSKPTKLRV VR*TPTSLKI
KWDAPAKTVD YYVITYGETG RGGYAWQRFE VPGSKRTATI KGLKPGVDYT
ITVYAGYKGY PTYYSSPISI NKRT (* = BocK);
NSa1(+17)-A13BeLaK
[SEQ. ID. NO: 18]
MGSSHHHHHHSSGTENLYFQG VKSKPTKLRV VR*TPTSLKI
SWKAPKKTVD YYVITYGKTG SGGYAWQRFR VPGSKRTAKI KGLKPGVDYT
ITVYAGYKGY PTYYSSPISI NYRT (* = BeLaK);
NSa1(+17)-A13BeLaK-Y92K
[SEQ. ID. NO: 19]
MGSSHHHHHHSSGTENLYFQG VKSKPTKLRV VR*TPTSLKI
SWKAPKKTVD YYVITYGKTG SGGYAWQRFR VPGSKRTAKI KGLKPGVDYT
ITVYAGYKGY PTYYSSPISI NKRT (* = BeLaK);
NSa1(+17)-A13BeLaK-Y92K-C1
[SEQ. ID. NO: 20]
MGSSHHHHHHSSGTENLYFQGC VKSKPTKLRV VR*TPTSLKI
SWKAPKKTVD YYVITYGKTG SGGYAWQRFR VPGSKRTAKI KGLKPGVDYT
ITVYAGYKGY PTYYSSPISI NKRT (* = BeLaK);
NSa1(+17)-A13BocK-Y92K-C1
[SEQ. ID. NO: 21]
MGSSHHHHHHSSGTENLYFQGC VKSKPTKLRV VR*TPTSLKI
SWKAPKKTVD YYVITYGKTG SGGYAWQRFR VPGSKRTAKI KGLKPGVDYT
ITVYAGYKGY PTYYSSPISI NKRT (* = BocK);
NSa1(+10)-A13BeLaK-C95
[SEQ. ID. NO: 22]
MGSSHHHHHHSSGTENLYFQG VSSKPTKLRV VR*TPTSLKI
KWDAPAKTVD YYVITYGETG RGGYAWQRFE VPGSKRTATI KGLKPGVDYT
ITVYAGYKGY PTYYSSPISI NYRTC (* = BeLaK);
NSa1(+10)-A13BeLaK-C1
[SEQ. ID. NO: 23]
MGSSHHHHHHSSGTENLYFQGC VSSKPTKLRV VR*TPTSLKI
KWDAPAKTVD YYVITYGETG RGGYAWQRFE VPGSKRTATI KGLKPGVDYT
ITVYAGYKGY PTYYSSPISI NYRT (* = BeLaK).

In various examples, a protein comprises (or consists of) at least 70%, at least 75%, at least 80%, at least 85%, at least 90%, or at least 95% of the sequence of a protein of this example. In various examples, a protein comprises (or consists of) at has at least 70%, at least 75%, at least 80%, at least 85%, or at least 90% homology a protein of this example. In various examples, a protein comprises (or consists of) at least 70%, at least 75%, at least 80%, at least 85%, at least 90%, or at least 95% of the sequence of a protein of this example and at has at least 70%, at least 75%, at least 80%, at least 85%, or at least 90% homology a protein of this example.

In various examples, a protein comprises (or consists of) at least 70%, at least 75%, at least 80%, at least 85%, at least 90%, or at least 95% of the sequence of a protein of the present disclosure. of this example. In various examples, a protein comprises (or consists of) at has at least 70%, at least 75%, at least 80%, at least 85%, or at least 90% homology a protein of the present disclosure. In various examples, a protein comprises (or consists of) at least 70%, at least 75%, at least 80%, at least 85%, at least 90%, or at least 95% of the sequence of a protein of the present disclosure and at has at least 70%, at least 75%, at least 80%, at least 85%, or at least 90% homology a protein of the present disclosure.

In various examples, a protein further comprises one or more therapeutic modalit(ies) (e.g., therapeutic compound(s), therapeutic group(s), or the like), one or more diagnostic modalit(ies) (e.g., diagnostic compound(s), diagnostic group(s), or the like), or the like, or any combination thereof. Non-limiting examples of therapeutic modalities include drug groups (such as, for example, groups formed from drugs (e.g., cytotoxins and the like)), radionuclides/radionuclide groups, and the like. Examples of suitable drugs/drug groups are known in the art. Examples of protein-drug conjugation methodologies are known in the art. Non-limiting examples of diagnostic modalities include fluorophores (such as, for example, fluorescent dyes, fluorescent nanoparticles, and the like), positron emission tomography probes, magnetic resonance imaging contrast agents, and groups formed therefrom, and the like. Examples of suitable fluorophores, positron emission tomography probes, and magnetic resonance imaging contrast agents are known in the art. Examples of protein conjugation with fluorophores, positron emission tomography probes, magnetic resonance imaging contrast agents are known in the art.

A protein can exhibit various bioactivit(ies) and/or comprise additional bioactive groups. In various examples, a protein further exhibits one or more biological activit(ies) (e.g., anticancer activit(ies) or the like). In various examples, a protein further comprises one or more therapeutic group(s), one or more prophylactic group(s), one or more diagnostic group(s), or the like, or any combination thereof.

A protein of the present disclosure can be made by various methods. In various examples, a protein is formed by a DNA-based recombinant method (e.g., genetic code expansion or the like), and where the first amino acid residue(s) (e.g., lysine derivative(s) or the like) is/are independently at each occurrence site-specifically incorporated into the protein via a wild-type or mutant pyrrolysine-tRNA synthetase/tRNA^{Pyl} pair.

In an aspect, the present disclosure also provides methods of making proteins (e.g., non-crosslinked proteins or the like) of the present disclosure. In various examples, a method comprises recombinant production of a protein of the present disclosure (e.g., a protein comprising one or more first amino acid residue(s) (e.g., one or more amino acid reside(s) each formed from a lysine derivative or the like), at a desired position or positions in the protein. In various examples, a protein is made by a method of the present disclosure. Non-limiting examples of methods of making proteins are described herein.

As used herein, unless otherwise stated, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

In various examples, a protein is formed by a DNA-based recombinant method (e.g., genetic code expansion or the like). In various examples, a DNA-based recombinant method forms a protein within one or more cells. In various examples, the DNA-based recombinant method comprises site-specific incorporation of a first amino acid residue(s) (e.g., a first lysine derivative(s) or the like) into the protein via a wild type or mutant pyrrolysine tRNA synthetase/tRNA^{Pyl} pair, or the like. In various examples, a protein spontaneously (or by subjecting the protein to appropriate conditions) forms a crosslinked protein.

In various examples, a protein or crosslinked protein is an engineered protein or crosslinked engineered protein. In various examples, an engineered protein is chosen from antibodies (such as, for example, monoclonal antibodies and the like), antibody fragments, single-chain variable fragments, fusion proteins, monobodies (which may also be referred to as Adnectins), nanobodies, affibodies, aptamers, affilins, affimers, affitins, alphabodies, anticalins, avimers, knottins, armadillo repeat proteins, designed ankyrin repeat proteins (DARPins), fynomers, gastrobodies, clostridal antibody mimetic proteins (nanoCLAMPs), optimers, repebodies, recombinant fibronectins (e.g., Pronectin™ and the like), centyrins, and obodies, and the like, and any portion thereof. In various examples, a protein further comprises one or more therapeutic compound(s).

A method can comprise incorporation (e.g., site-specific incorporation) of various lysine derivatives. In various examples, a lysine derivative forms a first amino acid residue. Non-limiting examples of lysine derivatives are disclosed herein.

Non-limiting examples of DNA-based recombinant methods for expression of proteins are known in the art (e.g., genetic code expansion or the like). Further, such methods are capable of modifying proteins to include non-canonical amino acids (ncAAs). Aminoacyl-tRNA synthetases (used interchangeably herein with AARS, RS or “synthetase”) catalyze the aminoacylation reaction for incorporation of amino acids into proteins via the corresponding transfer RNA molecules. Precise manipulation of synthetase activity can alter the aminoacylation specificity to stably attach ncAAs into the intended tRNA. Then, through codon-anticodon interaction between message RNA (mRNA) and tRNA, the ncAAs can be delivered into a growing polypeptide chain. Thus, incorporation of ncAAs into proteins relies on the manipulation of amino acid specificity of aminoacyl tRNA synthetases. The aminoacyl-tRNA synthetase used in certain methods disclosed herein can be a naturally occurring synthetase derived from an organism, whether the same (homologous) or different (heterologous), a mutated or modified synthetase, or a designed synthetase.

Aminoacyl-tRNA synthetases must perform their tasks with high accuracy. Many of these enzymes recognize their tRNA molecules using the anticodon. These enzymes make about one mistake in 10,000. A crystal structure defines the orientation of the natural substrate amino acid in the binding pocket of a synthetase, as well as the relative position of the amino acid substrate to the synthetase residues, especially those residues in and around the binding pocket. To design the binding pocket for the ncAAs, it is preferred that these ncAAs bind to the synthetase in the same orientation as the natural substrate amino acid, since this orientation may be important for the adenylation step. The crystal structures of nearly all 20 different AARS enzymes are currently available in the Brookhaven Protein Data Bank (PDB, see Bernstein et al., J. Mol. Biol. 112: 535-542, 1977). In addition, a database of known aminoacyl tRNA synthetases has been published by Maciej Szymanski, Marzanna A. Deniziak and Jan Barciszewski, in Nucleic Acids Res. 29:288-290, 2001 (titled “Aminoacyl-tRNA synthetases database”).

In various examples, the synthetase used can recognize the desired ncAA selectively over related amino acids available. For example, when the ncAA to be used is structurally related to a naturally occurring amino acid, the synthetase should charge the exogenous tRNA molecule with the desired ncAA with an efficiency at least substantially equivalent to that of, and more preferably at least about twice, 3 times, 4 times, 5 times or more than that of the naturally occurring amino acid. However, in cases in which a well-defined protein product is not necessary, the synthetase can have relaxed specificity for charging amino acids.

A synthetase can be obtained by a variety of techniques known to one of skill in the art, including combinations of such techniques as, for example, computational methods, selection methods, and incorporation of synthetases from other organisms (see, e.g., U.S. Pat. No. 8,980,581B2).

In various examples, synthetases can be used or developed that efficiently charge tRNA molecules that are not charged by synthetases of the host cell. For example, suitable pairs may be generally developed through modification of synthetases from organisms distinct from the host cell. In various examples, the synthetase can be developed by selection procedures. In various examples, the synthetase can be designed using computational techniques such as those described in Datta et al., J. Am. Chem. Soc. 124: 5652-5653, 2002, and in U.S. Pat. No. 7,139,665, hereby incorporated herein by reference.

There are a variety of computational methods that can be readily adapted for identifying the structure of ncAAs that would have appropriate steric and electronic properties to interact with the substrate binding site of a modified AARS (See, e.g., Cohen et al. (1990) J. Med. Cam. 33: 883-894; Kuntz et al. (1982) J. Mol. Biol 161: 269-288; DesJarlais (1988) J. Med. Cam. 31: 722-729; Bartlett et al. (1989) (Spec. Publ., Roy. Soc. Chem.) 78: 182-196; Goodford et al. (1985) J. Med. Cam. 28: 849-857; DesJarlais et al. J. Med. Cam. 29: 2149-2153).

Another example strategy used to generate a modified tRNA/RS pair involves importing a tRNA and/or synthetase from another organism into the translation system of interest, such as Escherichia coli. In this particular example, the heterologous synthetase candidate does not charge Escherichia coli tRNA reasonably well or not at all, and the heterologous tRNA is not acylated by Escherichia coli synthetase to a reasonable extent or not at all. Schimmel et al. reported that Escherichia coli GlnRS (EcGlnRS) does not acylate Saccharomyces cerevisiae tRNA^{Gln} (See, E. F. Whelihan and P. Schimmel, EMBO J., 16:2968 (1997)). Additionally, the Saccharomyces cerevisiae amber suppressor tRNA^{Gln} (SctRNA^{Gln}_{CUA}) was analyzed to determine whether it is also not a substrate for EcGlnRS. In vitro aminoacylation assays showed this to be the case; and in vitro suppression studies show that the SctRNA^{Gln}_{CUA} is competent in translation (see, e.g., Liu and Schultz, PNAS. USA, 96:4780 (1999)). RajBhandary and coworkers found that an amber mutant of human initiator tRNA^{fMet} is acylated by Escherichia coli GlnRS and acts as an amber suppressor in yeast cells only when EcGlnRS is coexpressed (see, Kowal, et al., PNAS USA, 98:2268 (2001)).

Genetic code expansion has been demonstrated for the site-specific incorporation of ncAAs into a polypeptide using an orthogonal codon which encodes an ncAA at a specific site in the polypeptide using a mutant pyrrolysyl-tRNA synthetase (PylRS) capable of charging the ncAA (the disclosures of which with respect to recombinant protein synthesis disclosed herein are hereby incorporated herein by reference). Suitable pyrrolysyl-tRNA synthetase (see U.S. Pat. No. 9,133,449, filed Apr. 8, 2014; U.S. Pat. Appl. Pub. No. 2015/0148525, filed May 15, 2013; and U.S. Pat. No. 7,993,872, filed Apr. 16, 2004) can be produced by mutagenesis, in various methods, of wild-type PylRS obtained from archaebacteria, particularly form methanogenic archaebacteria. Wild-type PylRS may be obtained from, but not restricted to, for example, Methanosarcina mazei (M. mazei), Methanosarcina barkeri (M. barkeri) and Methanosarcina acetivorans (M. acetivorans) and the like, which are methanogenic archaebacteria. Genomic DNA sequences of a lot of bacteria including those archaebacteria and amino acid sequences based on these nucleic acid sequences are known and it is also possible to obtain another homologous PylRS from public database such as GenBank by performing homology search for the nucleic acid sequences and the amino acid sequences, for example. M. mazei-derived PylRS, as typical examples, is deposited as Accession No. AAM31141, M. barkeri-derived PylRS is deposited as Accession No. AAL40867 and M. acetivorans-derived PylRS is deposited as accession No. AAM03608. M. mazei-derived PylRS as mentioned above is particularly preferred.

The practice of using orthogonal translation systems that are suitable for making proteins that comprise one or more unnatural amino acid is generally known in the art, as are the general methods for producing orthogonal translation systems. For example, see International Publication Numbers WO 2002/086075, entitled “METHODS AND COMPOSITION FOR THE PRODUCTION OF ORTHOGONAL tRNA-AMINOACYL-tRNA SYNTHETASE PAIRS;” WO 2002/085923, entitled “IN VIVO INCORPORATION OF UNNATURAL AMINO ACIDS;” WO 2004/094593, entitled “EXPANDING THE EUKARYOTIC GENETIC CODE;” WO 2005/019415, filed Jul. 7, 2004; WO 2005/007870, filed Jul. 7, 2004; WO 2005/007624, filed Jul. 7, 2004 and WO 2006/110182, filed Oct. 27, 2005, entitled “ORTHOGONAL TRANSLATION COMPONENTS FOR THE VIVO INCORPORATION OF UNNATURAL AMINO ACIDS.” Each of these applications is hereby incorporated herein by reference in its entirety. For additional discussion of orthogonal translation systems that incorporate unnatural amino acids, and methods for their production and use, see also, Wang and Schultz, “Expanding the Genetic Code,” Chem. Commun. (Camb.) 1:1-11 (2002); Wang and Schultz “Expanding the Genetic Code,” Angewandte Chemie Int. Ed., 44(1):34-66 (2005); Xie and Schultz, “An Expanding Genetic Code,” Methods 36(3):227-238 (2005); Xie and Schultz, “Adding Amino Acids to the Genetic Repertoire,” Curr. Opinion in Chemical Biology 9(6):548-554 (2005); Wang et al., “Expanding the Genetic Code,” Annu. Rev. Biophys. Biomol. Struct., 35:225-249 (2006); and Xie and Schultz, “A Chemical Toolkit for Proteins-an Expanded Genetic Code,” Nat. Rev. Mol. Cell. Biol., 7(10):775-782 (2006). Orthogonal AARSs that can attach a non-canonical amino acid (ncAA) to its cognate tRNA are known (see, e.g., U.S. Pat. No. 9,102,932B2; Cervettini D, Tang S, Fried S D, et al. Rapid discovery and evolution of orthogonal aminoacyl-tRNA synthetase-tRNA pairs. Nat Biotechnol. 2020; 38(8):989-999; Ding W, Zhao H, Chen Y, et al. Chimeric design of pyrrolysyl-tRNA synthetase/tRNA pairs and canonical synthetase/tRNA pairs for genetic code expansion. Nat Commun. 2020; 11(1):3154. Published 2020 Jun. 22; Melnikov S V, Soll D. Aminoacyl-tRNA Synthetases and tRNAs for an Expanded Genetic Code: What Makes them Orthogonal?Int J Mol Sci. 2019; 20(8):1929. Published 2019 Apr. 19; Chatterjee A, Xiao H, Schultz PG. Evolution of multiple, mutually orthogonal prolyl-tRNA synthetase/tRNA pairs for unnatural amino acid mutagenesis in Escherichia coli. Proc Natl Acad Sci USA. 2012; 109(37):14841-14846; Thibodeaux G N, Liang X, Moncivais K, et al. Transforming a pair of orthogonal tRNA-aminoacyl-tRNA synthetase from Archaea to function in mammalian cells. PLoS One. 2010; 5(6):e11263. Published 2010 Jun. 22; and Using a Quadruplet Codon to Expand the Genetic Code of an Animal, Zhiyan Xi, Lloyd Davis, Kieran Baxter, Ailish Tynan, Angeliki Goutou, Sebastian Greiss. bioRxiv 2021.07.17.452788).

In various examples, an engineered pyrrolysyl-tRNA synthetase comprises one or more amino acid mutations within a substrate-binding site as compared to a wild-type pyrrolysyl-tRNA synthetase, where the substrate-binding site comprises amino acid 306, amino acid 309, amino acid 348, amino acid 384 of SEQ ID NO: 24 or in corresponding positions thereto in a variant thereof. In various examples, the one or more amino acid mutation(s) comprise a Y306V, L309A, C348F, Y384F, or any combination thereof. In various examples, an engineered pyrrolysyl-tRNA synthetase comprises a substrate-binding site comprising a valine residue or the like at position 306, an alanine residue or the like at position 309, a phenylalanine residue or the like at position 348, and a phenylalanine residue or the like at position 384. In various examples, the engineered pyrrolysyl-tRNA synthetase is suitable for binding with (or binds) with a compound of the present disclosure (such as for example, a compound comprising a triazolyl group or the like). In various examples, the engineered pyrrolysyl-tRNA synthetase or variant thereof comprises 80%, 85%, 90%, or 95% up to but excluding 100% homology, with the wild-type pyrrolysyl-tRNA synthetase (SEQ ID NO: 24). The wild-type pyrrolysyl-tRNA synthetase comprises the following sequence:

(SEQ ID NO: 24)
MDKKPLNTLISATGLWMSRTGTIHKIKHHEVSRSKIYIEMACGDHLVVNN
SRSSRTARALRHHKYRKTCKRCRVSDEDLNKFLTKANEDQTSVKVKVVSA
PTRTKKAMPKSVARAPKPLENTEAAQAQPSGSKFSPAIPVSTQESVSVPA
SVSTSISSISTGATASALVKGNTNPITSMSAPVQASAPALTKSQTDRLEV
LLNPKDEISLNSGKPFRELESELLSRRKKDLQQIYAEERENYLGKLEREI
TRFFVDRGFLEIKSPILIPLEYIERMGIDNDTELSKQIFRVDKNFCLRPM
LAPNLYNYLRKLDRALPDPIKIFEIGPCYRKESDGKEHLEEFTMLNFCQM
GSGCTRENLESIITDFLNHLGIDFKIVGDSCMVYGDTLDVMHGDLELSSA
VVGPIPLDREWGIDKPWIGAGFGLERLLKVKHDFKNIKRAARSESYYNGI
STNL.

In various examples, the engineered pyrrolysyl-tRNA synthetase or variant thereof comprises or consists of a polypeptide comprising the following sequence:

(SEQ. ID. NO. 25)
MDKKPLNTLISATGLWMSRTGTIHKIKHHEVSRSKIYIEMACGDHLVVNN
SRSSRTARALRHHKYRKTCKRCRVSDEDLNKFLTKANEDQTSVKVKVVSA
PTRTKKAMPKSVARAPKPLENTEAAQAQPSGSKFSPAIPVSTQESVSVPA
SVSTSISSISTGATASALVKGNTNPITSMSAPVQASAPALTKSQTDRLEV
LLNPKDEISLNSGKPFRELESELLSRRKKDLQQIYAEERENYLGKLEREI
TRFFVDRGFLEIKSPILIPLEYIERMGIDNDTELSKQIFRVDKNFCLRPM
LAPNLVNYARKLDRALPDPIKIFEIGPCYRKESDGKEHLEEFTMLNFFQM
GSGCTRENLESIITDFLNHLGIDFKIVGDSCMVFGDTLDVMHGDLELSSA
VVGPIPLDREWGIDKPWIGAGFGLERLLKVKHDFKNIKRAARSESYYNGI
STNL.

In various examples, a complex comprises a variant pyrrolysyl-tRNA synthetase of the present disclosure and a compound of the present disclosure (such as for example, a compound comprising a beta-lactam group or the like). In various examples, a vector comprises a variant a variant pyrrolysyl-tRNA synthetase of the present disclosure. In various examples, cell comprises a variant a variant pyrrolysyl-tRNA synthetase of the present disclosure. In various examples, genome comprises a variant a variant pyrrolysyl-tRNA synthetase of the present disclosure. In various examples, a cell comprises the pyrrolysyl-tRNA synthetase, the vector, the genome, or the complex, or a combination of two or more thereof.

As used herein with reference to the relationship between DNA, cDNA, cRNA, RNA, protein/peptides, and the like “corresponding to” or “encoding” (used interchangeably herein), unless otherwise stated, refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, unless otherwise stated, the term “vector” or is used in reference to a vehicle used to introduce an exogenous nucleic acid sequence into a cell. A vector may include a DNA molecule, linear or circular (e.g., plasmids), which includes a segment encoding an RNA and/or polypeptide of interest operatively linked to additional segments that provide for its transcription and optional translation upon introduction into a host cell or host cell organelles. Such additional segments can include promoter and/or terminator sequences, and can also include one or more origins of replication, one or more selectable markers, an enhancer, a polyadenylation signal, etc. Expression vectors are generally derived from yeast or bacterial genomic or plasmid DNA, or viral DNA, or may contain elements of both. Expression vectors can be adapted for expression in prokaryotic or eukaryotic cells. Expression vectors can be adapted for expression in mammalian, fungal, yeast, or plant cells. Expression vectors can be adapted for expression in a specific cell type via the specific regulator or other additional segments that can provide for replication and expression of the vector within a particular cell type. Various vectors suitable for use in connection with the present disclosure are generally known in the art. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

In various examples, the vector is an expression vector that comprises one or more polynucleotides encoding one or more pyrrolysyl-tRNA synthetases described herein. In various examples, pyrrolysyl-tRNA synthetase encoding polynucleotide is codon optimized for expression in a particular cell type. Codon optimization is generally known in the art. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In various examples, the vector is a plasmid or the like. In various examples, the vector is a viral vector or the like. In various examples, the vector is a lentiviral vector or the like.

In various examples, a method of making a protein of the present disclosure comprises contacting a nucleic acid with a pyrrolysyl-tRNA synthetase (such as, for example, a pyrrolysyl-tRNA synthetase of the present disclosure or the like), a tRNA^{Pyl}, and a compound of the present disclosure, where the nucleic acid encodes a protein, and wherein the nucleic acid comprises at least one codon recognized by a tRNA^{Pyl}, thereby producing the protein. In various examples, the contacting is in vitro or in vivo. In various examples, the contacting is in a cell (such as, for example, a bacterial cell, a fungal cell, a plant cell, an archaeal cell, an animal cell, or the like).

In an aspect, the present disclosure provides crosslinked proteins. In various examples, a crosslinked protein comprises (or consists of) any non-crosslinked protein of the present disclosure, or at least a portion or all of sequence thereof, where the protein is crosslinked. Non-limiting examples of crosslinked proteins are disclosed herein.

A crosslinked protein can comprise various types and/or in the case of a crosslinked protein comprising a plurality of crosslinks, numbers and/or distributions of crosslinks. In various examples, the intramolecular crosslink(s) and/or intermolecular crosslink(s) are formed by a beta-lactam ring opening reaction, an acyl transfer reaction, or the like. In various examples, a crosslinked protein comprises one or more intramolecular crosslink(s) and/or one or more intermolecular crosslink(s). In various examples, each crosslink independently at each occurrence comprises the following structure:

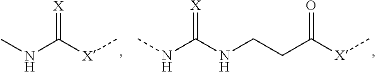

or the like, wherein X is independently at each occurrence an oxygen atom or a sulfur atom and X′ is independently at each occurrence an O atom, a S atom, a N atom, a NH group, or the like. In various examples, each crosslink independently at each occurrence comprises the following structure:

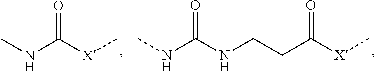

or the like, wherein X′ is independently at each occurrence an O atom, S atom, N atom, NH group, or the like. In various examples, each crosslink is formed (e.g., spontaneously formed or the like) between a first amino acid residue and a second amino acid residue (e.g., wherein

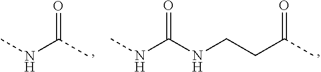

or the like) is formed from (or derived from) a side chain group of a first amino acid residue (which may be a first lysine derivative residue) of the protein, and wherein

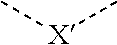

is formed from (or derived from) a side chain group of a second amino acid residue), or the like, or an analog or derivative thereof.

In various examples, a crosslinked protein comprises one or more intramolecular crosslink(s) and/or one or more intermolecular crosslink(s), the intramolecular crosslink(s) and/or the intermolecular crosslink(s) independently at each occurrence comprising the following structure:

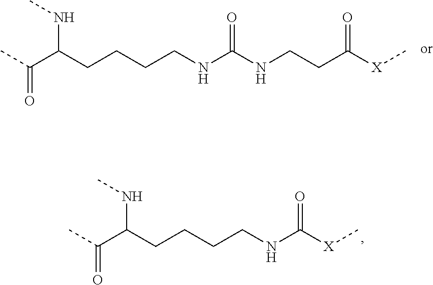

where X is independently at each occurrence an O atom, S atom, N atom, or NH group.

In various examples, a crosslinked protein comprises one or more intermolecular crosslink(s) between two separate polypeptide chains of the protein. Where the two separate chains are the same, a homodimer is formed. Where the two separate chains are different, a heterodimer is formed. In various examples, a crosslinked protein comprises one or more intermolecular crosslink(s) between two separate polypeptide chains of the protein, where both of the polypeptide chains of the protein are in solution or the like. In various examples, a crosslinked protein comprises one or more intermolecular crosslink(s) between two separate polypeptide chains of the protein, where one of the polypeptide chains of the protein is disposed on a surface of a cell or the like.

A crosslinked protein may comprise positively charged protein surface groups. A crosslinked protein can have various numbers of and/or distributions of positively charged protein surface groups. In various examples, a protein is supercharged (e.g., comprises one or more surface exposed positively charged amino acid residues or the like), In various examples, a protein comprises an overall net surface charge of from about +1 to about +20, including all integer values and ranges therebetween.

In various examples, a crosslinked protein is a crosslinked engineered protein. In various examples, a crosslinked engineered protein comprises an engineered protein chosen from antibodies, antibody fragments, fusion proteins, monobodies (which may also be referred to as adectins), nanobodies, affibodies, aptamers, affilins, affimers, affitins, alphabodies, anticalins, avimers, knottins, armadillo repeat proteins, DARPins, fynomers, gastrobodies, nanoCLAMPs, optimers, repebodies, Pronectin™, centyrins, obodies, and the like. In various examples, a crosslinked protein further comprises one or more therapeutic compound(s). In various examples, a crosslinked protein exhibits one or more biological activit(ies) (e.g., anticancer activit(ies) or the like). In various examples, a crosslinked protein is an antibody mimic.

In various examples, a crosslinked protein exhibits increased bioavailability (e.g., increased cellular uptake upon contact of the crosslinked protein with a cell or a population of cells, resistance to intracellular proteolytic degradation, or the like) as compared to a corresponding non-crosslinked protein (e.g., non-crosslinked protein that does not comprise the one or more crosslinked first amino acid(s), which may be the native amino acid(s)). In various examples, a crosslinked engineered protein exhibits increased bioavailability (e.g., increased cellular uptake upon contact of the crosslinked protein with a cell or a population of cells, resistance to intracellular proteolytic degradation, or the like) as compared to a corresponding non-crosslinked engineered protein (e.g., non-crosslinked engineered protein that does not comprise the one or more crosslinked first amino acid(s), which may be the native amino acid(s)).

In an aspect, the present disclosure also provides methods of making crosslinked proteins. Non-limiting examples of methods of making crosslinked proteins are disclosed herein.

A crosslinked protein can be formed by various methods. In various examples, a crosslinked protein is formed by the crosslinking of any non-crosslinked protein of the present disclosure (e.g., a protein formed by a DNA-based recombinant method (e.g., genetic code expansion or the like), optionally within one or more cells). In various examples, the crosslinked protein is formed spontaneously after formation of the non-crosslinked protein (e.g., within one or more cells or the like). In various examples, the crosslinking comprises reacting (e.g., spontaneously reacting or the like) a first reactive site of a first amino acid residue of the non-crosslinked protein and a reactive site of a second amino acid residue of the non-crosslinked protein in proximity thereto to form one or more intramolecular crosslink(s) and/or one or more intermolecular crosslink(s). In various examples, the intramolecular crosslink(s) and/or intermolecular crosslink(s) are formed by a beta-lactam ring opening reaction, an acyl transfer reaction, or the like. In various examples, the one or more intramolecular crosslink(s) and/or one or more intermolecular crosslink(s) is/are formed under neutral or basic pH conditions (e.g., about pH 7.0 or greater or about pH 7.4).

In various examples, a crosslinked protein is formed by the crosslinking of any non-crosslinked protein (e.g., a first protein or first polypeptide chain of the crosslinked protein or the like) of the present disclosure (e.g., a protein formed by a DNA-based recombinant method (e.g., genetic code expansion or the like) with a protein (e.g., a second protein or second polypeptide chain of the crosslinked protein or the like) disposed on a surface of a cell. In various examples, the crosslinking comprises reacting (e.g., spontaneously reacting or the like) a first reactive site of a first amino acid residue of the non-crosslinked protein and a reactive site of a second amino acid residue of the non-crosslinked protein disposed on a surface of a cell in proximity thereto to form one or more intermolecular crosslink(s).

In an aspect, the present disclosure provides cells. In various examples, a cell or a plurality of cells comprises one or more compound(s) of the present disclosure, one or more proteins(s) of the present disclosure, one or more crosslinked protein(s) of the present disclosure, or any combination thereof. Non-limiting examples of cells are disclosed herein.

In various examples, a compound or compounds is/are biosynthesized inside a cell, thereby generating a cell comprising the compound(s). In various examples, a compound or compounds is/are contained in a medium outside the cell and the compound(s) penetrate(s) into the cell, thereby generating a cell comprising the compound(s).

In various examples, a protein or proteins is/are biosynthesized inside a cell, thereby generating a cell comprising the protein(s). In various examples, a protein or proteins is/are contained in a medium outside the cell and the protein(s) penetrate(s) into the cell, thereby generating a cell comprising the proteins(s).

In various examples, a crosslinked protein or crosslinked proteins is/are formed on a surface of a cell or inside a cell, thereby generating a cell comprising the crosslinked protein(s). In various examples, a crosslinked protein or crosslinked proteins is/are contained in a medium outside the cell and the crosslinked proteins (s) penetrate(s) into the cell, thereby generating a cell comprising the crosslinked proteins(s).

A cell can be any prokaryotic or eukaryotic cell. In various examples, a cell is prokaryotic or the like. In various examples, a cell is eukaryotic or the like. In various examples, a cell is a bacterial cell, a fungal cell, a plant cell, an archaeal cell, an animal cell or the like. In various examples, an animal cell is an insect cell, a mammalian cell, or the like. In various examples, a cell is a human cell or the like. In various examples, a compound can be expressed in bacterial cells (such as, for example, E. coli or the like), insect cells, yeast or mammalian cells (such as, for example, HeLa cells, Chinese hamster ovary cells (CHO), COS cells, or the like), or the like. In various examples, a cell is a premature mammalian cell (e.g., a pluripotent stem cell or the like) or the like. In various examples, a cell is derived from human tissue or the like. Other suitable cells are known to those skilled in the art.

In an aspect, the present disclosure provides compositions comprising one or more crosslinked protein(s) of the present disclosure. Non-limiting examples of compositions are disclosed herein.

A composition may also comprise one or more additional component(s), one or more or all of which may be pharmaceutically acceptable components (such as, for example, pharmaceutically acceptable carriers, pharmaceutically acceptable excipients, pharmaceutically acceptable stabilizers, or the like, or any combination thereof). In various examples, a composition is a pharmaceutical composition comprising one or more pharmaceutically acceptable component(s). A pharmaceutical composition may comprise one or more other therapeutic agent(s) (therapeutic agent(s) other than protein(s) of the present disclosure).

Crosslinked protein(s) can be provided in pharmaceutical compositions for administration by combining them with any suitable pharmaceutically acceptable component(s). As used herein, unless otherwise stated, the term “pharmaceutically acceptable” refers to those components and dosage forms that are, within the scope of sound medical judgment, suitable for use in contact with the tissues of humans or animals without excessive toxicity, irritation, or other problem or complication, commensurate with a reasonable benefit/risk ratio. Non-limiting examples of materials that can be used as additional component(s) in a composition include sugars and other carbohydrates, such as, for example, monosaccharides (e.g., glucose and the like), disaccharides (e.g., lactose, sucrose, and the like), and other carbohydrates (e.g., mannose, dextrins, and the like), and the like; starches, such as, for example, corn starch, potato starch, and the like; cellulose, and its derivatives, such as, for example, sodium carboxymethyl cellulose, ethyl cellulose, cellulose acetate, and the like; powdered tragacanth; malt; gelatin; talc; excipients, such as, for example, cocoa butter, suppository waxes, and the like; oils, such as, for example, peanut oil, cottonseed oil, safflower oil, sesame oil, olive oil, corn oil, soybean oil, and the like; glycols, such as, for example, propylene glycol and the like; polyols, such as, for example, glycerin, sorbitol, mannitol, polyethylene glycol, and the like; esters, such as, for example, ethyl oleate, ethyl laurate, and the like; agar; amino acids such as, for example, glycine, glutamine, asparagine, histidine, arginine, lysine, and the like; buffering agents, such as, for example, magnesium hydroxide, aluminum hydroxide, and the like; alginic acid; pyrogen-free water; isotonic saline; Ringer's solution; ethyl alcohol; buffers such as, for example, acetate, Tris, phosphate, citrate, and other organic acid(s) buffer solutions; antioxidants, such as, for example, ascorbic acid, methionine, and the like; preservatives, such as, for example, octadecyldimethylbenzyl ammonium chloride and the like; chelating agents, such as, for example, EDTA and the like; tonicifiers, such as, for example, trehalose and sodium chloride; surfactants such as, for example, polysorbate, Tween, polyethylene glycol (PEG) and the like; and other non-toxic compatible substances employed in pharmaceutical formulations. Non-limiting examples of pharmaceutically acceptable carriers, excipients, stabilizers can be found in Remington: The Science and Practice of Pharmacy (2005) 21st Edition, Philadelphia, PA. Lippincott Williams & Wilkins.

In various examples, a composition is provided as single doses or in multiple doses covering the entire or partial treatment regimen. The compositions can be provided in liquid, solid, semi-solid, gel, aerosolized, vaporized, or any other form from which it can be delivered to an individual. In various examples, a composition is suitable for oral administration. In various examples, a composition is suitable for administration by injection.

Clinicians will be able to assess individuals who are in need of being treated for these conditions or individuals themselves may be able to assess a need for intake of these crosslinked protein(s) or compositions. The crosslinked proteins(s) or compositions may be used in combination with other therapeutic approaches for the conditions. In various examples, a method further comprises one or more additional therapeutic approach(es) (such as, for example other therapeutic approaches for treatment of cancer or the like). The additional therapeutic approaches can be carried out sequentially or simultaneously with the treatment involving the present compositions.

As used herein, unless otherwise stated, “treatment” of a condition, disease, or disease state, or the like, or any combination thereof. is not limited to treatment, but encompasses reduction or alleviation of one or more or all of the symptom(s) of a condition, disease, or disease state, and the like, or any composition thereof.

An individual may be a human or a non-human animal. An individual may be a mammal. Non-limiting examples of non-human animals (e.g., mammals) include cows, pigs, goats, mice, rats, rabbits, other agricultural mammals, cats, dogs, pets, service animals, and the like.

Administration of crosslinked protein(s) or compositions comprising crosslinked protein(s) as described herein can be carried out using any suitable route of administration known in the art. In various examples, the crosslinked protein(s) or the compositions are administered via intravenous, intramuscular, intraperitoneal, intracerobrospinal, subcutaneous, intra-articular, intrasynovial, oral, topical, inhalation routes, or the like. The compositions may be administered parenterally or enterically. In various examples, the crosslinked protein(s) or the compositions are administered orally or by injection. The compositions may be introduced as a single administration or as multiple administrations or may be introduced in a continuous manner over a period of time. In various examples, the administration(s) can be a pre-specified number of administrations or daily, weekly, or monthly administrations, which may be continuous or intermittent, as may be clinically needed and/or therapeutically indicated.

As used herein, unless otherwise stated, “effective amount” refers to the amount of the crosslinked protein(s) (one or more of which may be present in a composition) that achieve one or more therapeutic effect(s) or desired effect(s). A physician or veterinarian having ordinary skill in the art can readily determine and prescribe the effective amount of the compound(s) and/or composition(s)required. The selected effective amount can depend upon a variety of factors including, but not limited to, the activity of the particular composition employed, the time of administration, the rate of excretion or metabolism of the particular composition being employed, the rate and extent of absorption, the duration of the treatment, other drugs, compounds and/or materials used in combination with the particular composition employed, the age, sex, weight, condition, general health and prior medical history of the patient being treated, and like factors well known in the medical arts. For example, the physician or veterinarian could start doses of the composition employed at levels lower than that required in order to achieve the desired therapeutic effect and gradually increase the dosage until the desired effect is achieved.

In an aspect, the present disclosure provides uses for crosslinked proteins of the present disclosure (one or more or all of which may be present in a composition of the present disclosure and/or delivered by a method of the present disclosure). Crosslinked proteins can be used, for example, in cellular delivery, to treat various conditions (e.g., in various therapeutic methods), or the like. Non-limiting examples of conditions and therapeutic methods are disclosed herein. Non-limiting examples of uses of crosslinked protein(s) are disclosed herein.

In various examples, the present disclosure provides a method of cellular delivery, the method comprising: contacting one or more crosslinked protein(s) of the present disclosure with a cell or a population of cells, wherein the crosslinked protein(s) are delivered into the cell or the population of cells. In various examples, the method provides increased bioavailability (e.g., increased cellular uptake and/or increased intracellular proteolytic resistance) of the crosslinked protein(s) as compared to corresponding non-crosslinked protein(s). In various methods, the crosslinked protein(s) is/are crosslinked engineered protein(s). In various examples, the method is capable of increased bioavailability (e.g., increased cellular uptake and/or increased intracellular proteolytic resistance) of crosslinked engineered protein(s) as compared to corresponding non-crosslinked engineered protein(s).

In various examples, a crosslinked protein is or comprises a therapeutic, prophylactic, or diagnostic compound for a present or future condition, disease, or disease state, or the like, or any combination thereof. In various examples, a crosslinked protein(s) is/are used to treat, prevent, or diagnose a present or future condition, disease, or disease state, or the like, or any combination thereof. In various examples, the present disclosure provides methods of treating an individual in need of treatment, prevention, or diagnosis for a present or future condition, disease, or disease state, or the like, or any combination thereof. In various examples, a method of treating, preventing, or diagnosing the present or future condition, disease, or disease state, or the like, or any combination thereof in an individual (which may be an individual diagnosed with, suspected of having, or suspecting of developing one or more of the present disease states) comprises administration to an individual an effective amount of one or more crosslinked protein(s), which may be administered in the form of one or more composition(s).

An individual can be in in need of treatment, prevention, or diagnosis for various present or future conditions, diseases, disease states, or the like, or any combination thereof. In various examples, a condition, disease, or disease state is chosen from a cancer, an auto-immune disease, a metabolic disease, an infectious disease, or the like, or any combination thereof.

In various examples, the present disclosure provides a method of binding a target on a cell or a plurality of cells, the method comprising: contacting a cell or a plurality of cells with one or more protein(s) of the present disclosure, where the protein(s) is/are independently capable of specifically binding to the target on the surface of the cell or the individual surfaces of the cells of the plurality of cells, whereby the protein(s) and target forms one or more intermolecular crosslink(s) with the target(s) and a protein or proteins comprising the intermolecularly crosslinked protein(s) and target is/are formed. In various examples, wherein the intermolecular crosslink(s) (e.g., covalent bond(s)) is/are formed through a beta-lactam ring opening reaction or an acyl transfer reaction (such as, for example, a proximity-enabled beta-lactam ring opening or acyl transfer reaction or the like) or the like. In various examples, the intermolecular crosslink(s) independently at each occurrence comprises the following structure:

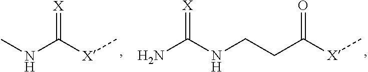

for the like, wherein X is independently at each occurrence an oxygen atom or a sulfur atom and X′ is independently at each occurrence an O atom, a S atom, a N atom, a NH group, or the like. In various examples, the intermolecular crosslink(s) independently at each occurrence comprises the following structure:

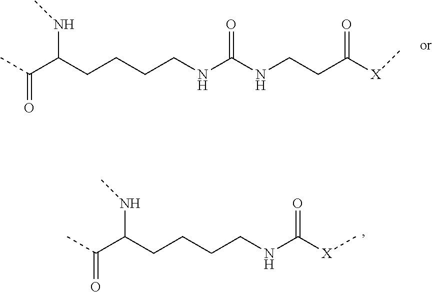

where X is independently at each occurrence an O atom, a S atom, a N atom, an NH group, or the like.

In various examples, a target is a protein, or the like, or a portion thereof. In various examples, a target is an intracellular protein or the like. Non-limiting examples of proteins include vascular endothelial growth factor receptor 2 (VEGFR2), proprotein convertase subtilisin kexin-9 (PCSK9), myostatin, BCR-ABL, aurora A kinase, SHP2, KRAS mutants, signal transducer and activator of transcription 3 (STAT3), and the like.

In various examples, a target is a receptor disposed on the surface of the cell. Non-limiting examples of receptors include membrane receptors, hormone receptors, and the like, and any combination thereof. Non-limiting examples of receptors include an acetylcholine receptor, an adenosine receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor (EGFR), a formyl peptide receptor, a free fatty acid receptor, a galanin receptor, a ghrelin receptor, a glycoprotein hormone receptor, a gonadotrophin-releasing hormone receptor, a G protein-coupled estrogen receptor, a histamine receptor, a hydroxycarboxylic acid receptor, human epidermal growth factor receptor 2 (HER2), a kisspeptin receptor, a leukotriene receptor, a lysophospholipid receptor, a lysophospholipid SiP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, a vasopressin receptor, and the like, and any combination thereof. In various examples, a target is PD-1, PD-L1, or the like, or any combination thereof.

In various examples a target is a cancer marker or the like. Non-limiting examples of cancer markers include EGFR, HER2, STEAP1, TROP2, PSMA, CD46, B7-H3, and the like, and any combination thereof. In various examples, a target is an antibody-drug conjugate target, a monobody target, or the like. In various examples, a target is a CD3 disposed on a surface of a T cell or the like. In various examples, an antibody-drug conjugate target, a monobody target, or the like.

In an aspect, the present disclosure provides kits. A kit comprises (or consists essentially of or consists of) one or more crosslinked protein(s) one or more of which may be present in a composition) and/or composition(s) of the present disclosure. In various examples, a kit comprises one or more crosslinked protein(s) and/or composition(s) (e.g., one or more pharmaceutical composition(s)). In various examples, a kit includes a closed or sealed package that contains the one or more crosslinked protein(s). In various examples, the package comprises one or more closed or sealed vial(s), bottle(s), blister (bubble) pack(s), or any other suitable packaging for the sale, distribution, or use of the one or more crosslinked protein(s) and/or composition(s). The printed material may include printed information. The printed information may be provided on a label, on a paper insert, printed on a packaging material, or the like. The printed information may include information that identifies the crosslinked protein(s) in the package, the amounts and types of other active and/or inactive ingredient(s) in the composition, and instructions for taking the crosslinked protein(s) and/or composition(s). The instructions may include information, such as, for example, the number of doses to take over a given period of time, and/or information directed to a pharmacist and/or another health care provider, such as, for example, a physician or the like, or a patient. The printed material may include an indication or indications that the one or more compound(s) and/or composition(s) and/or any other agent provided therein is for treatment of a subject. In various examples, the kit includes a label describing the contents of the kit and providing indications and/or instructions regarding use of the contents of the kit to treat a subject.

The following Statements describe various examples of compounds, proteins, crosslinked proteins, and methods of the present disclosure and are not intended to be in any way limiting:
Statement 1. A protein comprising one or more first amino acid residue(s) (which may be one or more first lysine derivative residue(s), or the like, or any combination thereof) comprising a reactive site (which may be a terminal group on the side chain of each first amino acid residue) comprising the following structure:

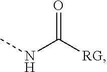

where RG is a reactive group independently at each occurrence comprising (or consisting of) the following structure:

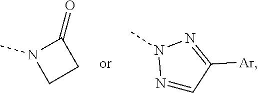

where Ar is an aromatic group (e.g., aromatic groups as shown in Examples 1 and 2 or the like), or any reactive group structure as shown in Examples 1 or 2, or the like, or an analog or derivative thereof; and one or more second amino acid residue(s) comprising a nucleophilic reactive site (which may be a nucleophilic terminal group (e.g., a hydroxyl group, a thiol group, a primary amine group, a secondary amine group, or the like) on the side chain of each second amino acid residue), where one or more or all of the first amino acid residue(s) is/are each in proximity to a second amino acid residue, such that the reactive site of each of the one or more or all first amino acid residue(s) is capable of reacting (e.g., spontaneously reacting or the like) with the reactive site of a second amino acid residue in proximity thereto to form one or more intramolecular crosslink(s) and/or one or more intermolecular crosslink(s).
Statement 2. A protein according to Statement 1, where Ar independently at each occurrence comprises (or has) the following structure:

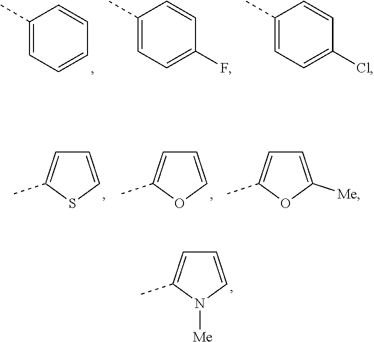

where Me is a methyl group, any other aromatic group structure shown in Examples 1 or 2, or the like, or an analog or derivative thereof.
Statement 3. A protein according to Statement 1 or Statement 2, where the second amino acid residue is independently at each occurrence chosen from lysine, tyrosine, histidine, cysteine, serine, and threonine.
Statement 4. A protein according to any one of Statements 1-3, where the protein is capable of forming the one or more intramolecular and/or one or more intermolecular crosslink(s) without interfering with (e.g., without reacting with) one or more cysteine disulfide bond(s) and/or one or more other cysteine residue(s) which are not second amino acid residue(s).
Statement 5. A protein according to any one of Statements 1-4, where the protein further comprises one or more cysteine disulfide bond(s).
Statement 6. A protein according to any one of Statements 1-5, where the protein is a single protein capable of forming one or more inter-strand intramolecular crosslink(s) and/or one or more intra-strand intramolecular crosslink(s).
Statement 7. A protein according to any one of Statements 1-6, where the protein is a complex of a plurality of single proteins (such as, for example, a dimer complex of two single proteins or the like), where each single protein of the plurality is capable of forming one or more inter-strand intramolecular crosslink(s) and/or one or more intra-strand intramolecular crosslink(s), and/or one or more intermolecular crosslink(s) with one or more other single protein(s) of the plurality of single proteins.
Statement 8. A protein according to any one of Statements 1-7, where the protein is capable of forming the one or more intramolecular crosslink(s) and/or one or more intermolecular crosslink(s) under neutral or basic pH conditions (e.g., about pH 7.0 or higher).
Statement 9. A protein according to any one of Statements 1-8, where the protein is supercharged (e.g., comprises one or more surface exposed positively charged amino acid residues or the like).
Statement 10. A protein according to any one of Statements 1-9, where the protein comprises an overall net surface charge of from about +1 to about +20.
Statement 11. A protein, according to any one of Statements 1-10, where the protein is an engineered protein.
Statement 12. A protein, according to Statement 11, where the engineered protein is chosen from antibodies, antibody fragments, fusion proteins, monobodies (which may also be referred to as adectins), nanobodies, affibodies, aptamers, affilins, affimers, affitins, alphabodies, anticalins, avimers, knottins, armadillo repeat proteins, DARPins, fynomers, gastrobodies, nanoCLAMPs, optimers, repebodies, Pronectin™, centyrins, obodies, and the like.
Statement 13. A protein according to any one of Statements 1-12, where the protein further comprises one or more therapeutic compound(s).
Statement 14. A protein according to any one of Statements 1-13, where the protein further comprises one or more biological activit(ies) (e.g., anticancer activit(ies) or the like).
Statement 15. A protein according to any one of Statements 1-14, where the protein is formed by a DNA-based recombinant method (e.g., genetic code expansion or the like), and where the first amino acid residue(s) (e.g., lysine derivative(s) or the like) is/are independently at each occurrence site-specifically incorporated into the protein via a wild-type or mutant pyrrolysine-tRNA synthetase/tRNA^{Pyl} pair.
Statement 16. A crosslinked protein comprising: one or more intramolecular crosslink(s) and/or one or more intermolecular crosslink(s), each crosslink independently at each occurrence comprising the following structure:

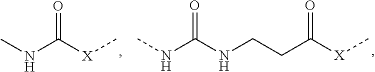

any other crosslink structure as shown in Example 1 or 2, or the like, where X is independently at each occurrence an O atom, S atom, N atom, NH group, or the like, (e.g., where

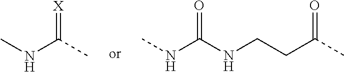

is formed from (or derived from) a side chain group of a first amino acid residue (which may be a first lysine derivative residue) of the protein, and where

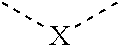

is formed from (or derived from) a side chain group of a second amino acid residue).
Statement 17. A crosslinked protein according to Statement 16, where the crosslinked protein comprises:
   one or more first amino acid residue(s) (e.g., one or more first lysine derivative residue(s), or the like) comprising a reactive site (which may be a terminal group on the side chain of each first amino acid residue) comprising the following structure:

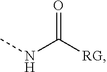

where RG is a reactive group independently at each occurrence comprising (or consisting of) the following structure:

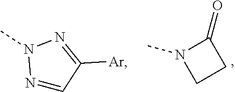

any other reactive group structure as shown in Example 1 or 2, or the like, or an analog or derivative thereof,
where Ar is an aromatic group (e.g., Ar groups as shown in Examples 1 and 2 or the like); and
   one or more second amino acid residue(s) comprising a nucleophilic reactive site (which may be a nucleophilic terminal group, such as, for example, a hydroxyl group, a thiol group, a primary amine group, a secondary amine group, and the like, on the side chain of each second amino acid residue), where one or more or all of the first amino acid residue(s) is/are each in proximity to a second amino acid residue, such that the one or more intramolecular crosslink(s) and/or the one or more intermolecular crosslink(s) are formed by the reaction (e.g., spontaneous reaction or the like) of the reactive site of each of the one or more or all first amino acid residue(s) with the reactive site of a second amino acid residue in proximity thereto.
Statement 18. A crosslinked protein according to Statement 16 or Statement 17, where the one or more intramolecular and/or one or more intermolecular crosslink(s) is/are formed under neutral pH conditions (e.g., about pH 7.0 or intracellular conditions).
Statement 19. A crosslinked protein according to any one of Statements 16-18, where the crosslinked protein is supercharged (e.g., comprises one or more surface exposed positively charged amino acid residues or the like).
Statement 20. A crosslinked protein according to any one of Statements 16-19, where the crosslinked protein comprises an overall net surface charge of from about +1 to about +20.
Statement 21. A crosslinked protein, according to any one of Statements 16-20, where the crosslinked protein is a crosslinked engineered protein.
Statement 22. A crosslinked protein, according to Statement 21, where the crosslinked engineered protein comprises an engineered protein chosen from antibodies, antibody fragments, fusion proteins, monobodies (which may also be referred to as adectins), nanobodies, affibodies, aptamers, affilins, affimers, affitins, alphabodies, anticalins, avimers, knottins, armadillo repeat proteins, DARPins, fynomers, gastrobodies, nanoCLAMPs, optimers, repebodies, Pronectin™, centyrins, obodies, and the like.
Statement 23. A crosslinked protein according to any one of Statements 16-22, where the crosslinked protein further comprises one or more therapeutic compound(s).
Statement 24. A crosslinked protein according to any one of Statements 16-23, where the crosslinked protein further comprises one or more biological activit(ies) (e.g., anticancer activit(ies) or the like).
Statement 25. A method of cellular delivery, the method comprising: contacting one or more crosslinked protein(s) of the present disclosure (e.g., a crosslinked protein of any one of Statements a crosslinked protein according to any one of Statements 16-24 or a crosslinked protein derived from the protein according to any one of Statements 1-15, where the method further comprises, prior to the contacting, the reactive site of each of the one or more or all first amino acid residue(s) reacts (e.g., spontaneously reacts or the like) with the reactive site of the second amino acid residue in proximity thereto, thereby forming the crosslinked protein) with a cell or a population of cells, where the crosslinked protein(s) are delivered into the cell or the population of cells.
Statement 26. A method according to Statement 25, where: the crosslinked protein is or comprises a therapeutic compound for a present condition, disease, or disease state, or the like, or any combination thereof, and where the contacting step occurs in an individual in need of treatment for the present condition, disease, or disease state, or the like, or any combination thereof; the crosslinked protein is or comprises a prophylactic compound for a potential condition, disease, disease state, or the like, or any combination thereof, and where the contacting step occurs in an individual in need of prophylaxis for the potential condition, disease, disease state, or the like, or any combination thereof, and/or the crosslinked protein is or comprises a diagnostic compound for a present or potential condition, disease, disease state, or the like, or any combination thereof, and where the contacting step occurs in an individual in need of diagnosis for the present or potential condition, disease, disease state, or the like, or any combination thereof.
Statement 27. A method according to Statement 25 or 26, where the condition, disease, or disease state is chosen from a cancer, an auto-immune disease, a metabolic disease, an infectious disease, or the like or any combination thereof, and where the individual has or is at risk of developing the condition, disease, disease state, or the like, or any combination thereof.

The steps of the methods described in the various examples disclosed herein are sufficient to carry out the methods of the present disclosure. Thus, in various examples, a method consists essentially of a combination of one or more step(s) of the methods disclosed herein. In various other examples, a method consists of such steps.

The following examples are presented to illustrate the present disclosure. They are not intended to be limiting in any manner.

### Example 1

This example provides a description of the preparation, characterization, and use of non-crosslinked proteins and crosslinked proteins of the present disclosure.

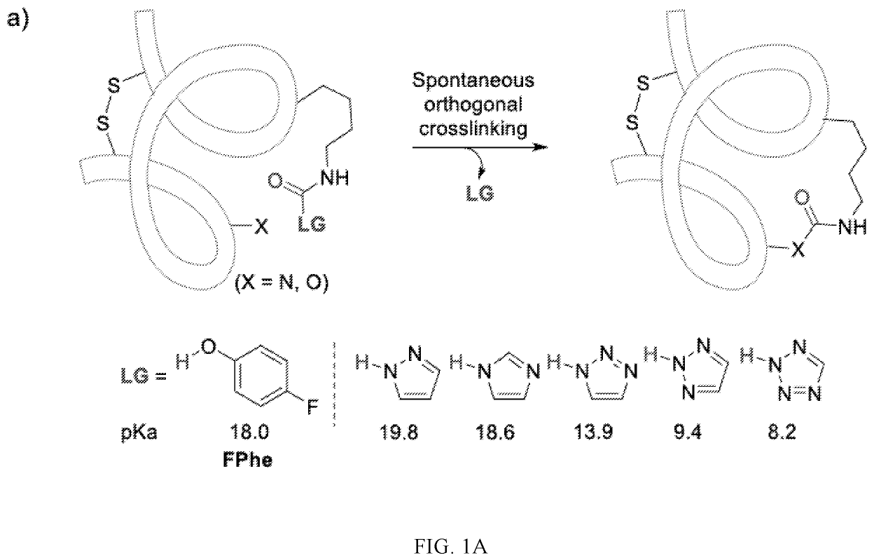

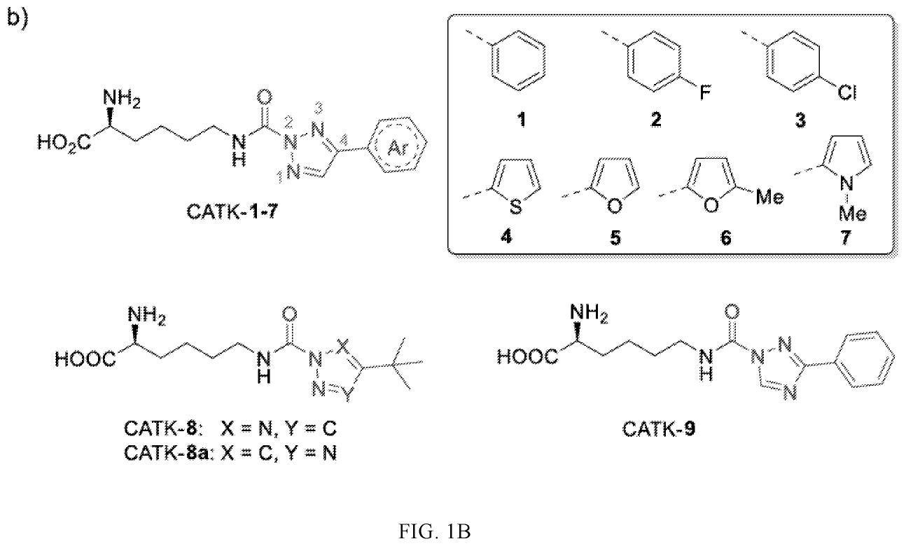

The formation of covalent crosslinks such as disulfide bonds within protein structure is vital to protein stability and function. To circumvent limitations of the prior art, an exogenous crosslink was designed that is orthogonal to the disulfide bond and generated spontaneously via a proximity-driven acyl transfer reaction inside bacterial cells (FIG. 1a). The design involves the introduction of a genetically encoded electrophilic amino acid site-specifically into a protein of interest, which then undergoes spontaneous, intra- or inter-molecular, proximity-driven crosslinking with a nearby nucleophilic residue. While several electrophilic amino acids have been incorporated into proteins site-specifically through genetic code expansion, including p-2′-fluoroacetyl-phenylalanine, bromoalkyl amino acids BprY and BrC6K, fluorosulfate-modified tyrosine (FSY) and lysine (FSK), and noncanonical amino acids containing perfluorobenzene and vinylsulfonamide, they preferentially react with cysteine and lack orthogonality to the disulfide bond.

After incubating the protein in HEPES buffer, pH 8.5, at 37° C. for 8-12 hours, intramolecular crosslinking with a nearby nucleophilic amino acid (Lys, Cys, Tyr) was observed with good yields. We envisioned this acyl transfer-based crosslinking could proceed under neutral conditions if we can identify an appropriate genetically encoded leaving group. To this end, we considered a panel of azoles with pKₐ values ranging from 19.8 to 8.2, and a varying degree of leaving group effect (FIG. 1a). We were particularly attracted to 1,2,3-triazoles because: 1) 2H-1,2,3-trizole is quite acidic with pKₐ value of 9.4, making it an excellent leaving group in the acyl transfer reaction; and 2) N²-carboxy-1,2,3-triazoles have been used in the literature as stable electrophiles for chemical proteomics studies. Thus, we designed a series of N-carboxy-4-aryl-1,2,3-triazole-containing lysines (CATK-1-7) as well as three analogous triazolyl lysines, CATK-8, -8a, and -9, for comparison purposes (FIG. 1b). For the synthesis of CATK-1-9, the critical step involved the triphosgene-mediated coupling of aryl- or alkyl-substituted triazoles with a protected lysine. While there was no apparent selectivity for N²-carbamoylated CATKs, the two regioisomers can be readily separated by flash chromatography. After deprotection, CATK-1-9 were obtained in 7-52% yields. Because the N¹ isomers showed poor water solubility, we proceeded with the N² isomers in our subsequent studies. Since analogous 1,2,4-triazoles have also been used in designing small-molecule probes for serine hydrolases, we synthesized 1,2,4-triazole-based CATK-9 in four steps with an overall yield of 43%. Importantly, in NMR-based stability assays, CATKs exhibited excellent stability toward the reduced glutathione.

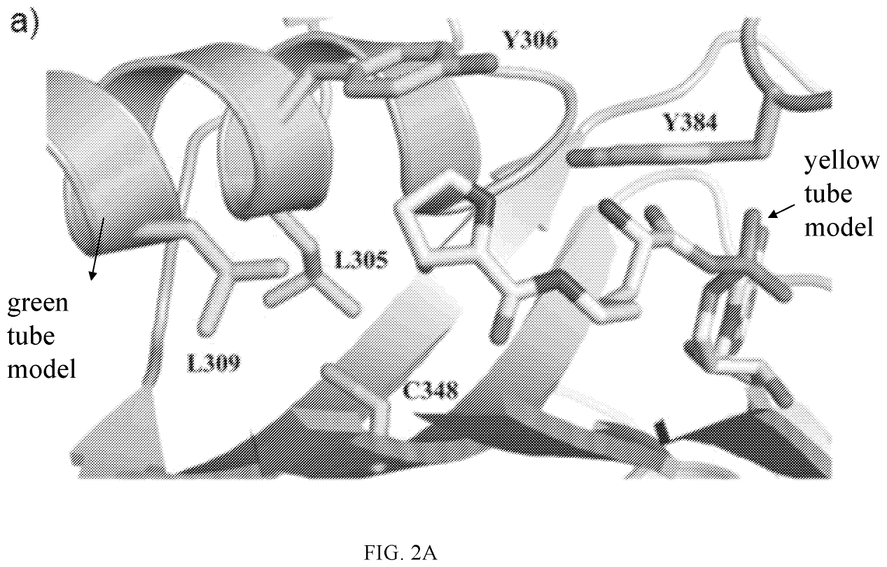

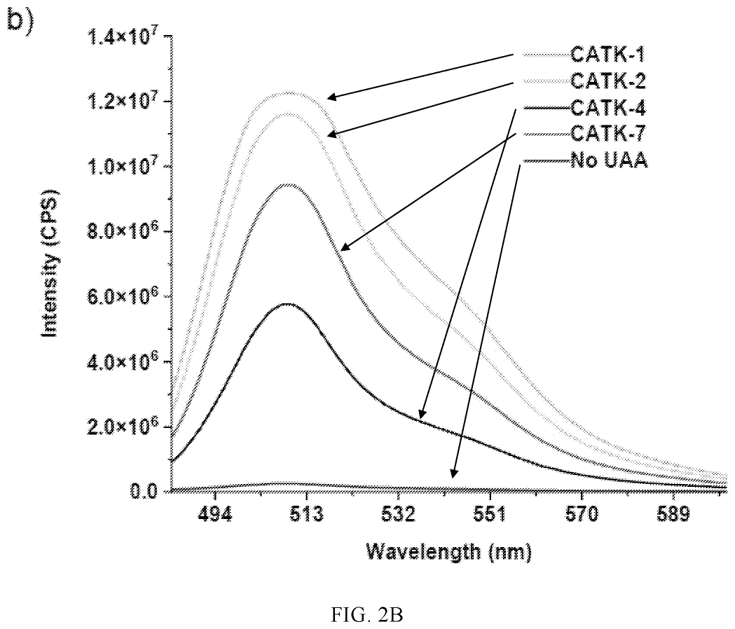

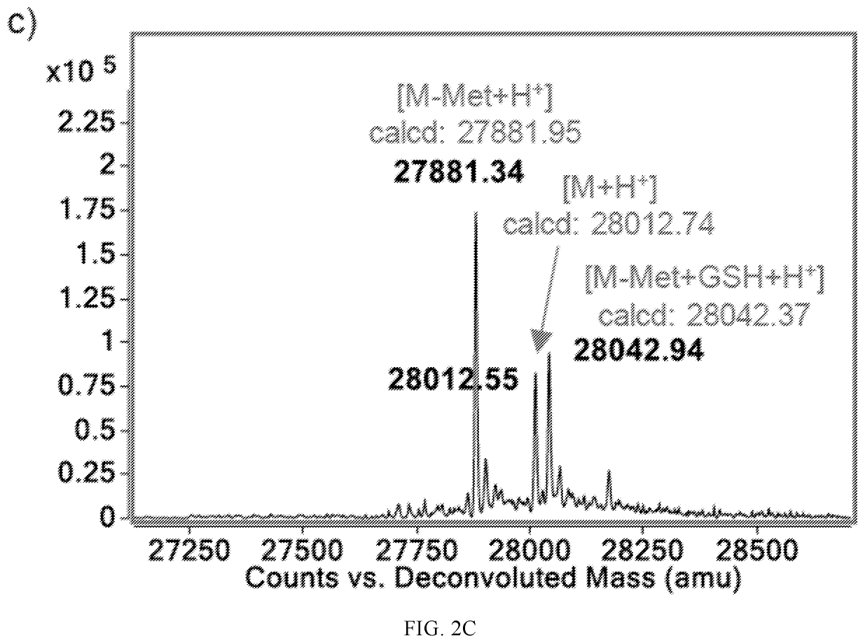

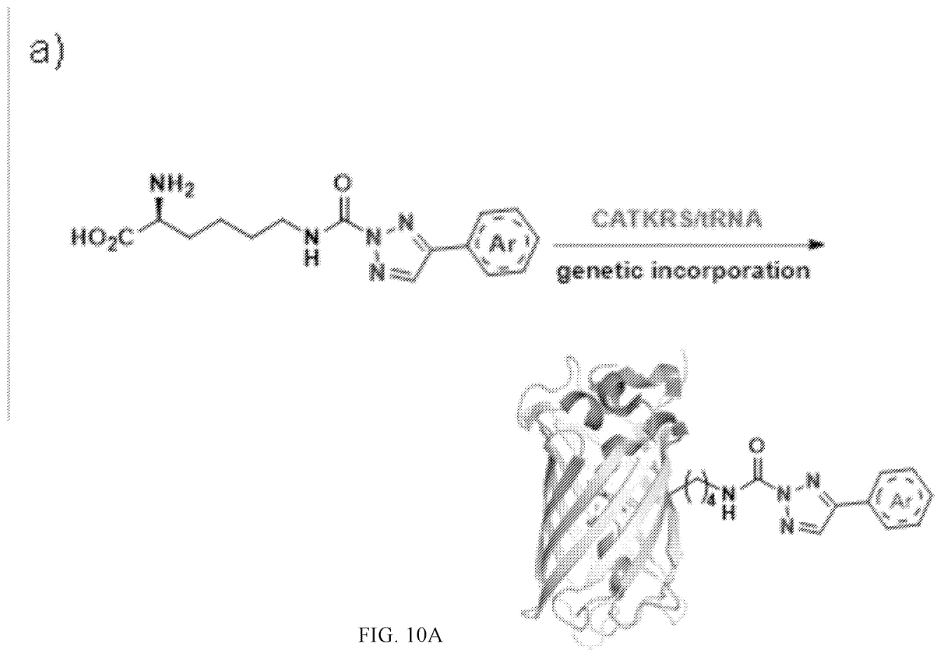

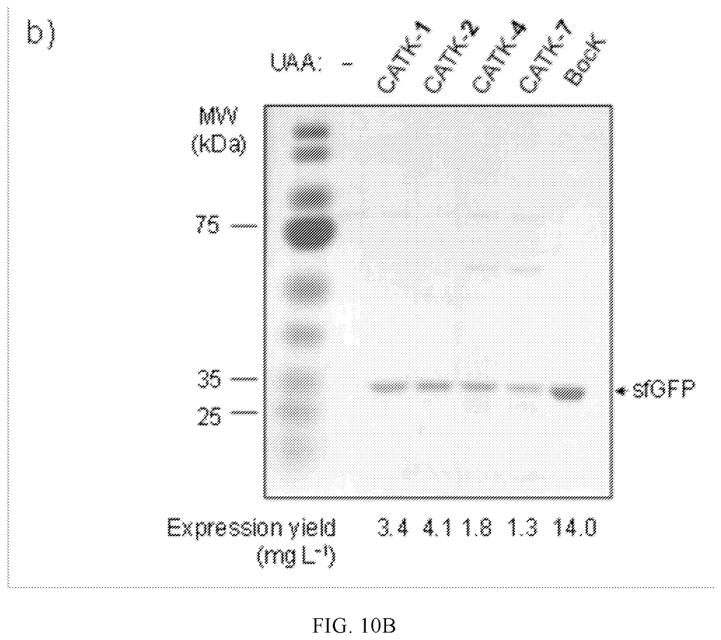

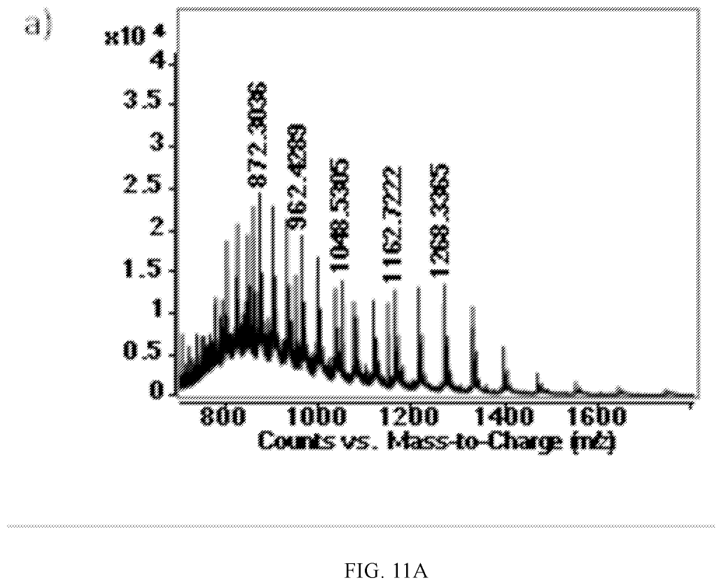

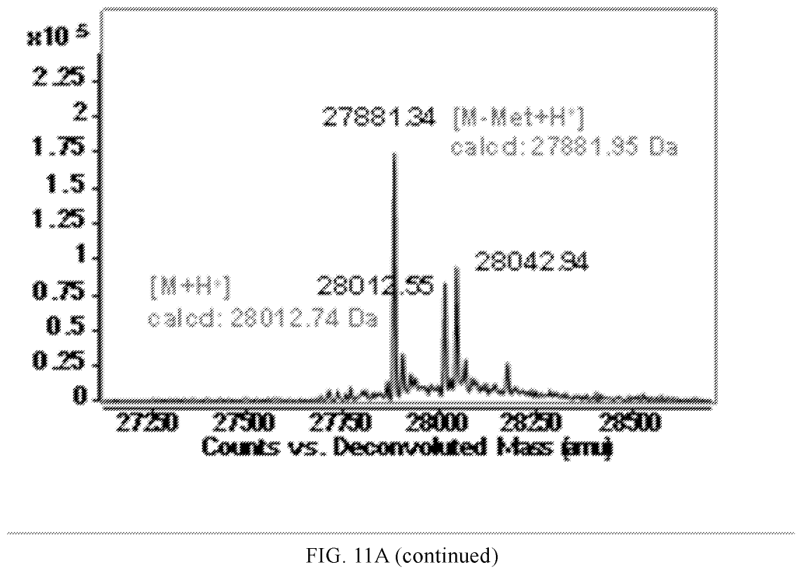

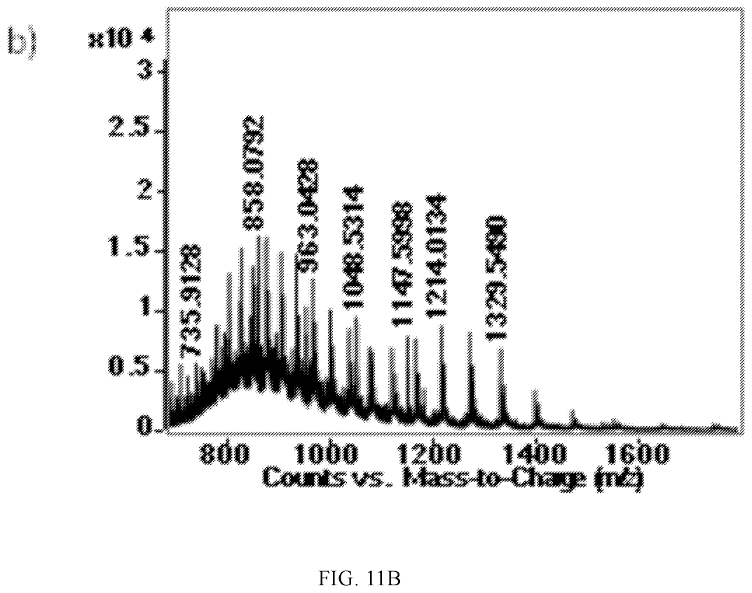

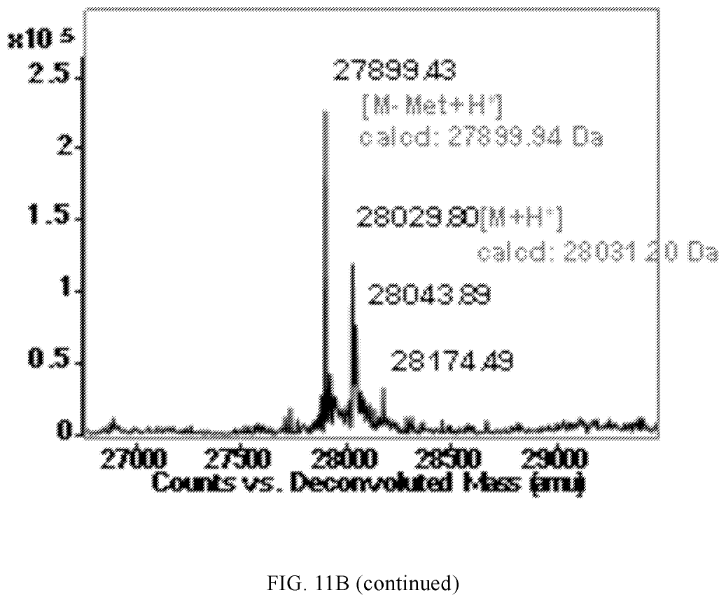

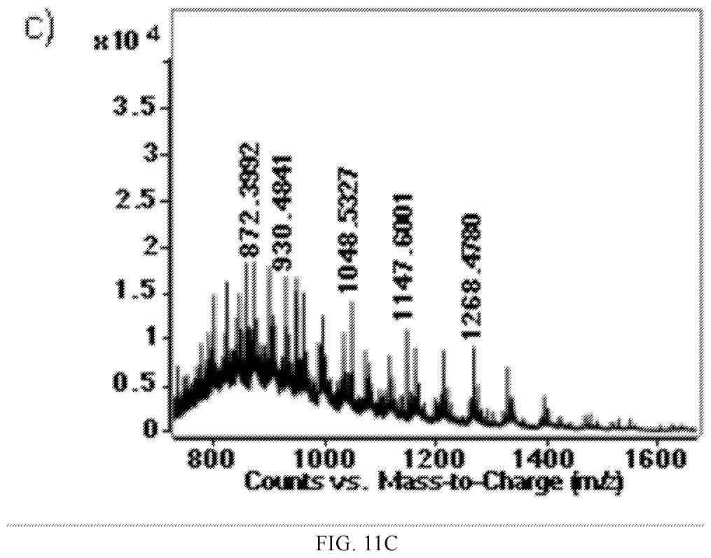

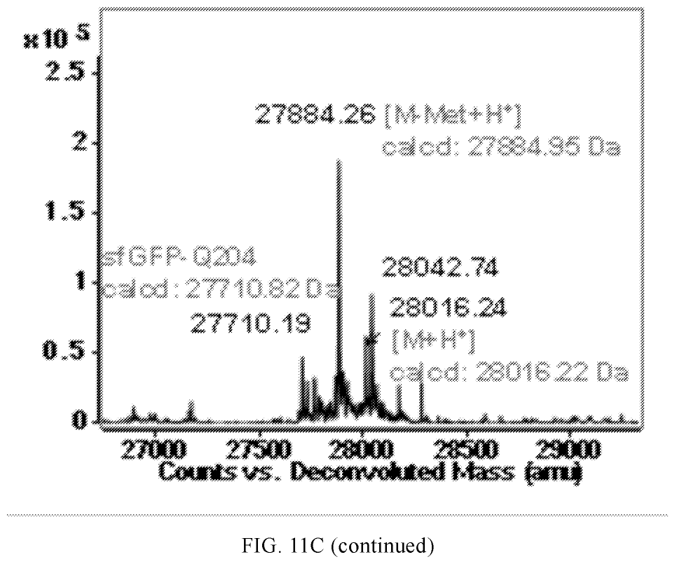

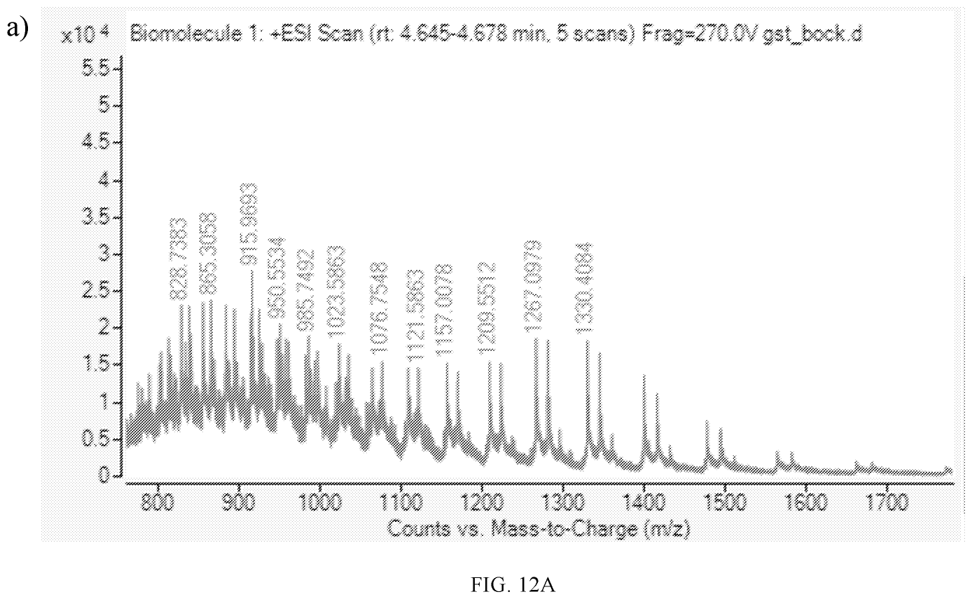

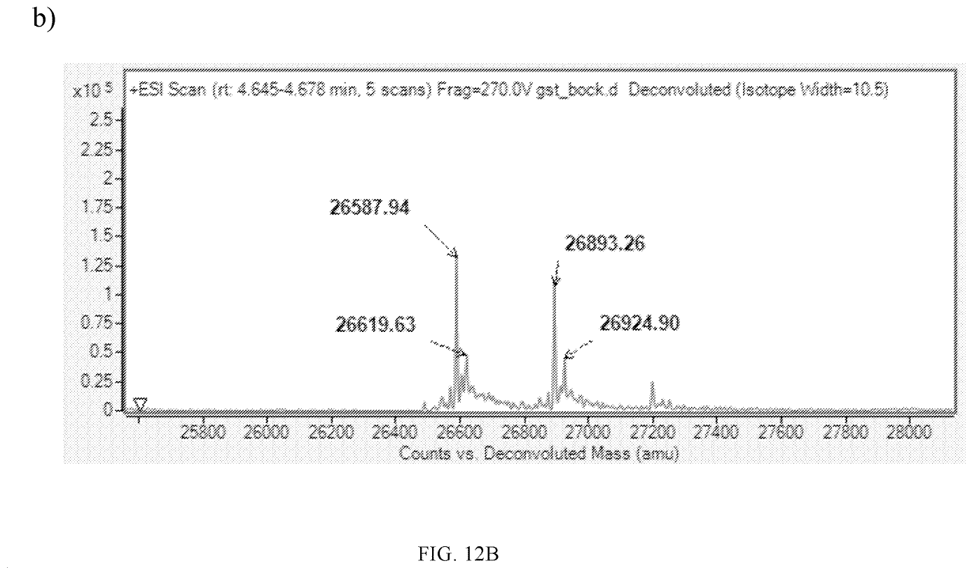

To identify pyrrolysine-tRNA synthetase (PylRS) variants that can charge CATKs, we co-transformed BL21(DE3) cells with two plasmids: pEVOL-PylRS encoding PylRS and tRNA_{CUA}, and pET-sfGFP-Q204TAG encoding sfGFP bearing an amber codon. We screened a panel of PylRS variants (Table 1), and found one carrying Y306V, L309A, C348F, and Y384F mutations, hereafter referred to as CATKRS, can charge CATK-1, -2, -4, and -7 site-specifically into sfGFP (FIGS. 2b and 10). The incorporations were also confirmed by SDS-PAGE (FIG. 10) and QTOF-LC/MS analyses (FIGS. 2c and 11). Some amount of GSH adducts (˜30%) were detected, presumably due to the high reactivity of CATKs at position-204, a site on sfGFP that is completely solvent exposed. However, no hydrolysis products were observed, indicating that CATKs are stable under bacterial culture conditions.

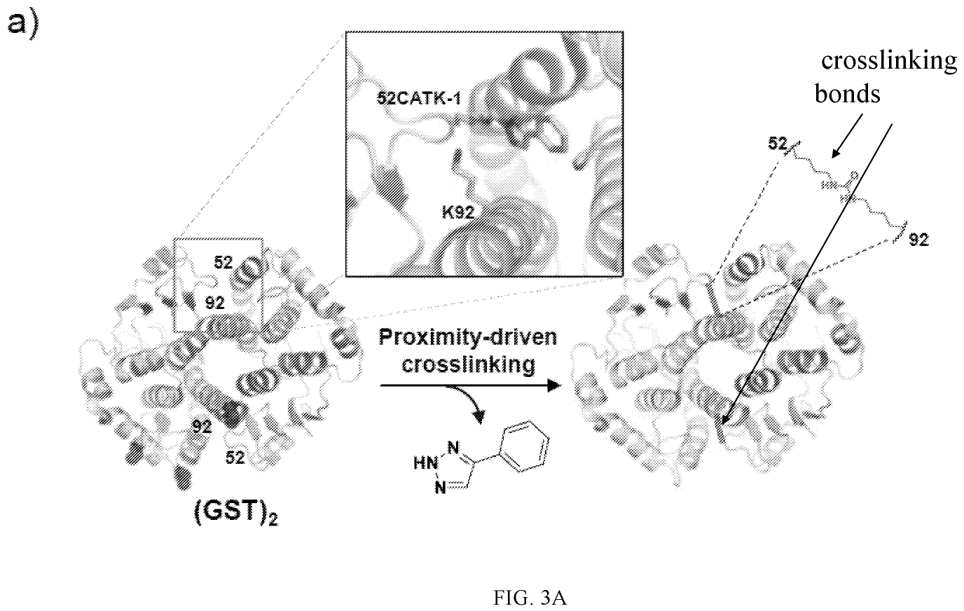

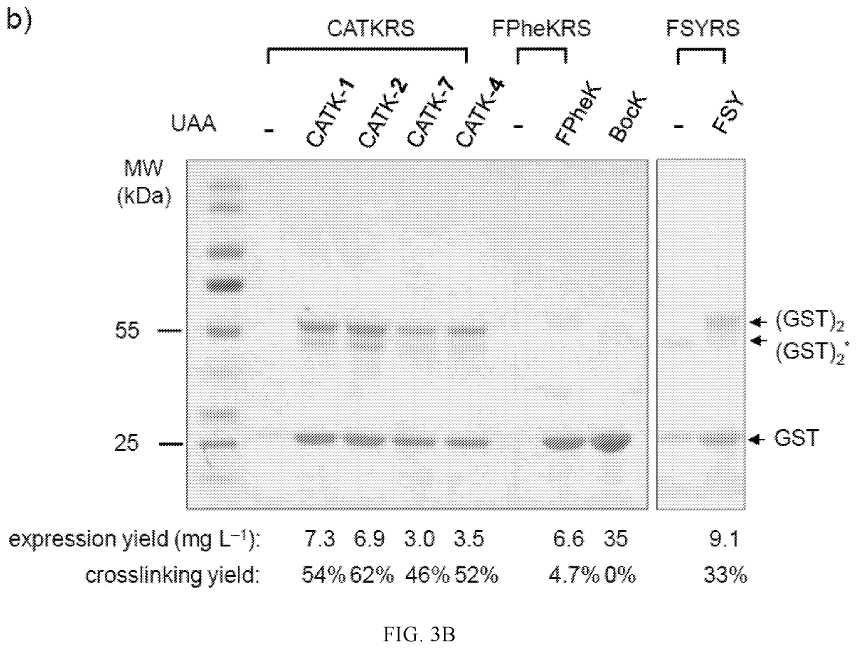

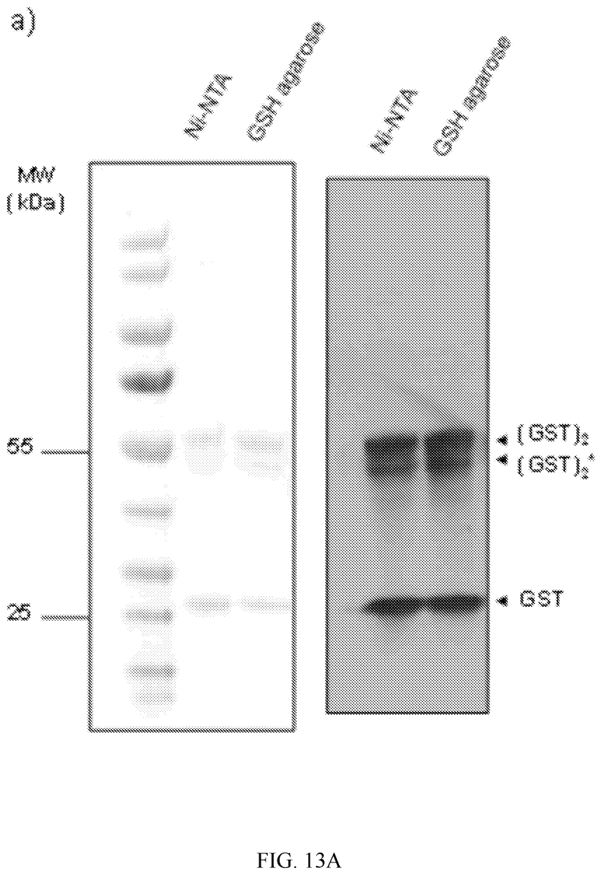

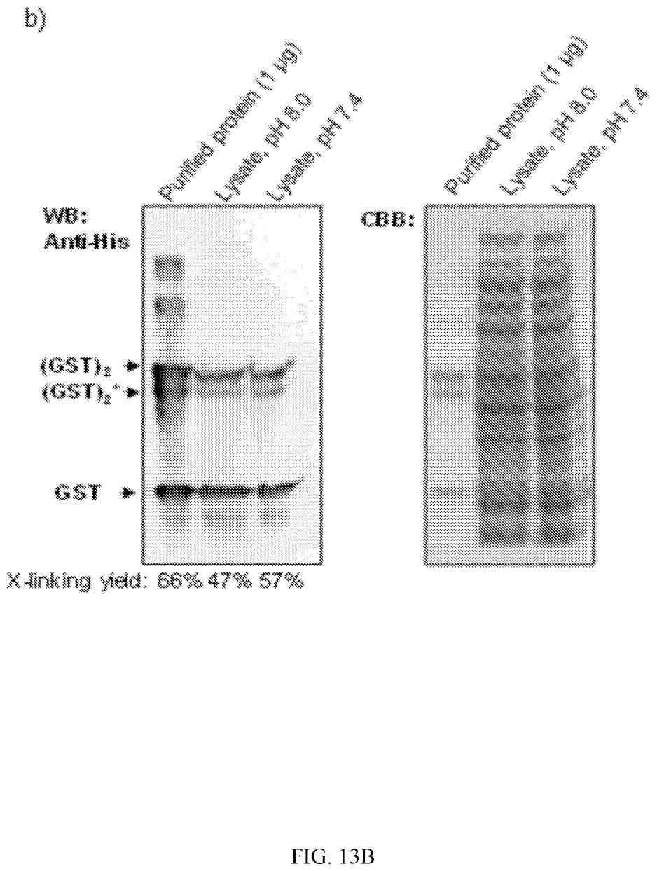

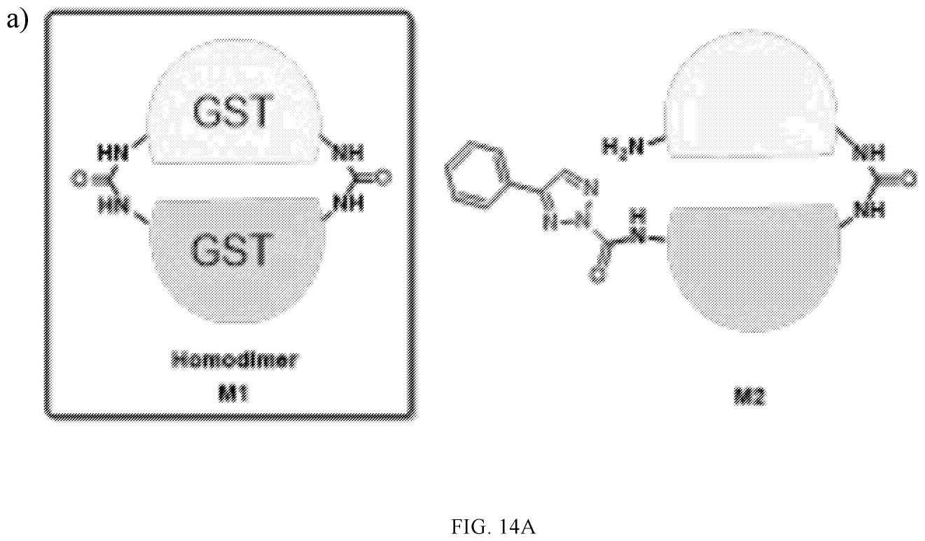

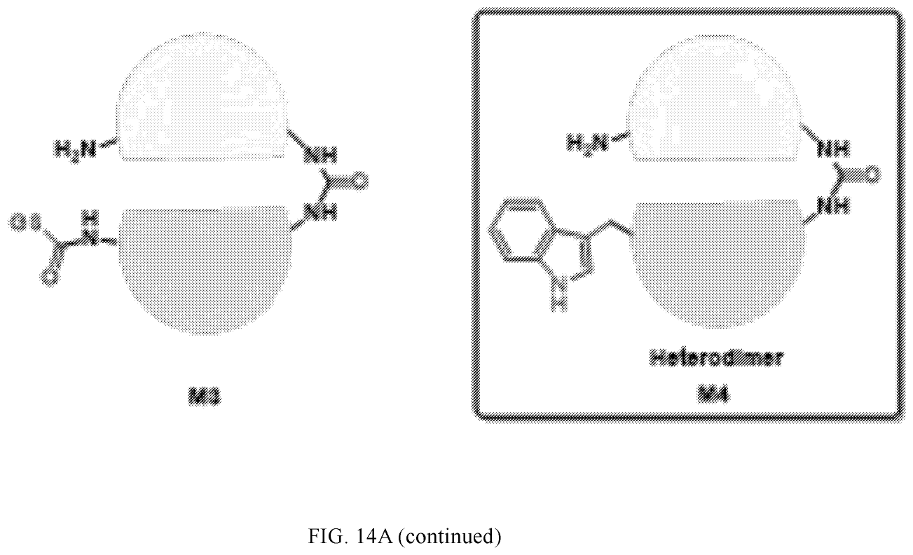

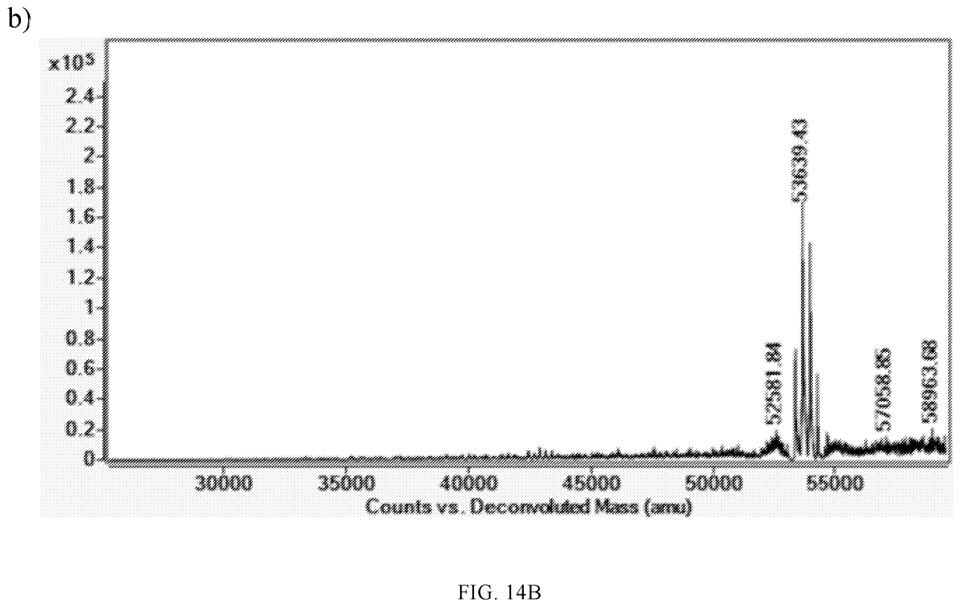

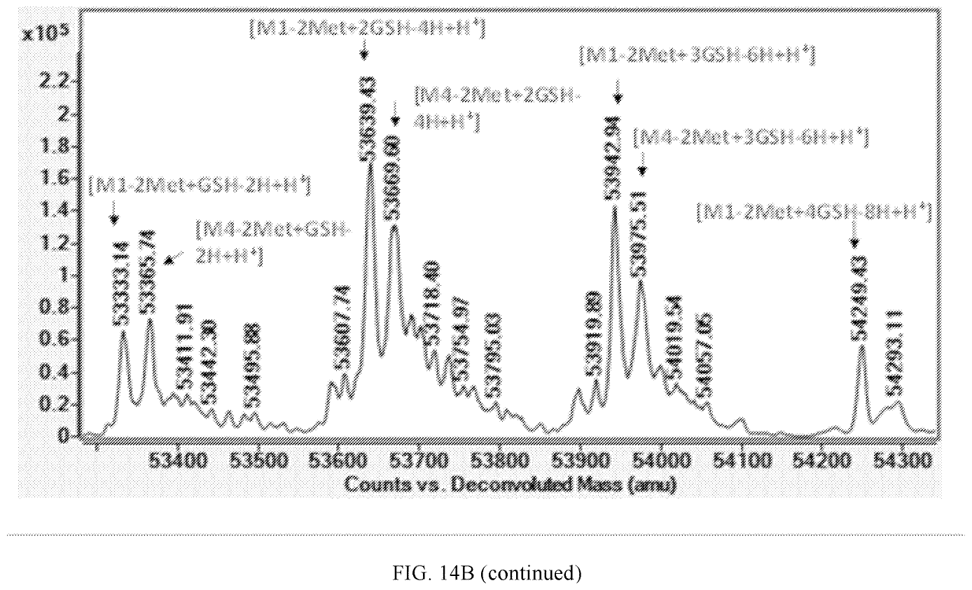

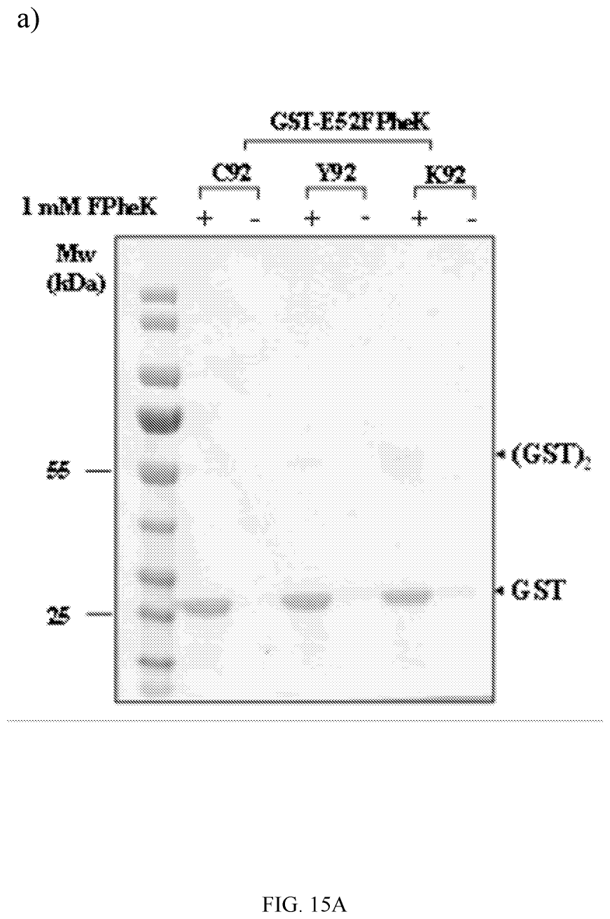

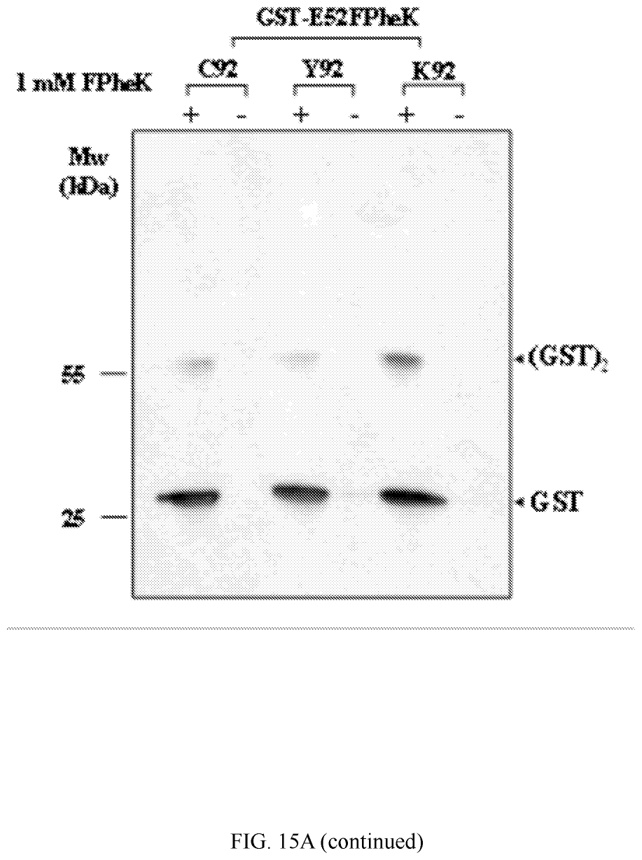

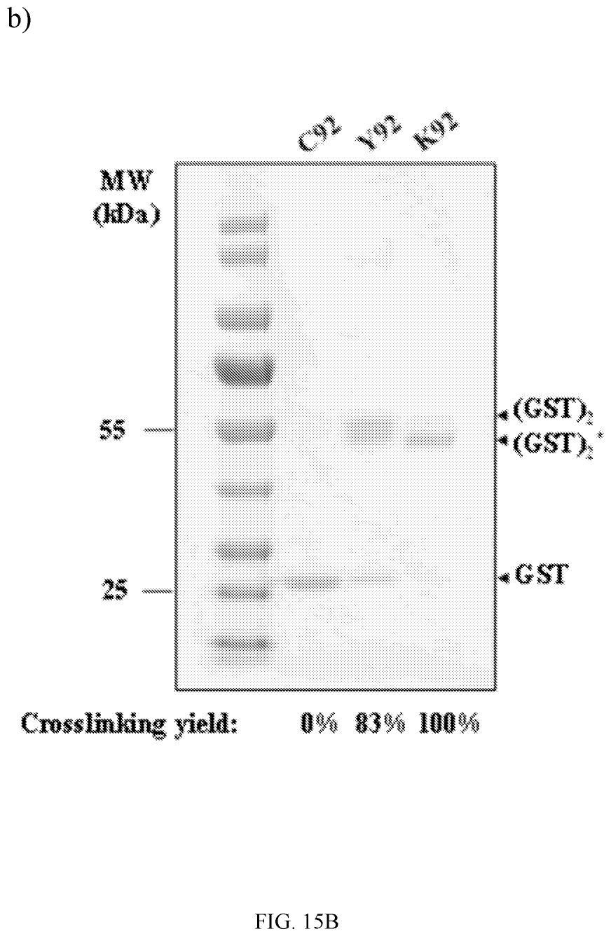

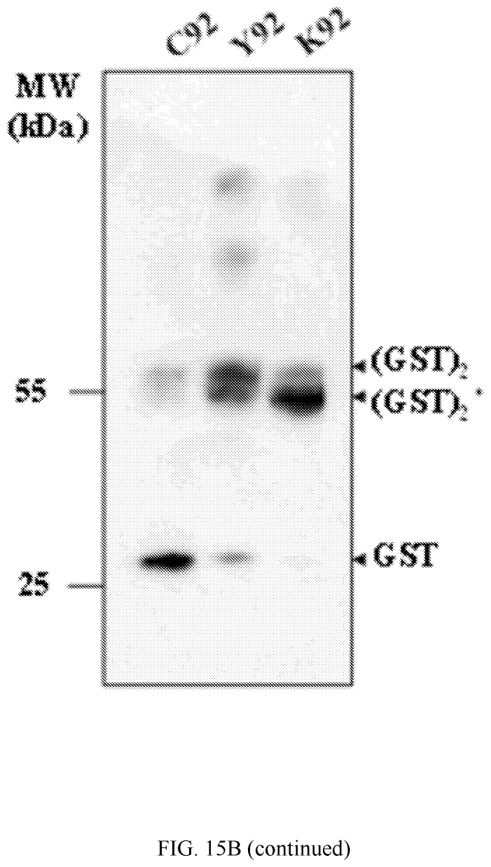

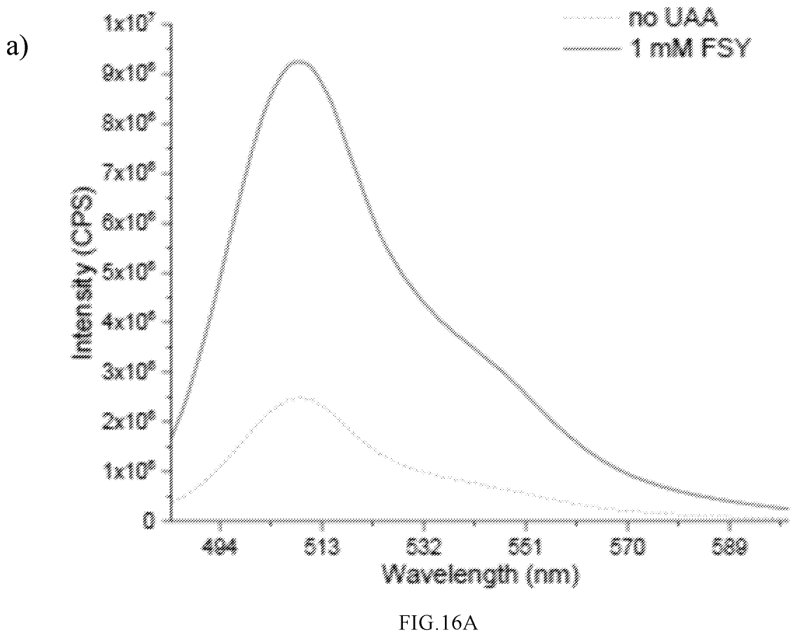

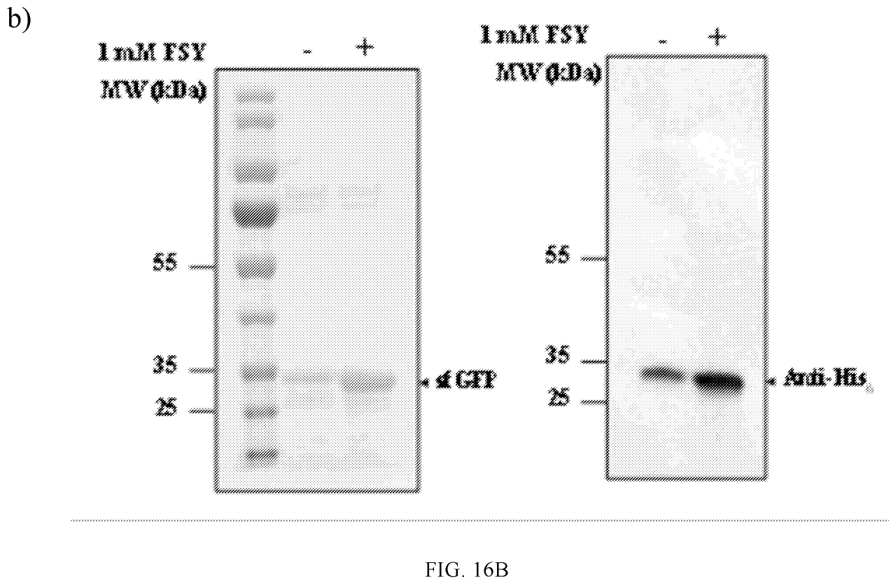

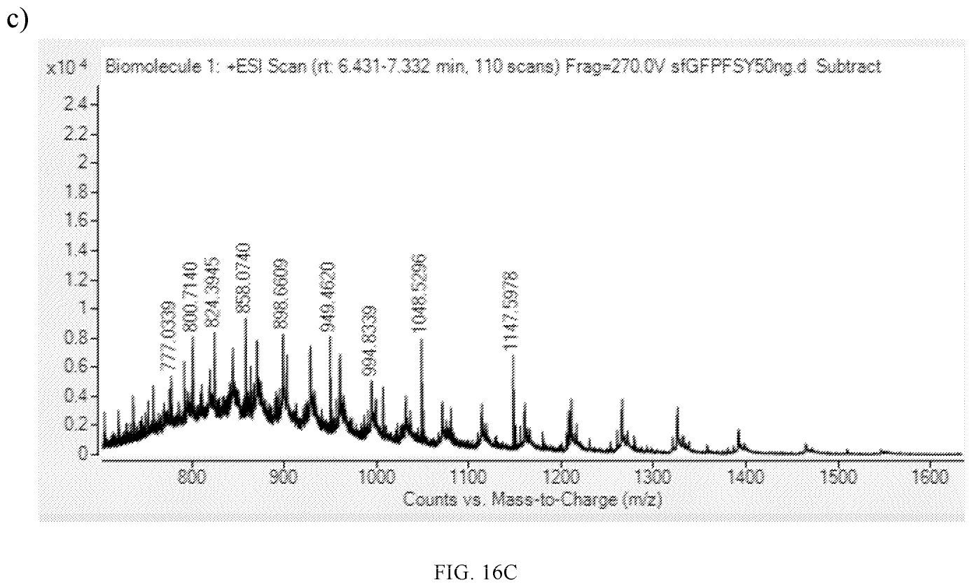

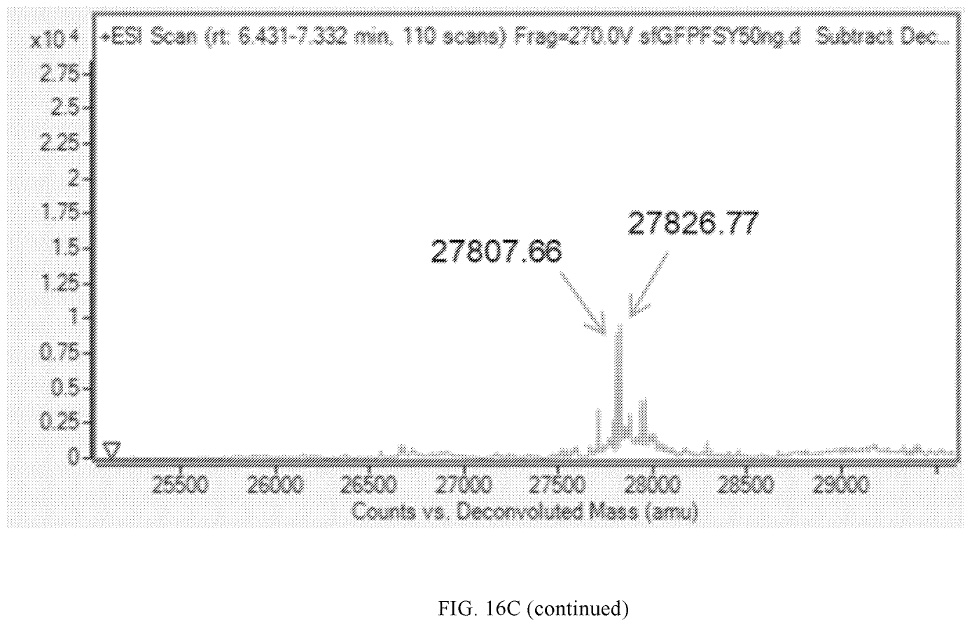

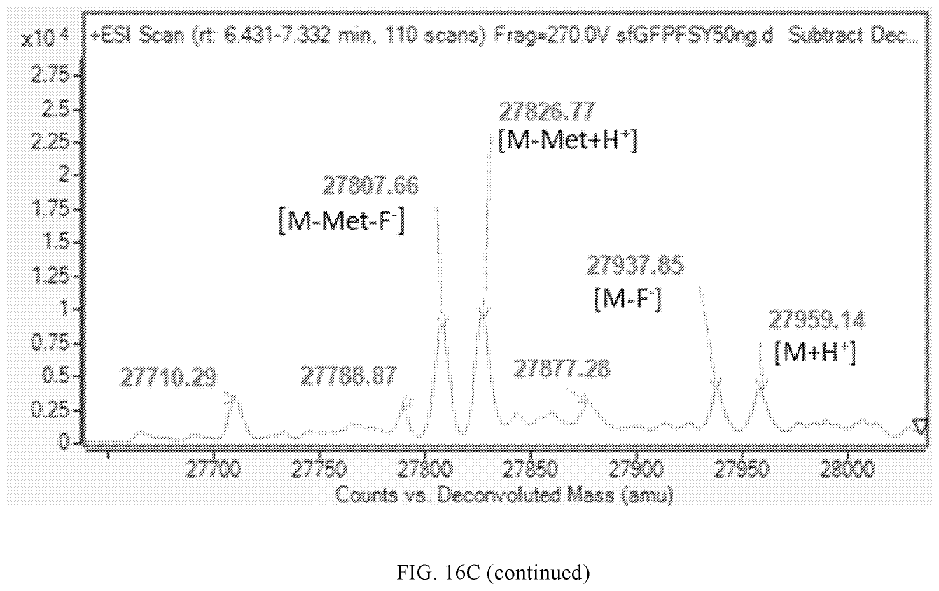

To assess CATK crosslinking reactivity, we decided to use the glutathione-S-transferase (GST) as a model because GST exists naturally as a homodimer and has been used previously for evaluating electrophilicity of noncanonical amino acids. Thus, we expressed GST mutants by placing CATK at position-52 and Lys at position-92 with anticipation that the flexible alkyl amine of Lys-92 will displace the triazole in a proximity-dependent acyl transfer reaction to generate the covalent GST dimer (FIG. 3a). The GST mutants encoding CATK-1, -2, -4, and -7 at position-52 were obtained in good yields (3.0-7.3 mg L⁻¹). To our satisfaction, prominent dimer bands were detected for all four CATK-encoded GST mutants on SDS-PAGE gel (FIG. 3b), which was corroborated by western blot analysis (FIG. 13a). Neither buffer exchange nor prolonged incubation was needed (FIG. 13b), suggesting that the crosslinking occurred inside bacterial cells. Notably, the four cysteines present in each GST monomer (FIG. 3a) do not interfere with CATK-1-mediated orthogonal crosslinking. As a control, N^{e}-(tert-butoxycarbonyl) lysine (BocK)-encoded GST mutant did not produce any covalent dimers, indicating that CATK is responsible for the cross-linking (FIG. 3b). In contrast, GST mutants encoding FPheK or FSY at position-52 showed lower covalent dimer formation under the same condition, suggesting that CATK is a superior crosslinking motif (FIGS. 3b, 15-17).

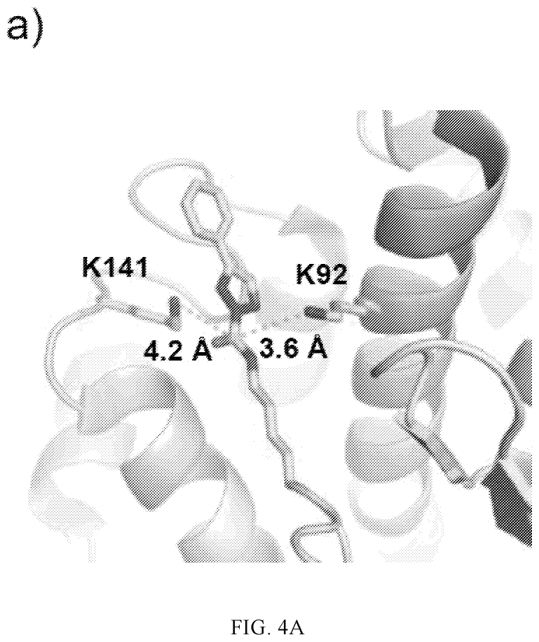

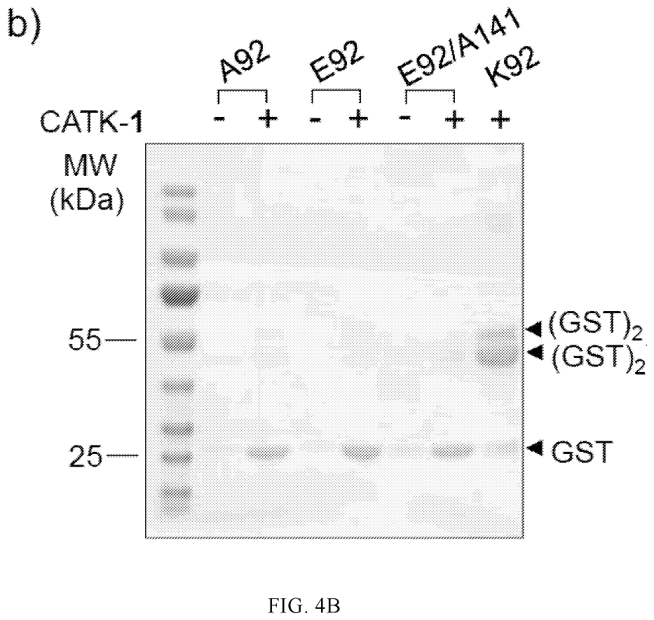

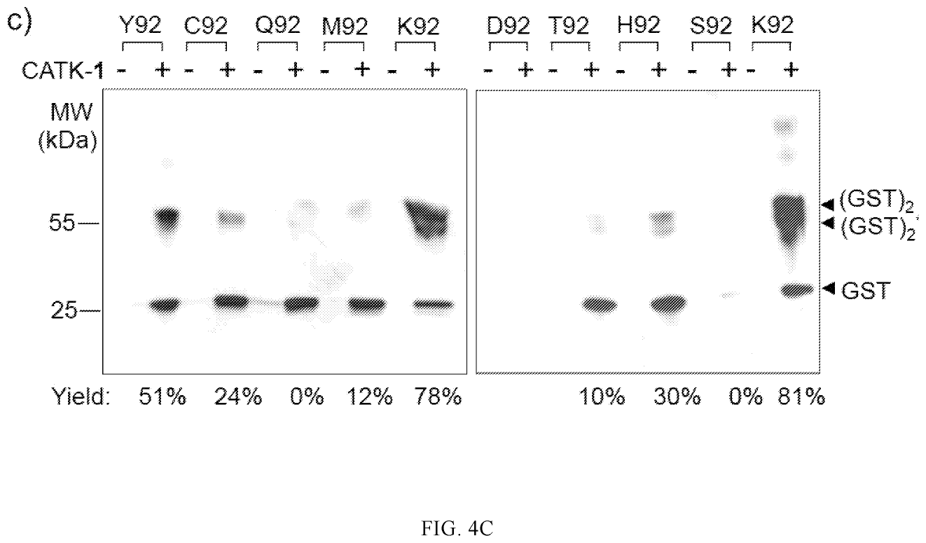

To identify residues responsible for crosslinking with CATK, we built a model of GST-E52CATK-1-K92 based on the GST dimer structure. Upon considering the distance and orientation of the residues surrounding CATK-1, we identified K92 and K141 as the plausible reaction partner (FIG. 4a). We then mutated K92 to either Ala or Glu and observed complete abolishment of dimer formation on the SDS-PAGE gel (FIG. 4b), indicating that K92 is responsible for the proximity-driven crosslinking. Finally, to examine whether amino acids other than Lys may participate in this proximity-driven crosslinking, we mutated Lys-92 to Tyr, Cys, Gln, Met, Asp, Thr, His, and Ser. Among six GST mutants that were expressed successfully, only the Tyr mutant gave a comparable crosslinking yield while the Cys and His mutants afforded modest crosslinking (FIG. 4c). Together, lysine and tyrosine appear to represent the two most suitable reaction partners for CATK-1 in the nucleophilic acyl transfer reaction, likely due to their extended side chains and high intrinsic reactivity.

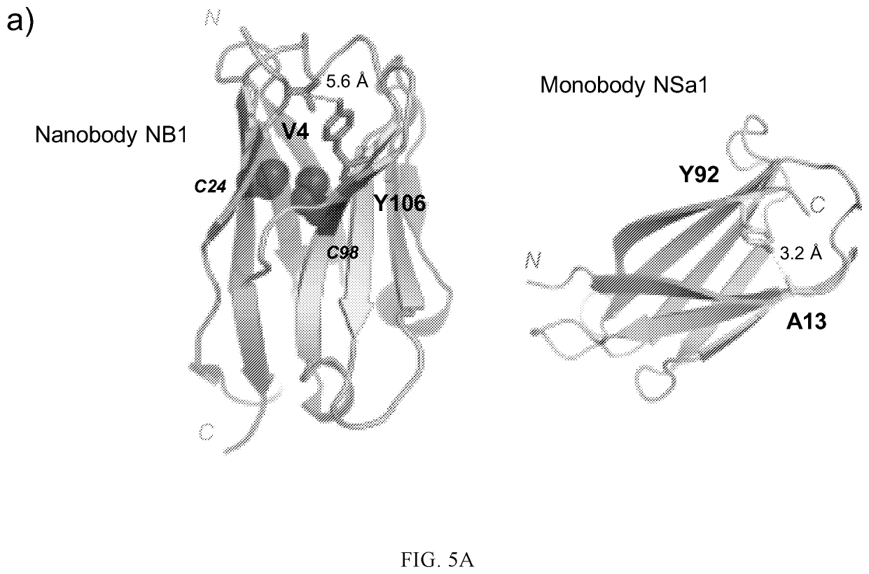

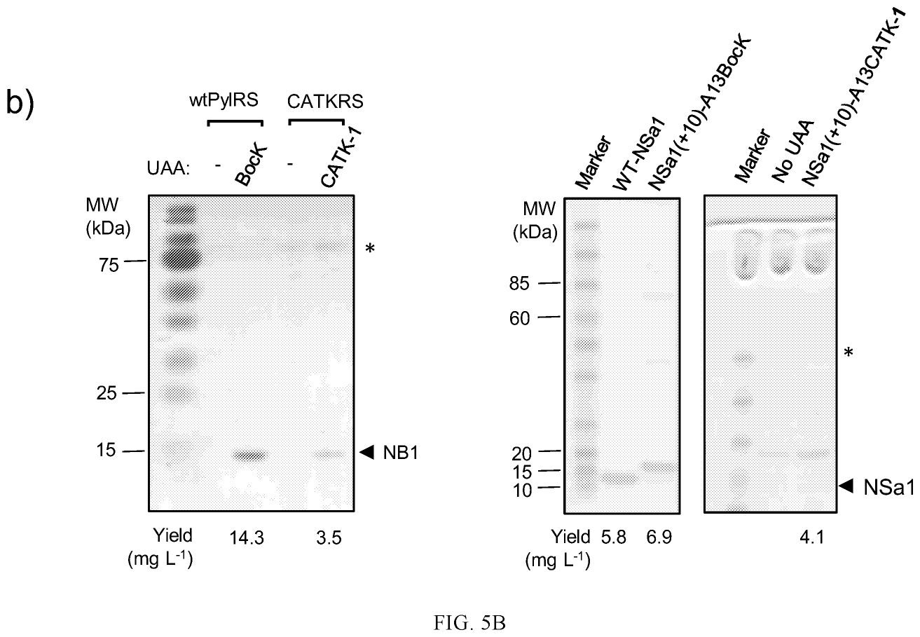

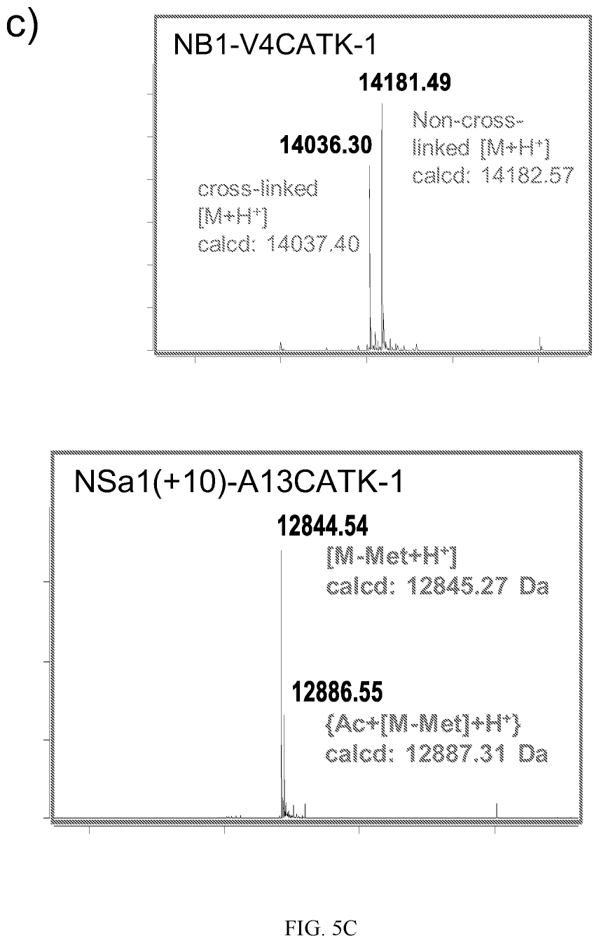

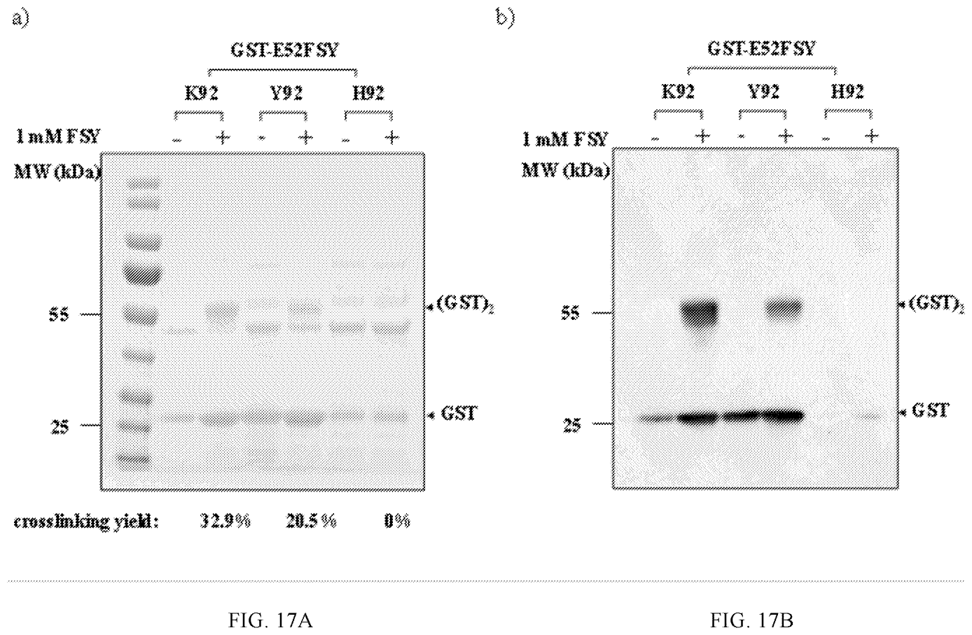

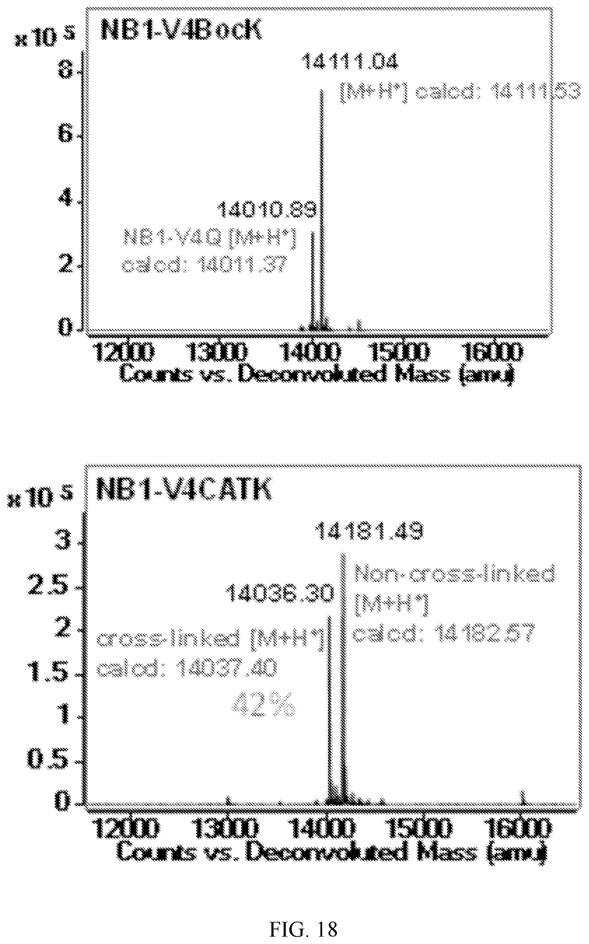

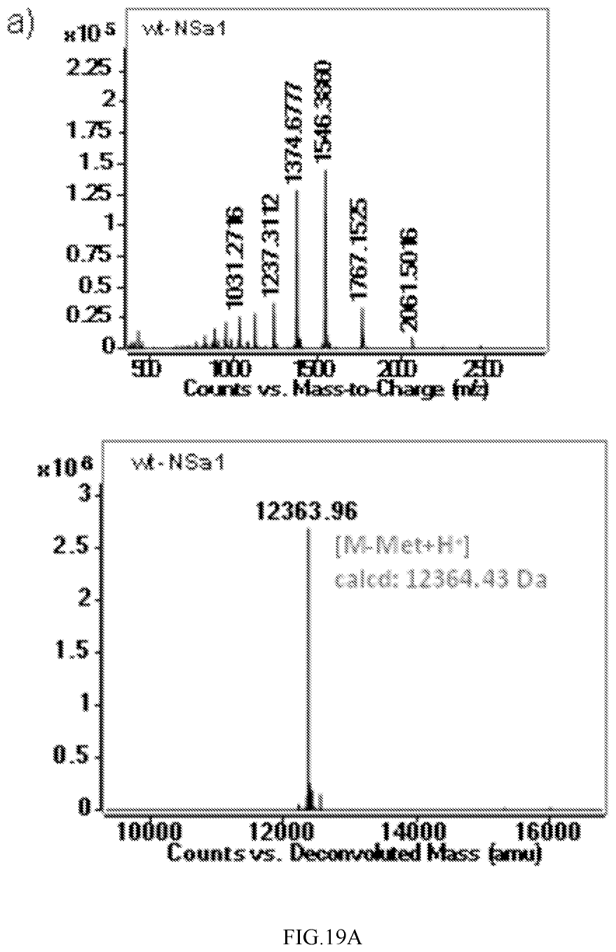

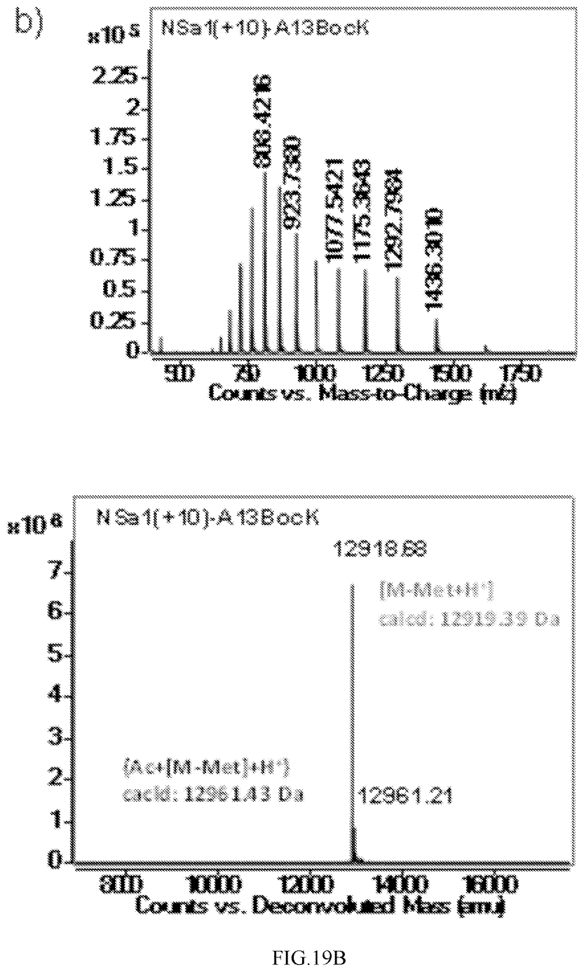

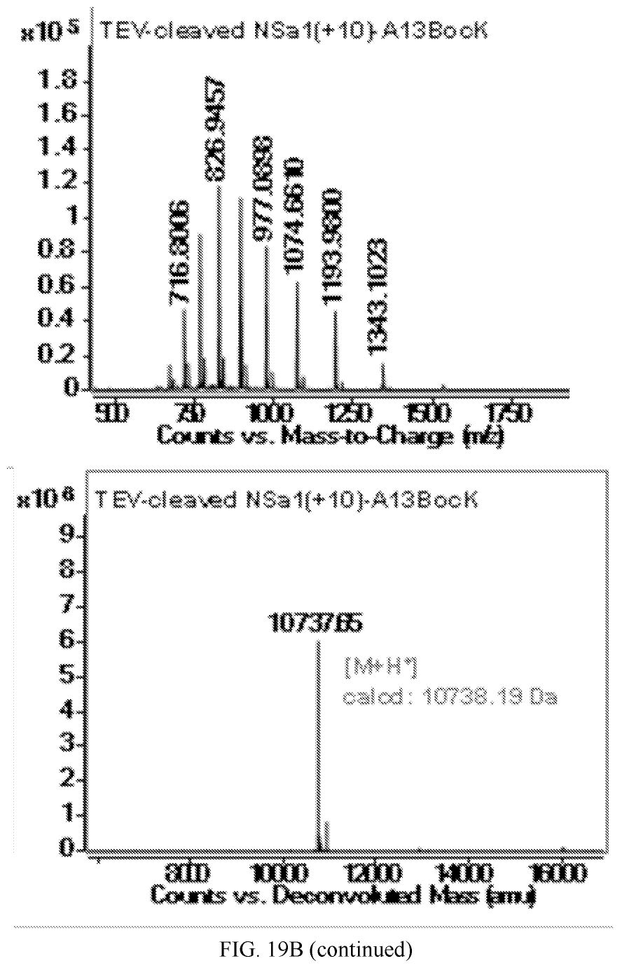

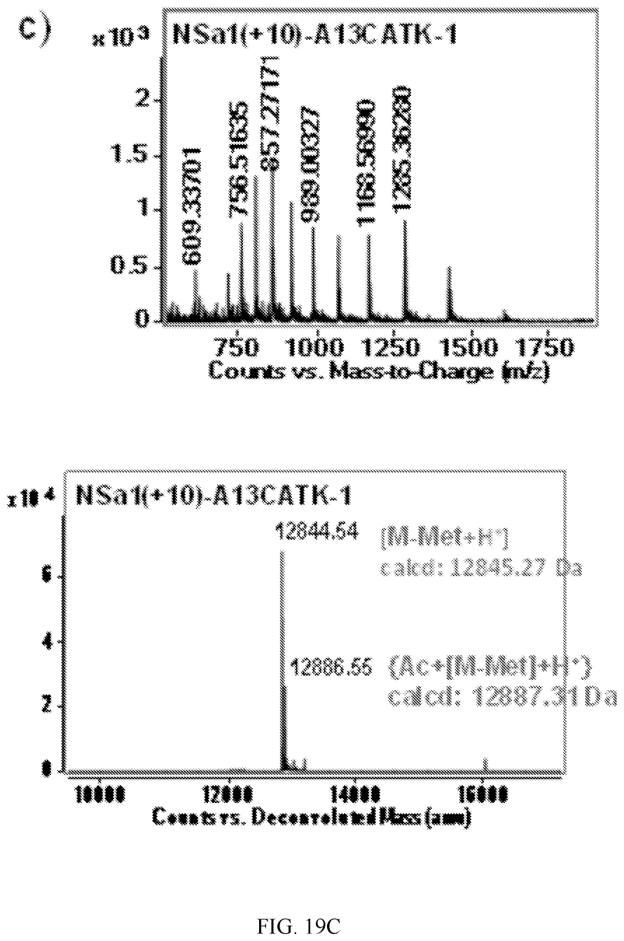

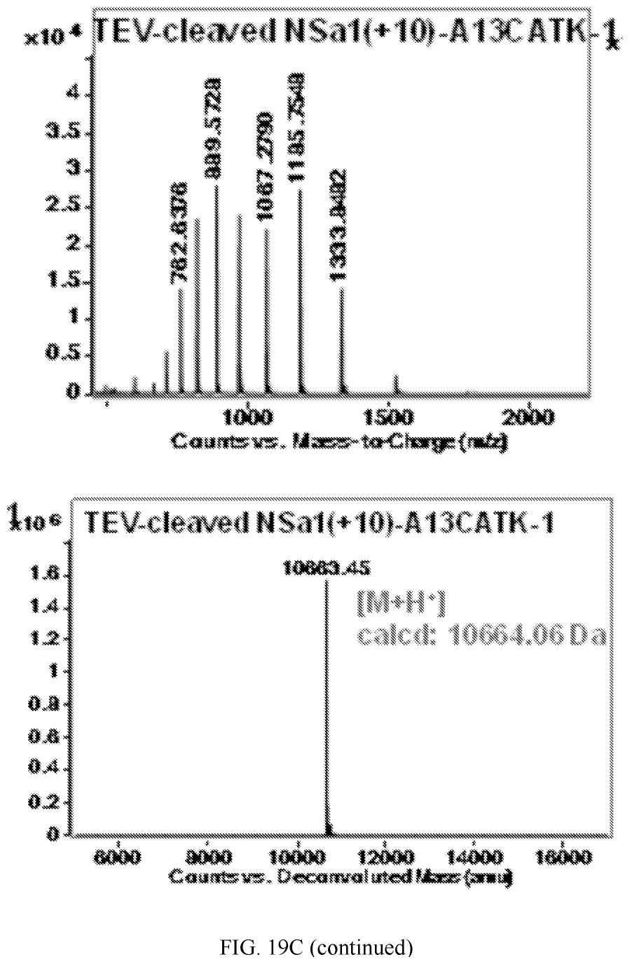

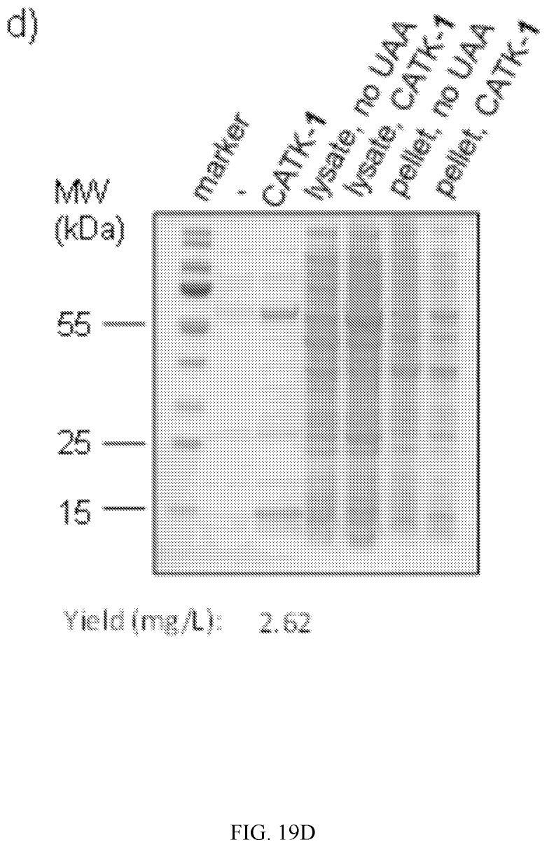

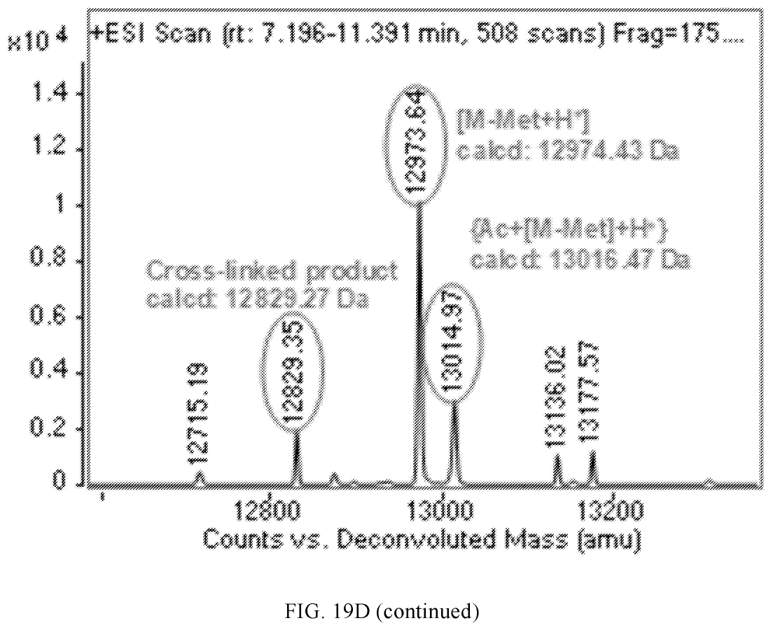

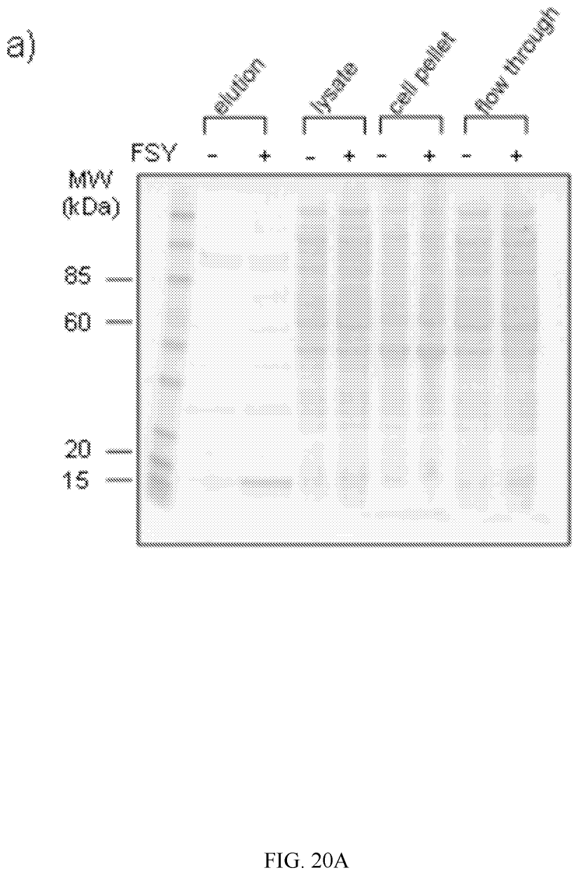

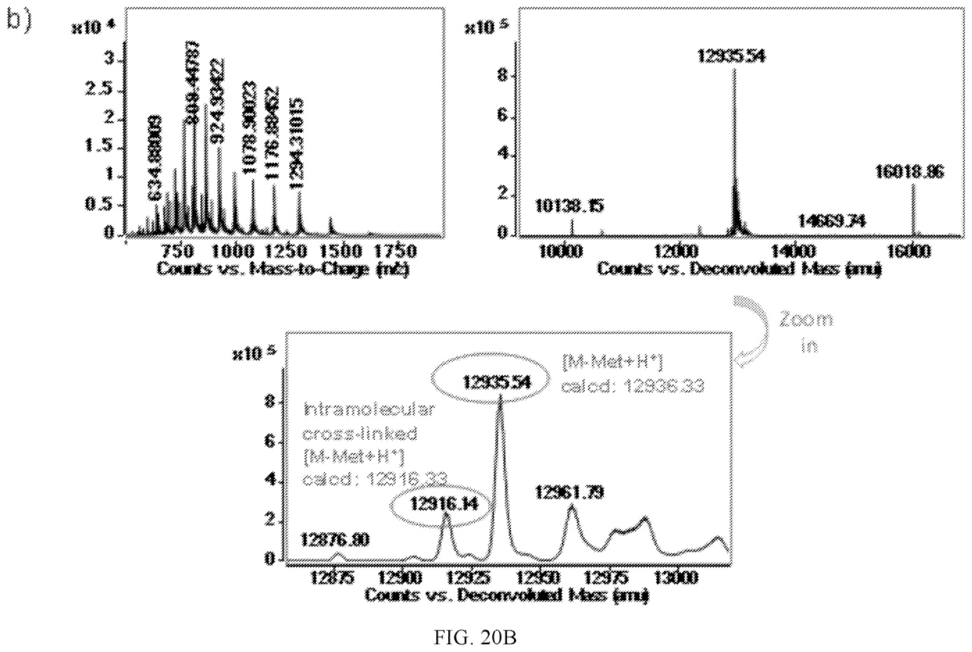

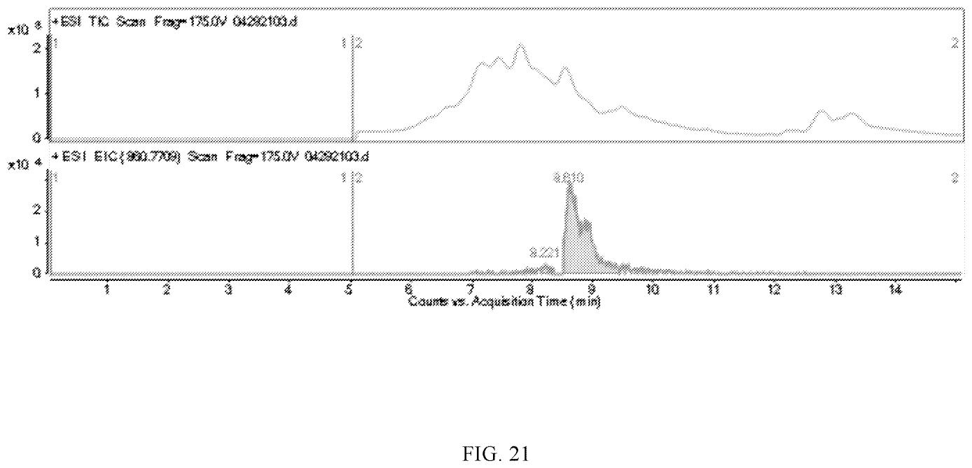

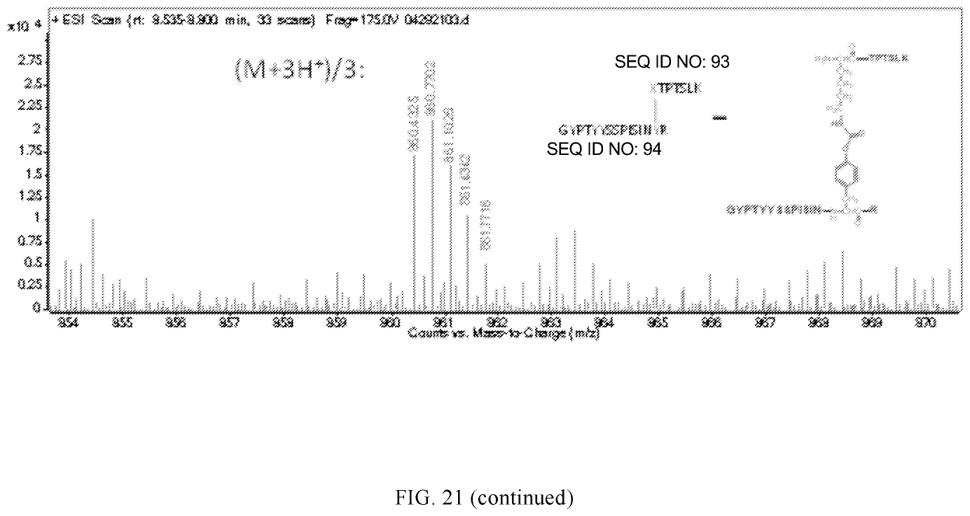

To probe whether CATK-1 is suitable for inter-strand cross-linking in proteins containing the disulfide bond, we selected a small protein called nanobody NB1, a prototypical single-chain VHH antibody that binds specifically to GFP protein. Based on NB1 structure, there is one disulfide bond formed between Cys-24 and Cys-98, close to a proposed orthogonal crosslinking site at Val-4 and Tyr-106 (FIG. 5a, left). To test orthogonal crosslinking, we placed CATK-1 at Val-4 position to target Tyr-106 located 5.6 Å away on the opposing strand. The BocK- and CATK-1-encoded NB1 were successfully expressed at yields of 14.3 mg L⁻¹ and 3.5 mg L⁻¹, respectively (FIG. 5b, left). The deconvoluted intact masses showed a 42% crosslinking yield (FIG. 5c, left; FIG. 17). Notably, no GSH adduct, hydrolysis product, or the side product from the cysteine reaction with CATK-1 was detected, indicating that CATK-1-mediated crosslinking is orthogonal to the disulfide bond. Separately, we also examined the utility of CATK-1 in effecting intramolecular crosslinking in an antibody mimic called monobody. Due to their lack of cysteine residues, small size (˜10 kDa), and evolvable binding affinity and specificity, monobodies represent an ideal protein scaffold for targeting protein-protein interactions in the cytosols of mammalian cells. However, monobodies are cell impermeable, severely limiting their potential. One strategy to potentially overcome this limitation is to combine protein surface supercharging with orthogonal crosslinking to increase stability in the endosomes and thus improve cytosolic delivery. To this end, we designed an overall +10 charged monobody NSa1, termed NSa1(+10), using the Supercharge protocol on ROSIE Rosetta Online Server and added an amber codon at Ala-13 position. Based on the NSa1 structure, A13CATK-1 is well-positioned to react with the proximal Tyr-92 on the opposing strand at C-terminus (FIG. 5a, right). Accordingly, the wild-type and NSa1(+10) mutant proteins encoding CATK-1 or BocK were expressed and purified in good yields (4.1-6.9 mg L¹; FIG. 5b, right). To our delight, mass spectrometry analysis indicated that the inter-strand cross-linking yield between CATK-1 and Tyr-92 was essentially quantitative (FIG. 5c, right; FIG. 19), which was substantially higher than the FSY mutant giving 27.5% yield (FIG. 20). The crosslink-containing fragment was identified by LC/MS after trypsin digestion (FIG. 21). Furthermore, when Tyr-92 was mutated to Phe, the crosslinking yield dropped to 9.5% (FIG. 19d), indicating that Tyr-92 is the primary site for the proximity-driven crosslinking.

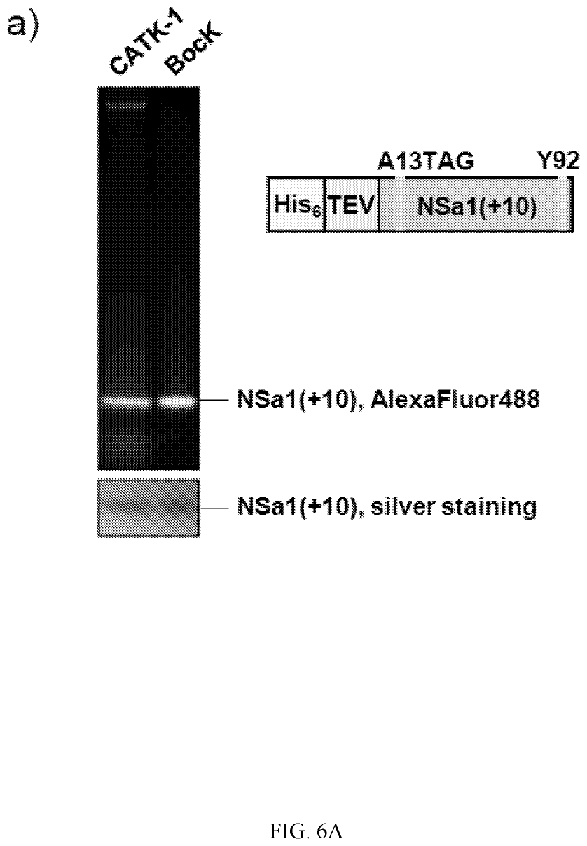

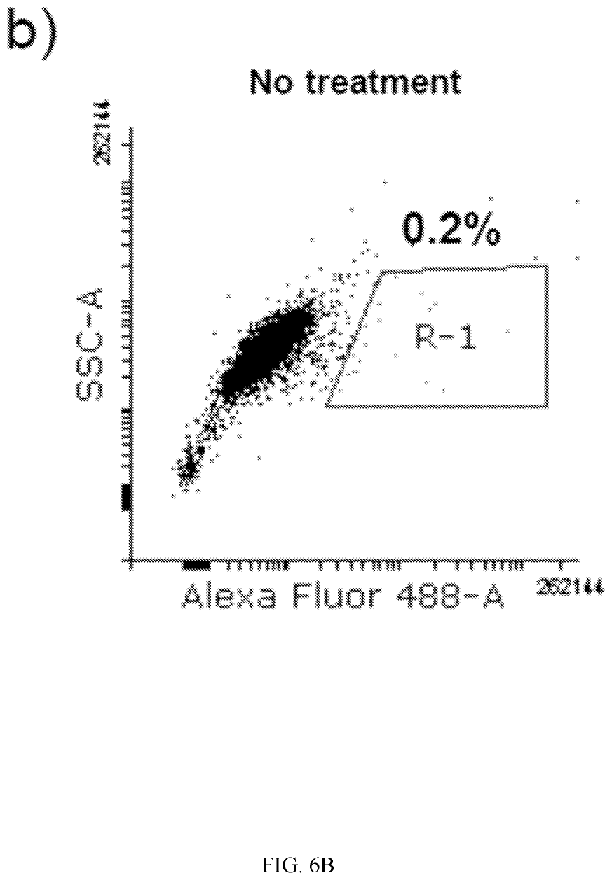

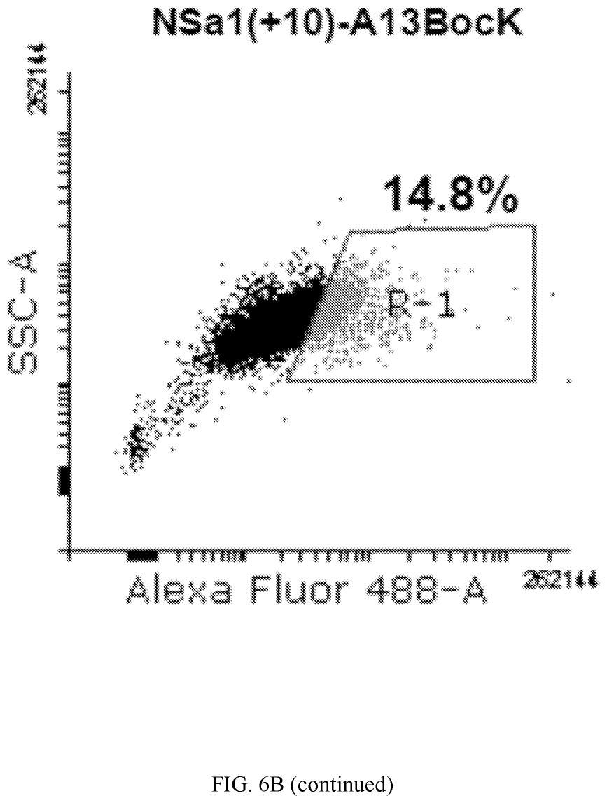

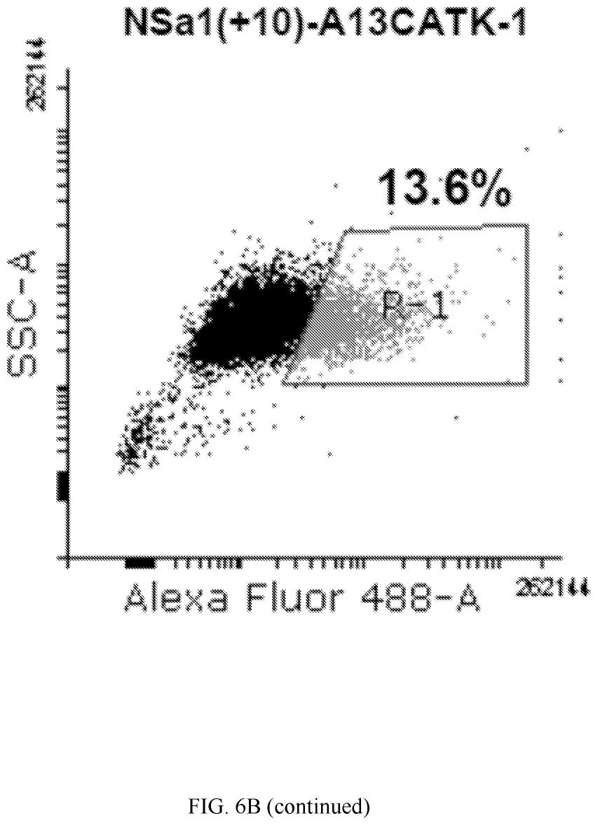

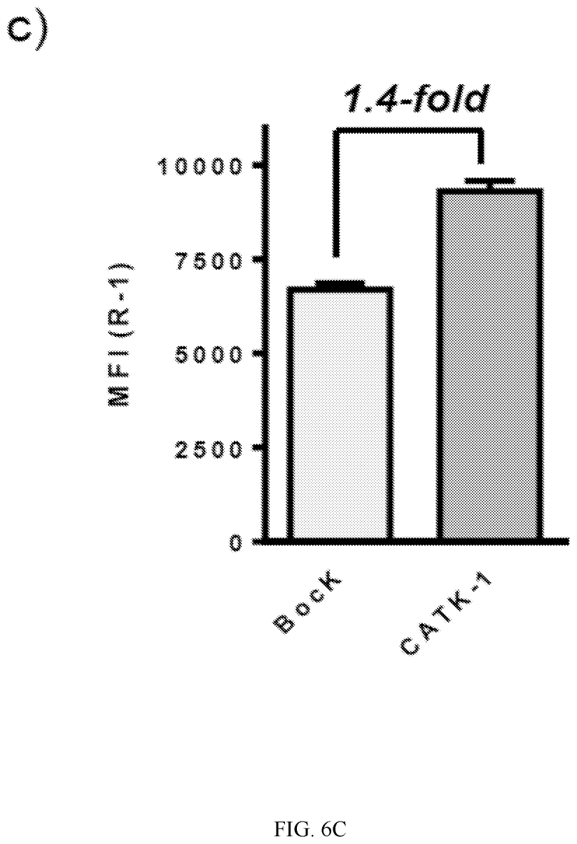

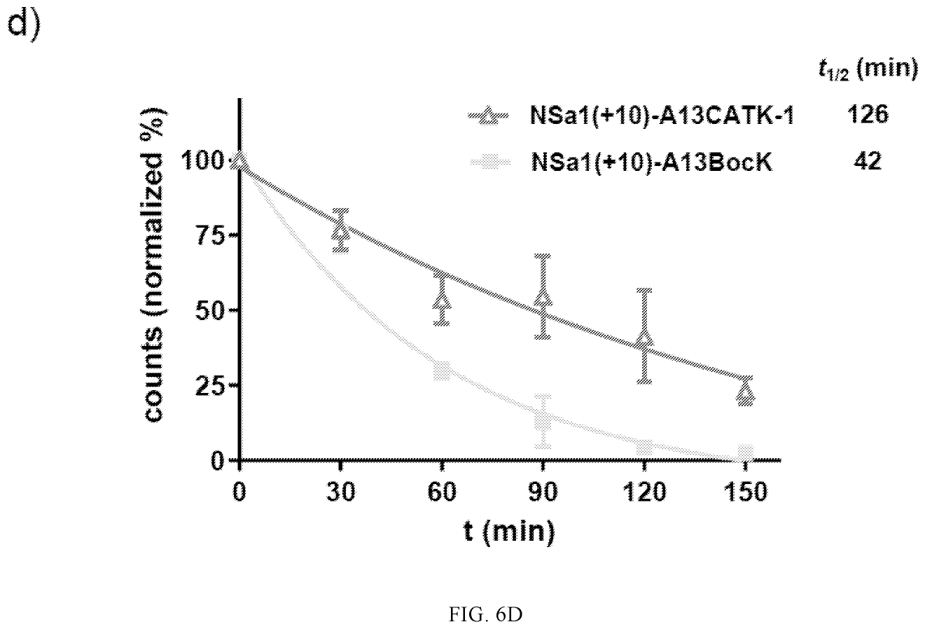

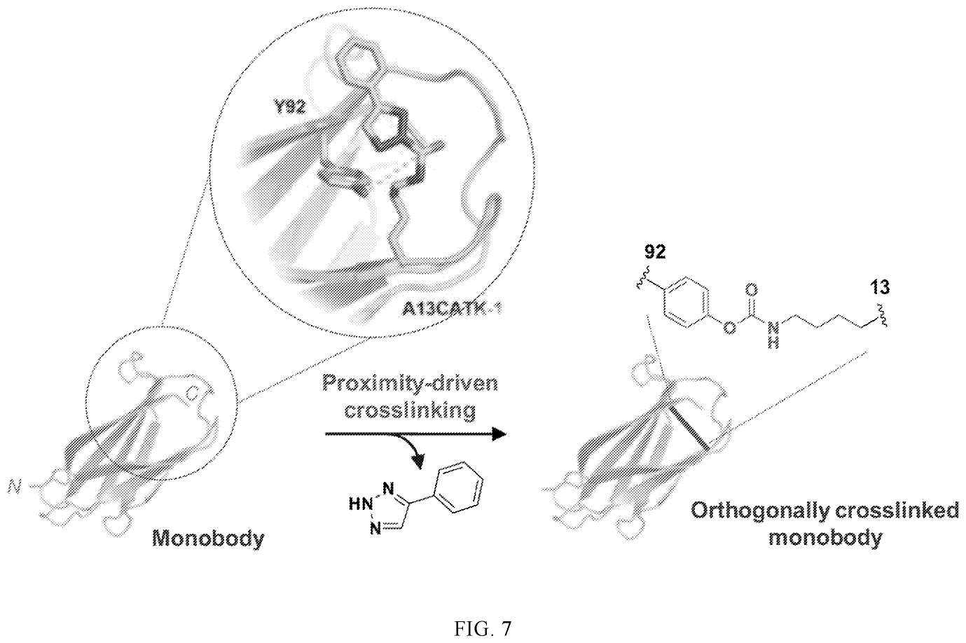

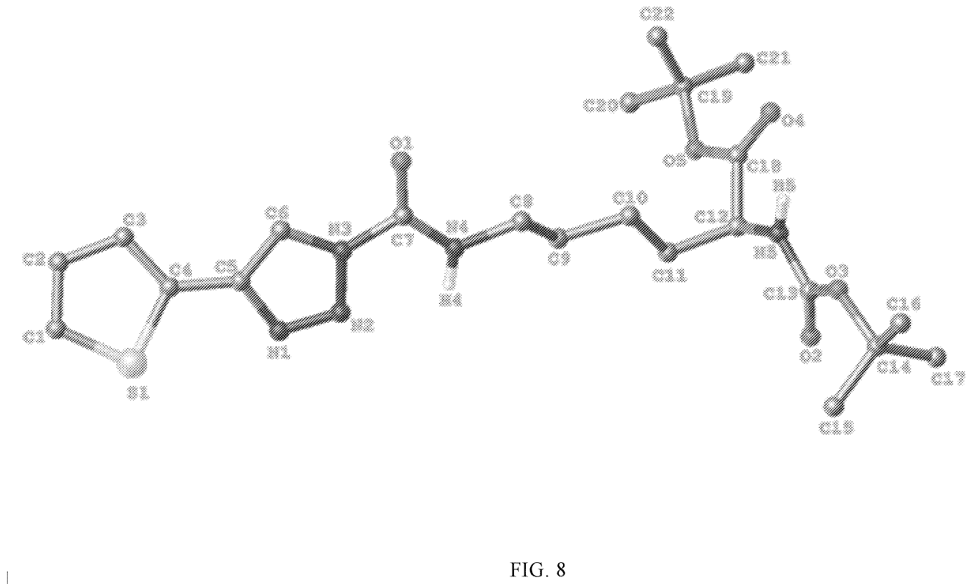

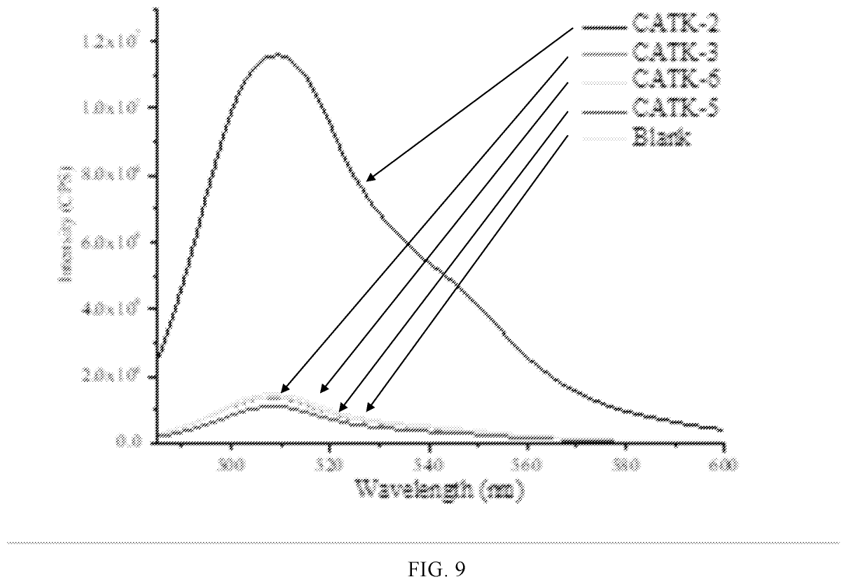

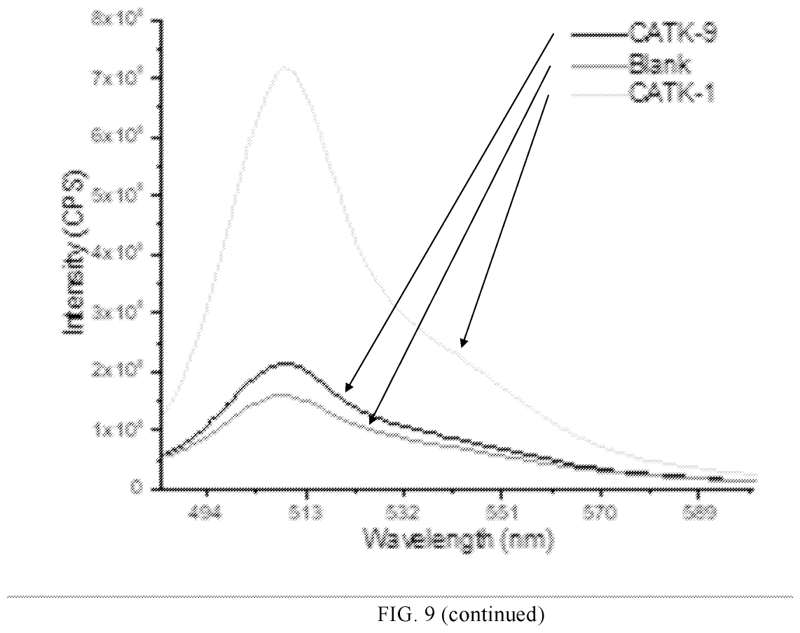

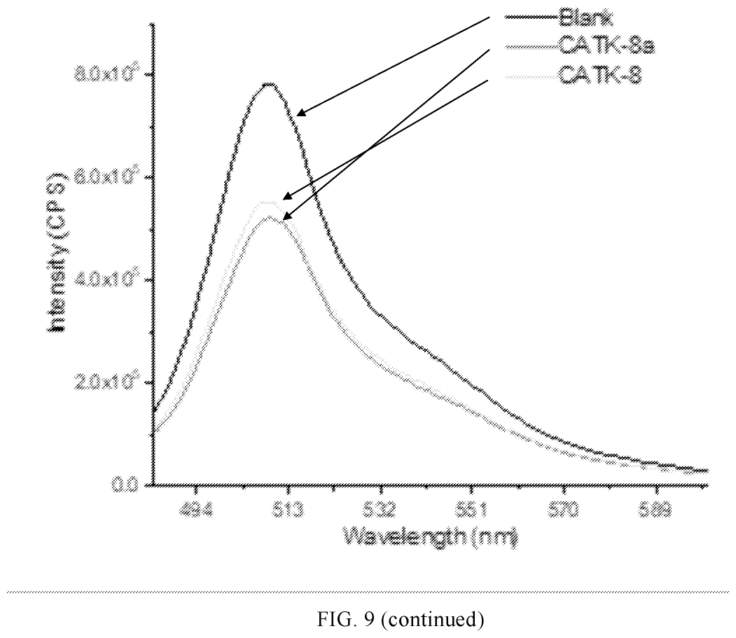

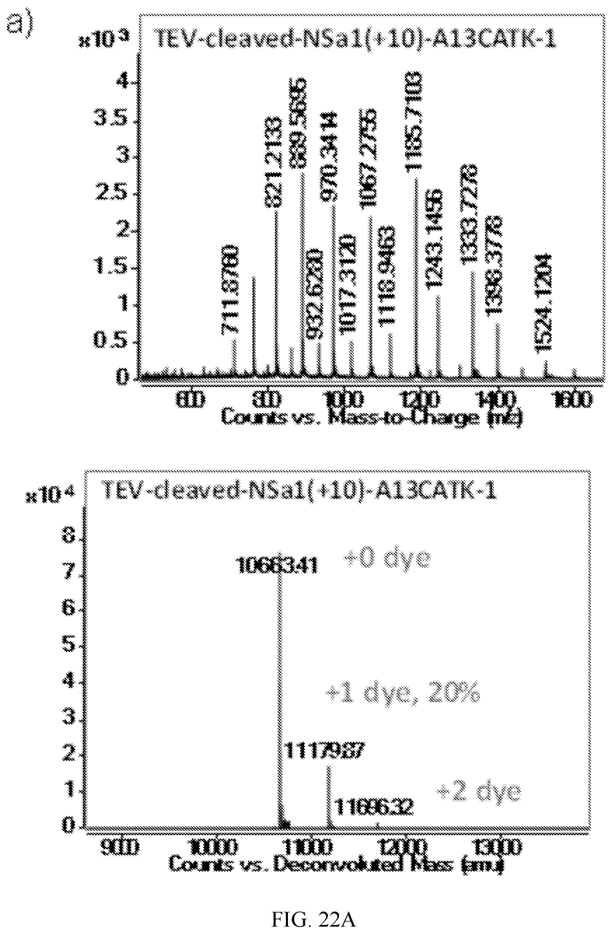

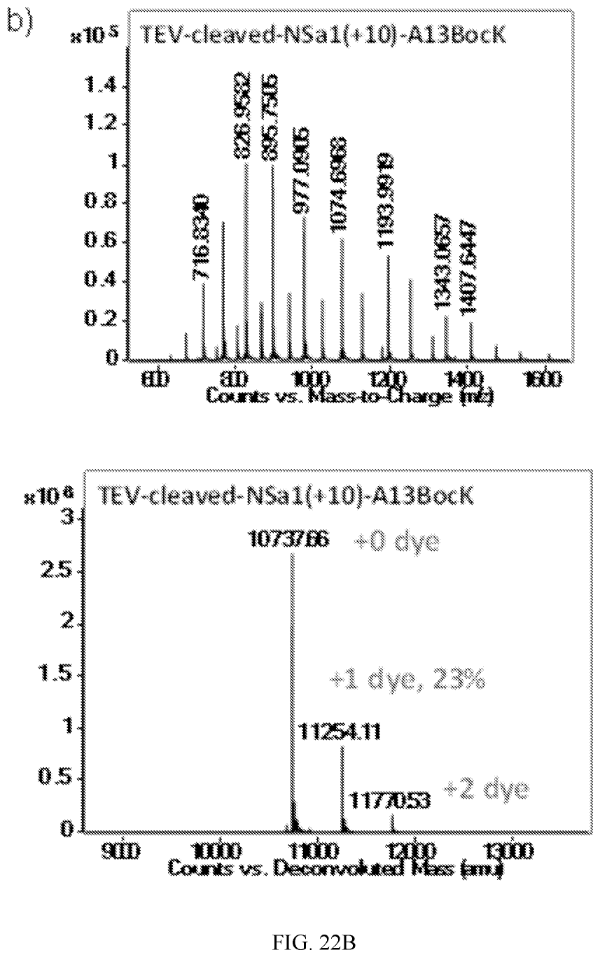

To assess cellular uptake of the supercharged NSa1 proteins, we first removed the N-terminal His-tag after TEV cleavage to obtain two intact NSa1(+10) mutants encoding either BocK or CATK-1. We then reacted the mutants with Alexa Fluor 488-NHS modest labeling yield of 20-23% (FIG. 22). We then carried out a flow cytometry assay to quantify the uptake efficiency of the NSa1(+10) mutants. In brief, HeLa cells were treated with 100 or 500 nM of NSa1(+10) proteins at 37° C. for 4 hours. After washing cells three times with PBS containing 20 U/mL heparin to remove the surface-bound proteins, cells were collected and analyzed by flow cytometry. We observed significant monobody uptake when protein concentrations reached 500 nM. While there is no significant difference in the percentage of fluorescent cells (13.6% vs. 14.8%; FIG. 6b), the NSa1(+10)-A13CATK-1 treated cells showed 40% higher mean fluorescence intensity than the NSa1(+10)-A13BocK treated ones (FIG. 6c; Table 2), indicating a more efficient uptake of the CATK-1-crosslinked monobody (FIG. 6b). Since the kinetically stable protein folds show enhanced proteolytic resistance due to their rigid conformations with limited local openings, we assessed the effect of orthogonal crosslinking on proteolytic stability of the monobody. Thus, we incubated the CATK-1-crosslinked NSa1(+10) with cathepsin B ¾ an enzyme responsible for the degradation of protein cargoes in the endosomes ¾ and monitored monobody stability by mass spectrometry. The CATK-1-crosslinked NSa1(+10) mutant gave a half-life of 126 min, three times longer than the non-crosslinked NSa1(+10)-A13BocK (FIG. 6d), confirming the enhanced kinetic stability afforded by orthogonal crosslinking.

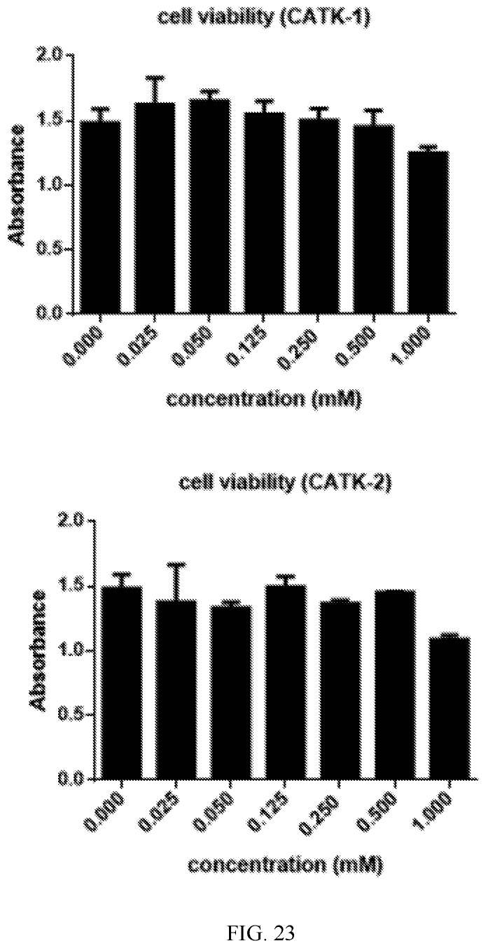

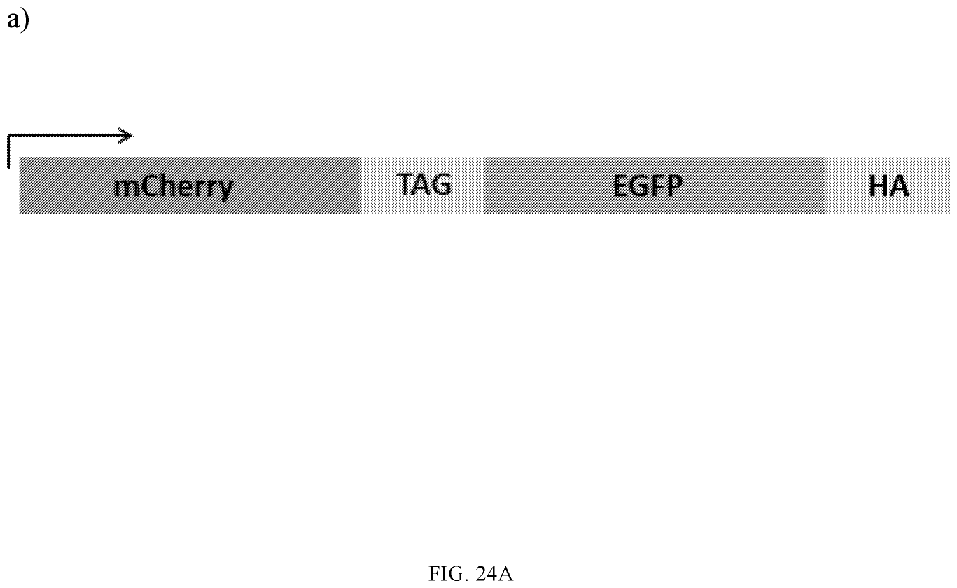

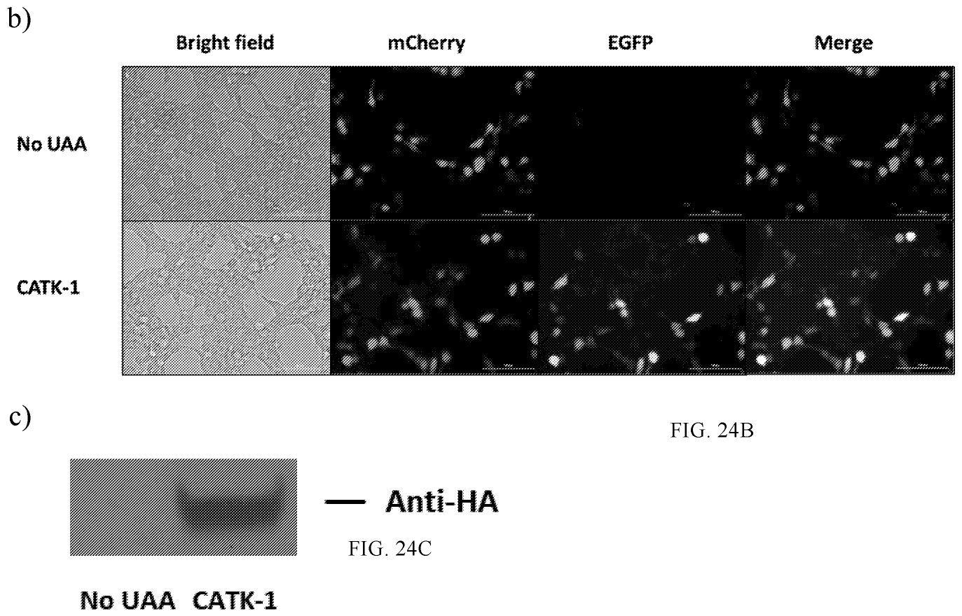

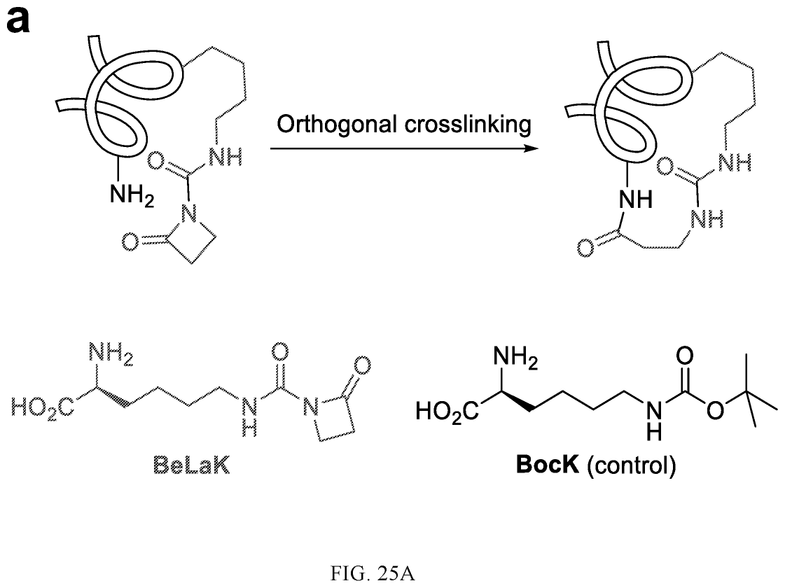

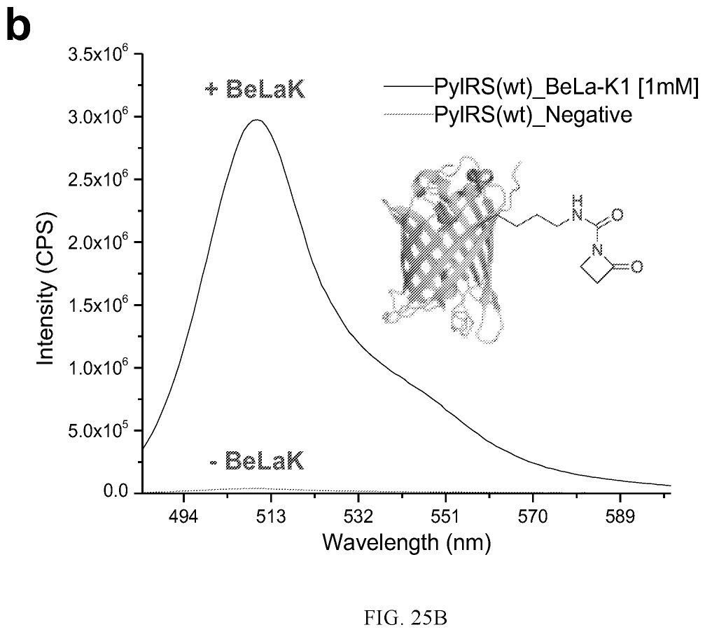

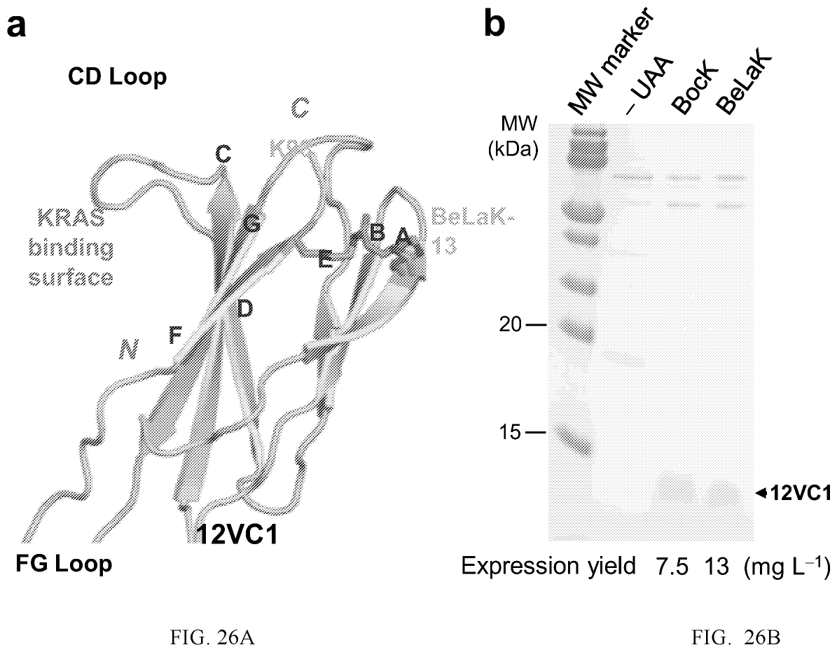

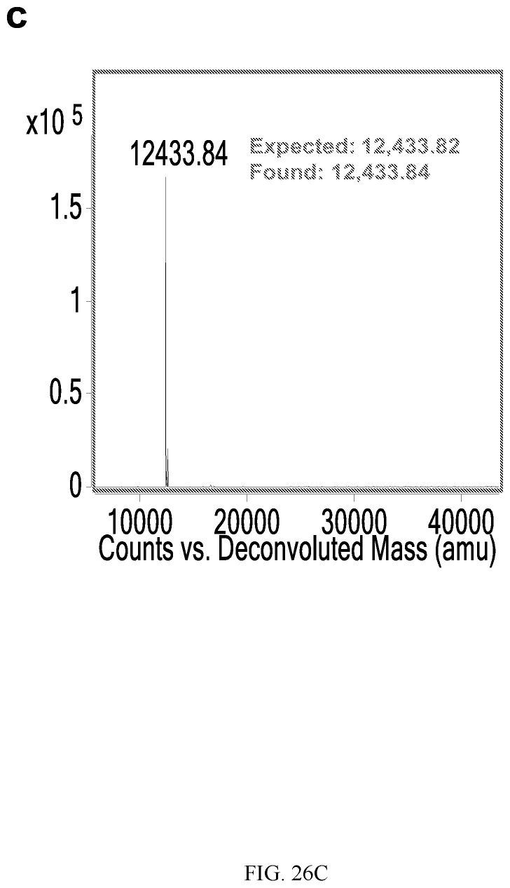

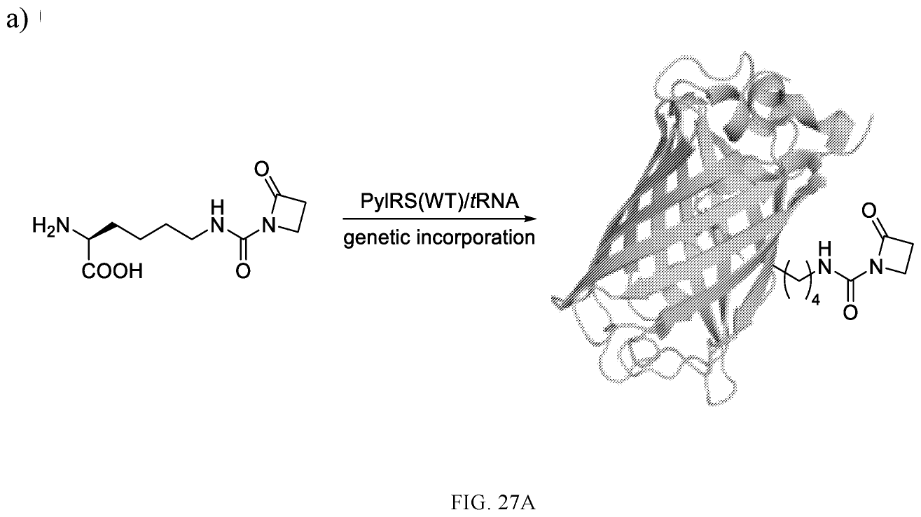

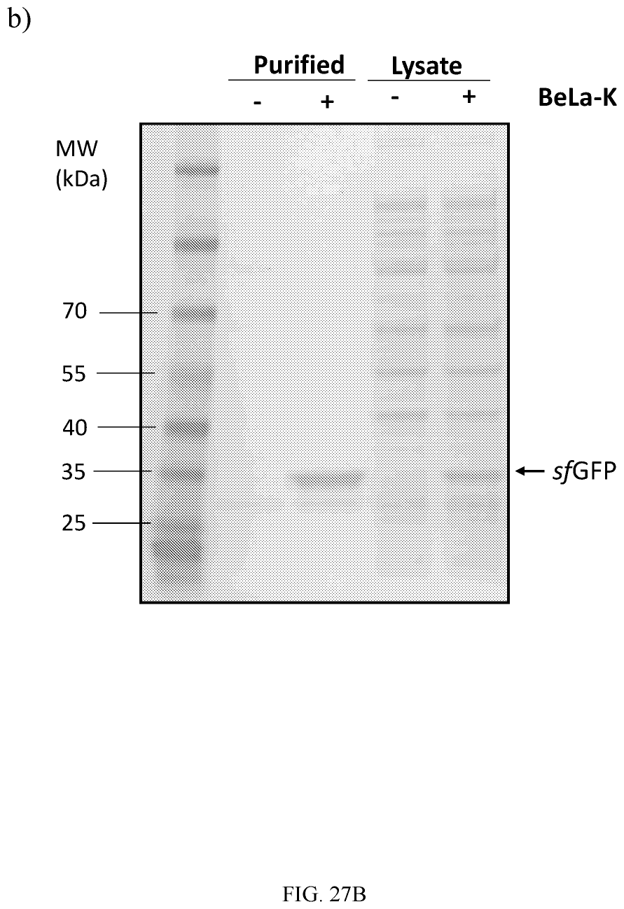

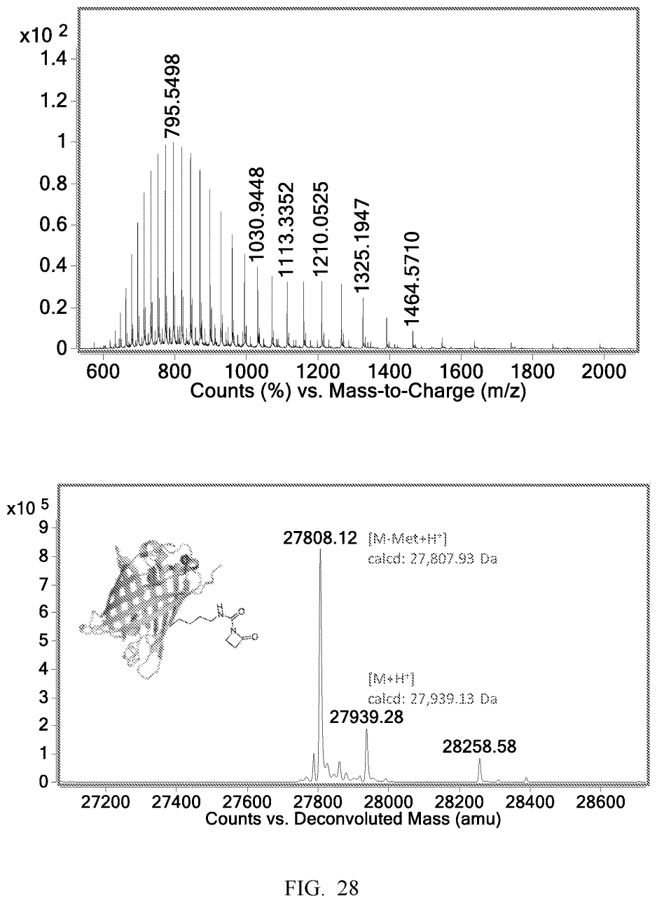

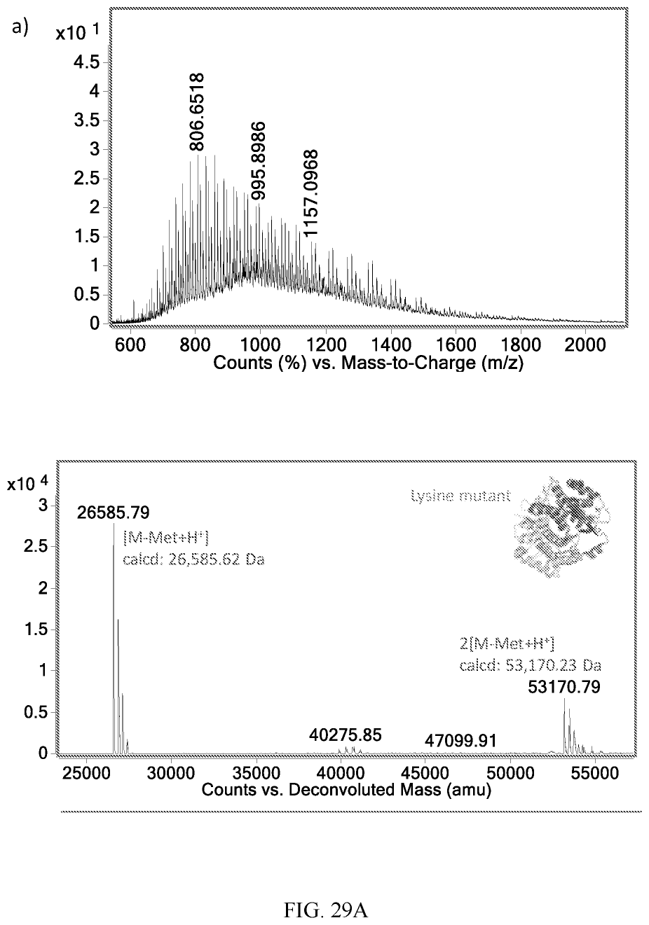

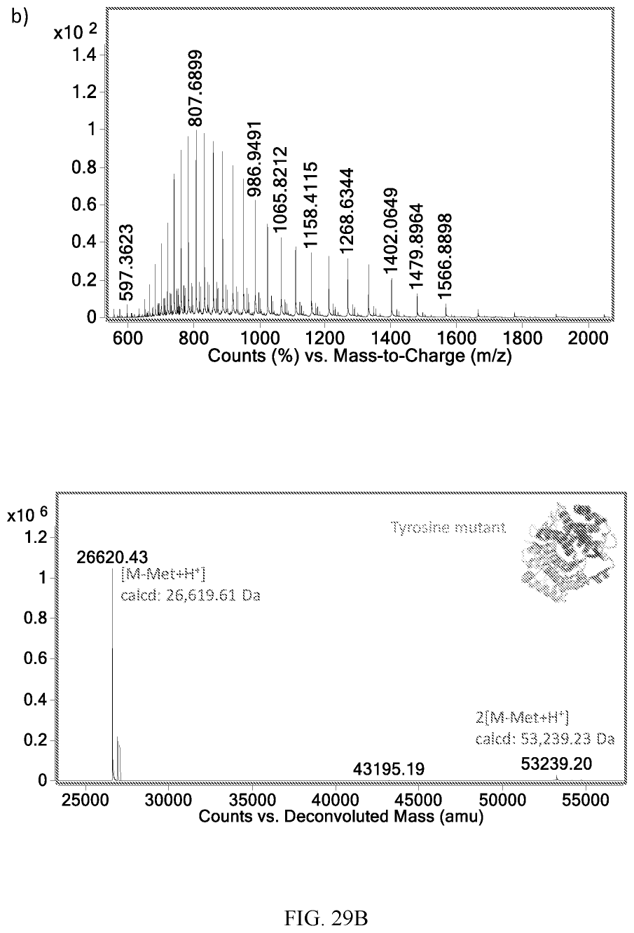

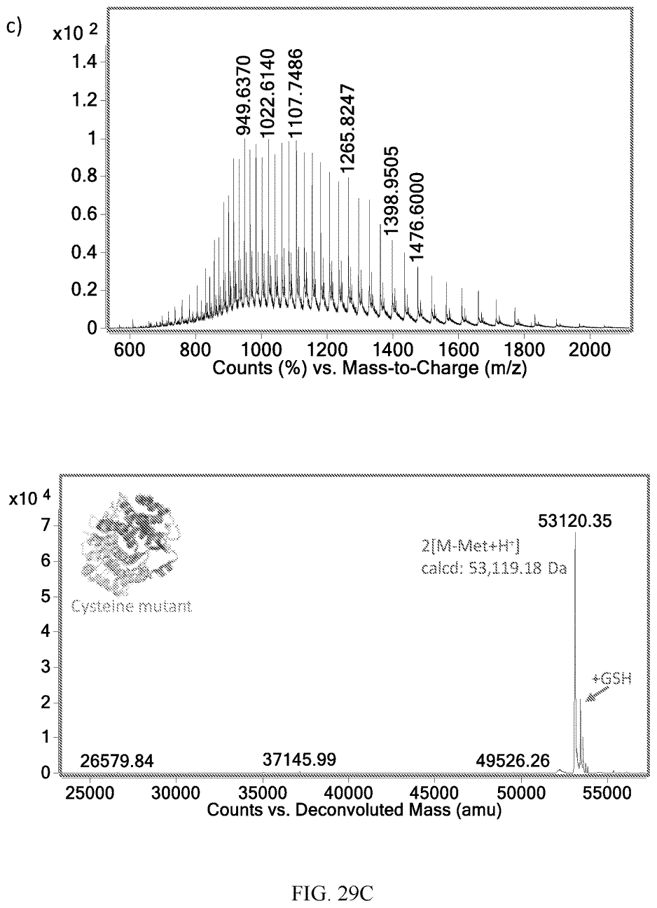

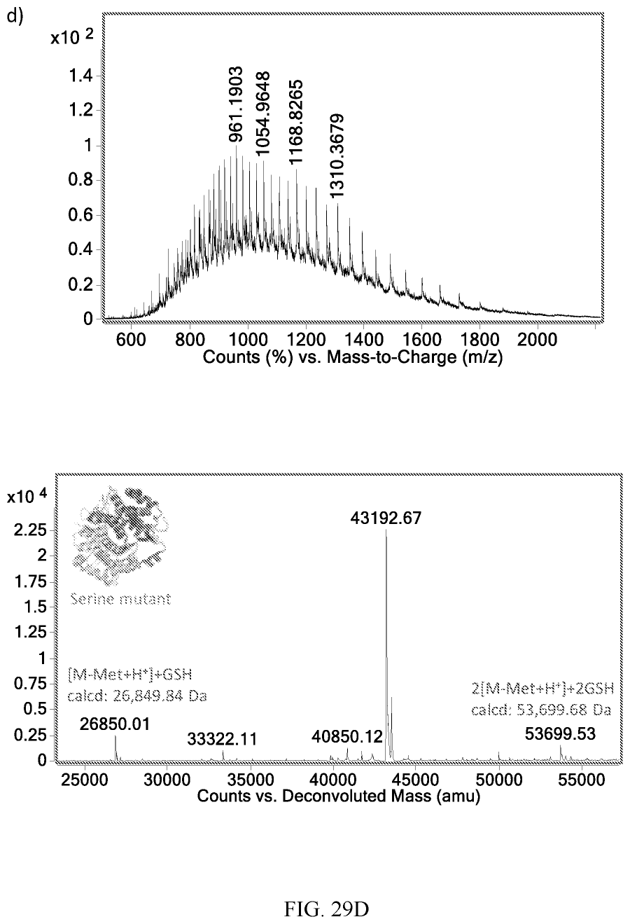

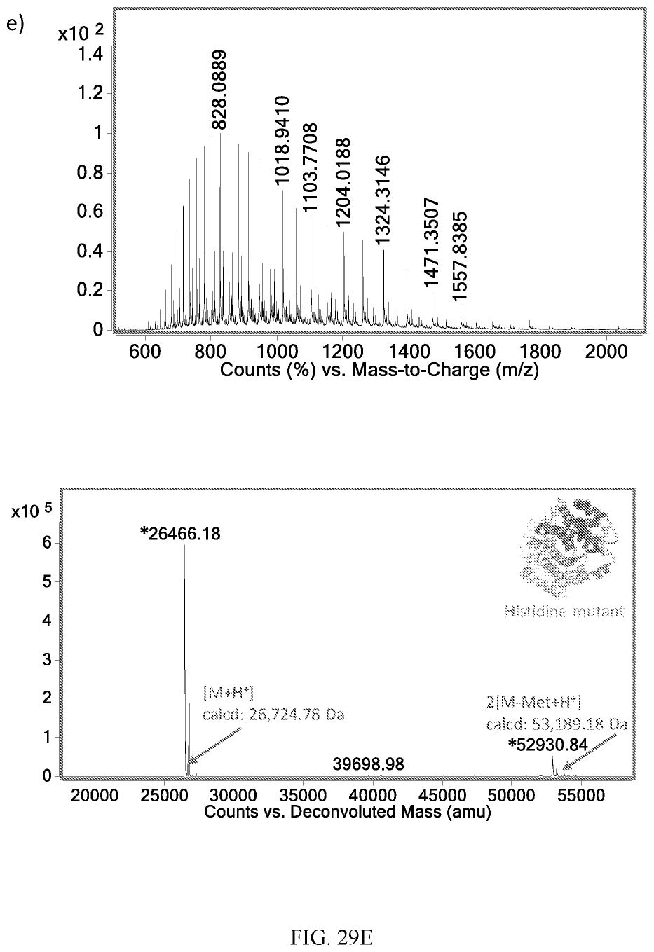

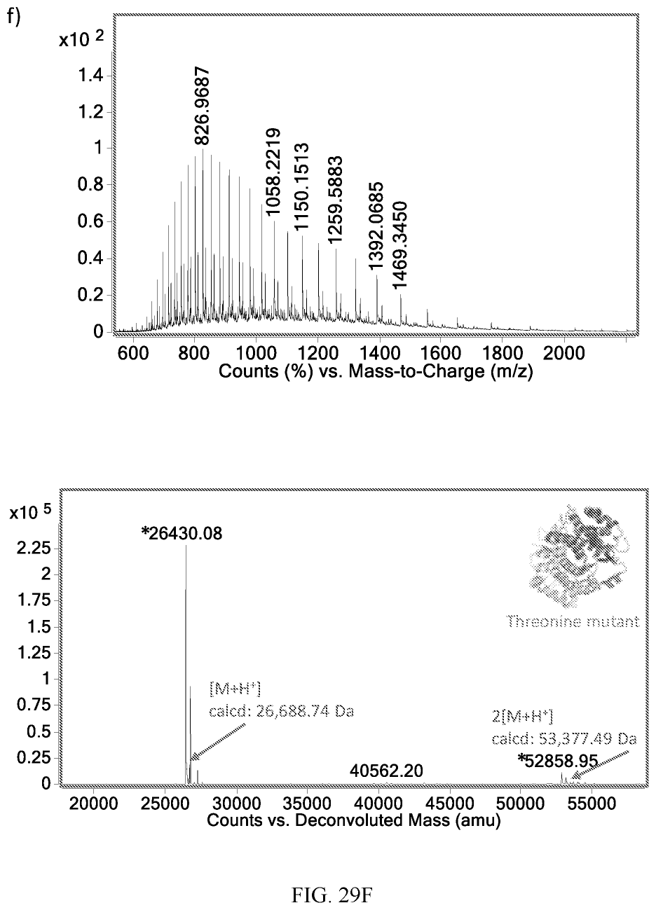

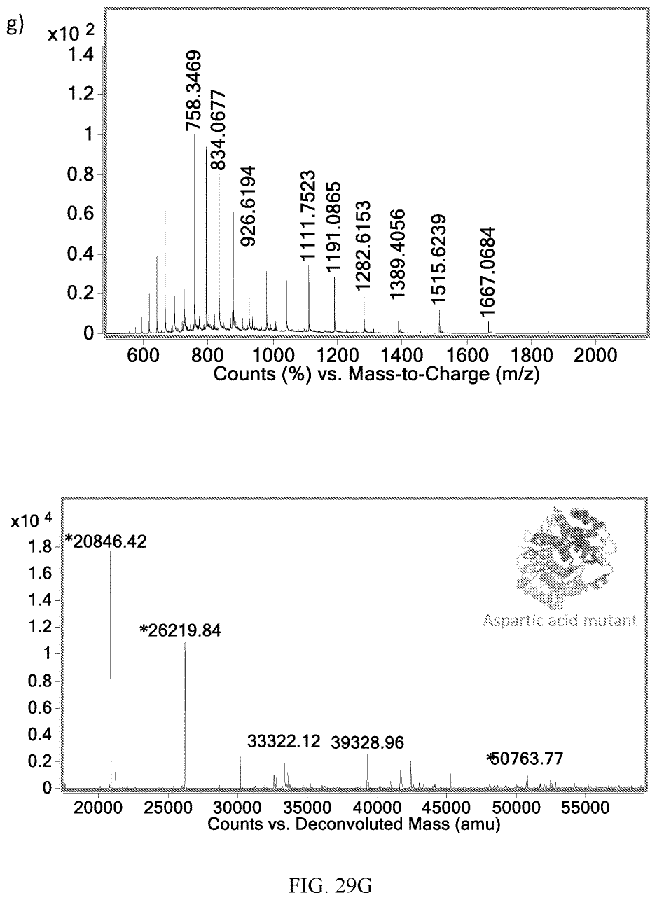

To examine whether CATKs are compatible with genetic code expansion in mammalian cells, we first performed a cell viability assay by treating HEK293T cells with CATK-1 and -2 at various concentrations. We did not detect cytotoxicity at concentrations≤500 μM (FIG. 23). We then cotransfected HEK293T cells with two plasmids: one encodes CATKRS/tRNA^{Pyl}, and the other encodes the mCherry-TAG-EGFP-HA reporter. The transfected cells were allowed to grow in DMEM supplemented with 10% FBS in the absence or presence of CATK-1. Fluorescence microscopy showed green fluorescence when CATK-1 was present, indicating successful CATK-1 incorporation into mCherry-TAG-EGFP-HA, which was also confirmed by western blot (FIG. 24).

In summary, a panel of N²-carboxy-4-aryl-1,2,3-triazole-lysines (CATKs) that can be incorporated into proteins site-specifically via genetic code expansion in E. coli and mammalian cells was designed. When introduced into the GST dimer interface, CATK-1, -2,-4, and -7 permitted spontaneous proximity-driven, site-selective crosslinking of the GST dimer in E. coli. Owing to its enhanced leaving group ability, phenyl-bearing CATK-1 exhibited higher crosslinking reactivity toward the proximal Lys and Tyr at neutral pH than FPheK and FSY, two genetically encoded noncanonical amino acids reported recently. When introduced into the N-terminal b-strand of either a single-chain VHH antibody or a supercharged monobody, CATK-1 enabled efficient site-specific, inter-strand, orthogonal crosslinking with a proximal Tyr located on the opposing b-strand. Compared with a non-crosslinked monobody, the orthogonally crosslinked monobody displayed improved cellular uptake and enhanced proteolytic resistance against an endosomal enzyme. The development of these triazole-based genetically encodable crosslinkers should facilitate the design of novel protein topologies containing orthogonal crosslinks akin to disulfide bonds, leading to potential new applications of protein-based materials.

TABLE 1
Panel of Methanosarcina mazei pyrrolysine-tRNA
synthetase (MmPylRS) variants used in the screen

| Residue MmPylRS | 305 | 306 | 309 | 348 | 384 |
|---|---|---|---|---|---|
| WT | L | Y | L | C | Y |
| V1 | I | Y | A | F | F |
| V2 | L | A | L | L | F |
| V3 (CATKRS) | L | V | A | F | F |
| V4 | L | V | A | L | F |
| V5 | L | Y | A | F | F |
| V6 | L | A | M | A | Y |
| V7 | L | A | L | C | F |

TABLE 2

| | MFI(R-1) | MFI(R-1) | MFI(R-1) |
|---|---|---|---|
| Protein (500 nM) | Trial 1 | Trial 2 | Trial 3 |
| NSa1(+10)-A13BocK | 6726.24 | 6507.23 | 6855.84 |
| NSa1(+10)-A13CATK-1 | 9561.61 | 9020.17 | 9337.41 |
| NSa1(+10)-A13BocK | 6726.24 | 6507.23 | 6855.84 |
| NSa1(+10)-A13CATK-1 | 9561.61 | 9020.17 | 9337.41 |

TABLE 3
Crystal data and structure refinement for S3-4a

| Identification code | S3-4a |
|---|---|
| Empirical formula | C₂₂H₃₃N₅O₅S |
| Formula weight | 479.59 |
| Temperature/K | 90 |
| Crystal system | orthorhombic |
| Space group | P2₁2₁2₁ |
| a/Å | 5.4191(3) |
| b/Å | 10.3725(4) |
| c/Å | 43.396(2) |
| α/° | 90 |
| β/° | 90 |
| γ/° | 90 |
| Volume/Å³ | 2439.3(2) |
| Z | 4 |
| ρ_{calc}g/cm³ | 1.306 |
| μ/mm¹ | 0.175 |
| F(000) | 1024.0 |
| Crystal size/mm³ | 0.16 × 0.02 × 0.02 |
| Radiation | MoKα (λ = 0.71073) |
| 2Θ range for data collection/° | 3.754 to 52.78 |
| Index ranges | −4 ≤ h ≤ 6, −11 ≤ |
| | k ≤ 12, −54 ≤ l ≤ 54 |
| Reflections collected | 20936 |
| Independent reflections | 4966 [Rᵢₙₜ = 0.0523, |
| | R_{sigma} = 0.0481] |
| Data/restraints/parameters | 4966/0/313 |
| Goodness-of-fit on F² | 1.065 |
| Final R indexes [I >= 2σ (I)] | R₁ = 0.0368, wR₂ = 0.0732 |
| Final R indexes [all data] | R₁ = 0.0474, wR₂ = 0.0766 |
| Largest diff. peak/hole/e Å⁻³ | 0.19/−0.23 |
| Flack parameter | 0.00(4) |

TABLE 4
Sequences of DNA oligonucleotides used in this Example.

| | Oligo | |
|---|---|---|
| Description | Type | Sequence |
| GST-E52TAG | Forward | 5′-aacaaaaagtttgaattgggtttgtagtttcccaatc-3′ (SEQ ID |
| | | NO: 26) |
| | Reverse | 5′-aatataataaggaagattgggaaactacaaacccaattc-3′ (SEQ |
| | | ID NO: 27) |
| GST-E52TAG-K92Y | Forward | 5′-agagcgtgcatatatttcaatgcttg-3′ (SEQ ID NO: 28) |
| | Reverse | 5′-tttggacaaccacccaac-3′ (SEQ ID NO: 29) |
| GST-E52TAG-K92C | Forward | 5′-agagcgtgcatgcatttcaatgcttg-3′ (SEQ ID NO: 30) |
| | Reverse | 5′-tttggacaaccacccaac-3′ (SEQ ID NO: 31) |
| GST-E52TAG-K92H | Forward | 5′-agagcgtgcacatatttcaatgcttg-3′ (SEQ ID NO: 32) |
| | Reverse | 5′-tttggacaaccacccaac-3′ (SEQ ID NO: 33) |
| GST-E52TAG-K92A | Forward | 5′-gagcgtgcagcgatttcaatg-3′ (SEQ ID NO: 34) |
| | Reverse | 5′-ttttggacaaccacccaac-3′ (SEQ ID NO: 35) |
| GST-E52TAG-K92S | Forward | 5′-agagcgtgcatctatttcaatgcttgaag-3′ (SEQ ID NO: 36) |
| | Reverse | 5′-tttggacaaccacccaac-3′ (SEQ ID NO: 37) |
| GST-E52TAG-K92D | Forward | 5′-agagcgtgcagatatttcaatgcttg-3′ (SEQ ID NO: 38) |
| | Reverse | 5′-tttggacaaccacccaac-3′ (SEQ ID NO: 39) |
| GST-E52TAG-K92T | Forward | 5′-agagcgtgcaaccatttcaatgcttg-3′ (SEQ ID NO: 40) |
| | Reverse | 5′-tttggacaaccacccaac-3′ (SEQ ID NO: 41) |
| GST-E52TAG-K92M | Forward | 5′-agagcgtgcaatgatttcaatgcttg-3′ (SEQ ID NO: 42) |
| | Reverse | 5′-tttggacaaccacccaac-3′ (SEQ ID NO: 43) |
| GST-E52TAG-K141A | Forward | 5′-tttatgtcatgcaacatatttaaatggtgatc-3′ (SEQ ID NO: |
| | | 44) |
| | Reverse | 5′-cgatcttcgaacattttcag-3′ (SEQ ID NO: 45) |
| NSa1(+10)-Y92F | Forward | 5′-tagcattaattttcgcacctaagg-3′ (SEQ ID NO: 46) |
| | Reverse | 5′-atcgggctactataatag-3′(SEQ ID NO: 47) |
| | Reverse | 5′-gccctggaaatacaggtttt-3′ (SEQ ID NO: 48) |

### Protein Sequences:

wild-type NSa1
(SEQ ID NO: 49)
MGSSHHHHHHSSGTENLYFQGVSSVPTKLEVVAATPTSLLISWDAPAVTV
DYYVITYGETGSGGYAWQEFEVPGSKSTATISGLKPGVDYTITVYAGYYG
YPTYYSSPISINYRT (TEV site underlined)
NSa1(+10)-A13TAG
(SEQ ID NO: 50)
MGSSHHHHHHSSGTENLYFQGVSSKPTKLRVVR*TPTSLKIKWDAPAKTV
DYYVITYGETGRGGYAWQRFEVPGSKRTATIKGLKPGVDYTITVYAGYKG
YPTYYSSPISINYRT (* = CATK or BocK)
NB1-V4TAG
(SEQ ID NO: 51)
MAQ*QLVESGGALVQPGGSLRLSCAASGFPVNRYSMRWYRQAPGKEREWV
AGMSSAGDRSSYEDSVKGRFTISRDDARNTVYLQMNSLKPEDTAVYYCNV
NVGFEYWGQGTQVTVSSLEHHHHHH (* = BocK or CATK)

General Information. Solvents and chemicals were purchased from commercial sources and used directly without further purification. Flash chromatography was performed with SiliCycle P60 silica gel (40-63 μm, 60 Å). 1H and 13C NMR spectra were recorded with Varian Mercury-300, Inova-400, or −500 MHz spectrometer. Chemical shifts were reported in ppm using either TMS or deuterated solvents as internal standards (TMS, 0.00; CDCl3, 7.26; CD3OD, 3.31; DMSO-d6, 2.50). Multiplicity was reported as follows: s=singlet, d=doublet, t=triplet, q=quartet, m=multiplet, brs=broad. 13C NMR spectra were recorded at 75.4, 101, or 126 MHz, and chemical shifts were reported in ppm using deuterated solvents as internal standards (CDCl3, 77.0; DMSO-d6, 39.5; CD3OD, 49.05). LC-MS analysis was performed using the Agilent 6530 Q-TOF mass spectrometer coupled with Agilent 1260 HPLC system. Protein liquid chromatography was performed using a Phenomenex Aeris C4 column (3.6 μm, 200 Å, 2.10×50 mm) with a flow rate of 0.3 mL/min and a linear gradient of 10-90% ACN/H2O containing 0.1% formic acid at 25° C. for 15 min or an Agilent PLRP-S column (5 μm, 1000 Å, 2.10×50 mm) with a flow rate of 0.5 mL/min and 5-95% ACN/H2O containing 0.1% formic acid at 60° C. for 10 min. Intact protein masses were obtained by deconvoluting charge ladders using BioConfirm 10.0 software (Agilent). High resolution mass spectrometry was performed on Agilent 6530 Q-TOF LC/MS. The expression plasmids for NSa1 were purchased from Gene Universal (Newark, DE).

Experimental Procedures and Characterization Data: General synthetic procedure. All triazoles were prepared by following the literature procedure.^{1,2} The 1/2-carboxy-4-aryltriazole lysine derivatives (CATKs) were synthesized using either Method A or Method B. The N¹ and N² regioisomers were separated by silica gel flash chromatography and characterized by NMR. The single crystal of tert-butyl N²-(tert-butoxycarbonyl)-N⁶-(4-(thiophen-2-yl)-1H-1,2,3-triazole-1-carbonyl)-L-lysinate (N1 product) was obtained from ethyl acetate/hexanes at room temperature, and the structure was unambiguously determined by X-ray crystallography (CCDC 1993355). The N¹ product showed a downfield shift in ¹H NMR signal for the triazole ring and faster migration on TLC compared to the N² product. For the extremely poor solubility of all N¹ products, the final N¹ products were characterized by NMR in CD₃OD with TFA-d₄ and excluded from further biological studies.

### Synthesis of 1/2-Carboxy-4-Aryltriazole Lysine (CATK) Derivatives

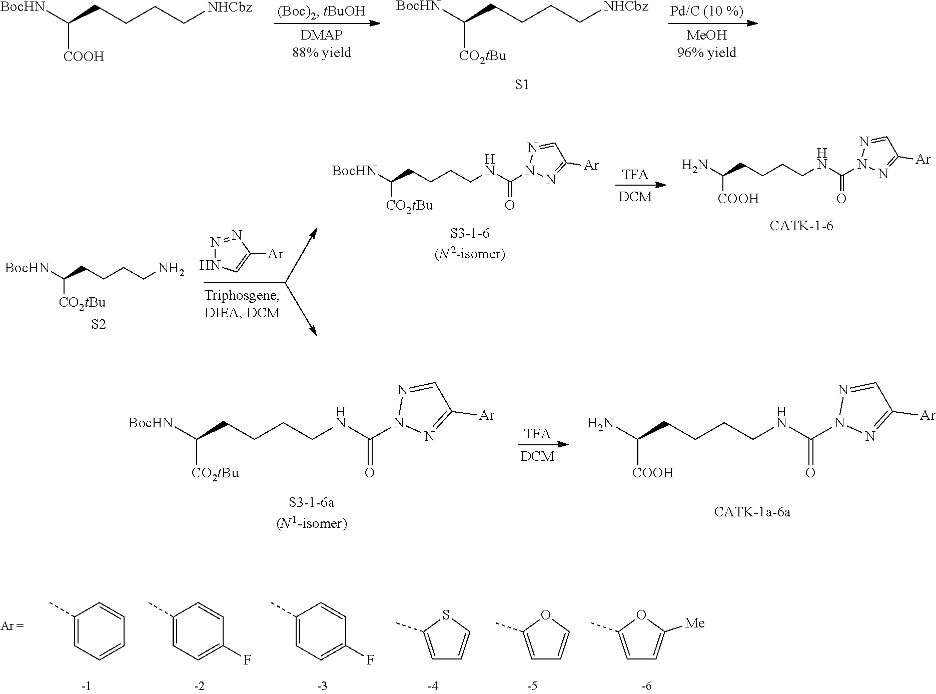

tert-Butyl N⁶-((benzyloxy)carbonyl)-N²-(tert-butoxycarbonyl)-L-lysinate (S1).

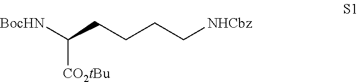 [SMILES: CC(C)(C)OC(=O)N[C@@H](CCCCNC(=O)OCc1ccccc1)C(=O)OC(C)(C)C]

To solution of Boc-L-Lys(Z)—OH (7.60 g, 20 mmol) dissolved in tBuOH (20.0 mL) at 30° C., (Boc)₂O (6.12 g, 1.4 equiv.) was added and stirred for 5 min. Then DMAP (0.73 g, 0.3 equiv.) was added, and the mixture was stirred overnight. The solvent was removed under reduced pressure and the residue was purified by silica gel flash chromatography (EtOAc/hexanes=0:100 to 1:4) to afford the title compound as a white solid (7.70 g, 88% yield): ¹H NMR (300 MHz, CDCl₃) δ 7.36-7.33 (m, 5H), 5.09 (s, 3H), 4.91 (s, 1H), 4.25-4.13 (m, 1H), 3.21-315 (m, 2H), 1.80-1.73 (m, 1H), 1.66-1.49 (m, 3H), 1.45 (s, 9H), 1.43 (s, 9H), 1.26-1.22 (m, 2H); HRMS (ESI) calcd for C₂₃H₃₆N₂O₆Na 459.2466 [M+Na⁺], found 459.2464.

tert-Butyl N²-(tert-butoxycarbonyl)-L-lysinate (S2).

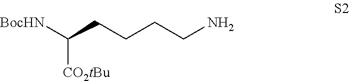 [SMILES: CC(C)(C)OC(=O)N[C@@H](CCCCN)C(=O)OC(C)(C)C]

To a solution of S1 (1.80 g, 4.10 mmol) in MeOH (10.0 mL) was added Pd/C (180.0 mg, 10%). The mixture was stirred with a hydrogen balloon at room temperature overnight. Pd/C was removed by filtering through celite, and the filtrate was concentrated to afford the title compound as a colorless oil (1.60 g, 96% yield): ¹H NMR (300 MHz, CDCl₃) δ 5.11-5.07 (m, 1H), 4.20-4.13 (m, 1H), 2.74-2.55 (m, 2H), 2.55 (s, 2H), 1.86-1.69 (m, 1H), 1.69-1.49 (m, 3H), 1.45 (d, J=6.3 Hz, 18H), 1.39-1.16 (m, 2H)

tert-Butyl N²-(tert-butoxycarbonyl)-N⁶-(4-phenyl-2H-1,2,3-triazole-2-carbonyl)-L-lysinate (S3-1).

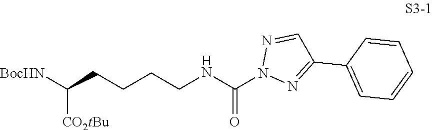 [SMILES: CC(C)(C)OC(=O)N[C@@H](CCCCNC(=O)n1ncc(-c2ccccc2)n1)C(=O)OC(C)(C)C]

To a solution of triphosgene (219.6 mg, 0.74 mmol) in DCM (4.0 mL) at 0° C. was added dropwise a solution of S2 (604.8 mg, 2.0 mmol) and DIEA (699 μL, 4.0 mmol) in DCM (7.0 mL). The mixture was stirred at 0° C. for 30 min. Then, a solution of 4-phenyl-1H-1,2,3-triazole (290.3 mg, 2.0 mmol) and DIEA (699 μL, 4.0 mmol) in DCM (7.0 mL) was added. The reaction mixture was stirred at room temperature for another 30 min. The solvent was removed under reduced pressure and the residue was dissolved in EtOAc. The organic layer was washed successively with saturated KHSO₄, saturated NaHCO₃, and brine, dried over anhydrous Na₂SO₄, filtered, and concentrated. The residue was purified by silica gel flash chromatography (EtOAc/hexanes=1:3) to afford the title compound as a colorless oil (295.0 mg, 31% yield): ¹H NMR (400 MHz, CDCl₃) δ 8.07 (s, 1H), 7.87 (d, J=6.7 Hz, 2H), 7.48-7.42 (m, 3H), 7.32-7.26 (m, 1H), 5.13 (d, J=8.3 Hz, 1H), 4.20 (d, J=6.7 Hz, 1H), 3.51 (q, J=6.7 Hz, 2H), 1.93-1.51 (m, 5H), 1.50-1.39 (m, 19H); ¹³C NMR (101 MHz, CDCl₃) δ 171.8, 155.4, 150.0, 147.5, 134.1, 129.6, 128.9, 128.7, 126.5, 81.8, 79.6, 53.7, 40.6, 32.6, 29.1, 28.3, 27.9, 22.4; HRMS (ESI) calcd for C₂₄H₃₅N₅O₅Na 496.2530 [M+Na⁺], found 496.2522.

tert-Butyl N²-(tert-butoxycarbonyl)-N⁶-(4-phenyl-1H-1,2,3-triazole-1-carbonyl)-L.-lysinate (S3-1a).

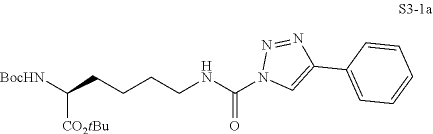 [SMILES: CC(C)(C)OC(=O)N[C@@H](CCCCNC(=O)n1cc(-c2ccccc2)nn1)C(=O)OC(C)(C)C]

The titled minor product was obtained after silica gel flash chromatography as a white solid (219.6 mg, 23% yield): ¹H NMR (300 MHz, CDCl₃) δ 8.47 (s, 1H), 7.87 (d, J=7.5 Hz, 2H), 7.48-7.35 (m, 4H), 5.08 (d, J=8.3 Hz, 1H), 4.23-4.16 (m, 1H), 3.52 (q, J=6.9 Hz, 2H), 1.91-1.50 (m, 5H), 1.54-1.40 (m, 19H); ¹³C NMR (75 MHz, CDCl₃) δ 171.8, 155.4, 148.2, 147.3, 129.4, 129.0, 128.8, 125.9, 117.8, 81.9, 79.7, 53.6, 40.5, 32.6, 29.0, 28.3, 28.0, 22.4; HRMS (ESI) calcd for C₂₄H₃₅N₅O₅Na 496.2530 [M+Na⁺], found 496.2527.

N⁶-(4-Phenyl-2H-1,2,3-triazole-2-carbonyl)-L-lysine (CATK-1).

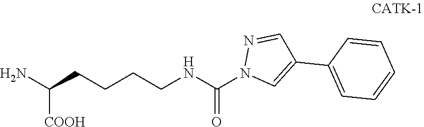 [SMILES: N[C@@H](CCCCNC(=O)n1cc(-c2ccccc2)cn1)C(=O)O]

To a solution of S3-1 (200.0 mg, 0.42 mmol) in DCM (2.0 mL) at 0° C. was added TFA (2.0 mL), and the mixture was stirred at room temperature for 6 h. Then, the solvent was removed under reduced pressure. The residue was washed with DCM and Et₂O, and purified by silica gel flash chromatography (MeOH) to give the desired product as a white solid (62 mg, 34% yield): ¹H NMR (400 MHz, CD₃OD/CF₃CO₂D=6:1) 6 8.20 (s, 1H), 7.83 (d, J=6.8 Hz, 2H), 7.40-7.33 (m, 3H), 3.87 (t, J=6.3 Hz, 1H), 3.40 (t, J=6.8 Hz, 2H), 1.97-1.86 (m, 2H), 1.70-1.63 (m, 2H), 1.54-1.43 (m, 2H); ¹³C NMR (101 MHz, CD₃OD/CF₃CO₂D=6:1) δ 172.0, 151.8, 150.1, 135.7, 130.9, 130.2, 127.6, 120.8, 118.0, 115.1, 53.9, 41.2, 31.1, 30.0, 23.2; HRMS (ESI) calcd for C₁₅H₂₀N₅O₃ 318.1561 [M+H⁺], found 318.1558.

N⁶-(4-Phenyl-1H-1,2,3-triazole-1-carbonyl)-L-lysine (CATK-1a).

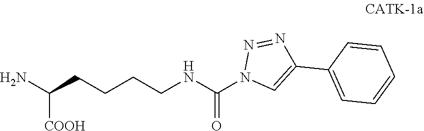 [SMILES: N[C@@H](CCCCNC(=O)n1cc(-c2ccccc2)nn1)C(=O)O]

To a solution of S3-1a (170.0 mg, 0.36 mmol) in DCM (2.0 mL) at 0° C. was added TFA (2.0 mL), and the mixture was stirred at room temperature for 6 h. Then, the solvent was removed under reduced pressure and the residue was washed successively with DCM, Et₂O, and water to afford the title compound as a white solid (100.0 mg, 64% yield): ¹H NMR (400 MHz, CD₃OD/CF₃CO₂D=6:1) δ 8.64 (s, 1H), 7.79 (d, J=7.1 Hz, 2H), 7.38-7.26 (m, 3H), 3.88 (t, J=6.2 Hz, 1H), 3.40 (t, J=6.8 Hz, 2H), 2.05-1.79 (m, 2H), 1.71-1.63 (m, 2H), 1.56-1.41 (m, 2H); ¹³C NMR (101 MHz, CD₃OD/CF₃CO₂D=6:1) δ 171.8, 149.4, 149.2, 130.8, 130.1, 129.9, 127.0, 119.8, 118.0, 115.1, 53.9, 41.2, 31.2, 29.9, 23.2; HRMS (ESI) calcd for C₁₅H₁₉N₅O₃Na 340.1380 [M+Na⁺], found 340.1379.

tert-Butyl N²-(tert-butoxycarbonyl)-N⁶-(4-(4-fluorophenyl)-2H-1,2,3-triazole-2-carbonyl)-L-lysinate (S3-2).

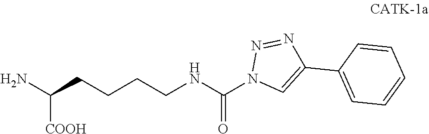 [SMILES: N[C@@H](CCCCNC(=O)n1cc(-c2ccccc2)nn1)C(=O)O]

To a solution of triphosgene (220.0 mg, 0.74 mmol) in DCM (4.0 mL) at 0° C. was added dropwise a solution of S2 (605.0 mg, 2.0 mmol) in DCM (7.0 mL) and DIEA (768 μL, 4.4 mmol), and the mixture was stirred at 0° C. for 30 min. Then, a solution of 4-(4-fluorophenyl)-1H-1,2,3-triazole (326.0 mg, 2.0 mmol) in DCM (7.0 mL) and DIEA (768 μL, 4.4 mmol) were added, and the mixture was stirred for another 0.5 h (h=hour(s)) at room temperature. The solvent was removed by reduced pressure and the residue was diluted with EtOAc. The organic layer was washed successively with saturated KHSO₄, saturated NaHCO₃, brine, and then dried over anhydrous Na₂SO₄, filtered, and concentrated. The residue was purified by silica gel flash chromatography (EtOAc/hexanes=1:2) to give the title compound as a colorless oil (193.0 mg, 39% yield): ¹H NMR (300 MHz, CDCl₃) δ 8.06 (s, 1H), 7.90-7.85 (m, 2H), 7.38-7.34 (m, 1H), 7.16 (t, J=8.5 Hz, 2H), 5.19 (d, J=8.4 Hz, 1H), 4.25-4.12 (m, 1H), 3.53 (q, J=6.7 Hz, 2H), 1.98-1.53 (m, 6H), 1.47 (s, 9H), 1.45 (s, 9H); ¹³C NMR (75 MHz, CDCl₃) δ 171.8, 165.1, 161.8, 155.4, 149.1, 147.4, 133.8, 128.4 (d, J=8.3 Hz), 124.9 (d, J=3.0 Hz), 116.0 (d, J=22 Hz), 81.8, 79.5, 53.7, 40.6, 32.5, 29.0, 28.2, 27.9, 22.4; HRMS (ESI) calcd for C₂₄H₃₄FN₅O₅Na 514.2436 [M+Na⁺], found 514.2456.

tert-Butyl N²-(tert-butoxycarbonyl)-N⁶-(4-(4-fluorophenyl)-1H-1,2,3-triazole-1-carbonyl)-L-lysinate (S3-2a).

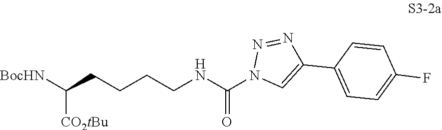 [SMILES: CC(C)(C)OC(=O)N[C@@H](CCCCNC(=O)n1cc(-c2ccc(F)cc2)nn1)C(=O)OC(C)(C)C]

The titled minor product was obtained after silica gel flash chromatography followed by recrystallization in EtOAc/hexanes as a white solid (115.0 mg, 12% yield): ¹H NMR (300 MHz, CDCl₃) δ 8.45 (s, 1H), 7.87-7.83 (m, 2H), 7.43 (t, J=6.1 Hz, 1H), 7.15 (t, J=8.6 Hz, 2H), 5.12 (d, J=8.3 Hz, 1H), 4.23-4.16 (m, 1H), 3.52 (q, J=6.8 Hz, 2H), 1.91-1.50 (m, 6H), 1.46 (s, 9H), 1.43 (s, 9H); ¹³C NMR (75 MHz, CDCl₃) δ 171.9, 164.7, 161.5, 155.5, 147.4 (d, J=4.5 Hz), 127.8 (d, J=9.0 Hz), 125.8 (d, J=3.0 Hz), 117.7, 116.1 (d, J=21.8 Hz), 82.0, 79.7, 53.8, 40.7, 32.7, 29.1, 28.4, 28.1, 22.6; HRMS (ESI) calcd for C₂₄H3₄FN₅O₅NK 530.2176 [M+K⁺], found 530.2188.

N⁶-(4-(4-fluorophenyl)-2H-1,2,3-triazole-2-carbonyl)-L-lysine (CATK-2).

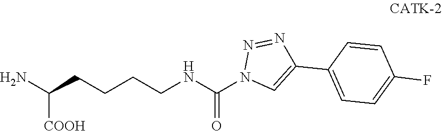 [SMILES: N[C@@H](CCCCNC(=O)n1cc(-c2ccc(F)cc2)nn1)C(=O)O]

To a solution of S3-2 (193.0 mg, 0.39 mmol) in DCM (2.0 mL) at 0° C. was added TFA (2.0 mL), and the mixture was stirred at room temperature for 6 h. Then, the solvent and excess TFA were removed under reduced pressure. The crude was recrystallized in MeOH/Et₂O to afford the title compound as a white solid (53.0 mg, 30% yield): ¹H NMR (400 MHz, CD₃OD) δ 8.31 (s, 1H), 8.00-7.95 (m, 2H), 7.23-7.18 (m, 2H), 3.96 (t, J=6.2 Hz, 1H), 3.47 (t, J=6.9 Hz, 2H), 2.12-1.87 (m, 2H), 1.78-1.69 (m, 2H), 1.62-1.49 (m, 2H); ¹³C NMR (101 MHz, CD₃OD/CF₃CO₂D=5:1) δ 171.8, 150.8, 150.0, 135.6, 129.8, 129.7, 118.0, 117.2, 117.0, 115.2, 53.8, 41.2, 31.2, 30.1, 23.2; HRMS (ESI) calcd for C₁₅H₁₉FN₅O₃ 336.1466 [M+H⁺], found 336.1463.

N⁶-(4-(4-Fluorophenyl)-1H-1,2,3-triazole-1-carbonyl)-L-lysine (CATK-2a).

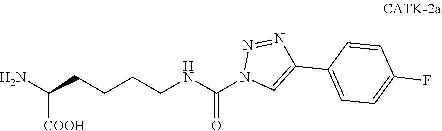 [SMILES: N[C@@H](CCCCNC(=O)n1cc(-c2ccc(F)cc2)nn1)C(=O)O]

To a solution of S3-2a (115.0 mg, 0.23 mmol) in DCM (2.0 mL) at 0° C. was added TFA (2.0 mL), and the mixture was stirred at room temperature overnight. Then, the solvent and excess TFA were removed under reduced pressure and the residue was washed successively with DCM, Et₂O, and water to give the title compound as a white solid (7.0 mg, 7% yield): ¹H NMR (300 MHz, CD₃OD/CF₃CO₂D=5:1) δ 8.62 (s, 1H), 7.84-7.79 (m, 2H), 7.12-7.06 (m, 2H), 3.87 (t, J=6.3 Hz, 1H), 3.40 (t, J=6.9 Hz, 2H), 1.97-1.82 (m, 2H), 1.72-1.63 (m, 2H), 1.56-1.40 (m, 2H); ¹³C NMR (75 MHz, CD₃OD/CF₃CO₂D=5:1) δ 171.9, 149.4, 148.4, 129.1, 129.0, 119.7, 117.9, 117.1, 116.8, 114.2, 53.9, 41.2, 31.2, 29.9, 23.2; HRMS (ESI) calcd for C₁₅H₁₈FN₅O₃Na 358.1286 [M+Na⁺], found 358.1284.

tert-Butyl N²-(tert-butoxycarbonyl)-N⁶-(4-(4-chlorophenyl)-2H-1,2,3-triazole-2-carbonyl)-L-lysinate (S3-3).

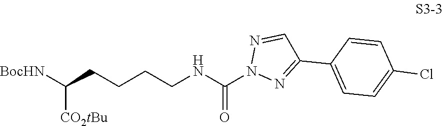 [SMILES: CC(C)(C)OC(=O)N[C@@H](CCCCNC(=O)n1ncc(-c2ccc(Cl)cc2)n1)C(=O)OC(C)(C)C]

To a solution of triphosgene (220.0 mg, 0.74 mmol) in DCM (4.0 mL) at 0° C. was added dropwise a solution of S2 (605.0 mg, 2.0 mmol) and DIEA (768 μL, 4.4 mmol) in DCM (7.0 mL). The reaction mixture was stirred at 0° C. for 30 min. Then, a solution of 4-(4-chlorophenyl)-1H-1,2,3-triazole (359.0 mg, 2.0 mmol) and DIEA (768 μL, 4.4 mmol) in DCM (7.0 mL) was added, and the mixture was stirred at room temperature for another 30 min (min=minute(s)). The solvent was removed under reduced pressure and the residue was dissolved with EtOAc. The organic layer was washed successively with saturated KHSO₄, saturated NaHCO₃, and brine, and then dried over anhydrous Na₂SO₄, filtered, and concentrated. The residue was purified by silica gel flash chromatography (EtOAc/hexanes=1:2) as a colorless oil (210.0 mg, 21% yield): ¹H NMR (300 MHz, CDCl₃) δ 8.06 (s, 1H), 7.85-7.77 (m, 2H), 7.43-7.41 (m, 2H), 7.34 (t, J=6.1 Hz, 1H), 5.17 (d, J=8.3 Hz, 1H), 4.20 (d, J=6.8 Hz, 1H), 3.52 (q, J=6.8 Hz, 2H), 1.85-1.48 (m, 6H), 1.44 (d, J=5.9 Hz, 18H); ¹³C NMR (75 MHz, CDCl₃) δ 171.7, 155.4, 148.9, 147.3, 135.5, 133.9, 129.2, 127.7, 127.2, 81.8, 53.7, 40.6, 32.5, 29.0, 28.2, 27.9, 22.4; HRMS (ESI) calcd for C₂₄H₃₄³⁵ClN₅O₅Na 530.2141 [M+Na⁺], found 530.2154; C₂₄H₃₄³⁷ClN₅O₅Na 532.2112 [M+Na⁺], found 532.2126.

tert-Butyl N²-(tert-butoxycarbonyl)-N⁶-(4-(4-chlorophenyl)-1H-1,2,3-triazole-1-carbonyl)-L-lysinate (S3-3a).

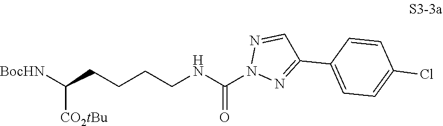 [SMILES: CC(C)(C)OC(=O)N[C@@H](CCCCNC(=O)n1ncc(-c2ccc(Cl)cc2)n1)C(=O)OC(C)(C)C]

The titled, minor product was obtained after silica gel flash chromatography (EtOAc/hexanes=1:3) as a mixture with 4-(4-chlorophenyl)-1H-1,2,3-triazole in a ratio of 85:15 based on ¹H NMR (230.0 mg, 14.6%): ¹H NMR (500 MHz, CDCl₃) δ 8.53 (s, 1H), 7.88-7.74 (m, 2H), 7.63-7.61 (m, 1H), 7.42-7.37 (m, 2H), 5.24 (d, J=8.2 Hz, 1H), 4.35-4.15 (m, 1H), 3.56-3.51 (m, 2H), 1.88-1.66 (m, 4H), 1.62-1.50 (m, 2H), 1.49-1.42 (m, 18H); ¹³C NMR (126 MHz, CDCl₃) δ 171.9, 155.4, 147.2, 147.0, 134.5, 129.1, 128.0, 127.1, 118.1, 81.8, 53.7, 40.6, 32.5, 29.0, 28.3, 27.9, 22.5; HRMS (ESI) calcd for C₂₄H₃₄³⁵ClN₅O₅Na 530.2141. [M+Na⁺], found 530.2149; C₂₄H₃₄³⁷ClN₅O₅Na 532.2112 [M+H⁺], found 532.2124.

N⁶-(4-(4-Chlorophenyl)-2H-1,2,3-triazole-2-carbonyl)-L-lysine (CATK-3).

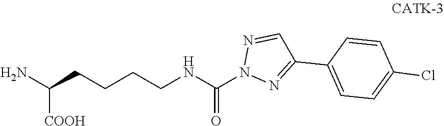 [SMILES: N[C@@H](CCCCNC(=O)n1ncc(-c2ccc(Cl)cc2)n1)C(=O)O]

To a solution of S3-3 (210.0 mg, 0.44 mmol) in DCM (3.0 mL) was added TFA (3.0 mL) at 0° C. The reaction mixture was stirred at room temperature for 6 h. Then, the solvent and excess TFA were removed under reduced pressure and the residue was purified by silica gel flash chromatography (MeOH/EtOAc=0:100 to 1:1) to afford the titled compound as a yellow solid (88.0 mg, 43% yield): ¹H NMR (400 MHz, CD₃OD/CF₃CO₂D=5:1) δ 8.37 (s, 1H), 7.96 (d, J=8.4 Hz, 2H), 7.50 (d, J=8.4 Hz, 2H), 3.99 (t, J=6.2 Hz, 1H), 3.48 (t, J=7.0 Hz, 2H), 2.07-1.92 (m, 2H), 1.80-1.73 (m, 2H), 1.64-1.48 (m, 2H); ¹³C NMR (75 MHz, CD₃OD/CF₃CO₂D=5:1) δ 171.9, 150.7, 150.0, 136.8, 135.7, 130.3, 129.1, 128.8, 53.9, 41.3, 39.1, 29.9, 23.2; HRMS (ESI) calcd for C₁₅H₁₉³⁵ClN₅O₃ 352.1176 [M+H⁺], found 352.1180; C₁₅H₁₉³⁷ClN₅O₃ 354.1142 [M+H⁺], found 354.1150.

N⁶-(4-(4-Chlorophenyl)-1H-1,2,3-triazole-1-carbonyl)-L-lysine (CATK-3a).

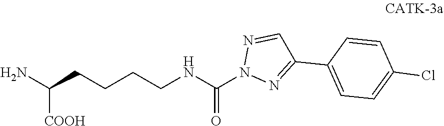 [SMILES: N[C@@H](CCCCNC(=O)n1ncc(-c2ccc(Cl)cc2)n1)C(=O)O]

To a mixture of S3-3a and 4-(4-chlorophenyl)-1H-1,2,3-triazole (230.0 mg, 85:15) in DCM (2.0 mL) was added TFA (2.0 mL) at 0° C. The reaction mixture was stirred at room temperature for 6 h. Then, the solvent and excess TFA were removed under reduced pressure and the residue was washed successively with DCM, Et₂O, and water to afford the titled compound as a yellow solid (34.0 mg, 18% yield): ¹H NMR (300 MHz, CD₃OD/CF₃CO₂D=5:1) δ 8.71 (s, 1H), 7.84 (d, J=8.5 Hz, 2H), 7.43 (d, J=8.5 Hz, 2H), 3.95 (t, J=6.3 Hz, 1H), 3.48 (t, J=6.8 Hz, 2H), 2.04-1.92 (m, 2H), 1.77-1.70 (m, 2H), 1.61-1.53 (m, 2H); ¹³C NMR (75 MHz, CD₃OD/CF₃CO₂D=5:1) δ 171.9, 149.3, 148.2, 135.8, 130.3, 129.6, 128.4, 120.1, 53.9, 41.2, 31.1, 29.9, 23.2; HRMS (ESI) calcd for C₁₅H₁₈³⁵ClN₅O₃Na 374.0991 [M+Na⁺], found 374.0988; C₁₅H₁₈³⁷ClN₅O₃Na 376.0961 [M+H⁺], found 376.0963.

tert-Butyl N²-(tert-butoxycarbonyl)-N⁶-(4-(thiophen-2-yl)-2H-1,2,3-triazole-2-carbonyl)-L-lysinate (S3-4).

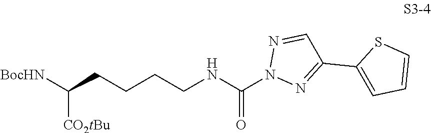 [SMILES: CC(C)(C)OC(=O)N[C@@H](CCCCNC(=O)n1ncc(-c2cccs2)n1)C(=O)OC(C)(C)C]

To a solution of triphosgene (329.4 mg, 1.11 mmol) in DCM (5.0 mL) at 0° C. was added dropwise a solution of S2 (907.3 mg, 3.0 mmol) and DIEA (629 μL, 3.6 mmol) in DCM (3.0 mL), and the mixture was stirred at 0° C. for 30 min. Then, a solution of 4-(thiophen-2-yl)-1H-1,2,3-triazole (453.0 mg, 3.0 mmol) and DIEA (629 μL, 3.6 mmol) in DCM (3.0 mL) was added, and the reaction mixture was stirred at room temperature for another 30 min. The solvent was removed under reduced pressure and the residue was dissolved with EtOAc. The organic layer was washed successively with saturated KHSO₄, saturated NaHCO₃, and brine, and then dried over anhydrous Na₂SO₄, filtered, and concentrated. The residue was purified by silica gel flash chromatography (EtOAc/hexanes=1:4) to give the title compound as a yellow oil (503.5 mg, 35% yield): ¹H NMR (400 MHz, CDCl₃) δ 7.97 (s, 1H), 7.51 (d, J=3.8 Hz, 1H), 7.41 (d, J=5.1 Hz, 1H), 7.36-7.30 (m, 1H), 7.12-7.10 (m, 1H), 5.17 (d, J=8.3 Hz, 1H), 4.22-4.17 (m, 1H), 3.53-3.48 (m, 2H), 1.84-1.66 (m, 5H), 1.46-1.44 (m, 19H); ¹³C NMR (101 MHz, CDCl₃) δ 171.8, 155.4, 147.3, 145.2, 133.9, 130.8, 127.9, 127.3, 126.7, 81.9, 79.7, 53.6, 40.7, 32.7, 29.1, 28.3, 28.0, 22.4; HRMS (ESI) calcd for C₂₂H₃₃N₅O₅SNa 502.2091 [M+Na⁺], found 502.2095.

tert-Butyl N²-(tert-butoxycarbonyl)-N⁶-(4-(thiophen-2-yl)-1H-1,2,3-triazole-1-carbonyl)-L-lysinate (S3-4a).

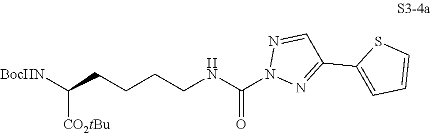 [SMILES: CC(C)(C)OC(=O)N[C@@H](CCCCNC(=O)n1ncc(-c2cccs2)n1)C(=O)OC(C)(C)C]

The titled minor product was obtained after silica gel flash chromatography as a yellow solid (273.3 mg, 19% yield): ¹H NMR (400 MHz, CDCl₃) δ 8.40 (s, 1H), 7.58-7.55 (m, 1H), 7.46 (d, J=3.7 Hz, 1H), 7.35 (d, J=5.0 Hz, 1H), 7.11-7.09 (m, 1H), 5.16 (d, J=8.3 Hz, 1H), 4.19 (d, J=6.8 Hz, 1H), 3.55-3.50 (m, 2H), 1.85-1.67 (m, 5H), 1.46-1.44 (m, 19H); ¹³C NMR (101 MHz, CDCl₃) δ 171.8, 155.4, 147.1, 143.3, 131.5, 127.7, 125.9, 125.1, 117.2, 81.8, 79.6, 53.7, 40.6, 32.5, 29.0, 28.3, 27.9, 22.4; HRMS (ESI) calcd for C₂₂H₃₃N₅O₅SNa 502.2091 [M+Na⁺], found 502.2088.

N⁶-(4-(Thiophen-2-yl)-2H-1,2,3-triazole-2-carbonyl)-L-lysine (CATK-4).

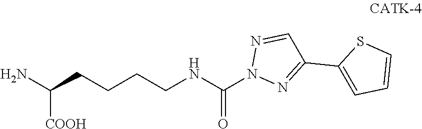 [SMILES: N[C@@H](CCCCNC(=O)n1ncc(-c2cccs2)n1)C(=O)O]

To a solution of S3-4 (450.0 mg, 0.94 mmol) in DCM (3.0 mL) was added TFA (3.0 mL) at 0° C. The reaction mixture was stirred at room temperature for 4 h. Then, solvent and excess TFA were removed under reduced pressure and the residue was purified by silica gel flash chromatography (MeOH/EtOAc=0:100 to 1:1) to give the titled compound as a white foam (32 mg, 8% yield): ¹H NMR (300 MHz, CD₃OD with one drop of CF₃CO₂D) δ 8.22 (s, 1H), 7.62 (d, J=3.0 Hz, 1H), 7.52 (d, J=6.0 Hz, 1H), 7.14-7.12 (m, 1H), 3.97 (t, J=6.2 Hz, 1H), 3.46 (t, J=6.8 Hz, 2H), 2.10-1.87 (m, 2H), 1.76-1.69 (m, 2H), 1.63-1.50 (m, 2H); ¹³C NMR (75 MHz, CD₃OD with one drop of CF₃CO₂D) δ 171.8, 149.8, 147.0, 135.3, 132.0, 129.0, 128.6, 128.4, 53.8, 41.2, 31.1, 30.0, 23.2; HRMS (ESI) calcd for C₁₃H₁₈N₅O₃S 324.1125 [M+H⁺], found 324.1125.

N⁶-(4-(Thiophen-2-yl)-1H-1,2,3-triazole-1-carbonyl)-L-lysine (CATK-4a).

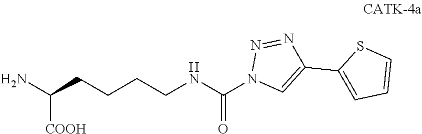 [SMILES: N[C@@H](CCCCNC(=O)n1cc(-c2cccs2)nn1)C(=O)O]

To a mixture of S3-4a (220.0 mg, 0.46 mmol) in DCM (2.0 mL) was added TFA (2.0 mL) at 0° C. The reaction mixture was stirred at room temperature for 5 h. Then, solvent and excess TFA were removed under reduced pressure and the residue was washed successively with DCM, Et₂O, and water to give the titled compound as a yellow solid (82 mg, 40% yield): ¹H NMR (300 MHz, CD₃OD with one drop of CF₃CO₂D) δ 8.57 (s, 1H), 7.43-7.37 (m, 2H), 7.03 (t, J=4.6 Hz, 1H), 3.89 (t, J=6.4 Hz, 1H), 3.39 (t, J=6.9 Hz, 2H), 1.99-1.79 (m, 2H), 1.71-1.62 (m, 2H), 1.53-1.41 (m, 2H); ¹³C NMR (75 MHz, CD₃OD with one drop of CF₃CO₂D) δ 171.8, 149.2, 144.4, 132.7, 128.9, 127.1, 126.4, 119.1, 53.8, 41.2, 31.2, 29.9, 23.2; HRMS (ESI) calcd for C₁₃H₁₇N₅O₃SNa 324.1125 [M+Na⁺], found 346.0944

tert-Butyl N²-(tert-butoxycarbonyl)-N⁶-(4-(furan-2-yl)-2H-1,2,3-triazole-2-carbonyl)-L-lysinate (S3-5).

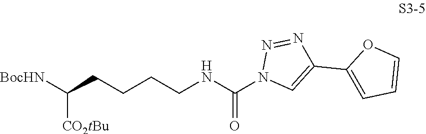 [SMILES: CC(C)(C)OC(=O)N[C@@H](CCCCNC(=O)n1cc(-c2ccco2)nn1)C(=O)OC(C)(C)C]

To a solution of triphosgene (211.0 mg, 0.71 mmol) in DCM (5.0 mL) cooled to 0° C. was added dropwise a solution of S2 (518.9 mg, 1.92 mmol) and DIEA (402 μL, 2.30 mmol) in DCM (3.0 mL). The reaction mixture was stirred at 0° C. for 30 min. Then, a solution of 4-(furan-2-yl)-1H-1,2,3-triazole (260.0 mg, 1.92 mmol) and DIEA (402 μL, 1.2 equiv.) in DCM (3.0 mL) was added, and the mixture was stirred at room temperature for another 30 min. The solvent was removed under reduced pressure and the residue was dissolved in EtOAc. The organic layer was washed successively with saturated KHSO₄, saturated NaHCO₃, and brine, and then dried over anhydrous Na₂SO₄, filtered, and concentrated. The residue was purified by silica gel flash chromatography (EtOAc/hexanes=1:4) to give the title compound as a yellow oil (326.1 mg, 37% yield): ¹H NMR (400 MHz, CDCl₃) δ 8.00 (s, 1H), 7.55 (s, 1H), 6.94 (d, J=3.4 Hz, 1H), 6.55-6.53 (m, 1H), 5.11 (d, J=8.3 Hz, 1H), 4.21-4.16 (m, 1H), 3.53-3.50 (m, 2H), 1.89-1.51 (m, 6H), 1.45 (s, 9H), 1.44 (s, 9H); ¹³C NMR (101 MHz, CDCl₃) δ 171.8, 155.4, 147.3, 144.4, 143.6, 142.3, 133.6, 111.8, 109.6, 81.9, 79.6, 53.6, 40.7, 32.6, 29.0, 28.3, 27.9, 22.4; HRMS (ESI) calcd for C₂₂H₃₃N₅O₆Na 486.2323 [M+Na⁺], found 486.2324.

tert-Butyl N²-(tert-butoxycarbonyl)-N⁶-(4-(furan-2-yl-1H-1,2,3-triazole-1-carbonyl)-L-lysinate (S3-5a).

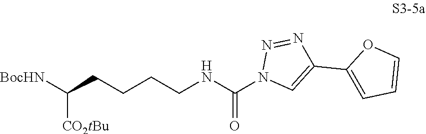 [SMILES: CC(C)(C)OC(=O)N[C@@H](CCCCNC(=O)n1cc(-c2ccco2)nn1)C(=O)OC(C)(C)C]

The titled minor product was obtained after silica gel flash chromatography as a yellow oil (223.4 mg, 25% yield): ¹H NMR (300 MHz, CDCl₃) δ 8.38 (s, 1H), 7.50 (d, J=1.8 Hz, 1H), 7.37 (t, J=6.1 Hz, 1H), 6.90 (d, J=3.4 Hz, 1H), 6.52-6.50 (m, 1H), 5.10 (d, J=8.3 Hz, 1H), 4.20-4.17 (m, 1H), 3.54-3.48 (m, 2H), 1.87-1.65 (m, 6H), 1.46 (s, 9H), 1.44 (s, 9H); ¹³C NMR (75 MHz, CDCl₃) δ 171.8, 155.4, 147.1, 145.0, 142.8, 140.9, 117.2, 111.5, 107.8, 81.9, 79.7, 53.6, 40.6, 32.6, 29.0, 28.3, 22.4; HRMS (ESI) calcd for C₂₂H₃₃N₅O₆Na 486.2323 [M+Na⁺], found 486.2340.

N⁶-(4-(Furan-2-yl)-2H-1,2,3-triazole-2-carbonyl)-L-lysine (CATK-5).

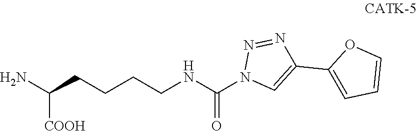 [SMILES: N[C@@H](CCCCNC(=O)n1cc(-c2ccco2)nn1)C(=O)O]

To a solution of S3-5 (271.1 mg, 0.59 mmol) in DCM (3.0 mL) at 0° C. was added TFA (3.0 mL). The reaction mixture was stirred at room temperature for 4 h. Then the solvent and excess TFA were removed under reduced pressure, and the residue was purified by silica gel flash chromatography (MeOH/EtOAc=0:100 to 1:1) to give the titled compound as a yellow solid (130 mg, 52% yield): ¹H NMR (300 MHz, CD₃OD with one drop of CF₃CO₂D) δ 8.21 (s, 1H), 7.68 (m, J=1.9, 0.8 Hz, 1H), 7.02 (dd, J=3.4, 0.8 Hz, 1H), 6.61 (dd, J=3.5, 1.9 Hz, 1H), 3.98 (t, J=6.3 Hz, 1H), 3.46 (t, J=6.8 Hz, 2H), 2.07-1.90 (m, 2H), 1.79-1.70 (m, 2H), 1.65-1.49 (m, 2H); ¹³C NMR (75 MHz, CD₃OD with one drop of CF₃CO₂D) δ 171.8, 149.8, 145.8, 145.4, 143.8, 135.0, 112.9, 110.9, 53.8, 41.2, 31.1, 30.0, 23.2; HRMS (ESI) calcd for C₁₃H₁₇N₅O₄Na 330.1173 [M+Na⁺], found 330.1170.

N⁶-(4-(Furan-2-yl)-1H-1,2,3-triazole-1-carbonyl)-L-lysine (CATK-5a). To a mixture of S3-5a (223.4 mg, 0.48 mmol) in DCM (2.0 mL) at 0° C. was added TFA (2.0 mL).

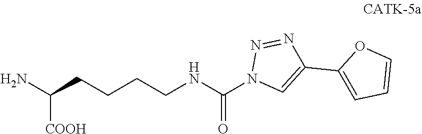 [SMILES: N[C@@H](CCCCNC(=O)n1cc(-c2ccco2)nn1)C(=O)O]

The reaction mixture was stirred at room temperature for 4 h. Then, the solvent and excess TFA were removed under reduced pressure and the residue was washed successively with DCM, Et₂O, and water to give the titled compound as a yellow solid (62 mg, 30% yield): ¹H NMR (300 MHz, CD₃OD with one drop of CF₃CO₂D) δ 8.48 (s, 1H), 7.51 (d, J=1.7 Hz, 1H), 6.80 (d, J=3.4 Hz, 1H), 6.47-6.45 (m, 1H), 3.89 (t, J=6.3 Hz, 1H), 3.39 (t, J=6.8 Hz, 2H), 1.99-1.81 (m, 2H), 1.71-1.61 (m, 2H), 1.54-1.39 (m, 2H); ¹³C NMR (75 MHz, CD₃OD with one drop of CF₃CO₂D) δ 171.8, 149.1, 146.3, 144.4, 141.7, 119.0, 112.6, 108.8, 53.8, 41.2, 31.1, 29.9, 23.2; HRMS (ESI) calcd for C₁₃H₁₇N₅O₄Na 330.1173 [M+Na⁺], found 330.1167.

tert-Butyl N²-(tert-butoxycarbonyl)-N⁶-(4-(5-methylfuran-2-yl)-2H-1,2,3-triazole-2-carbonyl)-L-lysinate (S3-6).

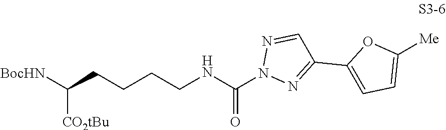 [SMILES: Cc1ccc(-c2cnn(C(=O)NCCCC[C@H](NC(=O)OC(C)(C)C)C(=O)OC(C)(C)C)n2)o1]

To a solution of triphosgene (183.0 mg, 0.62 mmol) in DCM (5.0 mL) at 0° C. was added dropwise a solution of S2 (505.0 mg, 1.67 mmol) and DIEA (352 μL, 2.0 mmol) in DCM (3.0 mL). The reaction mixture was stirred at 0° C. for 30 min. Then, a solution of 4-(5-methylfuran-2-yl)-1H-1,2,3-triazole (248.5 mg, 1.67 mmol) and DIEA (352 μL, 2.0 mmol) in DCM (3.0 mL) was added, and the reaction mixture was stirred at room temperature for another 30 min. The solvent was removed under reduced pressure and the residue was dissolved in EtOAc. The organic layer was washed successively with saturated KHSO₄ solution, saturated NaHCO₃ solution, and brine, and then dried over anhydrous Na₂SO₄, filtered, and concentrated. The residue was purified by silica gel flash chromatography (EtOAc/hexanes=1:4) as a yellow oil (225.2, 29% yield): ¹H NMR (400 MHz, CDCl₃) δ 7.95 (s, 1H), 7.25 (t, J=5.9 Hz, 1H), 6.81 (d, J=3.2 Hz, 1H), 6.12 (d, J=3.3 Hz, 1H), 5.13 (d, J=8.3 Hz, 1H), 4.19 (d, J=6.7 Hz, 1H), 3.52-3.47 (m, 2H), 2.38 (s, 3H), 1.83-1.79 (m, 1H), 1.76-1.62 (m, 3H), 1.52-1.48 (m, 2H), 1.45 (s, 9H), 1.44 (s, 9H); ¹³C NMR (101 MHz, CDCl₃) δ 171.8, 155.3, 153.8, 147.4, 142.5, 142.4, 133.4, 110.7, 108.0, 81.8, 53.6, 40.6, 32.5, 29.0, 28.2, 27.9, 22.4, 13.6; HRMS (ESI) calcd for C₂₃H₃₅N₅O₆Na 500.2480 [M+Na⁺], found 500.2485.

tert-Butyl N²-(tert-butoxycarbonyl)-N⁶-(4-(5-methylfuran-2-yl)-1H-1,2,3-triazole-1-carbonyl)-L-lysinate (S3-6a).

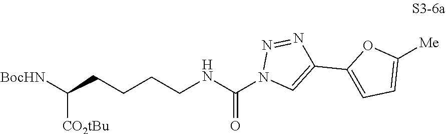 [SMILES: Cc1ccc(-c2cn(C(=O)NCCCC[C@H](NC(=O)OC(C)(C)C)C(=O)OC(C)(C)C)nn2)o1]

The titled minor product was obtained after silica gel flash chromatography as a pale-yellow solid (189.2 mg, 24% yield): ¹H NMR (400 MHz, CDCl₃) δ 8.33 (s, 1H), 7.49 (t, J=5.9 Hz, 1H), 6.77 (d, J=3.2 Hz, 1H), 6.09 (d, J=3.3 Hz, 1H), 5.16 (d, J=8.3 Hz, 1H), 4.19 (d, J=6.7 Hz, 1H), 3.54-3.49 (m, 2H), 2.36 (s, 3H), 1.86-1.51 (m, 6H), 1.45 (s, 9H), 1.43 (s, 9H), 1.27-1.21 (m, 1H); ¹³C NMR (101 MHz, CDCl₃) δ 171.8, 155.4, 152.8, 147.2, 143.2, 141.0, 116.6, 110.0, 108.7, 107.5, 81.8, 79.6, 53.7, 40.5, 32.5, 29.0, 28.2, 27.9, 22.4, 13.5; HRMS (ESI) calcd for C₂₃H₃₅N₅O₆Na 500.2480 [M+Na⁺], found 500.2489.

N⁶-(4-(5-Methylfuran-2-yl)-2H-1,2,3-triazole-2-carbonyl)-L-lysine (CATK-6).

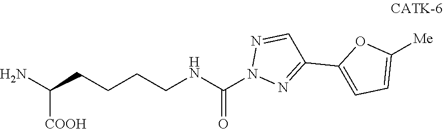 [SMILES: Cc1ccc(-c2cnn(C(=O)NCCCC[C@H](N)C(=O)O)n2)o1]

To a solution of S3-6 (162.0 mg, 0.34 mmol) in DCM (3.0 mL) at 0° C. was added TFA (3.0 mL). The reaction mixture was stirred at room temperature for 4 h. Then, the solvent and excess TFA were removed under reduced pressure and the residue was purified by silica gel flash chromatography (MeOH/EtOAc=0:100 to 1:1) to give the titled compound as a pale-yellow solid (10 mg, 13% yield): ¹H NMR (300 MHz, D₂O/CD₃CN=1:1) δ 8.12-8.10 (m, 1H), 6.89-6.87 (m, 1H), 6.24-6.21 (m, 1H), 3.72 (t, J=6.0 Hz, 1H), 3.46-3.40 (m, 2H), 2.36 (s, 3H), 1.94-1.89 (m, 2H), 1.73-1.68 (m, 2H), 1.53-1.49 (m, 2H); ¹³C NMR (75 MHz, D₂O/CD₃CN=1:1) δ 174.9, 155.6, 149.6, 143.3, 142.9, 134.8, 112.6, 109.0, 55.5, 41.1, 31.0, 29.2, 22.8, 13.6. HRMS (ESI) calcd for C₁₄H₁₉N₅O₄Na 344.1329 [M+Na⁺], found 344.1324

N⁶-(4-(5-Methylfuran-2-yl)-2H-1,2,3-triazole-2-carbonyl)-L-lysine (CATK-6a).

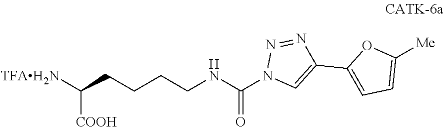

To a mixture of S3-6a (150.0 mg, 0.31 mmol) in DCM (2.0 mL) at 0° C. was added TFA (2.0 mL). The reaction mixture was stirred at room temperature for 5 h. Then, the solvent and excess TFA were removed under reduced pressure and the residue was washed with DCM, Et₂O, and water successively to give the titled compound as a pale-yellow solid (49 mg, 36% yield): ¹H NMR (300 MHz, CD₃OD with one drop of CF₃CO₂D) δ 8.50 (s, 1H), 6.76 (s, 1H), 6.15 (s, 1H), 3.98 (t, J=6.5 Hz, 1H), 3.47 (t, J=7.0 Hz, 2H), 2.35 (s, 3H), 2.03-1.92 (m, 2H), 1.78-1.70 (m, 2H), 1.61-1.49 (m, 2H). ¹³C NMR (75 MHz, CD₃OD with one drop of CF₃CO₂D) δ 171.8, 154.4, 149.1, 144.5, 141.9, 118.3, 109.8, 108.6, 53.8, 49.6, 41.2, 31.1, 29.9, 23.2, 13.4; HRMS (ESI) calcd for C₁₄H₂₀N₅O₄ 322.1515 [M+H⁺], found 322.1507.

### Synthesis of CATK-7

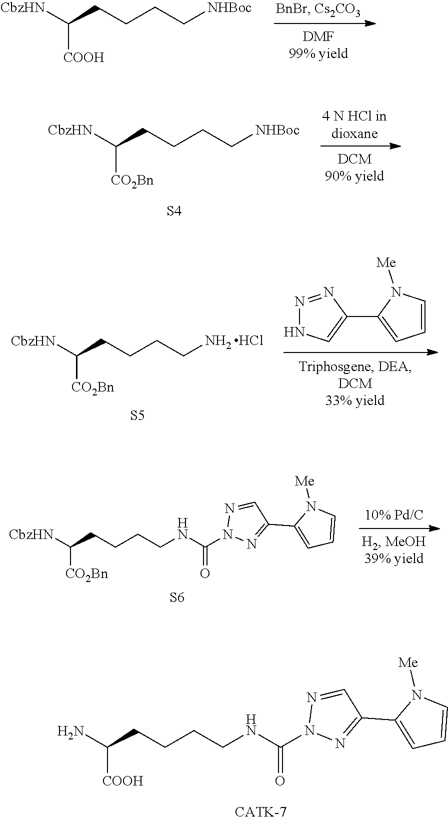

Benzyl N²-((benzyloxy)carbonyl)-N⁶-(tert-butoxycarbonyl)-L-lysinate (S4).

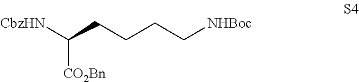 [SMILES: CC(C)(C)OC(=O)NCCCC[C@H](NC(=O)OCc1ccccc1)C(=O)OCc1ccccc1]

To a solution of Cbz-Lys(Boc)-OH (2.28 g, 6.0 mmol) in DMF (100.0 mL) was added Cs₂CO₃ (2.34 g, 7.2 mmol). The mixture was stirred for 30 min, and then benzyl bromide (0.86 mL, 7.2 mmol) was added dropwise at 0° C. The reaction mixture was stirred at room temperature for 2 h. The mixture was poured into water and extracted with EtOAc (10 mL×3). The combined organic layers were washed with brine and dried over anhydrous Na₂SO₄, filtered, and concentrated. The crude product was purified by silica gel flash chromatography (EtOAc/hexanes=1:2) to give the title compound as a colorless oil (2.90 g, 99% yield): ¹H NMR (400 MHz, CDCl₃) δ 7.36-7.32 (m, 10H), 5.40 (d, J=8.0 Hz, 1H), 5.23-5.09 (m, 5H), 4.52 (s, 1H), 4.42-4.37 (m, 1H), 3.07-3.02 (m, 2H), 1.86-1.81 (m, 1H), 1.72-1.67 (m, 2H), 1.42 (s, 9H), 1.37-1.19 (m, 3H); ¹³C NMR (101 MHz, CDCl₃) δ 172.2, 156.0, 128.6, 128.5, 128.3, 128.2, 128.1, 67.1, 67.0, 53.8, 40.0, 32.1, 29.5, 28.4, 22.2; HRMS (ESI) calcd for C₂₆H₃₄N₂O₆Na 493.2309 [M+Na⁺], found 493.2304.

Benzyl N²-((benzyloxy)carbonyl)-L-lysine hydrochloride (S5).

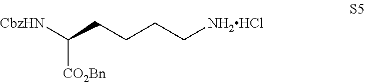

To a solution of S4 (6.0 mmol) in DCM (5.0 mL) at 0° C. was added dropwise 4 N HCl in dioxane (18.0 mL). The reaction mixture was stirred at room temperature for 4 h. The solvent was evaporated, and the residue was titrated with Et₂O to afford the title compound as a white sticky solid (2.20 g, 90% yield): ¹H NMR (500 MHz, DMSO-d₆) δ 8.03 (s, 3H), 7.81-7.79 (m, 1H), 7.39-7.29 (m, 8H), 5.16-5.00 (m, 4H), 4.08-4.05 (m, 1H), 2.72-2.68 (m, 2H), 1.76-1.52 (m, 4H), 1.39-1.33 (m, 2H); ¹³C NMR (126 MHz, DMSO-d₆) δ 172.2, 156.2, 136.8, 135.9, 128.4, 128.3, 128.0, 127.8, 127.8, 127.7, 65.9, 65.5, 53.9, 38.3, 30.0, 26.4, 22.4.

Benzyl N²-((benzyloxy)carbonyl)-N⁶-(4-(1-methyl-1H-pyrrol-2-yl)-2H-1,2,3-triazole-2-carbonyl)-L-lysinate (S6).

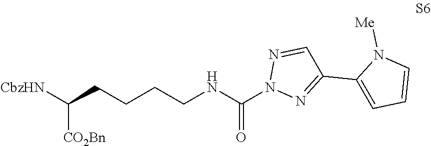 [SMILES: Cn1cccc1-c1cnn(C(=O)NCCCC[C@H](NC(=O)OCc2ccccc2)C(=O)OCc2ccccc2)n1]

To a solution of triphosgene (132.0 mg, 0.44 mmol.) in DCM (7.0 mL) at 0° C. was added dropwise a solution of S3 (488.0 mg, 1.2 mmol) and DIEA (630 μL, 3.6 mmol) in DCM (4.0 mL). The reaction mixture was stirred at 0° C. for 30 min. Then, a solution of 4-(1-methyl-1H-pyrrol-2-yl)-1H-1,2,3-triazole (178.0 mg, 1.2 mmol) in DCM (4.0 mL) and DIEA (630 μL, 3.6 mmol) were added, and the mixture was stirred at room temperature for another 30 min. The solvent was removed under reduced pressure and the residue was dissolved in EtOAc. The organic layer was washed successively with saturated KHSO₄ solution, saturated NaHCO₃ solution, and brine, and then dried over anhydrous Na₂SO₄, filtered, and concentrated, The residue was purified by silica gel flash chromatography (EtOAc/hexanes=1:4) to give the title compound as a colorless oil (213.3 mg, 33% yield): ¹H NMR (300 MHz, CDCl₃) δ 7.83 (s, 1H), 7.47-7.21 (m, 10H), 7.13 (t, J=6.1 Hz, 1H), 6.73-6.72 (m, 1H), 6.58-6.56 (m, 1H), 6.19-6.16 (m, 1H), 5.54 (d, J=8.3 Hz, 1H), 5.21-5.02 (m, 4H), 4.45-4.38 (m, 1H), 3.89 (s, 3H), 3.42-3.35 (m, 2H), 1.93-1.81 (m, 1H), 1.75-1.55 (m, 3H), 1.42-1.34 (m, 2H); ¹³C NMR (75 MHz, CDCl₃) δ 172.2, 147.7, 135.0, 128.6, 128.5, 128.3, 128.2, 128.0, 126.3, 121.9, 111.5, 108.4, 67.2, 67.0, 53.7, 40.5, 36.5, 32.1, 29.0, 22.4; HRMS (ESI) calcd for C₂₉H₃₃N₆O₅ 545.2512 [M+H⁺], found 545.2503.

N⁶-(4-(1-Methyl-1H-pyrrol-2-yl)-2H-1,2,3-triazole-2-carbonyl)-L-lysine (CATK-7).

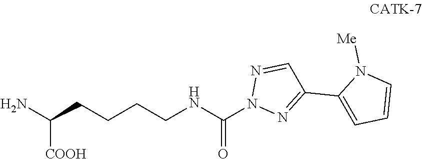 [SMILES: Cn1cccc1-c1cnn(C(=O)NCCCC[C@H](N)C(=O)O)n1]

To a solution of S6 (200.0 mg, 0.37 mmol) in MeOH (5.0 mL) was added 10% Pd/C (20.0 milligrams (milligram(s)=mg(s))). The mixture was stirred in a flask fitted with a hydrogen balloon at room temperature overnight. Then, Pd/C was removed by filtering the mixture through celite, and the filtrate was concentrated. The crude was recrystallized in MeOH/Et₂O to afford the title compound as a white solid (46.0 mg, 39% yield): ¹H NMR (400 MHz, D₂O) δ 8.02 (s, 1H), 6.88 (s, 1H), 6.62 (s, 1H), 6.26-6.15 (m, 1H), 3.82 (s, 3H), 3.76 (t, J=6.1 Hz, 1H), 3.39 (t, J=7.0 Hz, 2H), 1.95-1.89 (m, 2H), 1.71-1.66 (m, 2H), 1.52-1.47 (m, 2H); ¹³C NMR (101 MHz, D₂O) δ 174.6, 149.2, 143.9, 135.6, 127.0, 121.4, 111.4, 107.9, 54.6, 40.1, 35.7, 30.0, 28.2, 21.7; HRMS (ESI) calcd for C₁₄H₂₁N₆O₃ 321.1670 [M+H⁺], found 321.1665.

### Synthesis of CATK-8a, 8, 9

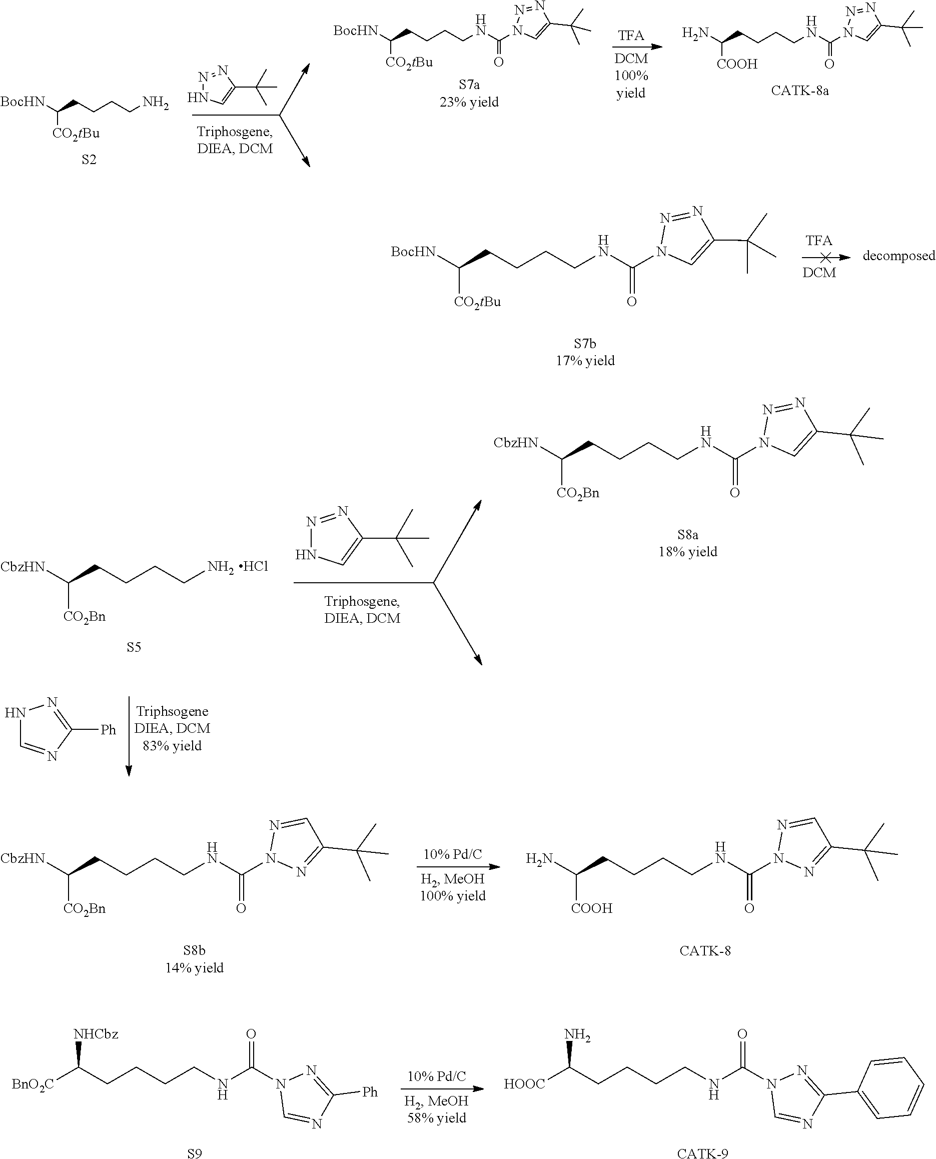

tert-Butyl N²-(tert-butoxycarbonyl)-N⁶-(4-tert-butyl)-1H-1,2,3-triazole-1-carbonyl)-L-lysinate (S7a).

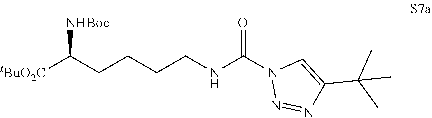 [SMILES: CC(C)(C)OC(=O)N[C@@H](CCCCNC(=O)n1cc(C(C)(C)C)nn1)C(=O)OC(C)(C)C]

Colorless oil, 210.0 mg, 23% yield. ¹H NMR (300 MHz, CDCl₃) δ 8.03 (s, 1H), 7.75 (t, J=6.0 Hz, 1H), 5.28 (d, J=9.0 Hz, 1H), 4.21-4.14 (m, 1H), 3.54-3.48 (m, 2H), 1.84-1.57 (m, 6H), 1.44-1.42 (m, 18H), 1.36 (s, 9H). ¹³C NMR (75 MHz, CDCl₃) δ 171.8, 157.9, 155.3, 147.7, 117.2, 81.5, 79.3, 53.7, 40.3, 32.3, 30.7, 29.9, 29.0, 28.2, 27.8, 22.3. HRMS (ESI) calcd for C₂₂H3₉N₅O₅ 476.2843 [M+Na⁺], found 476.2847.

tert-butyl N²-(tert-butoxycarbonyl)-N⁶-(4-(tert-butyl)-2H-1,2,3-triazole-2-carbonyl)-L-lysinate (S7b).

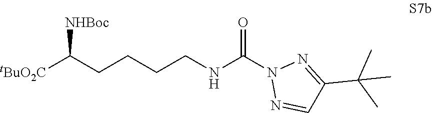 [SMILES: CC(C)(C)OC(=O)N[C@@H](CCCCNC(=O)n1ncc(C(C)(C)C)n1)C(=O)OC(C)(C)C]

Colorless oil, 150 mg, 17% yield. ¹H NMR (300 MHz, CDCl₃) δ 7.64 (s, 1H), 7.18 (t, J=6.0 Hz, 1H), 5.13 (d, J=9.0 Hz, 1H), 4.22-4.13 (m, 1H), 3.51-3.45 (m, 2H), 1.89-1.50 (m, 6H), 1.45-1.44 (m, 18H), 1.36 (s, 9H). ¹³C NMR (75 MHz, CDCl₃) δ 171.75, 160.19, 155.32, 147.67, 134.06, 81.76, 79.50, 53.64, 40.42, 32.55, 31.03, 29.91, 29.12, 28.24, 27.91, 22.41. HRMS (ESI) calcd for C₂₂H₃₉N₅O₅ 476.2843 [M+Na⁺], found 476.2844.

N⁶-(4-(tert-Butyl)-1H-1,2,3-triazole-1-carbonyl)-L-lysine (CATK-8a).

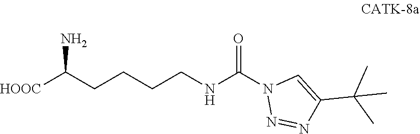 [SMILES: CC(C)(C)c1cn(C(=O)NCCCC[C@H](N)C(=O)O)nn1]

White solid, 144 mg, 100% yield. ¹H NMR (300 MHz, D₂O) δ 8.21 (s), 3.77-3.73 (m, 1H), 3.48-3.43 (m, 2H), 1.94-1.89 (m, 2H), 1.74-1.69 (m, 2H), 1.52-1.46 (m, 2H), 1.34 (s, 9H). ¹³C NMR (75 MHz, D₂O) δ 174.6, 158.1, 148.9, 118.6, 54.6, 40.0, 30.0, 29.1, 28.0, 21.6. HRMS (ESI) calcd for C13H₂₃N₅O₃Na 320.1693 [M+Na⁺], found 320.1680.

Benzyl N²-((benzyloxy)carbonyl)-N⁶-(4-(tert-butyl)-1H-1,2,3-triazole-1-carbonyl)-L-lysinate (S8a).

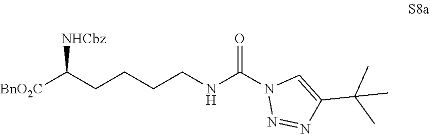

Colorless oil, 190 mg, 18% yield. ¹H NMR (300 MHz, CDCl₃) δ 7.97 (s, 1H), 7.47 (t, J=6.0 Hz, 1H), 7.32-7.27 (m, 10H), 5.62 (d, J=9.0 Hz, 1H), 5.25-5.14 (m, 2H), 5.08 (s, 2H), 4.46-4.39 (m, 1H), 4.41-3.34 (m, 2H), 1.91-1.44 (m, 6H), 1.35 (s, 9H). ¹³C NMR (75 MHz, CDCl₃) δ 172.2, 158.1, 156.0, 147.7, 136.2, 135.3, 128.6, 128.4, 128.4, 128.3, 128.1, 128.0, 117.2, 67.1, 66.9, 53.7, 40.2, 32.0, 30.8, 30.0, 28.8, 22.3. HRMS (ESI) calcd for C₂₈H₃₆N₅O₅ 522.2711 [M+H⁺], found 522.2713.

Benzyl N²-((benzyloxy)carbonyl)-N⁶-(4-(tert-butyl)-2H-1,2,3-triazole-2-carbonyl)-L-lysinate (S8b).

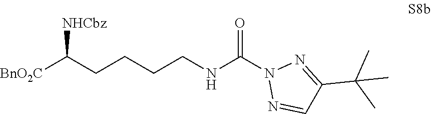

Colorless oil, 147.0 mg, 14% yield. ¹H NMR (300 MHz, CDCl₃) δ 7.62 (s, 1H), 7.34 (s, 10H), 7.05 (t, J=6.0 Hz, 1H), 5.38 (d, J=9.0 Hz, 1H), 5.17 (d, J=6.0 Hz, 2H), 5.10 (s, 2H), 4.47-4.40 (m, 1H), 3.42-3.35 (m, 2H), 1.88-1.57 (m, 6H), 1.36 (s, 9H). ¹³C NMR (75 MHz, CDCl₃) δ 172.2, 160.4, 156.0, 147.8, 136.3, 135.3, 134.3, 128.7, 128.6, 128.5, 128.3, 128.2, 67.3, 67.1, 53.8, 40.5, 32.4, 31.2, 30.1, 29.2, 22.5. HRMS (ESI) calcd for C₂₈H₃₆N₅O₅ 522.2711 [M+H⁺], found 522.2716.

N⁶-(4-(tert-Butyl)-2H-1,2,3-triazole-2-carbonyl)-L-lysine (CATK-8)

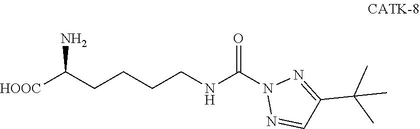 [SMILES: CC(C)(C)c1cnn(C(=O)NCCCC[C@H](N)C(=O)O)n1]

White powder, 60.0 mg, 100% yield. ¹H NMR (300 MHz, CD₃OD) δ 7.87 (s, 1H), 3.59-3.56 (m, 1H), 3.45-3.40 (m, 2H),1.94-1.84 (m, 3H), 1.73-1.65 (m, 3H), 1.57-1.48 (m, 3H), 1.37 (s, 9H). ¹³C NMR (75 MHz, CD₃OD) δ 173.1, 160.5, 148.7, 134.2, 54.7, 39.9, 30.7, 30.6, 29.2, 28.9, 22.1. δ HRMS (ESI) calcd for 320.1693 C₁₃H₂₃N₅O₃Na [M+Na⁺], found 320.1689.

Benzyl N²-((benzyloxy)carbonyl)-N-(3-phenyl-1H-1,2,4-triazole-1-carbonyl)-L-lysinate (S9).

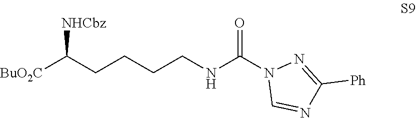

Colorless oil, 899.0 mg, 83% yield. ¹H NMR (300 MHz, CDCl₃) δ 8.84 (s, 1H), 8.14-8.11 (m, 2H), 7.44-7.40 (m, 3H), 7.29 (d, J=6.0 Hz, 10H), 7.12 (t, J=6.0 Hz, 1H), 5.69 (d, J=9.0 Hz, 1H), 5.14 (d, J=12.0 Hz, 2H), 5.07 (s, 2H), 4.44-4.41 (m, 1H), 3.35-3.28 (m, 2H), 1.88-1.83 (m, 1H), 1.72-1.51 (m, 3H), 1.40-1.32 (m, 2H). ¹³C NMR (75 MHz, CDCl₃) δ 172.1, 163.0, 156.0, 147.9, 144.1, 136.1, 135.2, 130.2, 129.6, 128.6, 128.5, 128.5, 128.4, 128.2, 128.1, 128.0, 126.8, 67.1, 66.9, 53.7, 40.0, 32.0, 28.8, 22.3. HRMS (ESI) calcd for C₃₀H₃₂N₅O₅ 542.2398 [M+H⁺], found 542.2401.

N⁶-(3-Phenyl-1H-1,2,4-triazole-1-carbonyl)-L-lysine (CATK-9).

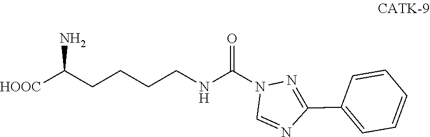 [SMILES: N[C@@H](CCCCNC(=O)n1cnc(-c2ccccc2)n1)C(=O)O]

White solid, 298.0 mg, 58% yield. ¹H NMR (300 MHz, DMSO-d₆) δ 9.18 (s, 1H), 8.76 (s, 1H), 8.12-8.09 (m, 2H), 7.56-7.47 (m, 6H), 3.31-3.26 (m, 1H), 3.1-3.11 (m, 1H), 1.80-1.70 (m, 2H), 1.67-1.52 (m, 3H), 1.44-1.32 (m, 2H). ¹³C NMR (101 MHz, DMSO-d₆) δ 170.3, 162.3, 148.2, 145.7, 130.6, 130.2, 129.3, 129.2, 126.9, 104.2, 54.5, 40.3, 31.2, 29.2, 22.9. HRMS (ESI) calcd for C₁₅H1₉N₅O₃Na 340.1380 [M+Na⁺], found 340.1381.

N⁶-((4-Fluorophenoxy)carbonyl)-L-lysine (FPheK).

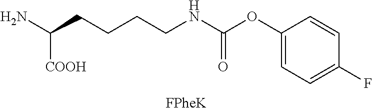 [SMILES: N[C@@H](CCCCNC(=O)Oc1ccc(F)cc1)C(=O)O ; "FPheK"]

FPheK was synthesized by following the literature procedure³ as a grey solid (50 mg, 60% yield): ¹H NMR (300 MHz, CD₃OD) δ 7.09 (d, J=6.4 Hz, 4H), 3.98 (t, J=6.3 Hz, 1H), 3.21 (t, J=6.7 Hz, 2H), 2.07-1.83 (m, 2H), 1.70-1.45 (m, 5H). HRMS (ESI) calcd for C₁₃H₁₈FN₂O₄ 285.1245 [M+H⁺], found 285.1255.

### Synthesis of FSY

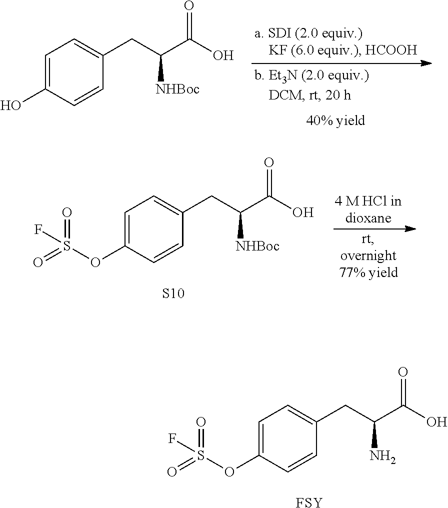

(S)-2-Amino-3-(4-((fluorosulfonyl)oxy)phenyl)propanoic acid (FSY).

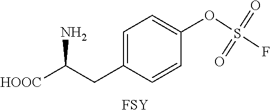

FSY was synthesized using a modified literature procedure. In brief, chamber A of a dried two-chamber reactor was filled with 1,1′-sulfonyldiimidazole (SDI, 141 mg, 0.71 mmol, 2.0 eq) and potassium fluoride (124 mg, 2.1 mmol, 6.0 eq). Boc-L-tyrosine (100 mg, 0.35 mmol, 1.0 eq), triethylamine (99 μL, 0.71 mmol, 2.0 eq) and DCM (4 mL) were added into chamber B. Then, 0.7 mL formic acid was injected into chamber A and the reaction was stirred at room temperature for 20 h. The solvent was removed under reduced pressure. The crude product was purified by flash column chromatography to give compound S10 in 40% yield (50 mg, 0.14 mmol). Next, S10 was treated with 4 N HCl in dioxane (5 mL) and the mixture was stirred overnight at room temperature. The solvent was removed under reduced pressure. The white residue was washed with cold ether to afford FSY as a white solid (32 mg, 77% yield): ¹H NMR (300 MHz, CD₃OD) δ 7.53-7.44 (m, 4H), 4.34-4.30 (m, 1H), 3.41-3.21 (m, 2H); HRMS (ESI) calcd for C₉H₁₁FNO₅S 264.0336 [M+H⁺], found 264.0327.

Site-specific incorporation of CATK into sfGFP. BL21(DE3) cells (50 μL) were co-transformed with the pET-sfGFP-Q204TAG and pEVOL-CATKRS plasmids using the heat shock method. The cells were recovered in 900 μL SOC at 37° C. for 1 hour before plating onto a Luria-Bertani (LB) agar plate containing 100 μg/mL ampicillin and 34 μg/mL chloramphenicol. A single colony from the plate was used to inoculate 6 mL LB broth containing 100 μg/mL ampicillin and 34 μg/mL chloramphenicol. One hundred twenty L overnight culture was used to inoculate 12 mL LB broth containing the same concentrations of antibiotics. The cells were grown until OD₆₀₀ reached ˜0.8 and the protein expression was induced by adding 0.2% arabinose and 1 mM isopropyl β-D-1-thiogalactopyranoside (IPTG). The culture was divided into two 6-mL portions. One portion of the culture was supplemented with 1 mM CATK, and the other portion served as a control without CATK. The cultures were incubated in an incubator-shaker (37° C., 280 rpm) for 8 hours. The cells were pelletized in 15 mL conical tubes and resuspended in 1.5 mL binding buffer (10 mM imidazole, 300 mM NaCl in Na₂HPO₄, pH 8.0) on ice for 15 min. The supernatant was directly used for fluorescence tests after sonication and centrifugation. The lysate was transferred into a 1.5 mL microcentrifuge tube containing 50 μL Ni-NTA agarose beads (Thermo HisPur™). The mixture was incubated for 2 hours with gentle shaking. The resin was centrifuged briefly and washed three times with washing buffer (50 mM imidazole, 300 mM NaCl in 50 mM Na₂HPO₄, pH 8.0). Finally, the protein was eluted with 500 μL elution buffer (250 mM imidazole, 300 mM NaCl in 50 mM Na₂HPO₄, pH 8.0). The protein yield was calculated based on the concentration determined using Pierce™ BCA protein assay kit (Thermo Fisher Scientific),

Site-specific incorporation of CATK into glutathione S-transferase (GST). BL21(DE3) cells (50 μL) were co-transformed with pET28a(+)-GST mutant and pEVOL-CATKRS plasmids using the heat shock method. The cells were recovered in 950 μL SOC media (New England Biolabs) and incubated at 37° C. for 1 hour before plating to a LB agar plate containing 50 μg/mL kanamycin and 34 μg/mL chloramphenicol. A single colony was used to inoculate 6 mL of LB containing 50 μg/mL kanamycin and 34 μg/mL chloramphenicol. Two hundred L aliquot of overnight culture was used to inoculate 20 mL LB medium containing the same concentrations of antibiotics. The cells were grown until OD₆₀₀ reached ˜0.8 and the protein expression was induced by adding 0.2% arabinose and 1 mM isopropyl β-D-1-thiogalactopyranoside (IPTG). The culture was divided into two 10-mL portions. One portion of the culture was supplemented with 1 mM CATK, and the other portion served as a control without CATK. The cultures were incubated overnight (25° C., 280 rpm, 16 hours). The cells were pelletized in 15 mL conical tubes and resuspended in 700 L BugBuster® Protein Extraction reagent (Millipore) before transferring into 1.5 mL microcentrifuge tube. The lysate was incubated for 20 min and then centrifuged before transferring to 1.5 mL microcentrifuge tube containing 50 μL Ni-NTA agarose beads (Thermo HisPur™). The mixture was diluted with 500 μL binding buffer (10 mM imidazole, 300 mM NaCl in 50 mM Na₂HIPO₄, pH 8.0) and incubated for 2 hours with gentle shaking at 4° C. The resin was centrifuged briefly and washed three times with washing buffer (50 mM imidazole, 300 mM NaCl in 50 mM Na₂HPO₄, pH 8.0). Finally, the protein was eluted with 1.0 mL elution buffer (250 mM imidazole, 300 mM NaCl in 50 mM Na₂HIPO₄, pH 8.0). The elution was concentrated using Amicon Ultra-0.5 mL Centrifugal Filter (MWCO 10 kDa; Millipore) followed by buffer exchange to a phosphate buffer (pH 7.4) to a final volume of 100 μL. The protein yield was calculated based on the concentration determined using Pierce™ BCA protein assay kit (Thermo Fisher Scientific).

The proteins were mixed with an equal amount of 2x SDS loading buffer and heated at 95° C. for 10 min before loading onto 4-12% SDS-PAGE gel (GenScript). The proteins were separated at 140 V for 60 min and detected using Coomassie blue staining. For western blot, the proteins were resolved by SDS-PAGE gel and transferred to a PVDF membrane (Thermo Fisher Scientific). The membrane was blocked in 1% casein in TBST (50 mM Tris, 150 mM NaCl, 0.05% Tween-20, pH 7.6) at 4° C. overnight, and then incubated with rabbit anti-His-tag antibody (1:1000, Abgent) in TBST at room temperature for 1 h. The membrane was washed with TBST (6×5 min) before the addition of the secondary goat anti-rabbit horseradish peroxidase conjugate (1:4000, Santa Cruz Biotech). After 30 minutes, the membrane was washed with TBST (6×5 min) and Tris buffer (100 mM, pH 9.5, 1×5 min). After the addition of Pierce™ ECL Western Blotting Substrate (Thermo Fisher Scientific), the membrane was incubated in dark for 5 min. Then the blot was exposed to an X-ray film (Phenix) to record the data.

Site-specific incorporation of FPheK or FSY into glutathione S-transferase (GST). BL21(DE3) cells (50 μL) were co-transformed with pET28a(+)-GST-E52TAG-E92K and pEVOL-FPheKRS or pEVOL-FSYRS plasmids using heat shock and recovered in 900 μL SOC media (New England Biolabs) and incubated at 37° C. for 1 hour before plating to Luria Broth (LB) agar plate containing 50 μg/mL kanamycin and 34 μg/mL chloramphenicol. A single colony from the plate was picked and used to inoculate 6 mL LB containing 50 μg/mL kanamycin and 34 μg/mL chloramphenicol. An aliquot of 200 μL from the overnight culture was used to inoculate a 20 mL culture of LB containing the same concentrations of antibiotics. Protein expression, purification, and mass spec determination were performed using the same procedure as those for the GST-CATK mutants.

Optimization of protein purification method for CATK-1-encoded SjGST protein. After protein expression, cells from 10-mL culture were harvested and resuspended in 700 μL BugBuster® Protein Extraction reagent (Millipore). The lysate was incubated at room temperature for 20 min and then centrifuged. The supernatant was collected and equally divided into two portions. One portion of the supernatant was transferred into 1.5 mL microcentrifuge tube containing 25 L Ni-NTA agarose beads (Thermo HisPur™) following the same purification procedure as above. Another portion of the supernatant was transferred into 1.5 mL microcentrifuge tube containing 25 μL glutathione agarose beads (Pierce© Glutathione Agarose). The mixture was diluted with 400 μL equilibration buffer (50 mM Tris, 150 mM NaCl, pH 8.0) and incubated for 2 hours with gentle shaking at 4° C. The resin was centrifuged briefly and washed four times with 200 μL washing buffer (50 mM Tris, 150 mM NaCl, pH 8.0). The supernatant was separately saved and monitored by measuring its absorbance at 280 nm until the baseline was reached. Proteins were eluted with 200 μL elution buffer (50 mM Tris, 150 mM NaCl, 10 mM reduced glutathione, pH 8.0) four times and protein elution was monitored by measuring the absorbance at 280 nm. Finally, the elution fractions were combined and concentrated using Amicon Ultra-0.5 mL Centrifugal Filter (MWCO 10 kDa; Millipore) followed by buffer exchange to a phosphate buffer (pH 7.4) to a final volume of 100 μL. The protein yield was calculated based on concentration determination using Pierce™ BCA protein assay kit (Thermo Fisher Scientific).

Site-specific incorporation of CATK-1 into NSa1 protein. BL21(DE3) cells (50 L) were co-transformed with pET28a(+)-NSa1 or pET28a(+)-NSa1(+10)-A13TAG and pEVOL-CATKRS plasmids using heat shock and recovered in 900 L SOC media (New England Biolabs) and incubated at 37° C. for 1 hour before plating to LB agar plate containing 50 μg/mL kanamycin and 34 μg/mL chloramphenicol. A single colony from the plate was picked and used to inoculate 6 mL LB containing 50 μg/mL kanamycin and 34 μg/mL chloramphenicol. An aliquot of 2 mL overnight culture was used to inoculate a 200 mL culture of LB containing the same concentrations of antibiotics. The cells were grown until OD₆₀₀ reached ˜0.8 and the protein expression was induced by adding 0.2% arabinose and 1 mM IPTG. The culture was divided into two 100-mL portions. One portion of the culture was supplemented with 1 mM CATK-1 and the other portion served as a control without CATK-1. The cultures were incubated overnight (25° C., 280 rpm, 16 hours). The cells were pelletized in 50 mL conical tubes and resuspended with 6 mL lysis buffer (50 mM Tris HCl, 0.5 M NaCl, pH 8.0) with protease inhibitor (Pierce™) on ice for 15 min. The cell was lysed by sonication on ice and centrifuged. The supernatant was transferred into 15 mL tube with 50 μL Ni-NTA agarose beads (Thermo HisPur™) and incubated for 2 hours with gentle shaking at 4° C. The resin was centrifuged briefly and washed three times with washing buffer (50 mM imidazole, 300 mM NaCl in 50 mM Na₂HPO₄, pH 8.0). Finally, the protein was eluted with 1.0 mL elution buffer (250 mM imidazole, 300 mM NaCl in 50 mM Na₂HPO₄, pH 8.0). The NSa1 protein encoding CATK-1 was dialyzed with starting buffer (50 mM Na₂HPO₄, 500 mM NaCl, pH 7.0), and further purified using cation ion-exchange chromatography (mono S 5/50G) with a NaCl gradient in 50 mM Na₂HIPO₄ buffer (pH 7.0).

Protein expression of NB1 encoding BocK and CATK-1. BL21(DE3) cells (50 μL) were co-transformed with pET28b(+)-NB1-V4TAG and pEVOL-CATKRS or pEVOL-wtPylRS plasmids using heat shock and recovered in 900 μL TB media and incubated at 37° C. for 1 hour before plating to LB agar plate containing 50 μg/mL kanamycin and 34 g/mL chloramphenicol. A single colony from the plate was picked and used to inoculate 6 mL LB containing 50 μg/mL kanamycin and 34 μg/mL chloramphenicol. An aliquot of 1 mL overnight culture was used to inoculate a 100 mL culture of TB containing the same concentrations of antibiotics. The cells were grown until OD₆₀₀ reached ˜0.8 and the protein expression was induced by adding 0.2% arabinose and 1 mM IPTG. The culture was divided into two 50-mL portions. One portion of the culture was supplemented with 1 mM CATK-1 or BocK and the other portion served as a control without unnatural amino acid. The cultures were incubated overnight (25° C., 280 rpm, 16 hours). The cells were pelletized in 50 mL conical tubes and resuspended with 4 mL lysis buffer (10 mM imidazole, 300 mM NaCl in 50 mM Na₂HPO₄, pH 8.0) with protease inhibitor (Pierce™) on ice for 15 min. The cell was lysed by sonication on ice and centrifuged. Next, the proteins were purified using Ni-NTA beads following the manufacturer's procedure.

Cell viability assay. One hundred μL exponentially growing HEK293T cells were seeded into a 96-well plate at a density of 5×10⁵ cells per ml. After 24 h, the cells were treated with varying concentrations of CATK amino acids and then incubated at 37° C. for 24 h. Then, 10 μL CCK-8 solution (Dojindo) was added to each well and the cells were further incubated in 37° C. incubator for 1 h. The plates were read immediately using Biotek microtiter plate reader at 450 nm.

Site-specific incorporation of CATK-1 into mCherry-TAG-EGFP in mammalian cells. Human Embryonic Kidney 293T (HEK293T) cells were seeded in a 12-well plate and grown in DMEM supplemented with 10% FBS (HyClone™ GE Healthcare Life Sciences) and 10 μg/mL Gentamycin (Gibco) and 2 μg/mL Plasmocin at 37° C., 5% CO₂ until ˜90% confluency. The medium was replaced with DMEM, and cells were transfected by using polyethylenimine (Sigma-Aldrich) in Opti-MEM® (Gibco) with two plasmids (one encoding CATKRS/tRNAPyl CUA pair and another encoding mCherry-TAG-EGFP-HA). Six hours post-transfection, the medium was replaced with fresh DMEM with 10% FBS in the presence or absence of 0.5 mM CATK-1. After 24 hours, live cell images were recorded using the Lionheart™ FX automated microscope (BioTek). The cells were lysed by modified RIPA buffer (25 mM Tris HCl, pH 7.4, 150 mM NaCl, 1% NP-40, 1% sodium deoxycholate, 0.1% SDS, 1 mM EDTA, 1 mM PSMF). 25 μL lysates were loaded to the 4-12% SDS-PAGE gel, separated at 140 V for 40 minutes, and then transferred to a PVDF membrane (Thermo Fisher Scientific). The membrane was blocked in 1% casein in TBST (50 mM Tris, 150 mM NaCl, 0.05% Tween-20, pH 7.6) at 4° C. overnight, and then incubated with mouse anti-HA tag antibody (1:10000, Thermo Fisher Scientific) in TBST at room temperature for 1 h. The membrane was washed with TBST (6×5 min) before the addition of the secondary goat anti-mouse horseradish peroxidase conjugate (1:5000, Santa Cruz Biotech). After 30 minutes, the membrane was washed with TBST (6×5 min) and incubated in 100 mM Tris buffer, pH 9.5 before the addition of Pierce™ ECL Western Blotting Substrate (Thermo Fisher Scientific) and incubation for 5 min. The blot was exposed to an X-ray film (Phenix).

NSa1 proteolytical stability assay. In a 1.5-mL microcentrifuge tube, TEV-cleaved, purified NSa1 (1.5 μM in 50 mM phosphate, 500 mM NaCl, pH 7.0) was incubated with Cathepsin B (Novus Biologicals; 0.065 μM) at 37° C. At various time points, every 3 μL reaction aliquots were taken out and mixed with 77 μL DPBS, and 60 μL solution was injected into QTOF-LC/MS for analysis.

Fluorescent labeling of NSa1 and flow cytometry. After TEV-cleavage and further cation exchange chromatography, the purified NSa1 proteins were buffer exchanged into the basic buffer (100 mM Pi, 450 mM NaCl, pH 8.3), and then incubated with AF488-NHS (Lumiprobe) (2-fold molar, optimized to afford non-labeled and mono-labeled protein as major species) at 4° C. with gentle shaking in darkness overnight. Then, thorough dialysis was employed to remove excess dye and protein concentrations were determined by Nanodrop (e_{NSa1}=28880 M⁻¹ cm⁻¹, e_{NSa1-plus10}=27390 M⁻¹ cm⁻¹; CF₂₈₀=0.11; Aₚᵣₒₜₑᵢₙ=A₂₈₀−A₄₉₃*CF₂80).

HeLa cells were seeded in a 48-well plate and grown in DMEM supplemented with 10% FBS and 10 μg/mL Gentamycin (Gibco) and 2 μg/mL Plasmocin at 37° C., 5% CO₂ until ˜₈₀% confluency. Cells were washed twice with pre-warmed PBS before switching to serum-free DMEM with Alexa-488 labeled protein. Cells were incubated at 37° C. for 4 hours. The cells were washed three times with PBS (including 20 U/mL heparin), trypsinized, and collected with 1.5 mL tubes. After brief centrifugation (400 g, 5 min) at room temperature, cells were collected and resuspended in PBS for flow cytometry analysis.

### Example 2

This example provides a description of the preparation, characterization, and use of non-crosslinked proteins and crosslinked proteins of the present disclosure.

The following proteins were made using methods described in Example 1:

12VC1-WT
[SEQ. ID. NO: 1]
MGSSHHHHHHSSGTENLYFQGVS SVPTKLEV VA*TPTSLLI
SWDAPAVTVF FYVITYGETG HGVGAFQAFK VPGSKSTATI SGLKPGVDYT
ITVYARGYSK QGPYKPSPIS INKRT (* = incorporation site for a first amino acid (e.g.,
BocK, BeLaK, or the like);
12VC1(+8)
[SEQ. ID. NO: 2]
MGSSHHHHHHSSGTENLYFQGVS SVPTKLKV VA*TPTSLLI
SWDAPAVTVF FYVITYGETG HGVGAFKAFK VPGSKSTATI SGLKPGVDYT
ITVYARGYSK KGPYKPSPIS INKRT (* = incorporation site for a first amino acid (e.g.,
BocK, BeLaK, or the like);
12VC1(+10)
[SEQ. ID. NO: 3]
MGSSHHHHHHSSGTENLYFQGVSKVPTKLEV VA*TPTSLLI
KWDAPAVTVK FYVITYGEKG HGVGAFQAFK VPGSKRTATI KGLKPGVDYT
ITVYARGYSK QGPYKPSPIS INKRT (* = incorporation site for a first amino acid (e.g.,
BocK, BeLaK, or the like)).
NSa1-Y92K-C1
[SEQ. ID. NO: 4]
MGSSHHHHHHSSGTENLYFQGC VSSVPTKLEV VAATPTSLLI
SWDAPAVTVD YYVITYGETG SGGYAWQEFE VPGSKSTATI SGLKPGVDYT
ITVYAGYYGY PTYYSSPISI NKRT;
NSa1-A13BocK-C1
[SEQ. ID. NO: 5]
MGSSHHHHHHSSGTENLYFQGC VSSVPTKLEV VA*TPTSLLI
SWDAPAVTVD YYVITYGETG SGGYAWQEFE VPGSKSTATI SGLKPGVDYT
ITVYAGYYGY PTYYSSPISI NYRT (* = BocK);
NSa1(+5)-A13BeLaK
[SEQ. ID. NO: 6]
MGSSHHHHHHSSGTENLYFQG VSSKPTKLRV VR*TPTSLKI
SWDAPAVTVD YYVITYGEKG SGGYAWQEFE VPGSKRTATI SGLKPGVDYT
ITVYAGYKGY PTYYSSPISI NYRT (* = BeLaK);
NSa1(+5)-A13BeLaK-Y92K
[SEQ. ID. NO: 7]
MGSSHHHHHHSSGTENLYFQG VSSKPTKLRV VR*TPTSLKI
SWDAPAVTVD YYVITYGEKG SGGYAWQEFE VPGSKRTATI SGLKPGVDYT
ITVYAGYKGY PTYYSSPISI NKRT (* = BeLaK);
NSa1(+5)-A13BocK-Y92K
[SEQ. ID. NO: 8]
MGSSHHHHHHSSGTENLYFQG VSSKPTKLRV VR*TPTSLKI
SWDAPAVTVD YYVITYGEKG SGGYAWQEFE VPGSKRTATI SGLKPGVDYT
ITVYAGYKGY PTYYSSPISI NKRT (* = BocK);
NSa1(+5)-A13BeLaK-Y92K-C1
[SEQ. ID. NO: 9]
MGSSHHHHHHSSGTENLYFQGC VSSKPTKLRV VR*TPTSLKI
SWDAPAVTVD YYVITYGEKG SGGYAWQEFE VPGSKRTATI SGLKPGVDYT
ITVYAGYKGY PTYYSSPISI NKRT (* = BeLaK);
NSa1(+5)-A13BocK-Y92K-C1
[SEQ. ID. NO: 10]
MGSSHHHHHHSSGTENLYFQGC VSSKPTKLRV VR*TPTSLKI
SWDAPAVTVD YYVITYGEKG SGGYAWQEFE VPGSKRTATI SGLKPGVDYT
ITVYAGYKGY PTYYSSPISI NKRT (* = BocK);
NSa1(+7)-A13BeLaK
[SEQ. ID. NO: 11]
MGSSHHHHHHSSGTENLYFQG VSSKPTKLRV VR*TPTSLKI
KWDAPAVTVD YYVITYGEKG RGGYAWQEFE VPGSKRTATI SGLKPGVDYT
ITVYAGYKGY PTYYSSPISI NYRT (* = BeLaK);
NSa1(+7)-A13BeLaK-Y92K-C1
[SEQ. ID. NO: 12]
MGSSHHHHHHSSGTENLYFQGC VSSKPTKLRV VR*TPTSLKI
KWDAPAVTVD YYVITYGEKG RGGYAWQEFE VPGSKRTATI SGLKPGVDYT
ITVYAGYKGY PTYYSSPISI NKRT (* = BeLaK);
NSa1(+7)-A13BocK-Y92K-C1
[SEQ. ID. NO: 13]
MGSSHHHHHHSSGTENLYFQGC VSSKPTKLRV VR*TPTSLKI
KWDAPAVTVD YYVITYGEKG RGGYAWQEFE VPGSKRTATI SGLKPGVDYT
ITVYAGYKGY PTYYSSPISI NKRT (* = BocK);
NSa1(+10)-A13BeLaK
[SEQ. ID. NO: 14]
MGSSHHHHHHSSGTENLYFQG VSSKPTKLRV VR*TPTSLKI
KWDAPAKTVD YYVITYGETG RGGYAWQRFE VPGSKRTATI KGLKPGVDYT
ITVYAGYKGY PTYYSSPISI NYRT (* = BeLaK);
NSa1(+10)-A13BeLaK-C1
[SEQ. ID. NO: 15]
MGSSHHHHHHSSGTENLYFQGC VSSKPTKLRV VR*TPTSLKI
KWDAPAKTVD YYVITYGETG RGGYAWQRFE VPGSKRTATI KGLKPGVDYT
ITVYAGYKGY PTYYSSPISI NYRT (* = BeLaK);
NSa1(+10)-A13BeLaK-Y92K-C1
[SEQ. ID. NO: 16]
MGSSHHHHHHSSGTENLYFQGC VSSKPTKLRV VR*TPTSLKI
KWDAPAKTVD YYVITYGETG RGGYAWQRFE VPGSKRTATI KGLKPGVDYT
ITVYAGYKGY PTYYSSPISI NKRT (* = BeLaK);
NSa1(+10)-A13BocK-Y92K-C1
[SEQ. ID. NO: 17]
MGSSHHHHHHSSGTENLYFQGC VSSKPTKLRV VR*TPTSLKI
KWDAPAKTVD YYVITYGETG RGGYAWQRFE VPGSKRTATI KGLKPGVDYT
ITVYAGYKGY PTYYSSPISI NKRT (* = BocK);
NSa1(+17)-A13BeLaK
[SEQ. ID. NO: 18]
MGSSHHHHHHSSGTENLYFQG VKSKPTKLRV VR*TPTSLKI
SWKAPKKTVD YYVITYGKTG SGGYAWQRFR VPGSKRTAKI KGLKPGVDYT
ITVYAGYKGY PTYYSSPISI NYRT (* = BeLaK);
NSa1(+17)-A13BeLaK-Y92K
[SEQ. ID. NO: 19]
MGSSHHHHHHSSGTENLYFQG VKSKPTKLRV VR*TPTSLKI
SWKAPKKTVD YYVITYGKTG SGGYAWQRFR VPGSKRTAKI KGLKPGVDYT
ITVYAGYKGY PTYYSSPISI NKRT (* = BeLaK);
NSa1(+17)-A13BeLaK-Y92K-C1
[SEQ. ID. NO: 20]
MGSSHHHHHHSSGTENLYFQGC VKSKPTKLRV VR*TPTSLKI
SWKAPKKTVD YYVITYGKTG SGGYAWQRFR VPGSKRTAKI KGLKPGVDYT
ITVYAGYKGY PTYYSSPISI NKRT (* = BeLaK);
NSa1(+17)-A13BocK-Y92K-C1
[SEQ. ID. NO: 21]
MGSSHHHHHHSSGTENLYFQGC VKSKPTKLRV VR*TPTSLKI
SWKAPKKTVD YYVITYGKTG SGGYAWQRFR VPGSKRTAKI KGLKPGVDYT
ITVYAGYKGY PTYYSSPISI NKRT (* = BocK);
NSa1(+10)-A13BeLaK-C95
[SEQ. ID. NO: 22]
MGSSHHHHHHSSGTENLYFQG VSSKPTKLRV VR *TPTSLKI
KWDAPAKTVD YYVITYGETG RGGYAWQRFE VPGSKRTATI KGLKPGVDYT
ITVYAGYKGY PTYYSSPISI NYRTC (* = BeLaK);
NSa1(+10)-A13BeLaK-C1
[SEQ. ID. NO: 23]
MGSSHHHHHHSSGTENLYFQGC VSSKPTKLRV VR*TPTSLKI
KWDAPAKTVD YYVITYGETG RGGYAWQRFE VPGSKRTATI KGLKPGVDYT
ITVYAGYKGY PTYYSSPISI NYRT (* = BeLaK).

TABLE 5
MS characterization of UAA-encoded monobodies.

| | Calculated | Observed | | |
|---|---|---|---|---|
| | mass | mass | Expression | |
| | [M-Met + | [M-Met + | yield | X-linking |
| Name | H⁺] | H⁺] | (mg L⁻¹) | yield |
| NSa1-A13BeLaK | 12,517.58 | 12,517.71 | 13.9 | 0% |
| NSa1(+5)-A13BeLaK | 12,734.99 | 12,735.06 | 17.5 | 43% |
| NSa1(+7)-A13BeLaK | 12,845.20 | 12,845.37 | 23.4 | 61% |
| NSa1(+10)-A13BeLaK | 12,915.34 | 12,915.57 | 19.4 | 86% |
| NSa1(+17)-A13BeLaK | 12,969.61 | 12969.85 | 6.7 | 87% |
| NSa1(+10)-A13BeLaK-C95 | 13,018.48 | 13,018.63 | 25.7 | 22% |
| NSa1(+10)-A13BeLaK-C1 | 13,018.48 | 13,018.57 | 7.1 | 97% |
| NSa1(+5)-A13BeLaK-Y92K | 12,699.99 | 12,700.36 | 37.3 | 53% |
| NSa1(+17)-A13BeLaK-Y92K | 12,934.60 | 12,935.01 | 6.7 | 89% |
| NSa1-A13BeLaK-Y92K-C1 | 12,585.72 | 12,585.87 | 9.6 | 20% |
| NSa1(+5)-A13BeLaK-Y92K-C1 | 12,803.14 | 12,803.25 | 14.6 | 25% |
| NSa1(+7)-A13BeLaK-Y92K-C1 | 12,913.34 | 12,913.44 | 17.0 | 60% |
| NSa1(+10)-A13BeLaK-Y92K-C1 | 12,983.48 | 12,983.58 | 32.1 | 100% |
| NSa1(+17)-A13BeLaK-Y92K-C1 | 13,037.75 | 13,037.92 | 12.4 | 87% |
| NSa1(+5)-A13BocK-Y92K | 12,703.04 | 12,703.13 | — | N/A |
| NSa1-A13BocK-Y92K-C1 | 12,588.77 | 12,588.91 | 4.8 | N/A |
| NSa1(+5)-A13BocK-Y92K-C1 | 12,806.18 | 12,806.48 | 22.5 | N/A |
| NSa1(+7)-A13BocK-Y92K-C1 | 12,916.38 | 12,916.62 | 11.2 | N/A |
| NSa1(+10)-A13BocK-Y92K-C1 | 12,986.52 | 12,985.48 | 28.8 | N/A |
| NSa1(+17)-A13BocK-Y92K-C1 | 13,040.79 | 13,041.01 | 12.7 | N/A |
| 12VC1-A13BeLaK-Y92K | 12,433.82 | 12,433.94 | 12.9 | 88% |
| 12VC1(+8)-A13BeLaK-Y92K | 12,432.97 | 12,432.87 | 12.0 | 86% |
| 12VC1(+10)-A13BeLaK-Y92K | 12,634.28 | 12,634.41 | 63.8 | 83% |
| 12VC1-A13BocK-Y92K | 12,436.87 | 12,437.06 | 7.5 | N/A |
| 12VC1(+8)-A13BocK-Y92K | 12,436.01 | 12,436.08 | 6.5 | N/A |
| 12VC1(+10)-A13BocK-Y92K | 12,637.33 | 12,637.56 | 17.25 | N/A |

### Scheme for BeLaK Synthesis.

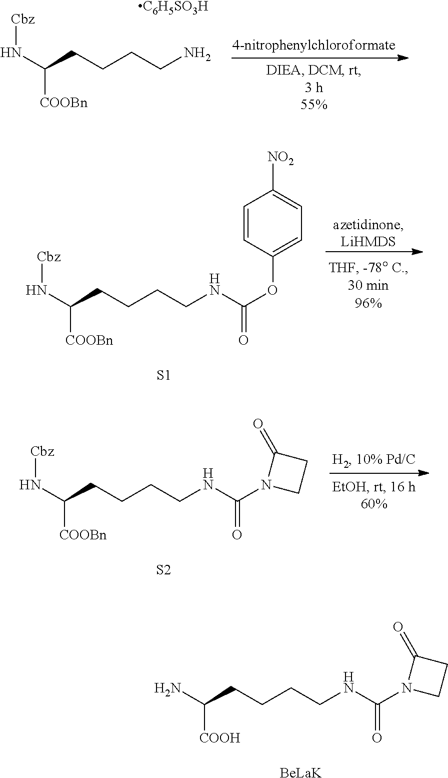

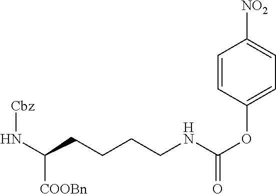

Benzyl N²-((benzyloxy)carbonyl)-N⁶-((4-nitrophenoxy)carbonyl)-L-lysinate (Si): A solution of 4-nitrophenylchloroformate (42 mg, 0.21 mmol) in 2 mL dichloromethane in a round-bottom flask was stirred at 0° C. under argon. Then, a solution of Na—Z-L-lysine benzyl ester benzenesulfonate salt (100 mg, 0.189 mmol) and N,N-diisopropylethylamine (83 μL, 0.47 mmol) in 3 mL dichloromethane was added to the round bottom flask using a syringe pump at a rate of 0.6 mL/min. The mixture was stirred under argon at room temperature for 3 hours before addition of a saturated aqueous NH₄Cl solution (0.2 mL). The mixture was extracted with dichloromethane and the organic layer was separated, dried over Na₂SO₄, filtered, and concentrated. The residue was purified by silica gel flash chromatography (hexanes/EtOAc=2:1) to afford the titled compound as a white solid (56 mg, 55% yield): ¹H NMR (400 MHz, CD₃Cl) δ 8.20 (d, J=9.2 Hz, 2H), 7.39-7.30 (m, 10H), 7.28 (d, J=9.1 Hz, 2H), 5.39 (d, J=8.3 Hz, 1H), 5.25-5.12 (m, 3H), 5.10 (s, 2H), 4.44 (m, 1H), 3.21 (q, J=6.9 Hz, 2H), 1.89 (m, 1H), 1.71 (m, 1H), 1.56 (m, 2H), 1.46-1.31 (m, 2H); ¹³C NMR (101 MHz, CDCl₃) δ 172.14, 156.05, 155.97, 153.17, 144.74, 136.15, 135.24, 128.68, 128.60, 128.57, 128.39, 128.27, 128.09, 125.10, 121.93, 67.27, 67.12, 53.62, 40.91, 32.33, 28.90, 22.20. HRMS calcd for C₂₈H₃₀N₃O₈ 536.2027 [M+H⁺], found 536.2006.

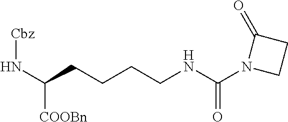

Benzyl N²-((benzyloxy)carbonyl)-N⁶-(2-oxoazetidine-1-carbonyl)-L-lysinate (S2): To a stirred solution of azetidinone (140 mg, 1.97 mmol) in 19 mL anhydrous THE in an oven-dried round-bottom flask at −78° C. under argon was added dropwise a 1M solution of lithium bis(trimethylsilyl)amide) in THF (2.17 mL, 2.17 mmol). The mixture was stirred at −78° C. for 15 minutes before a solution of S1 (528 mg, 0.987 mmol) in 2 mL anhydrous THE under argon was added slowly. Then, the mixture was stirred under argon for 30 minutes allowing to warm up to room temperature. A saturated aqueous NH₄Cl solution (3 mL) was added to the mixture and stirred for 30 minutes at room temperature. THE was removed using reduced pressure before extracting with EtOAc. The organic layer was dried over anhydrous Na₂SO₄, filtered, and concentrated. The residue was purified by silica gel flash chromatography (hexanes/EtOAc=1:1) to afford the titled compound as a light brown oil (444 mg, 96%): ¹H NMR (400 MHz, CDCl₃) δ 7.39-7.29 (m, 10H), 6.47 (t, J=6.0 Hz, 1H), 5.44-5.35 (m, 1H), 5.23-5.12 (m, 2H), 5.10 (s, 2H), 4.39 (m, 1H), 3.57 (t, J=4.8 Hz, 2H), 3.22 (m, 2H), 2.99 (t, J=4.8 Hz, 2H), 1.86 (m, 1H), 1.76-1.63 (m, 1H), 1.51 (m, 2H), 1.42-1.28 (m, 2H); ¹³C NMR (101 MHz, CDCl₃) δ 172.21, 167.00, 155.95, 150.72, 136.31, 135.35, 128.64, 128.53, 128.48, 128.32, 128.18, 128.13, 67.15, 67.01, 53.83, 39.20, 37.10, 35.95, 32.03, 29.33, 22.24. HRMS calcd for C₂₅H₃₀N₃O₆ 468.2129 [M+H⁺], found 468.2862.

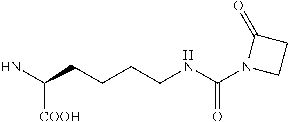 [SMILES: N[C@@H](CCCCNC(=O)N1CCC1=O)C(=O)O]

N⁶-(2-Oxoazetidine-1-carbonyl)-L-lysine (BeLaK): To a solution of S2 (1.7 g, 3.63 mmol) in ethanol (30 mL) was added Pd/C on (150 mg, 10%). The round bottom flask was filled with hydrogen and stirred at room temperature for 16 hours. The Pd/C was removed by filtering through celite and the filtrate was concentrated to afford the titled compound as an off-white solid (520 mg, 60% yield): ¹H NMR (500 MHz, D₂O) δ 3.68-3.63 (m, 1H), 3.57 (t, J=4.8 Hz, 2H), 3.20 (t, J=6.9 Hz, 2H), 3.04 (t, J=4.8 Hz, 2H), 1.80 (m, 2H), 1.53 (m, 2H), 1.41-1.27 (m, 2H); ¹³C NMR (126 MHz, D₂O) δ 174.87, 169.66, 152.32, 54.68, 39.13, 37.74, 35.43, 30.11, 28.48, 21.61. HRMS calcd for C₁₀H₁₇N₃NaO₄ 266.1111 [M+Na⁺], found 266.1167.

TABLE 6
MS characterization of UAA-encoded GST proteins. Dimer formation was
determined by comparing GST-monomer to dimer bands in western blot.

| | Calculated dimer | Observed dimer | Expression | |
|---|---|---|---|---|
| | mass (Da) | mass (Da) | yield | Dimer |
| Name | 2[M-Met + H⁺] | 2[M-Met + H⁺] | (mg L⁻¹) | formation |
| GST-E52BeLaK-E92K | 53,170.23 | 53,170.72 | 29.6 | 83% |
| GST-E52BeLaK-E92Y | 53,239.23 | 53,239.08 | 37.6 | 20% |
| GST-E52BeLaK-E92C | 53,119.18 | 53,120.38 | 12.5 | 37% |
| GST-E52BeLaK-E92H | 53,189.18 | 53,189.16 | 18 | 6% |
| GST-E52BeLaK-E92T | 53,377.49 | 53,376.15 | 7 | 15% |
| GST-E52BeLaK-E92S | *53,699.68 | 53,699.47 | 1.9 | 80% |
| GST-E52BeLaK-E92D | 53,143.06 | N/A | 1.82 | 0% |

*protein expression yield was low for this mutant

### Example 3

This example provides a description of the preparation, characterization, and use of non-crosslinked proteins and crosslinked proteins of the present disclosure.

Design of Cell-Penetrating Monobodies via Genetic Supercharging and Orthogonal Crosslinking.

Domain antibodies such as monobodies provide an attractive immunoglobin fold for evolving high-affinity binders targeting the intracellular proteins implicated in cell signaling. However, it remains challenging to endow cell permeability to these small and versatile protein binders. A streamlined strategy combining orthogonal crosslinking mediated by a genetically encoded β-lactam-lysine (BeLaK) and genetic supercharging to generate cell-penetrating monobodies is described. When BeLaK was introduced site-specifically to the N-terminal β-strand of a panel of supercharged monobodies, it enabled efficient interstrand crosslinking with a nearby lysine, generating the rigidified analogs. Compared to the non-crosslinked counterparts, the BeLaK-crosslinked supercharged monobodies exhibited higher thermostability and enhanced cellular uptake at concentrations as low as 40 nM. Most significantly, a +11 charged, orthogonally crosslinked monobody showed significant endosomal escape after endocytosis. The discovery of this stabilized immunoglobin fold should facilitate the design of cell-permeable domain antibodies for targeting intracellular proteins.

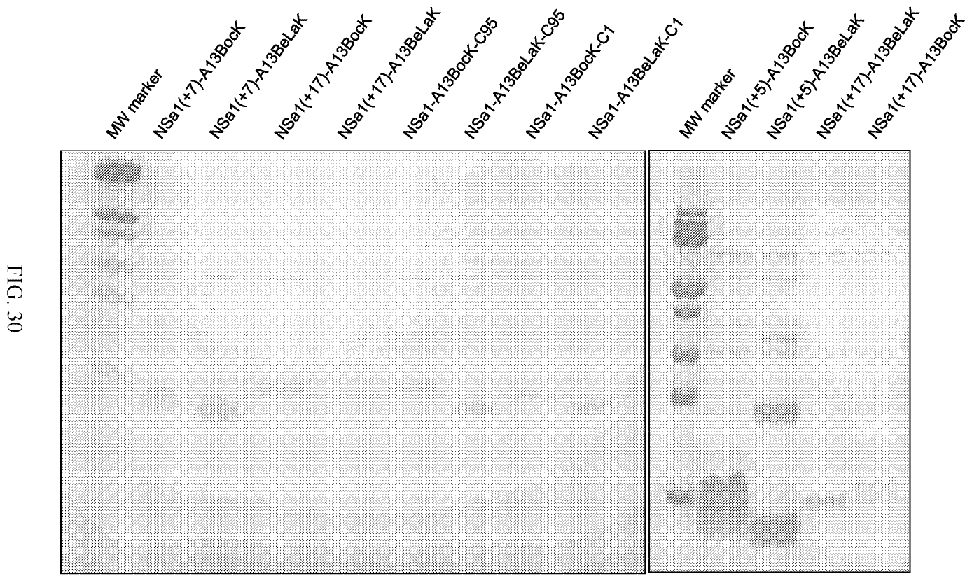

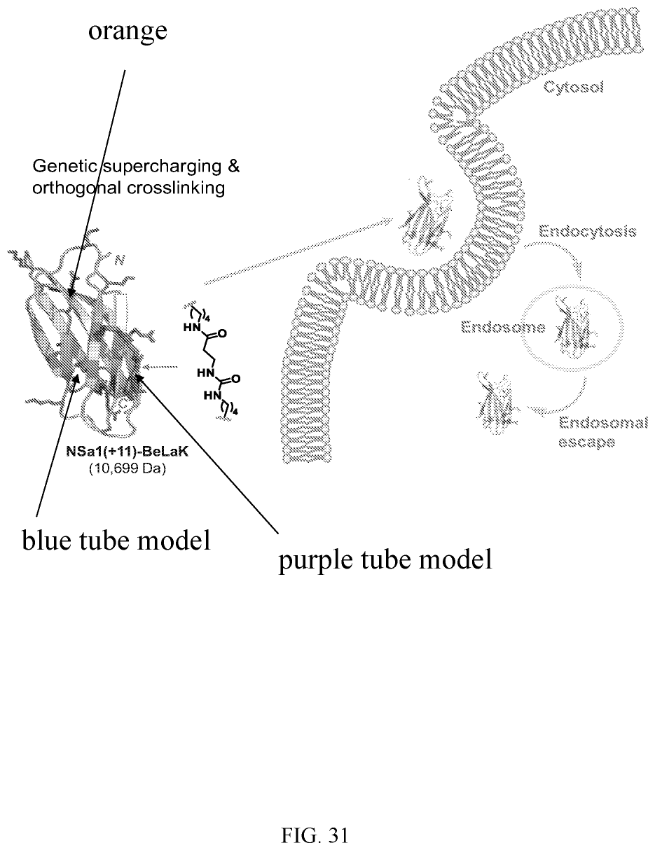

Orthogonal crosslinking was combined with genetic supercharging to generate cell-penetrating monobodies (FIG. 31). Specifically, we identified a β-lactam-containing lysine that can be incorporated site-specifically into the monobodies via genetic code expansion and observed robust proximity-driven orthogonal crosslinking with the nearby lysines. The resulting supercharged monobodies with the rigidified scaffold displayed higher thermostability and enhanced cytosolic uptake compared to their non-crosslinked counterparts.

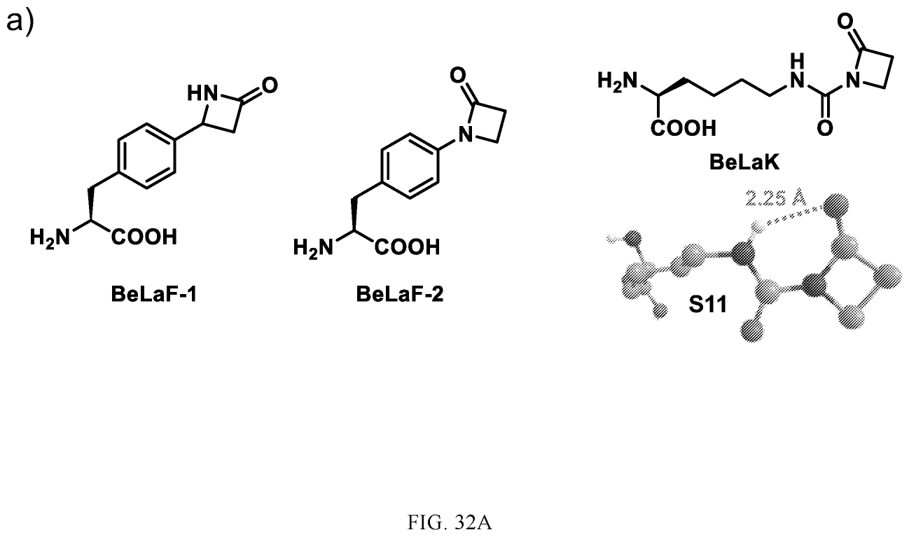

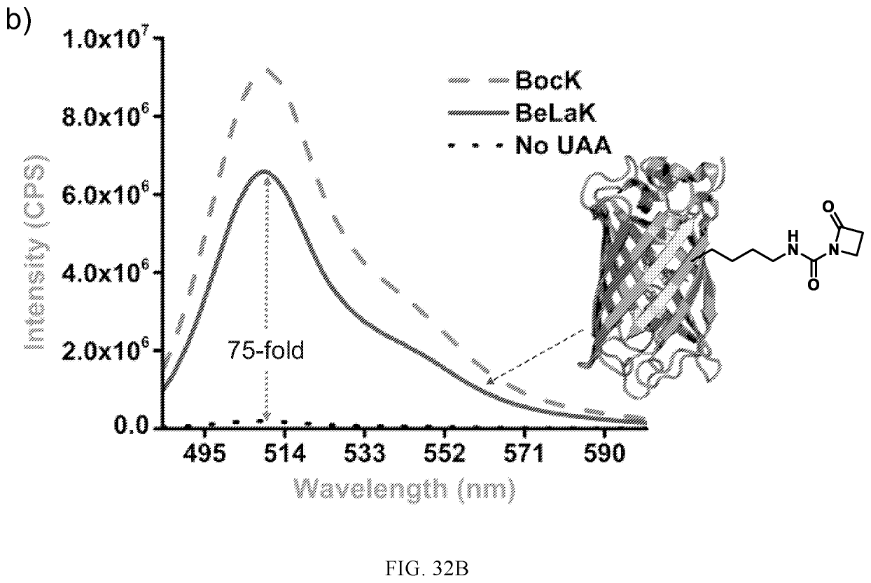

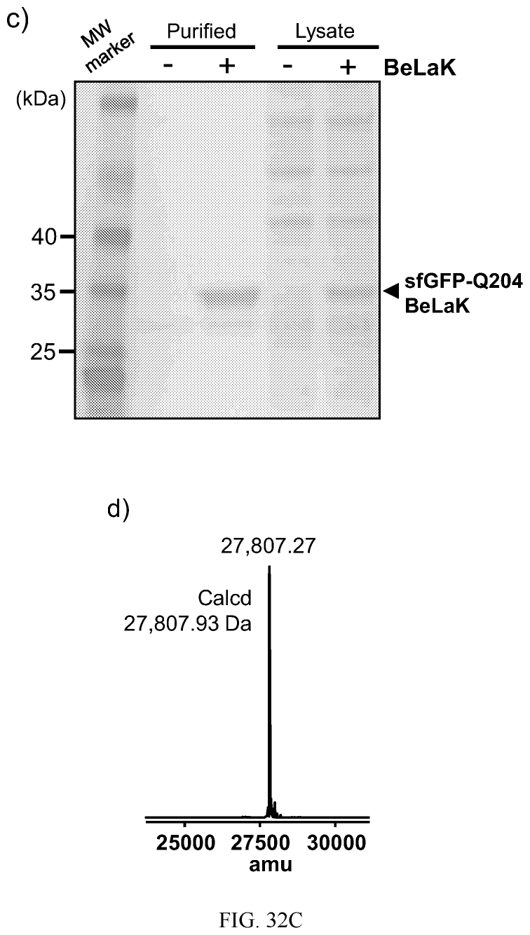

In our efforts to identify genetically encoded amino acids that are stable under physiological conditions yet reactive upon photoactivation or through proximity effect, we were intrigued by β-lactam, a venerable chemical moiety found in penicillin and other β-lactam class of antibiotics. Indeed, β-lactam has been employed in designing chemical probes for activity-based protein profiling, indicating a balanced reactivity and stability in the biological milieu. Accordingly, we designed three pi-lactam amino acids by appending β-lactam to either the para-position of phenylalanine or the lysine side chain (FIG. 32a). The phenylalanine analogs, BeLaF-1 and -2, were synthesized through the lactamization routes, while the lysine analog, BeLaK, was prepared from a protected lysine and azetidinone via a three-step synthetic procedure with an overall yield of 31%. The NMR-based stability studies showed that the more reactive BeLaF-2 and BeLaK remained intact after incubation with 10 mM glutathione in PBS for 3 days, confirming their stability toward a biological nucleophile.

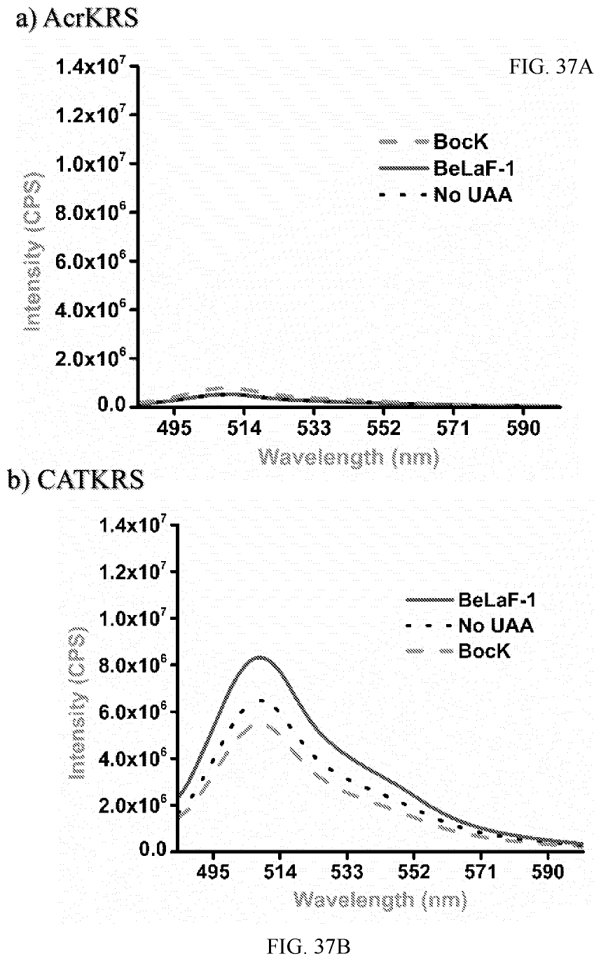

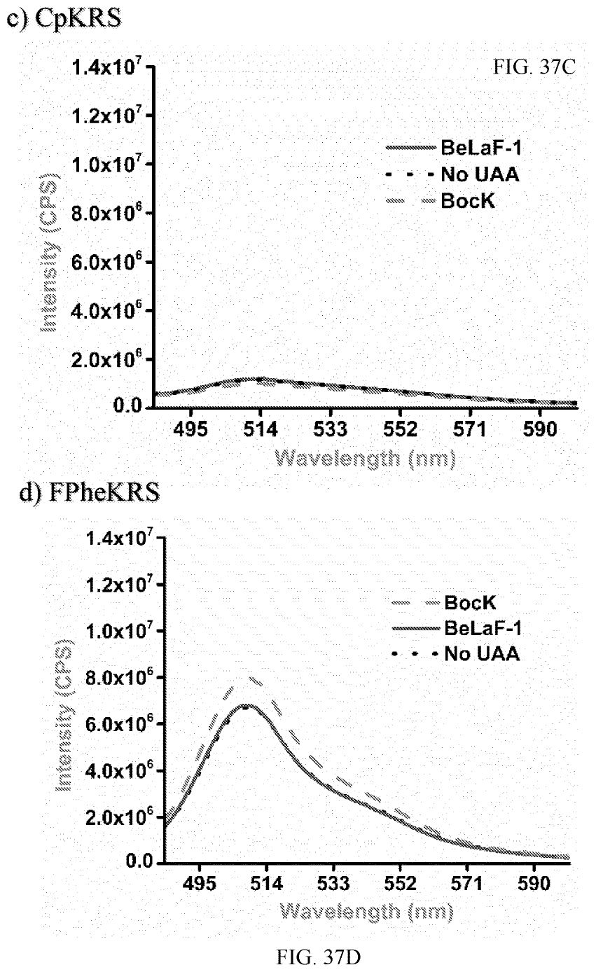

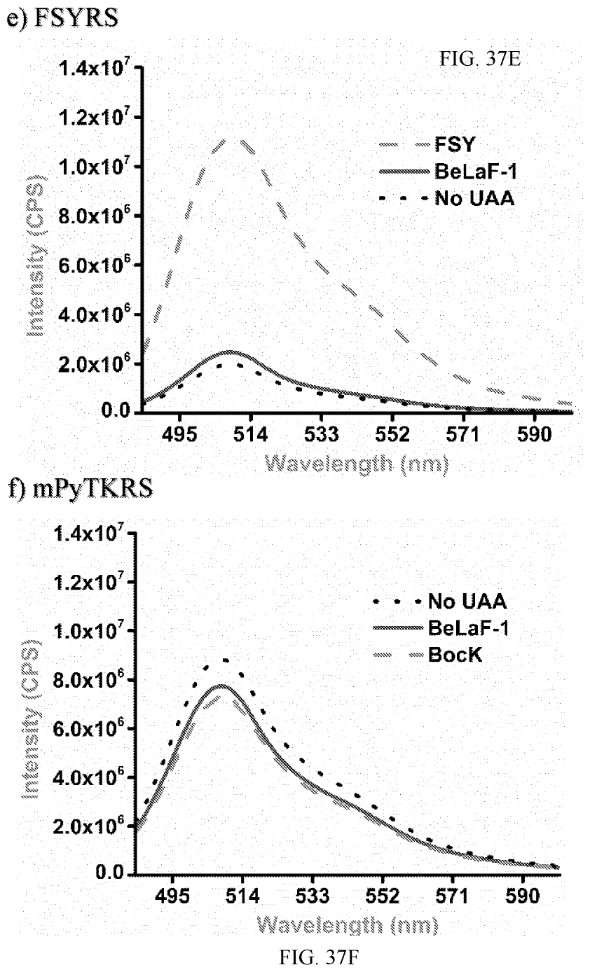

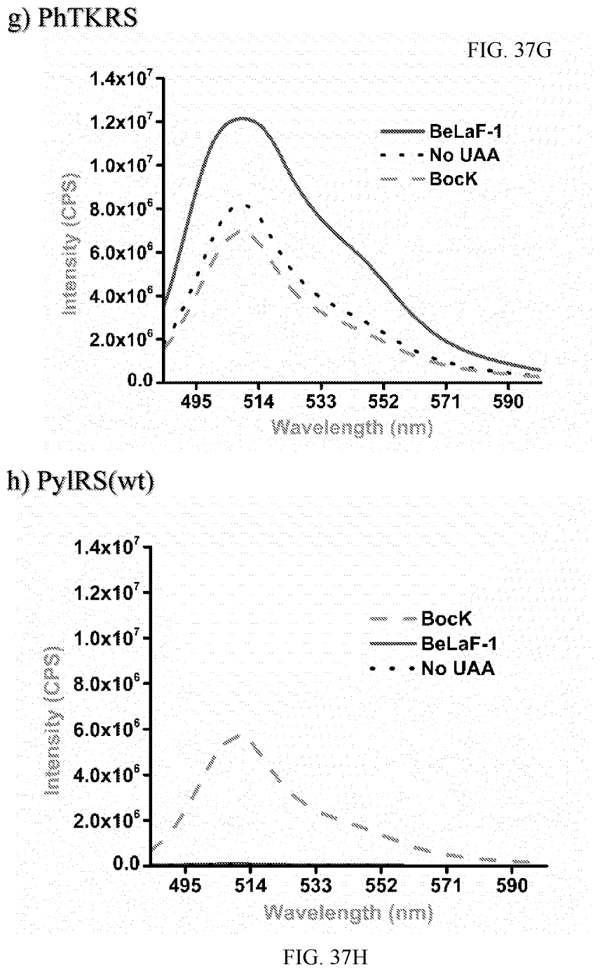

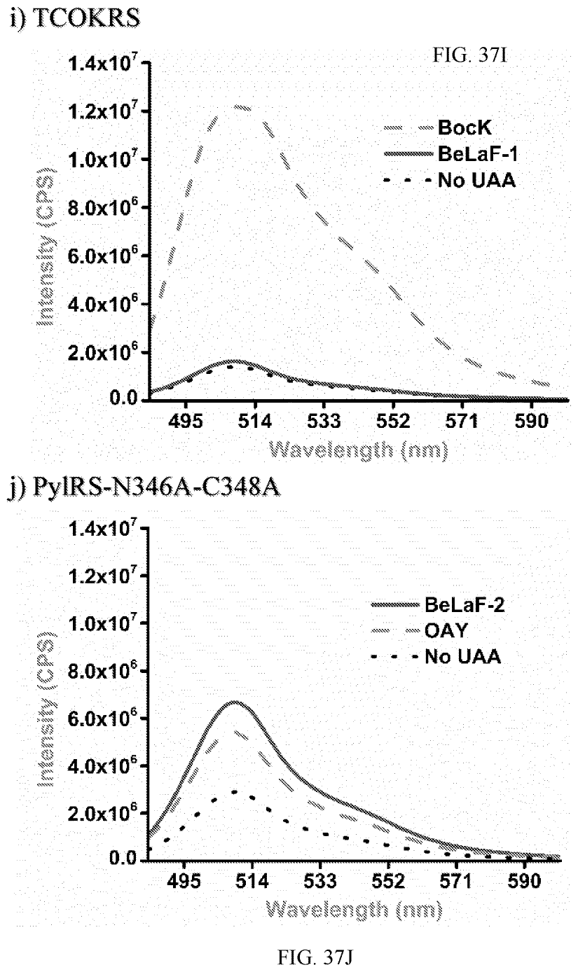

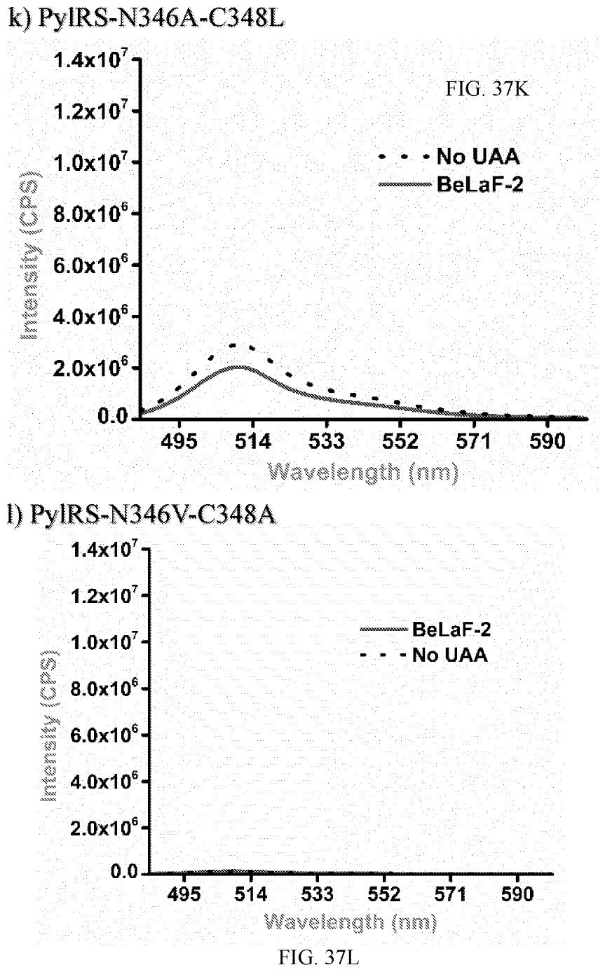

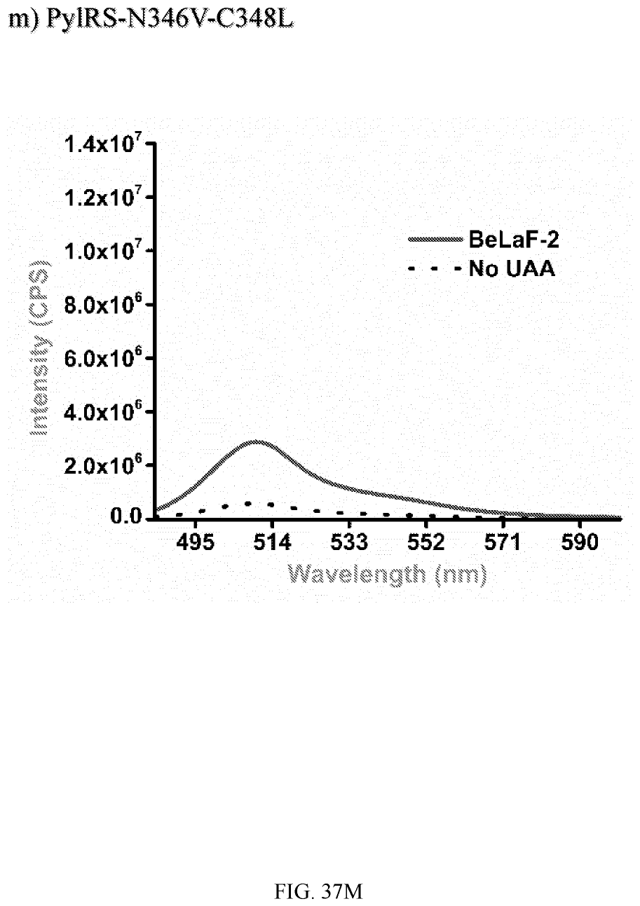

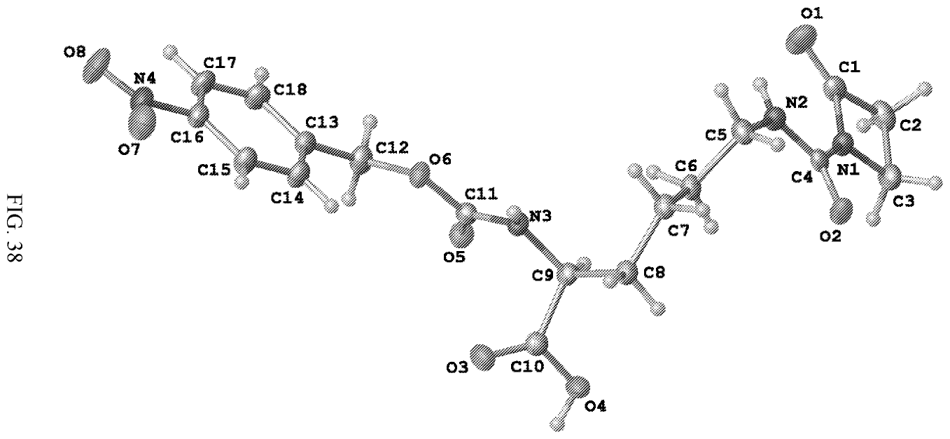

To identify an aminoacyl-tRNA synthetase/tRNA pair for charging two phenylalanine derivatives, we screened our collection of pyrrolysine-tRNA synthetases (PylRS) (Table 1) using superfolder green fluorescent protein bearing an amber codon at position-204 (sfGFP-Q204TAG) as a reporter without success (FIG. 37). In parallel, to our delight, we found wild-type PylRS efficiently charges BeLaK into sfGFP-Q204TAG, with the cell lysate showing a 75-fold increase in fluorescence over the background, similar to BocK, a known substrate for wild-type PylRS (FIG. 32b). We also obtained the crystal structure of a protected BeLaK analog S11 (FIG. 38). We note the lactam ring forms an intramolecular H-bond with the lysine ε-N—H (FIG. 32a), mimicking the pyrroline ring in pyrrolysine—the native substrate of PylRS. This structural resemblance may explain the superb substrate properties of BeLaK, as revealed by the high expression yield of 28 mg L⁻¹ (FIG. 32c) and clean intact mass (FIG. 32d), confirming that BeLaK is stable under bacterial culture conditions.

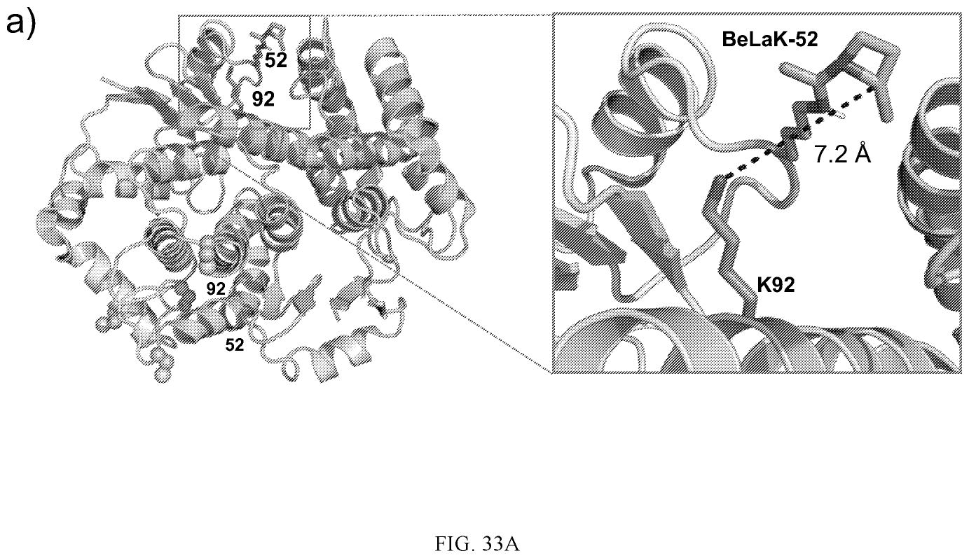

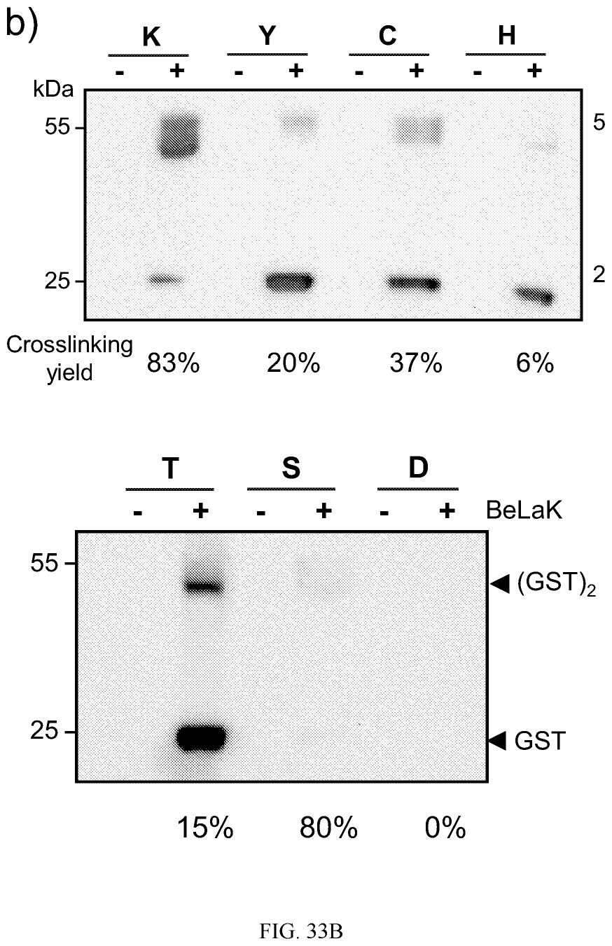

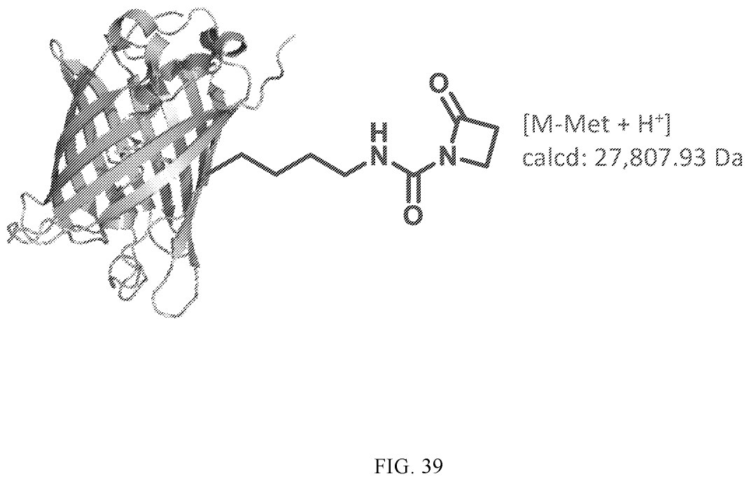

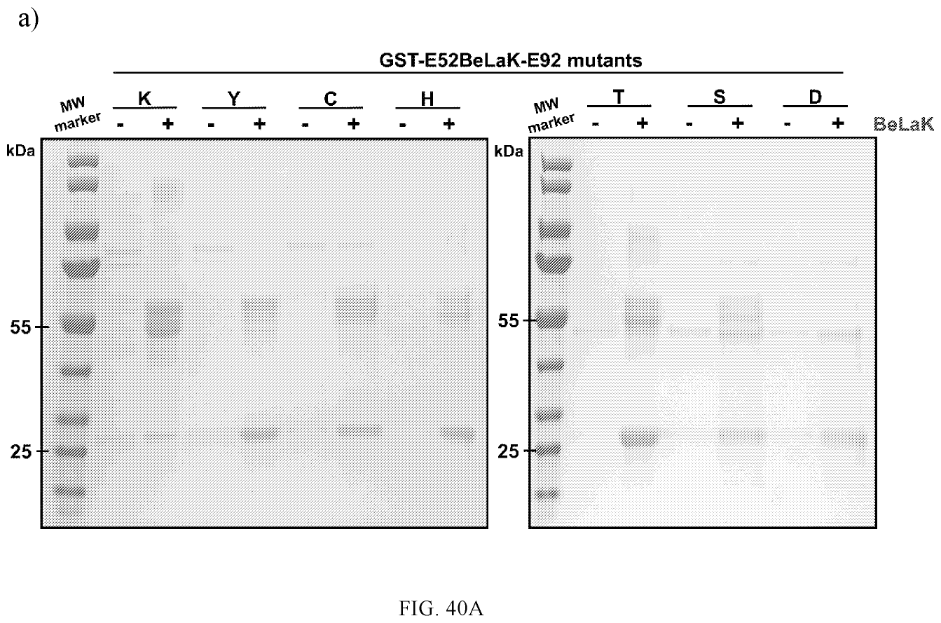

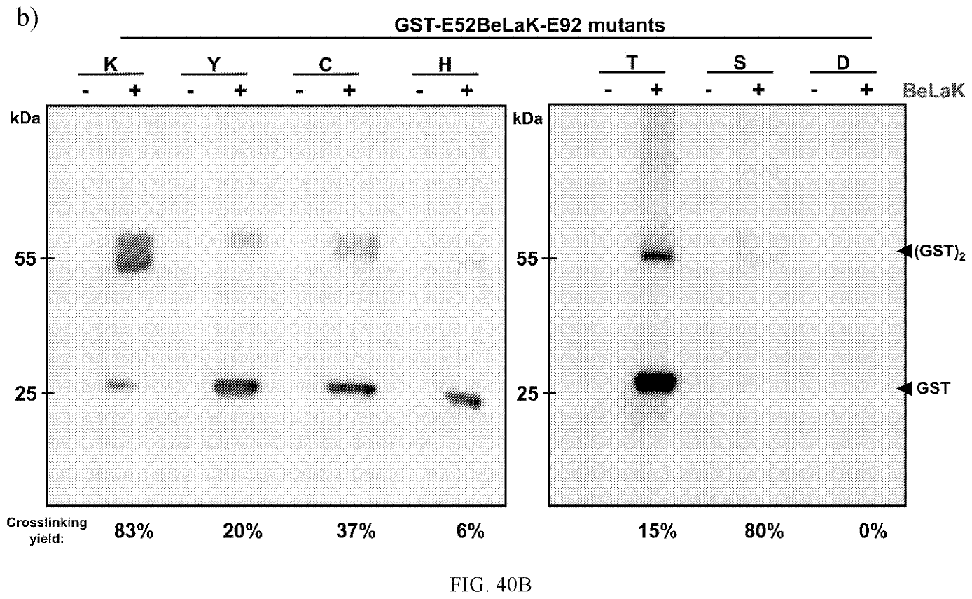

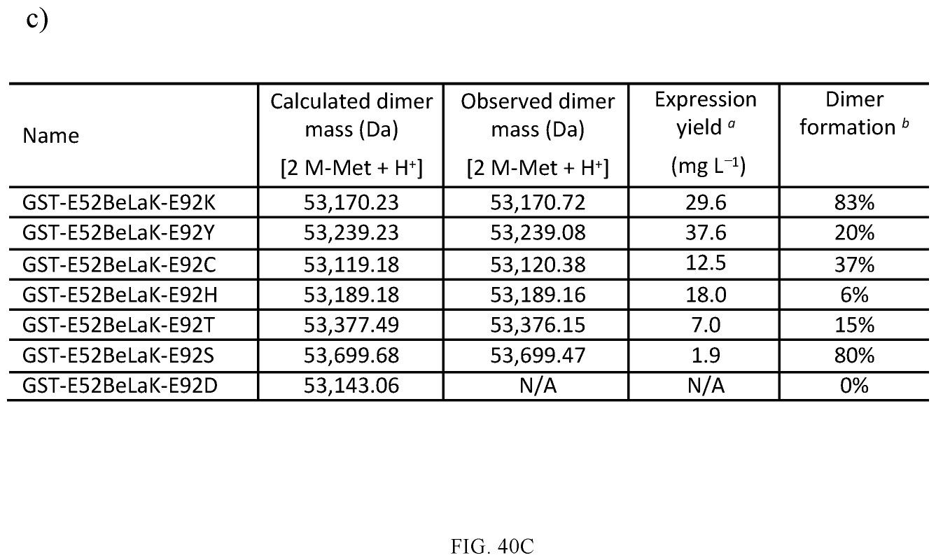

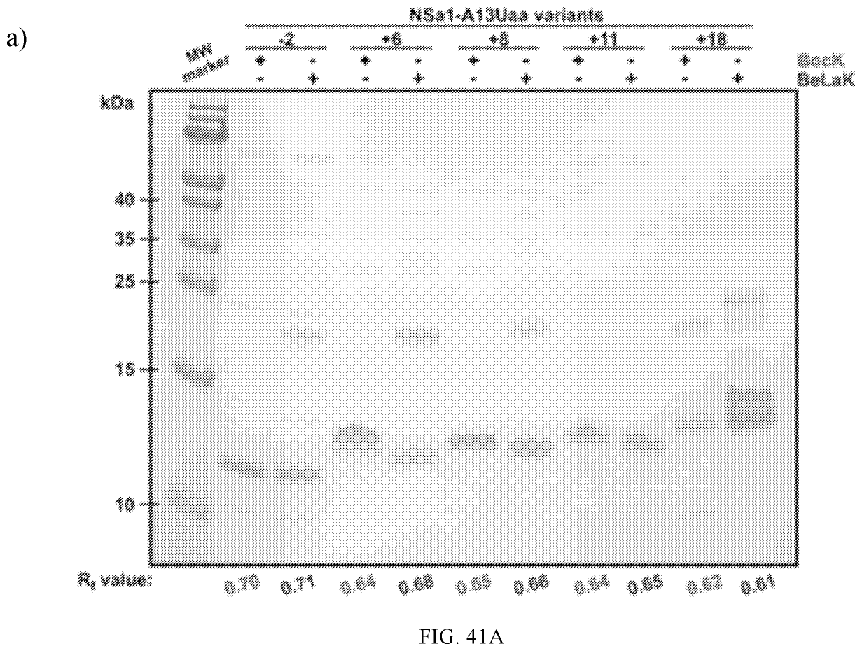

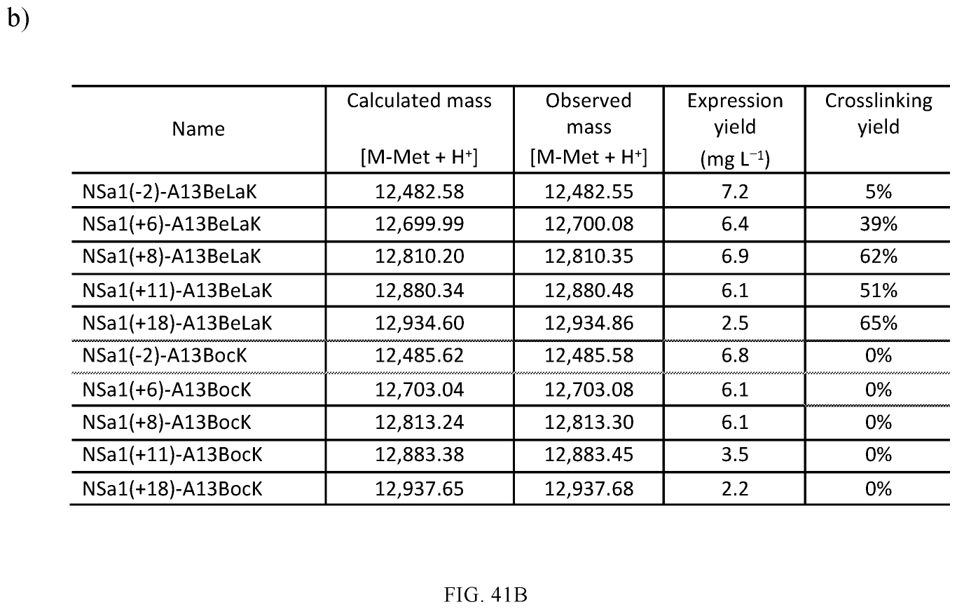

To probe if BeLaK possesses the requisite crosslinking reactivity, we placed BeLaK at position-52 of glutathione-S-transferase (GST). Modeling of BeLaK-52 onto the GST dimer structure indicated that BeLaK in one monomer is located ˜7.2 Å away from Lys-92 from the other monomer (FIG. 33a). Therefore, we placed a panel of nucleophilic residues at position-92 and examined their reactivity toward BeLaK-52 based on covalent GST dimer formation. Six of the seven GST mutants encoding BeLaK-52 were successfully expressed at yields of 1.9˜38 mg L⁻¹ (FIG. 38-40). Among them, Lys-92 gave the highest crosslinking yield, followed by Ser, Cys, Tyr, Thr, and His; however, the Ser mutant gave a barely detectable dimer band (FIG. 3b) and the lowest expression yield of 1.9 mg L⁻¹ (FIG. 40). The high reactivity of Lys is attributed to its long and flexible side chain that can provide an optimal orientation for the nucleophilic addition/lactam ring opening reaction.

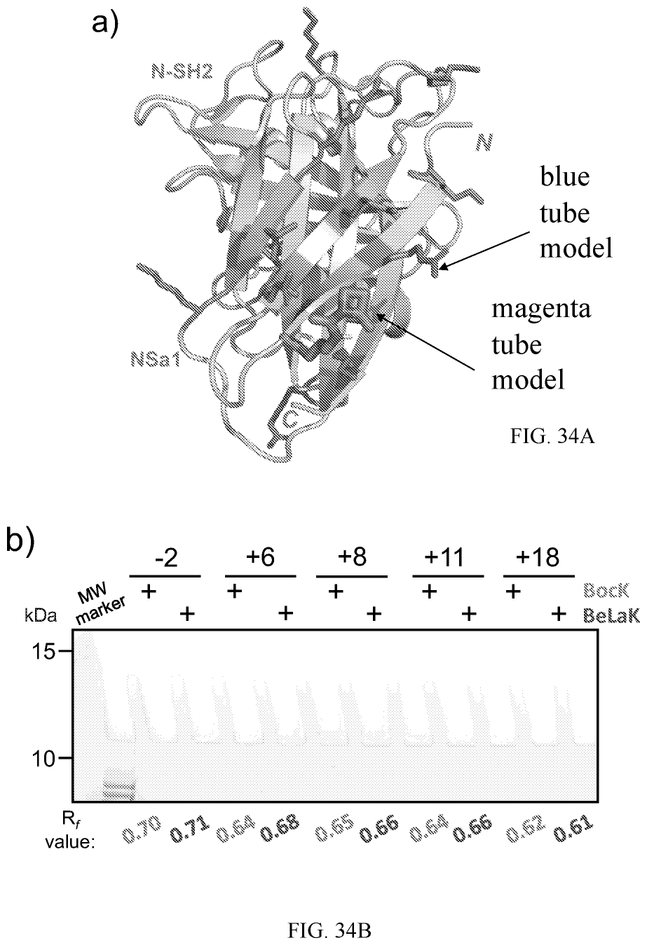

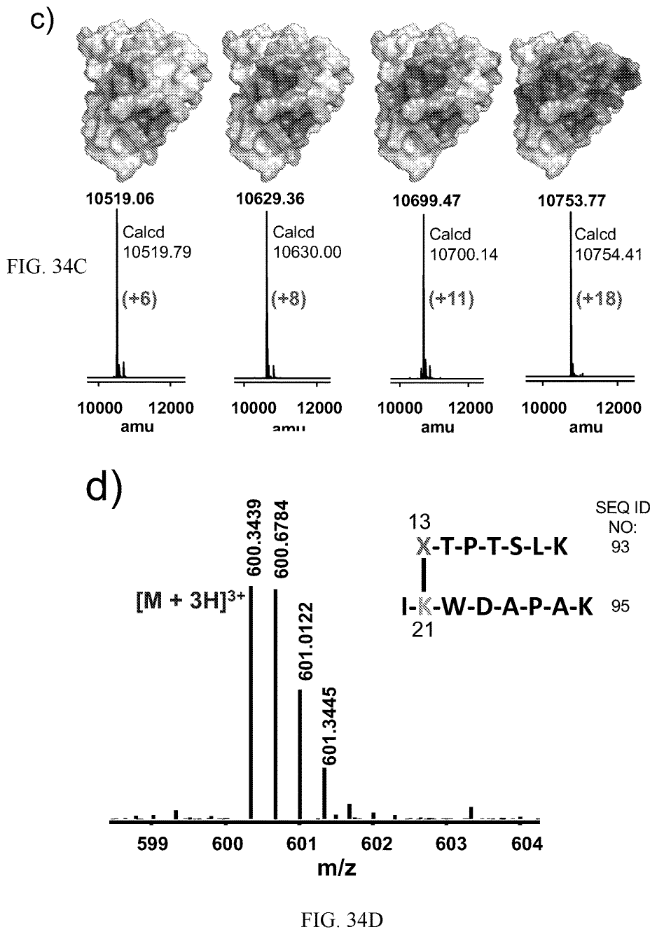

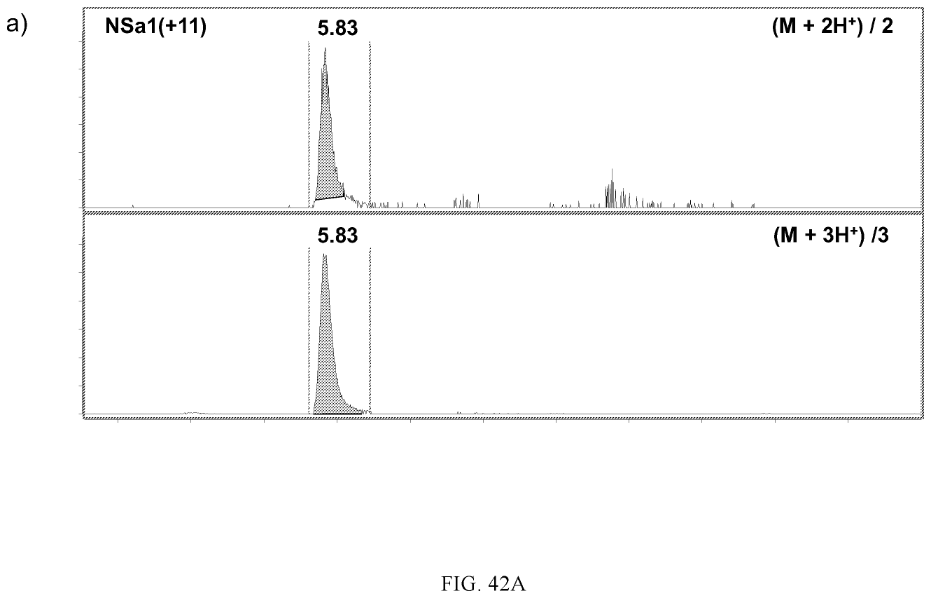

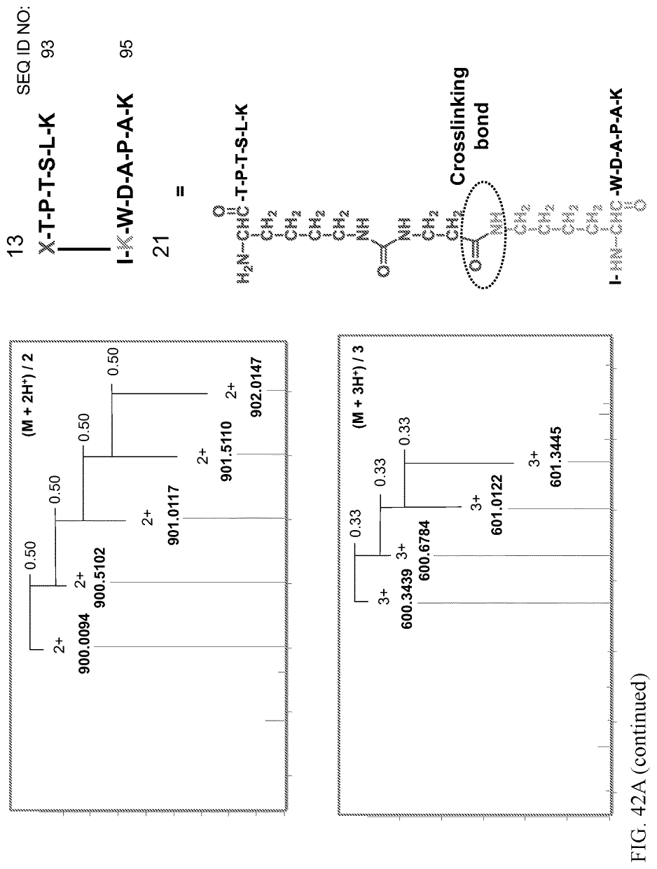

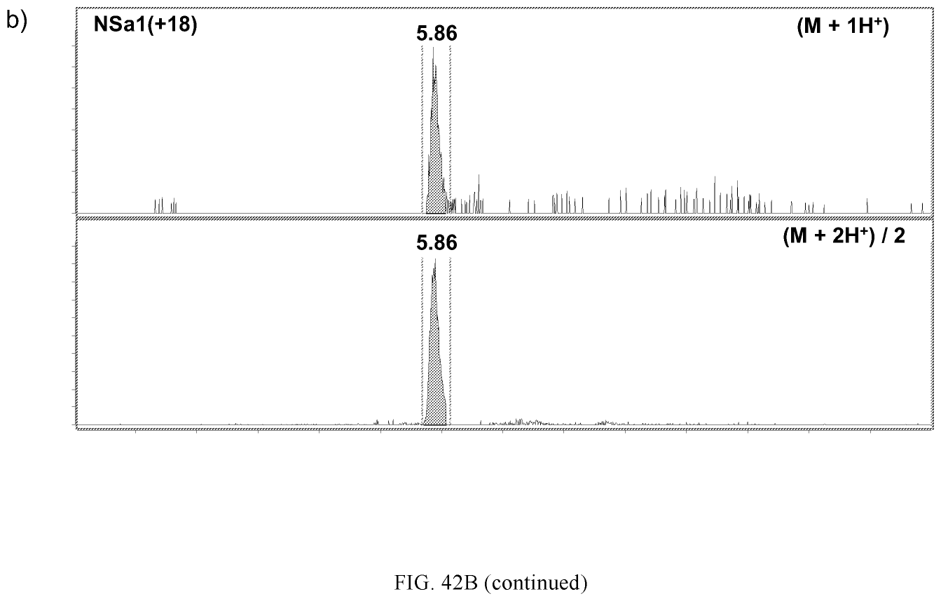

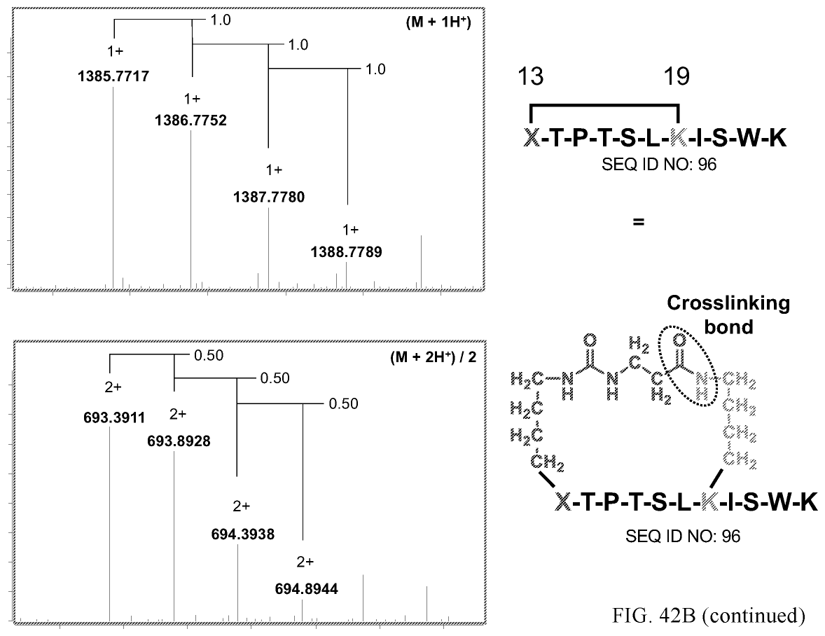

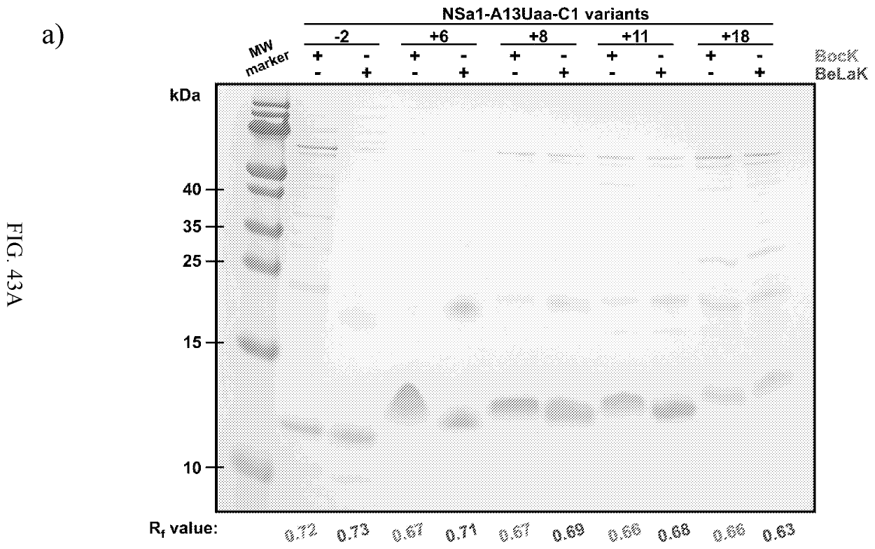

To assess if BeLaK is suitable for orthogonal crosslinking of domain antibodies, we selected NSa1, a monobody-based SHP2 inhibitor, and placed BeLaK at position-13 of the N-terminal β-strand. A panel of supercharged NSa1 mutants carrying overall charges of +6, +8, +11, and +18, respectively, were designed using Supercharge protocol on ROSIE Rosetta Online Server with native NSa1 (˜2 charge) as a template. Notably, both BeLaK and the exogenous lysines and arginines are located on the non-binding surface (FIG. 34a), and the ratios of positive charge to molecular weight (in kDa) for +11 and +18 mutants are 1.03, and 1.67, respectively, greater than 0.75, a threshold deemed necessary for cell penetration. Thus, the supercharged monobodies encoding either BeLaK or BocK were expressed in good yields (2.2˜7.2 mg L⁻¹; FIG. 42). SDS-PAGE analysis revealed that the majority of BeLaK-encoded mutants except +18 migrate faster than the BocK-encoded non-crosslinked counterparts (FIG. 34b), in agreement with the formation of an internal crosslink that reduces overall protein surface area and thus decreases interactions with the gel matrix during electrophoresis. Furthermore, as the positive charge increases, the mobility decreases, likely due to reduced overall negative charge after association with the SDS molecules. MS analysis further confirmed the identities of the supercharged NSa1 mutants (FIG. 34c). Since the unreacted BeLaK-encoded monobodies share the same mass as the crosslinked ones, we treated the purified monobodies with excess β-mercapto-ethanol for 7 days, which accelerates the hydrolysis of the unreacted β-lactam and in turn adds +18 Da to protein mass, and monitored the intact mass change. Using this method, we determined orthogonal crosslinking yields to range from 5% for native NSa1 to more than 50% for +8/+11/+18 mutants (FIG. 42). We attribute the higher yields to the increased conformational dynamics, which promotes proximity-driven crosslinking reactions. The crosslinking sites in +11 and +18 mutants were mapped to Lys-21 and Lys-19, respectively, residues on the nearby β-strand, based on the identified fragment masses after trypsin digestion (FIG. 34d, FIG. 50).

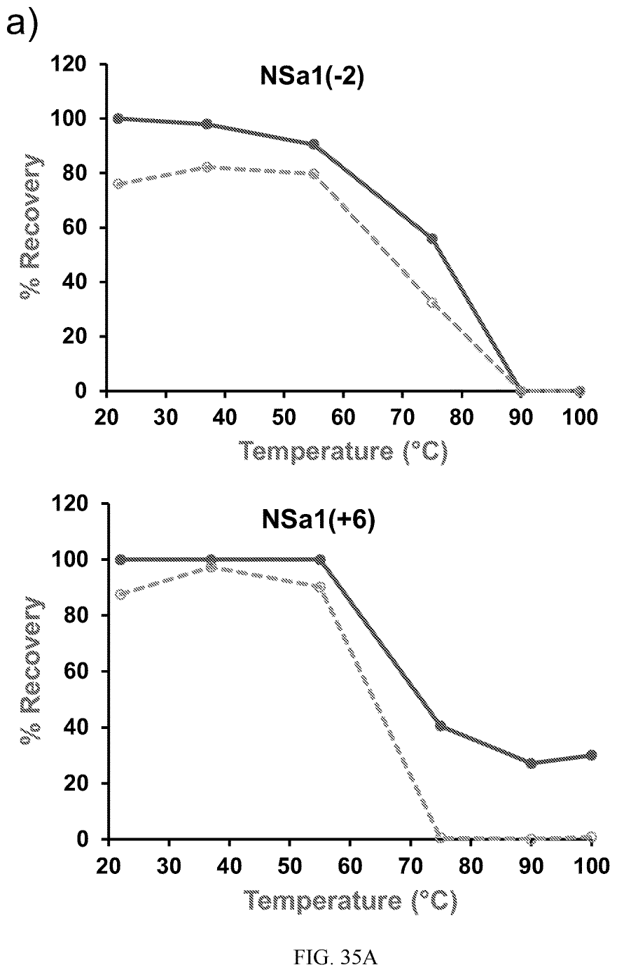

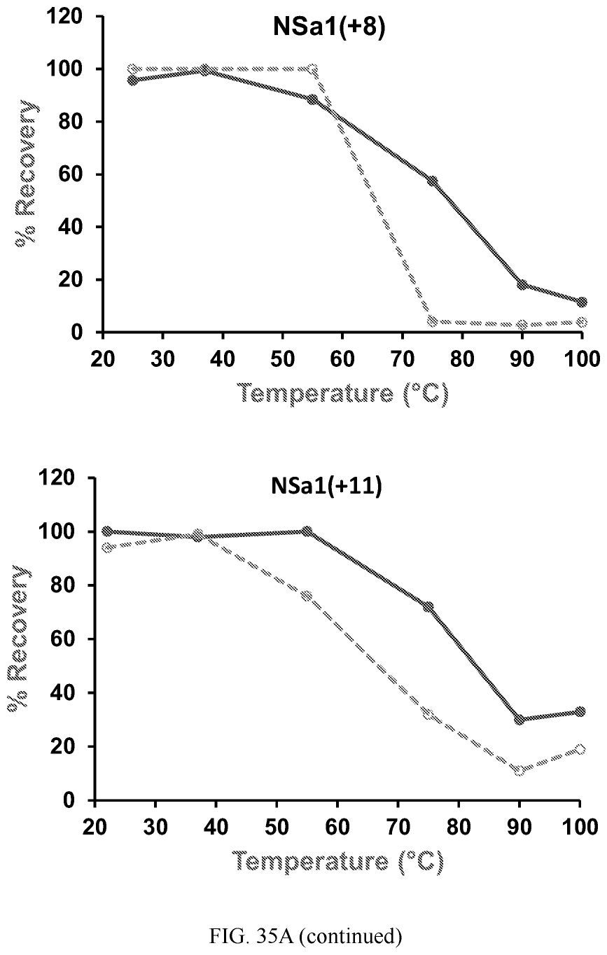

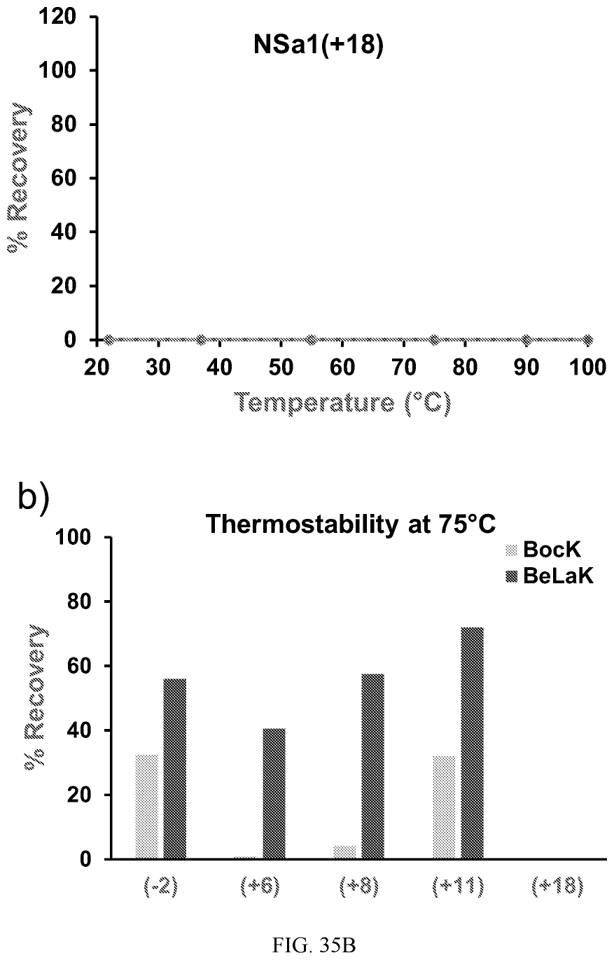

To probe the effect of orthogonal crosslinking on protein stability, we heated NSa1 mutants at various temperatures for 10 minutes, followed by centrifugation to pellet the insoluble protein aggregates. We used SDS-PAGE to quantify the soluble fraction in the supernatant following a literature procedure. As expected, the orthogonally crosslinked NSa1 mutants exhibited significant thermal denaturation resistance compared to their non-crosslinked counterparts, with +6 and +8 mutants giving the most pronounced effect at 75° C. (FIG. 35). However, the +18 mutants appeared to form aggregates even at room temperature, presumably due to the destabilization caused by extensive mutagenesis.

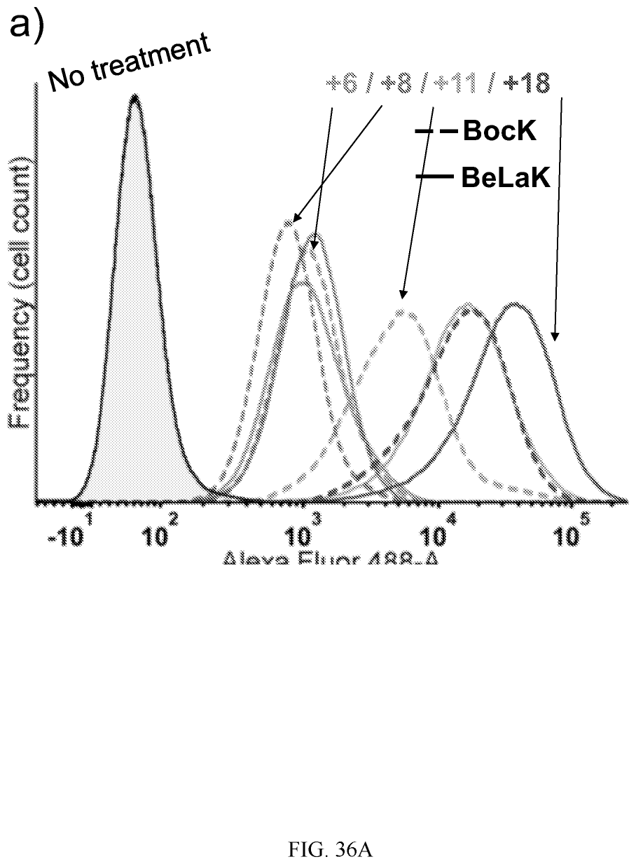

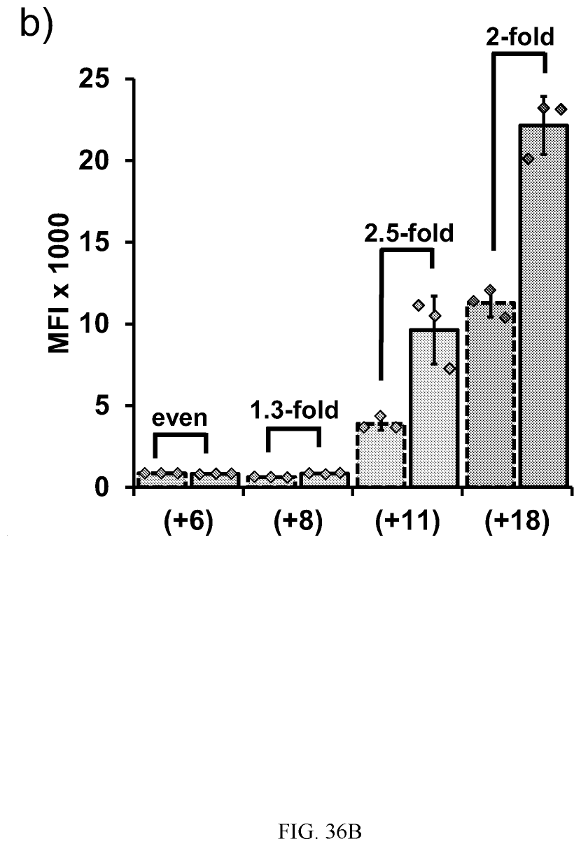

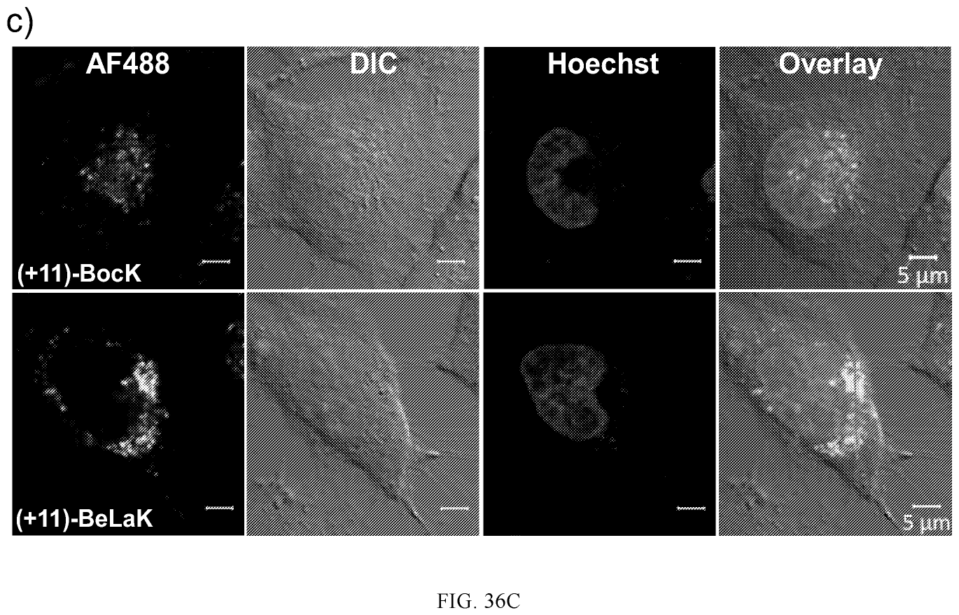

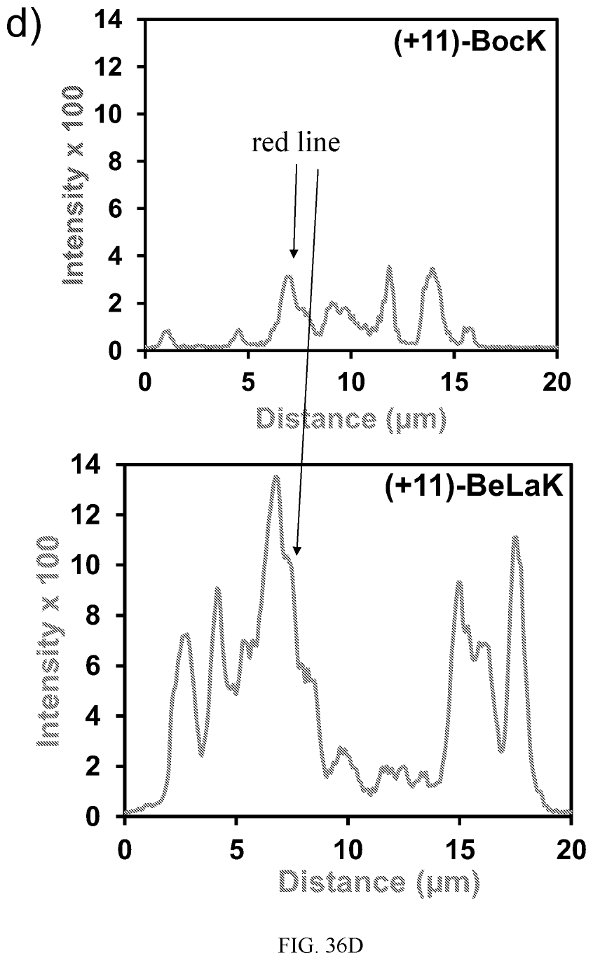

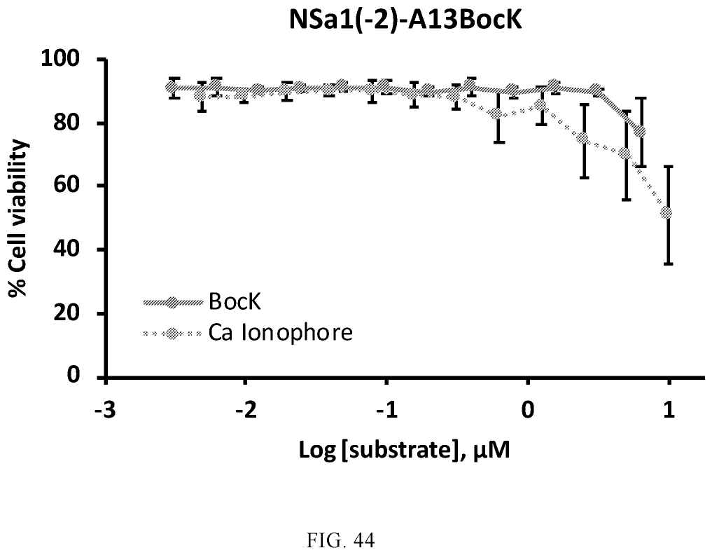

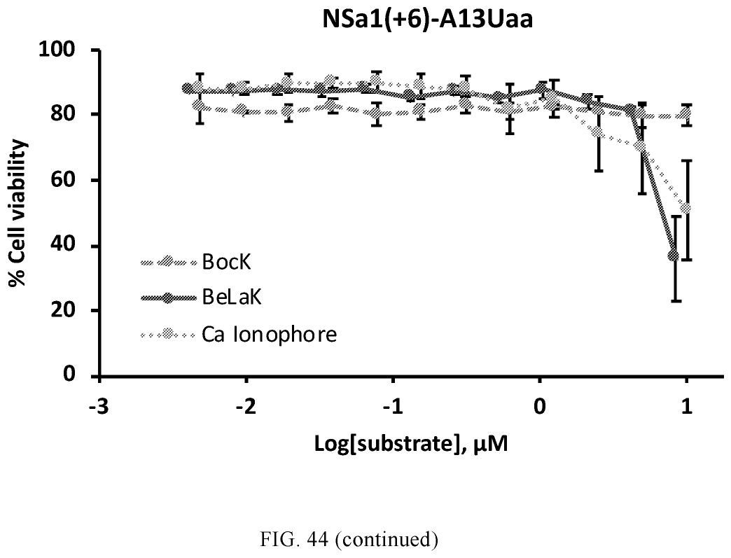

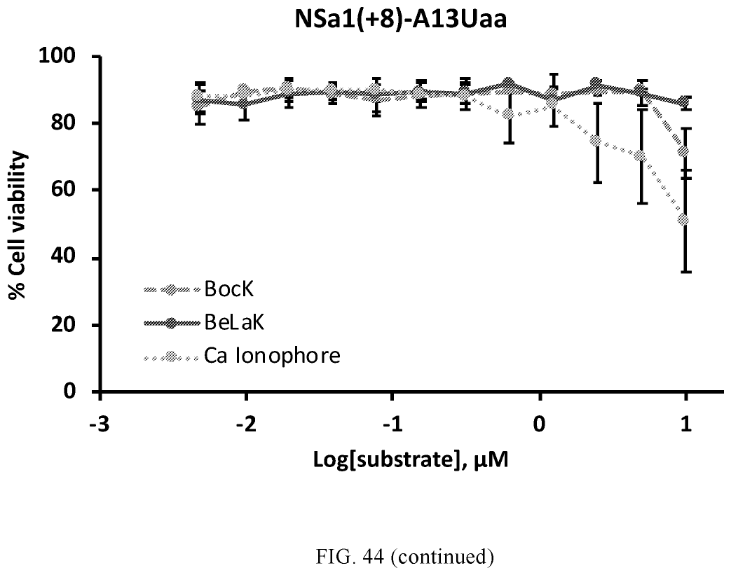

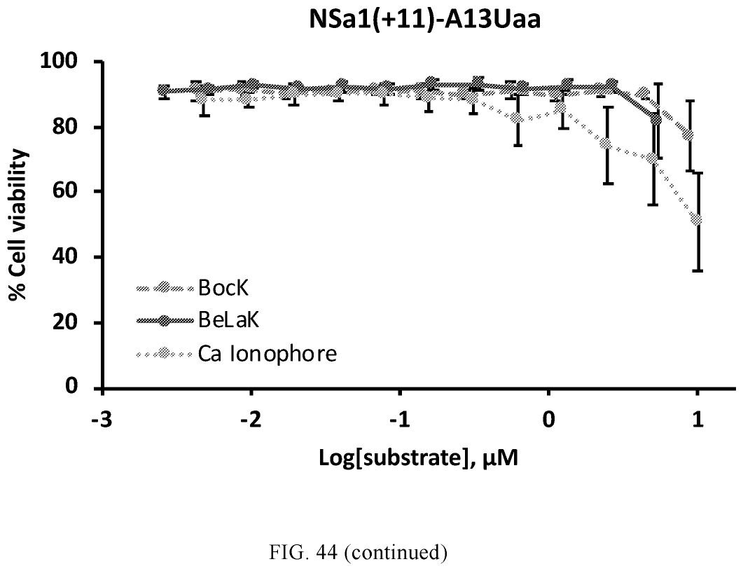

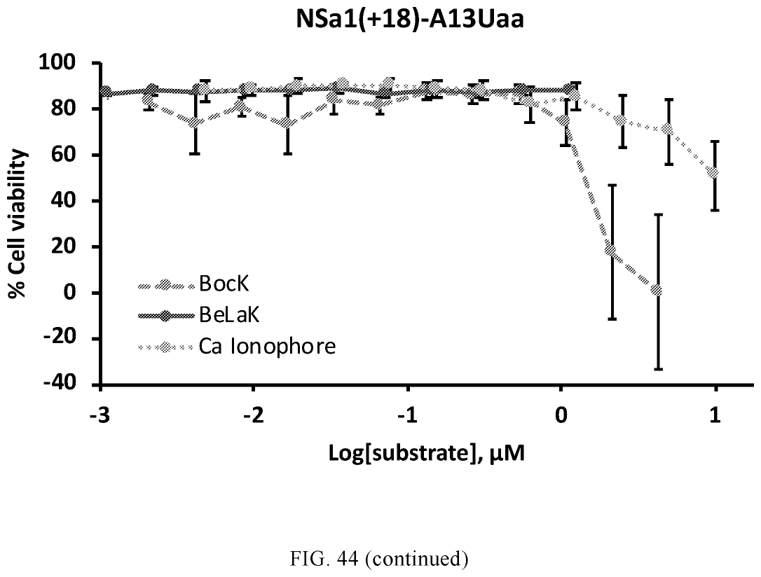

To examine if orthogonal crosslinking enhances cytosolic uptake of the supercharged NSa1 mutants, we prepared the fluorescent NSa1 mutants by inserting a Cys at the N-terminus for selective conjugation with AF488 maleimide (FIG. 52). Our cytotoxicity assay did not reveal any apparent toxicity of the supercharged monobodies in HeLa cells at monobody concentrations 1 μM (FIG. 44). We then performed flow cytometry analysis of the AF488-modified supercharged NSa1 monobodies to quantify their cytosolic uptake. Briefly, HeLa cells were treated with 40 nM NSa1 mutants at 37° C. for 5 hours, and surface-bound fluorescent supercharged NSa1 monobodies were removed by washing the cells with PBS containing 20 U mL⁻¹ heparin. A progressive increase in fluorescence was observed as the charge increases (FIG. 36a). Moreover, the crosslinked +11 and +18 charged monobodies exhibited 2.5 and 2-fold greater cellular uptake than their non-crosslinked counterparts, respectively (FIG. 36b), indicating that the rigidified scaffolds are beneficial for cellular uptake of the highly charged monobodies.

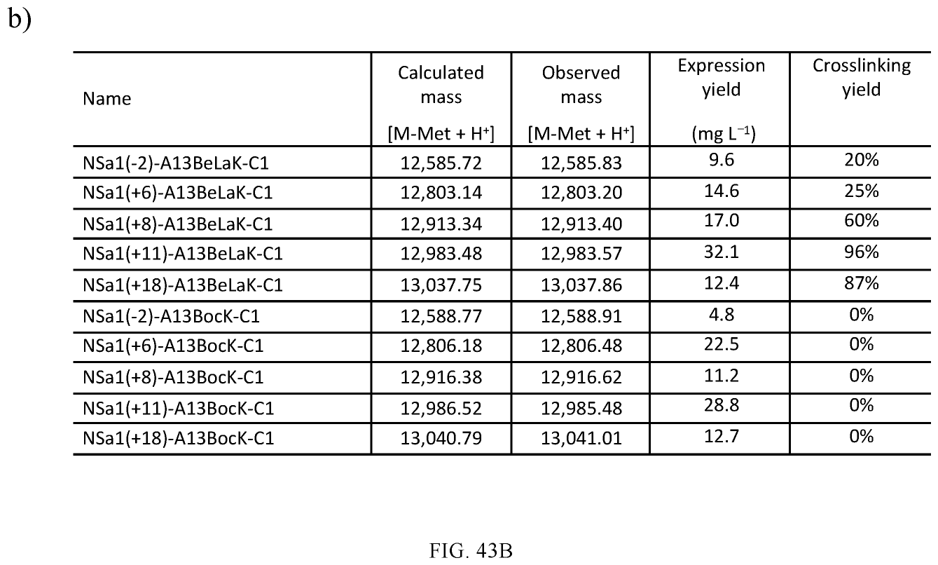

To gain a better understanding of cytosolic uptake and subcellular distribution of supercharged NSa1 mutants, we performed time-dependent confocal microscopy of the NSa1 mutants encoding either BocK or BeLaK. In general, the supercharged monobodies exhibited time-dependent accumulation inside HeLa cells, and the BeLaK-crosslinked NSa1 mutants showed greater cellular uptake than their non-crosslinked counterparts, in agreement with the flow cytometry results (FIG. 36b). Notably, AF488-NSa1(+11)-BeLaK displayed not only higher overall fluorescence intensity (FIG. 36c) but also more significant endosomal escape as indicated by high fluorescence intensity outside of the endosomes compared to its non-crosslinked counterpart (FIG. 36d). We attribute this effect to NSa1(+11)-BeLaK's high crosslinking yield of 96% (FIG. 43b) and high charge-to-mass ratio of 1.03. In contrast, +18 charged mutants are localized predominantly in the endosomes as indicated by the punctate green fluorescence in the cytosol regardless of the crosslinking status, which we attribute to their kinetic instability as a result of extensive mutagenesis.

In summary, we have identified a strained electrophilic amino acid, pi-lactam-lysine (BeLaK), that can be efficiently and site-specifically incorporated into proteins in E. coli via genetic code expansion. BeLaK displayed remarkable stability in bacterial culture and yet underwent efficient proximity-driven crosslinking of the GST dimer when placed at the dimer interface, preferably with lysine. When BeLaK was introduced site-specifically to the N-terminal β-strand of the supercharged monobodies, it allowed efficient interstrand orthogonal crosslinking with a nearby lysine, generating a rigidified protein scaffold. Compared to the non-crosslinked monobodies, the BeLaK-crosslinked supercharged mutants afforded higher thermostability and enhanced cytosolic uptake. Most significantly, +11 charged, orthogonally crosslinked monobody showed significant endosomal escape after endocytosis. Efforts to further increase cytosolic transport efficiency of the supercharged monobodies, including identifying additional orthogonal crosslinking sites and exploring genetic fusion with short endosomal escape domains, are ongoing and will be reported in due course.

TABLE 6
Sequences of DNA oligonucleotides used in this Example.

| Description | Primer Type | Sequence |
|---|---|---|
| PylRS-N346A-C348A | Forward | 5′-cgcacagatgggatcgggatgt-3′ (SEQ ID NO: 52) |
| | Reverse | 5′-aacgccagcatggtaaactcttcg-3′ (SEQ ID NO: |
| | | 53) |
| PylRS-A346V | Forward | 5′-taccatgctggtgttcgcacaga-3′ (SEQ ID NO: 54) |
| | Reverse | 5′-aactcttcgaggtgttctttgc-3′(SEQ ID NO: 55) |
| PylRS-A348L | Forward | 5′-gctggcgttcctgcagatgggatc-3′ (SEQ ID NO: |
| | Reverse | 56) |
| | | 5′-atggtaaactcttcgagg-3′ (SEQ ID NO: 57) |
| PylRS-N346V-C348L | Forward | 5′-cctgcagatgggatcgggatgt-3′ (SEQ ID NO: 58) |
| | Reverse | 5′-aagaccagcatggtaaactcttcg-3′ (SEQ ID NO: |
| | | 59) |
| GST-E52TAG | Forward | 5′-aacaaaaagtttgaattgggtttgtagtttcccaatc-3′ (SEQ |
| | | ID NO: 60) |
| | Reverse | 5′-aatataataaggaagattgggaaactacaaacccaattc- |
| | | 3′(SEQ ID NO: 61) |
| GST-E52TAG-K92Y | Forward | 5′-agagcgtgcatatatttcaatgcttg-3′ (SEQ ID NO: |
| | | 62) |
| | Reverse | 5′-tttggacaaccacccaac-3′ (SEQ ID NO: 63) |
| GST-E52TAG-K92C | Forward | 5′-agagcgtgcatgcatttcaatgcttg-3′ (SEQ ID NO: |
| | | 64) |
| | Reverse | 5′-tttggacaaccacccaac-3′ (SEQ ID NO: 65) |
| GST-E52TAG-K92H | Forward | 5′-agagcgtgcacatatttcaatgcttg-3′ (SEQ ID NO: |
| | | 66) |
| | Reverse | 5′-tttggacaaccacccaac-3′ (SEQ ID NO: 67) |
| GST-E52TAG-K92S | Forward | 5′-agagcgtgcatctatttcaatgcttgaag-3′ (SEQ ID |
| | | NO: 68) |
| | Reverse | 5′-tttggacaaccacccaac-3′ (SEQ ID NO: 69) |
| GST-E52TAG-K92D | Forward | 5′-agagcgtgcagatatttcaatgcttg-3′ (SEQ ID NO: |
| | | 70) |
| | Reverse | 5′-tttggacaaccacccaac-3′ (SEQ ID NO: 71) |
| GST-E52TAG-K92T | Forward | 5′-agagcgtgcaaccatttcaatgcttg-3′ (SEQ ID NO: |
| | Reverse | 72) |
| | | 5′-tttggacaaccacccaac-3′ (SEQ ID NO: 73) |
| NSa1(-2)-C1 | Forward | 5′-tgcgtgagcagcgtgccgacc-3′ (SEQ ID NO: 74) |
| | Reverse | 5′-gccctgaaaatacagattttcggtacc-3′ (SEQ ID NO: |
| | | 75) |
| NSa1(+6)-C1 | Forward | 5′-tgcgttagtagtaaaccgacc-3′ (SEQ ID NO: 76) |
| | Reverse | 5′-accctggaaatacaggttttc-3′ (SEQ ID NO: 77) |
| NSa1(+8)-C1 | Forward | 5′-tgcgttagtagtaaaccgacc-3′ (SEQ ID NO: 78) |
| | Reverse | 5′-accctggaaatacaggttttc-3′ (SEQ ID NO: 79) |
| NSa1(+11)-C1 | Forward | 5′-tgcgtgagcagtaaaccgacc -3′ (SEQ ID NO: 80) |
| | Reverse | S′-gccctggaaatacaggtttt -3′ (SEQ ID NO: 81) |
| NSa1(+18)-C1 | Forward | 5′-tgcgtgaaaagcaaaccgacc-3′ (SEQ ID NO: 82) |
| | Reverse | 5′-accctggaaatacaggttttc-3′ (SEQ ID NO: 83) |
| NSa1(+6)-Y92K | Forward | 5′-tagtattaaacgtacctaagg-3′ (SEQ ID NO: 84) |
| | Reverse | 5′-atcgggctgctataatag-3′ (SEQ ID NO: 85) |
| NSa1(+8)-Y92K | Forward | 5′-tagtattaataaacgtacctaagg-3′(SEQ ID NO: 86) |
| | Reverse | 5′-atcgggctgctataatag-3′ (SEQ ID NO: 87) |
| NSa1(+18)-Y92K | Forward | 5′-tagcattaataaacgcacctaag-3′(SEQ ID NO: 88) |
| | Reverse | S′-atcggactgctataatag-3′(SEQ ID NO: 89) |

General Information. Solvents and chemicals were purchased from commercial sources and used directly without further purification. Flash chromatography was performed with SiliCycle P60 silica gel (40-63 μm, 60 Å). ¹H and ¹³C NMR spectra were recorded with Varian Mercury-300, Inova-400, or −500 MHz spectrometer. Chemical shifts were reported in ppm using either TMS or deuterated solvents as internal standards (TMS, 0.00; CDCl₃, 7.26; CD₃OD, 3.31; DMSO-d₆, 2.50). Multiplicity was reported as follows: s=singlet, d=doublet, t=triplet, q=quartet, m=multiplet, brs=broad. ¹³C NMR spectra were recorded at 75.4, 101, or 126 MHz, and chemical shifts were reported in ppm using deuterated solvents as internal standards (CDCl₃, 77.0; DMSO-d₆, 39.5; CD₃OD, 49.05). LC-MS analysis was performed using an Agilent 6530 QTOF mass spectrometer coupled with Agilent 1260 HPLC system. Protein liquid chromatography was performed using a Phenomenex Aeris C4 column (3.6 μm, 200 Å, 2.10×50 mm) with a flow rate of 0.3 mL/min and a gradient of 10-90% ACN/H₂O containing 0.1% formic acid at 25° C. for 15 min or an Agilent PLRP-S column (5 μm, 1000 Å, 2.10×50 mm) with a flow rate of 0.5 mL/min and a gradient of 5-95% ACN/H₂O containing 0.1% formic acid at 60° C. for 10 min. Intact protein masses were obtained by deconvoluting charge ladders using BioConfirm 10.0 software (Agilent). High resolution mass spectrometry was performed on Agilent 6530 QTOF-LC/MS. NSa1 expression plasmids were purchased from Gene Universal (Newark, DE).

### Experimental Procedures and Characterization Data

### Scheme for Synthesis of BeLaF-1

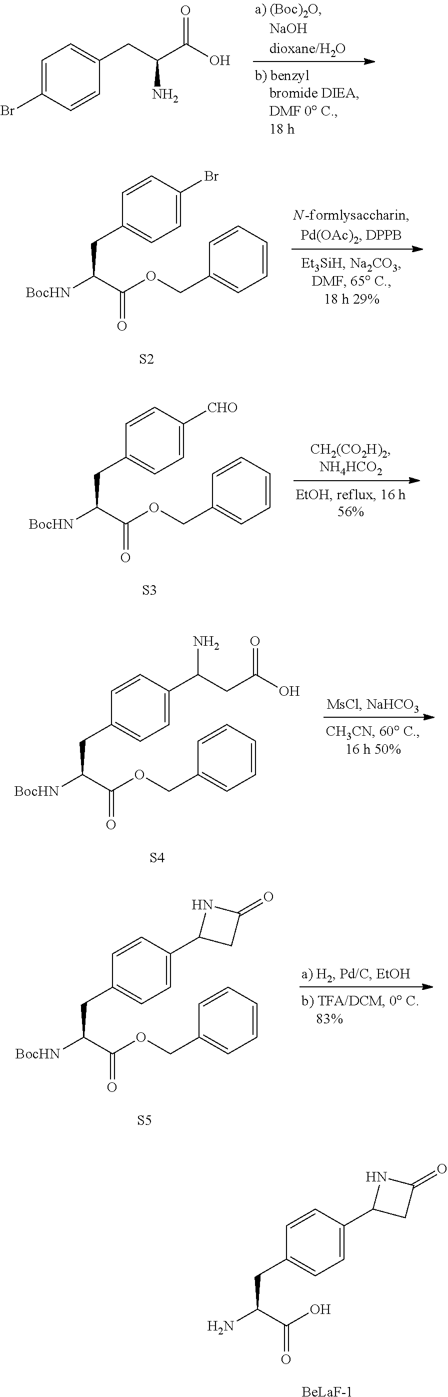

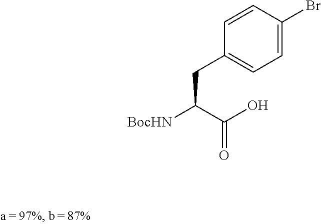 [SMILES: CC(C)(C)OC(=O)N[C@@H](Cc1ccc(Br)cc1)C(=O)O]

(S)-3-(4-Bromophenyl)-2-((tert-butoxycarbonyl)amino)propanoic acid (S1): To 4-bromo-L-phenylalanine (5 g, 20.4 mmol) in dioxane/H₂O (1:1, 80 mL) was added 1 M NaOH (20 mL) and di-tert-butyl dicarbonate (4.89 g, 22.44 mmol). The mixture was stirred at room temperature for 16 hours. Then, 1 M KHSO₄ solution was added to adjust pH=2-3, and the mixture was extracted with EtOAc (30 mL×2). The organic layers were combined, dried over anhydrous Na₂SO₄, filtered, and concentrated under reduced pressure to afford the title compound as a white solid (6.9 g, 97% yield). ¹H NMR (300 MHz, DMSO-d₆) δ 7.46-7.43 (m, 2H), 7.20-7.17 (m, 2H), 4.10-4.03 (m, 1H), 2.98 (dd, J=13.8, 4.7 Hz, 1H), 2.77 (dd, J=13.8, 10.4 Hz, 1H), 1.29 (s, 9H); HRMS calcd for C₁₄H₁₇BrNO₄ 342.0346 [M−H]⁻, found 342.0360.

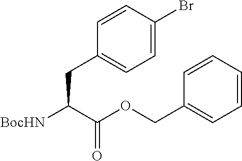 [SMILES: CC(C)(C)OC(=O)N[C@@H](Cc1ccc(Br)cc1)C(=O)OCc1ccccc1]

Benzyl (S)-3-(4-bromophenyl)-2-((tert-butoxycarbonyl)amino)propanoate (S2): To a solution of Si (7.05 g, 21.09 mmol) in DMF (75 mL) was added N,N-diisopropylethylamine (5.45 g, 42.18 mmol). The mixture was stirred at 0° C. before adding benzyl bromide (7.35 g, 43.02 mmol), and the stirring continued at room temperature for 18 hours. The solution was then diluted with saturated NH₄Cl, and the mixture was extracted with EtOAc (50 mL×3). The organic layers were combined, washed with brine, dried over anhydrous Na₂SO₄, and filtered, and concentrated. The residue was purified by silica gel flash chromatography (EtOAc/hexanes=1:2) to afford the title compound as a white solid (7.67 g, 87% yield). ¹H NMR (300 MHz, CDCl₃) δ 7.41-7.32 (m, 3H), 7.32-7.25 (m, 3H), 6.88 (d, J=8.0 Hz, 2H), 5.23-5.04 (m, 2H), 4.99 (d, J=8.3 Hz, 1H), 4.59 (t, J=7.0 Hz, 1H), 3.03 (t, J=5.9 Hz, 2H), 1.42 (s, 9H); ¹³C NMR (75 MHz, CDCl₃) δ 171.39, 135.01, 134.88, 131.56, 131.06, 128.66, 128.64, 128.59, 120.98, 80.06, 67.22, 54.23, 37.74, 28.27; HRMS calcd for C₂₁H₂₄BrNNaO₄ 456.0781 [M+Na⁺], found 456.0787.

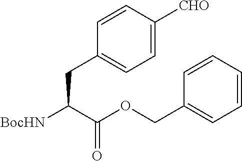 [SMILES: CC(C)(C)OC(=O)N[C@@H](Cc1ccc(C=O)cc1)C(=O)OCc1ccccc1]

Benzyl (S)-2-((tert-butoxycarbonyl)amino)-3-(4-formylphenyl)propanoate (S3): Following a published procedure, a mixture of S2 (200 mg, 0.46 mmol), Pd(OAc)₂ (3.1 mg, 0.014 mmol), 1,4-bis(diphenylphosphino)butane (8.8 mg, 0.021 mmol), N-formylsaccharin (291.4 mg, 1.38 mmol), and Na₂CO₃ (170.6 mg, 1.61 mmol) were added to a Schlenk tube. The tube was evacuated and backfilled with argon three times. Then, a degassed solution of Et₃SiH (80.23 mg, 0.69 mmol) in anhydrous DMF (2 mL) was added under argon. The mixture was stirred at room temperature for 10 minutes before stirring at 65° C. under argon for 16 hours. The mixture was cooled down, diluted with EtOAc, filtered through a layer of celite, and concentrated. The residue was purified by silica gel flash chromatography (EtOAc/hexanes=1:3) to afford the title compound as a brown oil (51 mg, 29% yield). ¹H NMR (300 MHz, CDCl₃) δ 9.90 (s, 1H), 7.68 (d, J=8.0 Hz, 2H), 7.39-7.23 (m, 5H), 7.17 (d, J=7.6 Hz, 2H), 5.16 (dd, J=11.9, 2.9 Hz, 2H), 5.06 (dd, J=12.1, 2.8 Hz, 1H), 4.70-4.53 (m, 1H), 3.12 (dd, J=15.9, 6.1 Hz, 2H), 1.37 (s, 9H); ¹³C NMR (75 MHz, CDCl₃) δ 191.83, 171.25, 162.56, 154.98, 143.32, 135.16, 134.97, 130.05, 129.82, 128.64, 128.61, 80.06, 67.25, 54.21, 38.45, 28.24; HRMS calcd for C₂₂H₂₅NNaO₅ 406.1625 [M+Na⁺], found 406.1672.

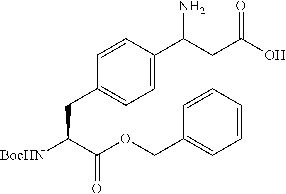 [SMILES: CC(C)(C)OC(=O)N[C@@H](Cc1ccc(C(N)CC(=O)O)cc1)C(=O)OCc1ccccc1]

3-Amino-3-(4-((S)-3-(benzyloxy)-2-((tert-butoxycarbonyl)amino)-3-oxo propyl)phenyl)propanoic acid (S4): Following a published procedure, a solution of S3 (400 mg, 1.04 mmol) in ethanol (5 mL) was added malonic acid (108 mg, 1.04 mmol) and ammonium formate (131.2 mg, 2.08 mmol). The mixture was stirred at room temperature for 16 hours before heating to 80° C. for five hours. Then, ethanol was removed under reduced pressure. The residue was purified by silica gel flash chromatography (MeOH/DCM=1:9) to afford the title compound as a pale-yellow solid (260 mg, 56% yield). ¹H NMR (300 MHz, CD₃OD) δ 7.34 (d, J=3.2 Hz, 7H), 7.25 (d, J=8.0 Hz, 2H), 5.14 (d, J=2.4 Hz, 2H), 4.49 (dd, J=9.5, 4.6 Hz, 1H), 4.37 (dd, J=9.2, 5.4 Hz, 1H), 3.14 (dd, J=13.8, 5.5 Hz, 1H), 2.99-2.89 (m, 1H), 2.70 (m, J=16.8, 7.0 Hz, 2H), 1.36 (s, 9H); ¹³C NMR (75 MHz, CD₃OD) δ 171.92, 156.44, 138.17, 135.74, 130.04-125.71 (m), 79.28, 66.57, 55.22, 52.59, 36.60, 29.33, 27.28; HRMS calcd for C₂₄H₃₁N₂O₆ 443.2177 [M+H⁺], found 443.2161.

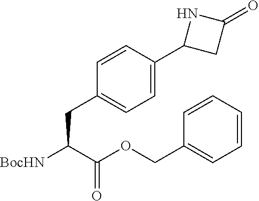 [SMILES: CC(C)(C)OC(=O)N[C@@H](Cc1ccc(C2CC(=O)N2)cc1)C(=O)OCc1ccccc1]

Benzyl (2S)-2-((tert-butoxycarbonyl)amino)-3-(4-(4-oxoazetidin-2-yl)phenyl) propanoate (S5): Following a published procedure, to a solution of S4 (10 mg, 0.023 mmol) in acetonitrile (3 mL) was added NaHCO₃ (11.5 mg, 0.138 mmol) and methanesulfonyl chloride (10.54 mg, 0.092 mmol). The mixture was stirred at 60° C. for 16 hours. Then, acetonitrile was removed under reduced pressure and the residue was purified by silica gel flash chromatography (EtOAc/hexanes=9:1) to afford the title compound as a brown solid (4.8 mg, 50% yield). ¹H NMR (300 MHz, CDCl₃) δ 7.41-7.28 (m, 5H), 7.21 (d, J=7.7 Hz, 2H), 7.04 (d, J=7.7 Hz, 2H), 6.32 (s, 1H), 5.21-5.07 (m, 2H), 5.02 (d, J=8.4 Hz, 1H), 4.66 (dd, J=5.3, 2.4 Hz, 1H), 4.63-4.51 (m, 1H), 3.41 (ddd, J=14.9, 5.2, 2.4 Hz, 1H), 3.08 (s, 2H), 2.82 (d, J=15.0 Hz, 1H), 1.41 (s, 9H); ¹³C NMR (75 MHz, CDCl₃) δ 171.61, 168.16, 155.03, 138.80, 136.07, 135.12, 129.84, 128.66, 128.60, 128.58, 128.56, 125.82, 80.03, 67.18, 54.42, 50.11, 47.87, 37.95, 29.68, 28.27; LRMS calcd for C₂₄H₂₈N₂NaO₅ 447.49 [M+Na⁺], found 447.41.

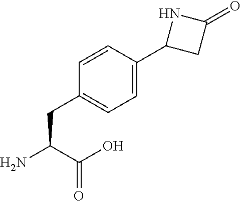 [SMILES: N[C@@H](Cc1ccc(C2CC(=O)N2)cc1)C(=O)O]

(2S)-2-Amino-3-(4-(4-oxoazetidin-2-yl)phenyl)propanoic acid (BeLaF-1): To S5 (28 mg, 0.066 mmol) in EtOH (2 mL) was added 10% Pd on carbon (3 mg). The mixture was filled with hydrogen and stirred at room temperature for 12 hours. Pd/C was removed by filtration through a layer of celite. The filtrate was concentrated to afford (2S)-2-((tert-butoxycarbonyl)amino)-3-(4-(4-oxoazetidin-2-yl)phenyl) propanoic acid (S6) as a white solid (22.05 mg, 88% yield). ¹H NMR (300 MHz, CD₃OD) δ 7.26 (d, J=4.2 Hz, 2H), 7.13 (s, 2H), 4.70 (dd, J=5.4, 2.3 Hz, 1H), 4.29-4.20 (m, 1H), 3.38 (dd, J=15.0, 5.3 Hz, 1H), 3.13 (td, J=14.1, 4.8 Hz, 1H), 2.86 (t, J=7.9 Hz, 1H), 2.46 (dd, J=9.0, 6.7 Hz, 1H), 1.36 (s, 9H); ¹³C NMR (75 MHz, CD₃OD) δ 169.70, 156.06, 138.90, 138.66, 129.52, 129.20, 127.78, 125.06, 78.77, 56.00, 49.49, 41.85, 36.96, 27.31; LRMS calcd for C₁₇H₂₂N₂O₅ 334.37 [M⁻], found 334.32. To above compound (S6) (19.4 mg, 0.058 mmol) in 2 mL DCM was added trifluoroacetic acid (0.4 mL) while stirring at 0° C. The mixture was stirred at room temperature for two hours. Then, DCM was removed under reduced pressure. The residue was washed with ice cold anhydrous diethyl ether (2 mL×2) and lyophilized to afford the title compound as a pale-yellow solid (16.8 mg, 83% yield): ¹H NMR (300 MHz, CD₃OD) δ 7.39 (d, J=8.0 Hz, 2H), 7.32 (d, J=8.1 Hz, 1H), 7.22 (d, J=2.5 Hz, 1H), 4.75 (dd, J=5.2, 2.3 Hz, 1H), 4.28-4.17 (m, 1H), 3.13 (ddd, J=18.0, 14.6, 7.8 Hz, 1H), 2.90 (dd, J=8.7, 6.7 Hz, 1H), 2.78-2.67 (m, 1H), 2.50 (t, J=7.6 Hz, 1H); ¹³C NMR (75 MHz, CD₃OD) δ 169.55, 140.54, 140.35, 129.49, 129.14, 128.72, 125.94, 53.74, 49.33, 41.84, 35.57; HRMS calcd for C₁₂H₁₅N₂O₃ 235.1077 [M+H⁺], found 235.1099.

### Scheme for Synthesis of BeLaF-2

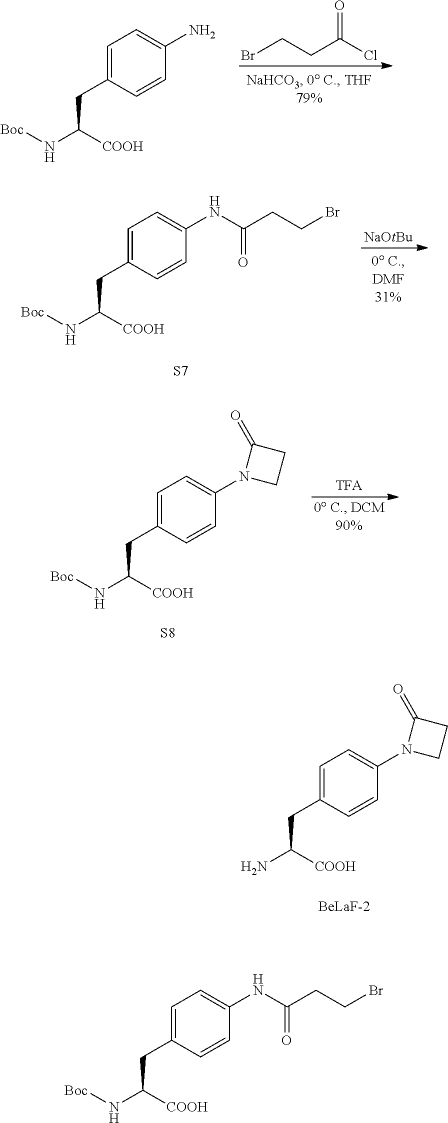

(S)-3-(4-(3-Bromopropanamido)phenyl)-2-((tert-butoxycarbonyl)amino) propanoic acid (S7): To a solution of (S)-3-(4-aminophenyl)-2-((tert-butoxy carbonyl)amino)propanoic acid (1 g, 3.56 mmol) in 50 mL anhydrous THF was added NaHCO₃ (600 mg, 7.13 mmol). The mixture was stirred in a round-bottom flask at 0° C. Then, 3-bromopropanoyl chloride (611.4 mg, 3.56 mmol) was added slowly and the reaction stirred for 10 minutes at 0° C. before removing the ice bath and allowing the mixture to stir for 2 hours, warming up to room temperature. Then THE was removed using reduced pressure before the addition of 1 N HCl to acidify the solution to pH 3. The mixture was extracted with ethyl acetate (3×), checking that pH=3 before each extraction. The organic layers were collected, dried over Na₂SO₄, filtered, and concentrated. The residue was purified by silica gel flash chromatography (MeOH/DCM=1:2) to afford the title compound as a pale-yellow solid (1.47 g, 79% yield). ¹H NMR (300 MHz, DMSO-d₆) δ 12.50 (s, 1H), 9.97 (s, 1H), 7.48 (d, J=8.1 Hz, 2H), 7.15 (d, J=8.1 Hz, 2H), 7.02 (d, J=8.3 Hz, 1H), 4.03 (m, 1H), 3.71 (t, J=6.3 Hz, 2H), 2.96 (m, 1H), 2.91 (t, J=6.3 Hz, 2H), 2.75 (dd, J=13.8, 10.1 Hz, 1H), 1.31 (s, 9H); ¹³C NMR (75 MHz, DMSO-d₆) δ 174.03, 168.41, 155.88, 137.76, 133.31, 129.78, 119.37, 78.49, 55.68, 39.81, 36.37, 29.71, 28.61; HRMS calcd for C₁₇H₂₃BrN₂NaO₅ 437.0683 [M+Na⁺], found 437.0831.

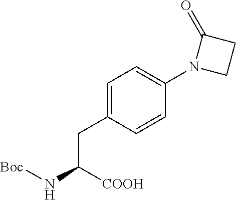 [SMILES: CC(C)(C)OC(=O)N[C@@H](Cc1ccc(N2CCC2=O)cc1)C(=O)O]

(S)-2-((tert-Butoxycarbonyl)amino)-3-(4-(2-oxoazetidin-1-yl)phenyl)propanoic acid (S8): A solution of S7 (0.5 g, 1.2 mmol) in 8 mL anhydrous DMF in a round-bottom flask was stirred at 0° C. under argon before adding potassium tert-butoxide (149 mg, 1.32 mmol) in one portion. The mixture was stirred under argon, allowing to warm up to room temperature for 16 hours. Then DMF was removed using reduced pressure before the slow addition of 1 N HCl (aqueous) to acidify the solution to pH=4-5. The mixture was extracted with ethyl acetate (3×), checking that pH=4-5 before each extraction. The organic layers were collected, dried over Na₂SO₄, filtered, and concentrated. The residue was purified by silica gel flash chromatography (MeOH/DCM=1:3) to afford the title compound as a light-brown oil (124 mg, 31% yield). ¹H NMR (300 MHz, CD₃OD) δ 7.30 (d, J=8.1 Hz, 2H), 7.22 (d, J=8.2 Hz, 2H), 4.30 (dd, J=9.0, 5.0 Hz, 1H), 3.64 (t, J=4.5 Hz, 2H), 3.18-3.10 (m, 1H), 3.07 (t, J=4.5 Hz, 2H), 2.89 (d, J=9.3 Hz, 1H), 1.38 (s, 9H); ¹³C NMR (75 MHz, CD₃OD) δ 174.07, 165.53, 156.37, 137.16, 133.05, 129.67, 115.94, 79.07, 55.01, 37.91, 36.81, 35.02, 27.26; LRMS calcd for C₁₇H₂₂N²NaO₅ 357.1421 [M+Na⁺], found

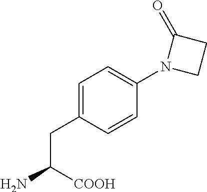 [SMILES: N[C@@H](Cc1ccc(N2CCC2=O)cc1)C(=O)O]

357.12.

(S)-2-amino-3-(4-(2-oxoazetidin-1-yl)phenyl)propanoic acid (BeLaF-2): To a solution of S8 (24 mg, 0.071 mmol) in 2 mL DCM was added trifluoroacetic acid (0.4 mL) while stirring at 0° C. After 30 minutes, the mixture was stirred at room temperature for two hours. Then, DCM was removed under reduced pressure. The residue was washed with ice cold anhydrous diethyl ether (2 mL×2) and lyophilized to afford the title compound as a pale-yellow solid (15.1 mg, 90% yield): ¹H NMR (300 MHz, CD₃OD) δ 7.38 (d, J=8.3 Hz, 2H), 7.28 (d, J=8.2 Hz, 2H), 4.22 (dd, J=7.6, 5.5 Hz, 1H), 3.68 (t, J=4.4 Hz, 2H), 3.25 (d, J=5.5 Hz, 1H), 3.14 (d, J=7.8 Hz, 1H), 3.10 (t, J=4.6 Hz, 2H); ¹³C NMR (75 MHz, CD₃OD) δ 169.81, 165.67, 138.07, 129.89, 129.65, 116.51, 53.69, 37.97, 35.41, 35.15; HRMS calcd for C₁₂H₁₄N²NaO₃ 257.0897 [M+Na⁺], found 257.0896.

### Scheme for Synthesis of BeLaK

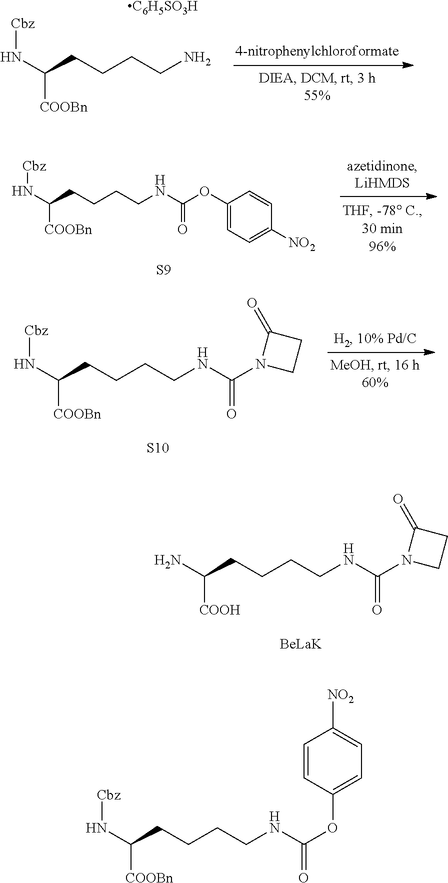

Benzyl N²-((benzyloxy)carbonyl)-N⁶-((4-nitrophenoxy)carbonyl)-L-lysinate (S9): A solution of 4-nitrophenylchloroformate (42 mg, 0.21 mmol) in 2 mL dichloromethane in a round-bottom flask was stirred at 0° C. under argon. Then, a solution of N^{α}-Z-L-lysine benzyl ester benzenesulfonate salt (100 mg, 0.189 mmol) and N,N-diisopropylethylamine (83 μL, 0.47 mmol) in 3 mL dichloromethane was added to the round bottom flask using a syringe pump at a rate of 0.75 mL/min. The mixture was stirred under argon at room temperature for 3 hours before addition of a saturated NH₄Cl solution (0.2 mL). The mixture was extracted with dichloromethane and the organic layer was separated, dried over Na₂SO₄, filtered, and concentrated. The residue was purified by silica gel flash chromatography (EtOAc/hexanes=1:2) to afford the title compound as a white solid (56 mg, 55% yield). ¹H NMR (400 MHz, CDCl₃) δ 8.20 (d, J=9.2 Hz, 2H), 7.40-7.30 (m, 10H), 7.30-7.26 (m, 2H), 5.40 (d, J=8.2 Hz, 1H), 5.27-5.21 (m, 1H), 5.16 (t, J=12.2 Hz, 2H), 5.10 (s, 2H), 4.44 (m, 1H), 3.21 (q, J=7.3 Hz, 2H), 1.89 (m, 1H), 1.72 (m, 1H), 1.62-1.49 (m, 2H), 1.38 (m, 2H); ¹³C NMR (101 MHz, CDCl₃) δ 172.15, 156.05, 155.95, 153.16, 144.71, 136.12, 135.21, 128.67, 128.59, 128.57, 128.38, 128.27, 128.08, 125.10, 121.93, 67.27, 67.11, 53.59, 40.89, 32.32, 28.87, 22.18; HRMS calcd for C₂₈H₃₀N₃O₈ 536.2027 [M+H⁺], found 536.2006.

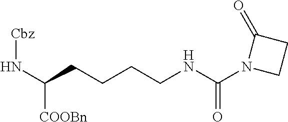

Benzyl N²-((benzyloxy)carbonyl)-N⁶-(2-oxoazetidine-1-carbonyl)-L-lysinate (S10): Following a published procedure, a stirred solution of azetidinone (140 mg, 1.97 mmol) in 19 mL anhydrous THE in an oven-dried round-bottom flask was added dropwise a 1M solution of lithium bis(trimethylsilyl)amide) in THE (2.17 mL, 2.17 mmol) at −78° C. under argon. The mixture was stirred at −78° C. for 15 minutes before a solution of S9 (528 mg, 0.987 mmol) in 2 mL anhydrous THE under argon was added slowly. Then, the mixture was stirred under argon for 30 minutes at −78° C. After 30 minutes, the mixture was allowed to stir at room temperature for 10 minutes. A saturated aqueous NH₄Cl solution (3 mL) was then added to the mixture and stirred for 30 minutes at room temperature. THE was removed using reduced pressure before extracting with EtOAc. The organic layer was dried over anhydrous Na₂SO₄, filtered, and concentrated. The residue was purified by silica gel flash chromatography (EtOAc/hexanes=1:1) to afford the title compound as a colorless oil (444 mg, 96% yield). ¹H NMR (400 MHz, CDCl₃) δ 7.39-7.29 (m, 10H), 6.47 (t, J=6.0 Hz, 1H), 5.44-5.35 (m, 1H), 5.23-5.12 (m, 2H), 5.10 (s, 2H), 4.39 (m, 1H), 3.57 (t, J=4.8 Hz, 2H), 3.22 (m, 2H), 2.99 (t, J=4.8 Hz, 2H), 1.86 (m, 1H), 1.76-1.63 (m, 1H), 1.51 (m, 2H), 1.42-1.28 (m, 2H); ¹³C NMR (101 MHz, CDCl₃) δ 172.23, 167.02, 155.95, 150.70, 136.27, 135.32, 128.62, 128.52, 128.46, 128.31, 128.16, 128.12, 67.12, 66.97, 53.79, 39.16, 37.09, 35.93, 31.96, 29.30, 22.22; HRMS calcd for C₂₅H₃₀N₃O₆ 468.2129 [M+H⁺], found 468.2862.

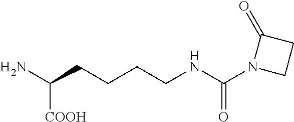 [SMILES: N[C@@H](CCCCNC(=O)N1CCC1=O)C(=O)O]

N⁶-(2-Oxoazetidine-1-carbonyl)-L-lysine (BeLaK): To a solution of S10 (1.7 g, 3.63 mmol) in methanol (30 mL) was added Pd/C (150 mg, 10%). The round bottom flask was filled with hydrogen and stirred at room temperature for 16 hours. The Pd/C was removed by washing with excess methanol while filtering through celite. The filtrate was concentrated to afford the title compound as an off-white solid (520 mg, 60% yield). ¹H NMR (500 MHz, D₂O) δ 3.68-3.63 (m, 1H), 3.57 (t, J=4.8 Hz, 2H), 3.20 (t, J=6.9 Hz, 2H), 3.04 (t, J=4.8 Hz, 2H), 1.80 (m, 2H), 1.53 (m, 2H), 1.41-1.27 (m, 2H); ¹³C NMR (126 MHz, D₂O) δ 174.87, 169.66, 152.32, 54.68, 39.13, 37.74, 35.43, 30.11, 28.48, 21.61; HRMS calcd for C₁₀H₁₇N₃NaO₄ 266.1111 [M+Na⁺], found 266.1167.

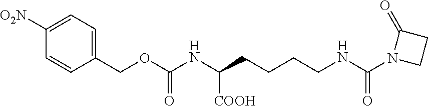 [SMILES: O=C(N[C@@H](CCCCNC(=O)N1CCC1=O)C(=O)O)OCc1ccc([N+](=O)[O-])cc1]

N²-(((4-Nitrobenzyl)oxy)carbonyl)-N⁶(2-oxoazetidine-1-carbonyl)-L-lysine (S11): To a solution of BeLaK (50 mg, 0.2 mmol) in a mixture of H₂O/dioxane (1:1, 2 mL) was added NaHCO₃ (34 mg, 0.41 mmol). The mixture was stirred at 0° C. for 5 minutes. Then, 4-nitrobenzylchloroformate (53 mg, 0.246 mmol) was added in one portion and the mixture was stirred at 0° C. for 2 hours followed at room temperature by 16 hours. The mixture was transferred into a separatory funnel and washed with diethyl ether (2×). The aqueous phase fractions were combined and acidified to pH=4 using 1 N HCl. Then the mixture was extracted using EtOAc (3×). Organic fractions were combined and washed with H₂O, brine, and dried over anhydrous NaSO₄. The organic fraction was filtered and concentrated using reduced pressure. The residue was purified by recrystallization (ethanol/hexanes=1:1) to afford the title compound as colorless needle crystals (80 mg, 95% yield). ¹H NMR (400 MHz, CD₃OD) δ 8.12 (d, J=8.8 Hz, 2H), 7.50 (d, J=8.6 Hz, 2H), 5.12 (s, 2H), 4.04 (dd, J=9.3, 4.7 Hz, 1H), 3.46 (t, J=4.8 Hz, 2H), 3.15 (t, J=6.9 Hz, 2H), 2.94 (t, J=4.8 Hz, 2H), 1.83-1.72 (m, 1H), 1.62 (m, 1H), 1.46 (m, 1H), 1.36 (m, 1H); ¹³C NMR (101 MHz, CD₃OD) δ 174.38, 167.35, 156.87, 151.39, 147.50, 144.69, 127.66, 123.18, 64.77, 53.89, 38.99, 36.89, 35.26, 30.86, 28.92, 22.72; HRMS calcd for C₁₈H₂₂N₄NaO₈ 445.1330 [M+Na⁺], found 445.1352.

Site-directed mutagenesis to generate pEVOL-PylRS-N³⁴⁶ Å-C348 Å. The plasmid carrying wild-type PylRS gene with a C-terminal His-tag (pEVOL-PylRS) was purchased from Gene Universal Inc. The asparagine and cysteine codons in position 346 and 348, respectively, were mutated to alanine using Q5 Site-Directed Mutagenesis Kit (New England Biolabs) with the following primers (Forward: cgcaCAGATGGGATCGGGATGT (SEQ ID NO: 90); Reverse: aacgcCAGCATGGTAAACTCTTCG (SEQ ID NO: 91)) to obtain the pEVOL-PylRS-N346A-C348 Å fragment. The PCR product was subjected to kinase, ligase, and dNP's (KLD buffer, KLD enzyme) treatment to obtain the pEVOL-PylRS-N346A-C348 Å pDNA product. Then, 5 μL of KLD mixtures were transformed into chemically component DH5a cells (New England Biolabs, Ipswich, MA) and the transformants were recovered in TB medium at 37° C. for 1 hour and plated onto an LB/agar plate containing 34 μg/mL chloramphenicol. After overnight incubation at 37° C., the surviving colonies were collected from the plates and allowed to grow in LB medium containing 34 μg/mL chloramphenicol at 37° C. overnight. The PylRS-N346A-C348 Å plasmid was purified using a plasmid mini-prep kit. The concentration of the plasmid was determined by using Nanodrop 2000c spectroscopy (Thermo Fisher Scientific, Waltham, MA). The plasmids were sent for Sanger sequencing (Genewiz, Inc.) and the results were compared to the original PylRS template to confirm the mutations.

sfGFP fluorescence measurement. BL21(DE3) cells (50 μL) were co-transformed with the pET-sfGFP-Q204TAG and pEvol-PylRS-N346A-C348 Å plasmids using heat shock and recovered in 950 μL Terrific Broth (TB) and incubated at 37° C. for 1 hour before plating to Luria-Bertani (LB) agar plate containing 100 μg/mL ampicillin and 34 μg/mL chloramphenicol. A single colony from the plate was picked and used to inoculate 5-mL LB broth containing 100 μg/mL ampicillin and 34 μg/mL chloramphenicol. Two hundred μL overnight culture was then used to inoculate 20 mL LB broth containing the same concentrations of antibiotics. The cells were grown until OD₆₀₀reached ˜0.7 and the protein expression was induced by adding 0.2% arabinose and 1 mM isopropyl 3-D-1-thiogalactopyranoside (IPTG). The culture was divided into three 5-mL portions. One portion of the culture was supplemented with 1 mM β-Lactam UAA, the second portion served as a positive control with 1 mM O-allyl-tyrosine, and the third portion served as a control without adding any β-Lactam UAA. The cultures were incubated for 16 hours (25° C., 280 rpm). The cells were pelletized in 15-mL conical tubes and resuspended in 1.0 mL binding buffer (10 mM imidazole, 300 mM NaCl in 50 mM Na₂HIPO₄, pH 8.0). The cell suspensions were then sonicated at 0° C. before being spun down using a swinging bucket centrifuge (Beckman Coulter, Allegra™ X-22R). The supernatant containing the lysates was transferred to a quartz cuvette where the fluorescence emission intensities of these proteins under 470 nm irradiation were measured using a FluoroMax-4 spectrofluorometer (Horiba Scientific).

Site-specific incorporation of BeLaK into sfGFP. BL21(DE3) cells (50 μL) were co-transformed with the pET-sfGFP-Q204TAG and pEVOL-PylRS plasmids using the heat shock method. The cells were recovered in 900 μL SOC at 37° C. for 1 hour before plating onto a Luria-Bertani (LB) agar plate containing 100 μg/mL ampicillin and 34 μg/mL chloramphenicol. A single colony from the plate was used to inoculate 6 mL LB broth containing 100 μg/mL ampicillin and 34 μg/mL chloramphenicol. 120 μL overnight culture was used to inoculate 12 mL LB broth containing the same concentrations of antibiotics. The cells were grown until OD₆₀₀ reached ˜0.6 and protein expression was induced by adding 0.2% arabinose and 1 mM isopropyl β-D-1-thiogalacto pyranoside (IPTG). The culture was divided into two 6-mL portions. One portion of the culture was supplemented with 1 mM BeLaK, and the other portion served as a control without BeLaK. The cultures were incubated in an incubator-shaker (37° C., 280 rpm) for 8 hours. The cells were pelletized in 15 mL conical tubes and resuspended in 1.5 mL native binding buffer (10 mM imidazole, 300 mM NaCl in Na₂HPO₄, pH 8.0) containing protease inhibitor cocktail (Pierce™) on ice for 15 min. The supernatant was directly used for fluorescence tests after sonication and centrifugation. The lysate was transferred into a 1.5 mL microcentrifuge tube containing 20 L Ni-NTA agarose beads (Thermo HisPur™). The mixture was incubated for 2 hours with gentle shaking. The resin was centrifuged briefly and washed three times with native washing buffer (50 mM imidazole, 300 mM NaCl in 50 mM Na₂HPO₄, pH 8.0). Finally, the protein was eluted with 500 μL native elution buffer (250 mM imidazole, 300 mM NaCl in 50 mM Na₂HPO₄, pH 8.0). The protein yield was calculated based on the concentration determined using Pierce™ BCA protein assay kit (Thermo Fisher Scientific).

Expression and purification of BeLaK-encoded glutathione S-transferase (GST) mutants. BL21(DE3) cells (50 μL) were co-transformed with pET28a(+)-GST mutant and pEVOL-PylRS plasmids using the heat shock method. The cells were recovered in 950 L SOC medium (New England Biolabs) and incubated at 37° C. for 1 hour before plating to a LB agar plate containing 50 μg/mL kanamycin and 34 μg/mL chloramphenicol. A single colony was used to inoculate 6 mL of LB containing 50 μg/mL kanamycin and 34 μg/mL chloramphenicol. Two hundred L aliquot of overnight culture was used to inoculate 20 mL LB medium containing the same concentrations of antibiotics. The cells were grown until OD₆₀₀ reached ˜0.7 and protein expression was induced by adding 0.2% arabinose and 1 mM isopropyl β-D-1-thiogalacto pyranoside (IPTG). The culture was divided into two 10-mL portions. One portion of the culture was supplemented with 1 mM BeLaK, and the other portion served as a control without BeLaK. The cultures were incubated overnight (25° C., 280 rpm, 16 hours). The cells were pelletized in 15 mL conical tubes and resuspended in 700 L BugBuster® Protein Extraction reagent (Millipore) before transferring into 1.5 mL microcentrifuge tube. The lysate was incubated for 20 min and then centrifuged before transferring to 1.5 mL microcentrifuge tube containing 50 μL Ni-NTA agarose beads (Thermo HisPur™). The mixture was diluted with 500 μL native binding buffer (10 mM imidazole, 300 mM NaCl in 50 mM Na₂HIPO₄, pH 8.0) and incubated for 2 hours with gentle shaking at 4° C. The resin was centrifuged briefly and washed three times with native washing buffer (50 mM imidazole, 300 mM NaCl in 50 mM Na₂HPO₄, pH 8.0). Finally, the protein was eluted with 1.0 mL native elution buffer (250 mM imidazole, 300 mM NaCl in 50 mM Na₂HPO₄, pH 8.0). The elution was concentrated using Amicon Ultra-0.5 mL Centrifugal Filter (MWCO 10 kDa; Millipore) followed by buffer exchange to a phosphate buffer (pH 7.4) to a final volume of 100 L. The protein yield was calculated based on concentration determined using Pierce™ BCA protein assay kit (Thermo Fisher Scientific).

SDS-PAGE and western blot analysis of BeLaK-encoded glutathione S-transferase (GST) mutants. The proteins were mixed with an equal amount of 2x SDS loading buffer and heated at 95° C. for 10 min before loading onto 4-12% SDS-PAGE gel (GenScript). The proteins were separated at 140 V for 60 min and detected using Coomassie blue staining. For western blot, proteins were resolved by SDS-PAGE and transferred to a PVDF membrane (ThermoFisher Scientific). The membrane was blocked in 1% casein in TBST (50 mM Tris, 150 mM NaCl, 0.05% Tween-20, pH 7.6) at 4° C. overnight, and then incubated with anti-6×His epitope tag (rabbit) antibody (1:1000, Rockland) in TBST at room temperature for 1 h. The membrane was washed with TBST (5 min×6) before the addition of the anti-rabbit IgG horseradish peroxidase conjugate antibody (1:4000, Promega). After 30 minutes, the membrane was washed with TBST (5 min×6) followed by a single wash using a Tris buffer, pH=9.5 (100 mM, 5 min). After addition of Pierce™ ECL Western Blotting Substrate (Thermo Fisher Scientific), the membrane was incubated in dark for 5 min before capturing an image using a BioRad ChemiDoc™ MP imaging instrument.

Expression and purification of BeLaK-encoded NSa1 proteins. BL21(DE3) cells (50 μL) were co-transformed with pET28a(+)-NSa1-A13TAG (variants) and pEVOL-PylRS(WT) plasmids using heat shock and recovered in 900 μL SOC media (New England Biolabs) and incubated at 37° C. for 1 hour before plating to LB agar plate containing 50 g/mL kanamycin and 34 μg/mL chloramphenicol. A single colony from the plate was picked and used to inoculate 6 mL LB containing 50 μg/mL kanamycin and 34 μg/mL chloramphenicol. A 2 mL suspension of overnight culture was used to inoculate a 200 mL culture of LB containing the same concentrations of antibiotics. The cells were grown until OD₆₀₀ reached ˜0.6 and the protein expression was induced by adding 0.2% arabinose and 1 mM IPTG. The culture was divided into two 100-mL portions. One portion of the culture was supplemented with 1 mM BeLaK and the other portion served as a control with 2 mM BocK. The cultures were incubated overnight (25° C., 280 rpm, 16 hours). The cells were pelletized in 50 mL conical tubes and resuspended with 6 mL lysis buffer (50 mM Tris HCl, pH 8.0, 0.5 M NaCl) containing protease inhibitor cocktail (Pierce™) on ice for 15 min. The cells were lysed by sonication on ice and then centrifuged (4° C., 8,000 RPM, 25 min). The supernatant was transferred into 15 mL tubes with 40 L Ni-NTA agarose beads (Thermo HisPur™) and incubated for 2 hours with gentle shaking at 4° C. The resin was centrifuged briefly and washed three times with native washing buffer (50 mM Na₂IPO₄, pH 8.0, 300 mM NaCl, 50 mM imidazole). Finally, the protein was eluted with 0.5 mL elution buffer (50 mM Na₂IPO₄, pH 7.4, 300 mM NaCl, 250 mM imidazole). Immediately following, the BeLaK-encoded NSa1 proteins were subjected directly to TEV protease cleavage reaction (1 TEV: 11 protein) for 16 hours at 4° C. with gentle mixing. Then the reaction mixture was concentrated using Pall Nanosep with 3K Omega centrifugal devices (4° C., 10,000×g, 5 min) and then diluted into FPLC start buffer (50 mM Na₂HPO₄, pH 7.0) supplemented with 5% glycerol. The mixture was spun down (4° C., 10,000×g, 10 min) to remove any precipitate before FPLC purification using cation-exchange chromatography (monoS 5/50 GL, Cytiva) with NaCl gradient in 50 mM Na₂HPO₄ buffer (pH 7.0).

Expression and purification of BeLaK-encoded NSa1-C1 proteins. BL21(DE3) cells (50 μL) were co-transformed with pET28a(+)-NSa1-C1-A13TAG (variants) and pEVOL-PylRS(WT) plasmids using heat shock and recovered in 900 L SOC media (New England Biolabs) and incubated at 37° C. for 1 hour before plating to LB agar plate containing 50 μg/mL kanamycin and 34 μg/mL chloramphenicol. A single colony from the plate was picked and used to inoculate 6 mL LB containing 50 μg/mL kanamycin and 34 g/mL chloramphenicol. A 2 mL suspension of overnight culture was used to inoculate a 200 mL culture of LB containing the same concentrations of antibiotics. The cells were grown until OD₆₀₀ reached ˜0.6 and the protein expression was induced by adding 0.2% arabinose and 1 mM IPTG. The culture was divided into two 100-mL portions. One portion of the culture was supplemented with 1 mM BeLaK and the other portion served as a control with 2 mM BocK. The cultures were incubated overnight (25° C., 280 rpm, 16 hours). The cells were pelletized in 50 mL conical tubes and resuspended with 6 mL lysis buffer (50 mM Tris HCl, pH 8.0, 0.5 M NaCl, 1 mM TCEP) containing protease inhibitor cocktail (Pierce™) on ice for 15 min. The cell was lysed by sonication on ice and then centrifuged (4° C., 8,000 RPM, 25 min). The supernatant was transferred into 15 mL tubes with 40 L Ni-NTA agarose beads (Thermo HisPur™) and incubated for 2 hours with gentle shaking at 4° C. The resin was centrifuged briefly and washed three times with native washing buffer (50 mM Na₂IPO₄, pH 8.0, 300 mM NaCl, 50 mM imidazole). Finally, the protein was eluted with 0.5 mL elution buffer (50 mM Na₂IPO₄, pH 7.4, 300 mM NaCl, 250 mM imidazole, 1 mM TCEP). Immediately following, the BeLaK-encoded NSa1-C1 proteins were subjected directly to TEV protease cleavage reaction (1 TEV: 11 protein) for 16 hours at 4° C. with gentle mixing. Then the reaction mixture was concentrated using Pall Nanosep with 3K Omega centrifugal devices (4° C., 10,000×g, 5 min) and then diluted into FPLC start buffer (50 mM Na₂HPO₄, pH 7.0) supplemented with 5% glycerol. The mixture was spun down (4° C., 10,000×g, 10 min) to remove any precipitate before FPLC purification using cation-exchange chromatography (monoS 5/50 GL, Cytiva) with NaCl gradient in 50 mM Na₂IPO₄ buffer (pH 7.0).

Crosslinking yield determination of BeLaK-encoded NSa1 variants. To quantify the extent of crosslinking, β-mercaptoethanol (20 mM) was added at a final concentration of 2.58 mM (100 equiv, 3 μL) to a solution of purified NSa1 variants (25 μM, 30 μL) in elution buffer (50 mM Na₂HPO₄, pH=7.4, 300 mM NaCl, 250 mM imidazole). The mixture was incubated at 37° C. overnight. Afterwards, the mixture was removed from the incubator and allowed to incubate at 25° C. for 1 week. The NSa1 protein mass was then investigated using Agilent QTOF-LC/MS wherein the comparison of the protein mass peak areas between the hydrolyzed BeLaK and the intact BeLaK revealed the extent of crosslinking.

Thermostability assay of NSa1 proteins. The assay was performed following a literature protocol.⁵ NSa1 protein variants (5 M, 20 μL) in PBS (pH 7.4) were incubated at 25, 37, 55, 75, 90, or 100° C. for 10 min and then quickly placed on ice. The samples were spun at 15,000×g at 4° C. for 30 min and then part of the supernatant was removed. 5×SDS loading buffer was added to the supernatant and the samples were then heated at 95° C. for 10 min using a dry bath incubator (Boekel Scientific) before loaded onto a 12% SDS-PAGE gel (Genscript). The proteins were separated at 140 V for 60 min and detected using Coomassie blue staining. Each gel contained a control sample of protein that had been left on ice throughout the experiment. Protein percent recovery was calculated from the band intensity relative to the control sample on that gel, defined as 100%.

Cytotoxicity assay of NSa1 proteins in mammalian cells. Protocols were followed as provided by the manufacturer, Promega CytoTox-Glo™ Cytotoxicity Assay kit. NSa1 protein variants were serially diluted two-fold from a stock solution in Dulbecco's modified eagle medium (DMEM, Life Technologies) supplemented with 10% (v/v) fetal bovine serum (FBS, Life Technologies) in 12.5 L volumes into a 384-plate (Corning). HeLa cells were added at 10,000 cells/well in a 12.5 L volume. The plate was briefly mixed manually and then incubated for 18 hours at 37° C. in 5% CO₂. The CytoTox-Glom Cytotoxicity Assay Reagent was prepared, and then 12.5 L was added to each well. After another brief mix, the 384-plate was incubated at room temperature for 15 minutes and the luminescence signal was measured using a Synergy H1 microplate reader (BioTek).

Fluorescent labeling of NSa1-C1 proteins. Following FPLC cation exchange chromatography, the purified NSa1-C1 proteins were buffer exchanged into a slightly basic buffer (50 mM phosphate buffer, pH=7.6, 500 mM NaCl, 1 mM TCEP, 3% glycerol) and incubated with Alexa-Fluor™ 488-C₅ maleimide (ThermoFisher) at 4° C. with gentle shaking in the absence of light for 16 hours. Afterwards, thorough dialysis (D-Tube™, Novagen) was carried out to remove excess dye reagent and the solution was exchanged into a suitable buffer (50 mM Na₂HPO₄, pH 7.4, 400 mM NaCl) for cell culture. The protein concentrations were determined using NanoDrop;

$$[ protein ( M ) ] = \frac{( {A}_{280} - ( {A}_{493} \times {CF}_{280} ) )}{{ε}_{NSa 1}} ( {ε}_{NSa 1} = 28 , 880 {M}^{- 1} {cm}^{- 1} , {ε}_{NSa 1 ( + 6 , + 8 , + 11 , + 18 )} = 27 , 390 {M}^{- 1} {cm}^{- 1} ; {CF}_{280} = 0.11 ) .$$

Flow cytometry of mammalian cells treated with NSa1-AF488 proteins. HeLa cells were maintained in growth medium containing Dulbecco's modified eagle medium (DMEM, Life Technologies) supplemented with 10% (v/v) fetal bovine serum (FBS, Life Technologies) and 10 μg/mL Gentamycin (Gibco) and 2 μg/mL Plasmocin (InvivoGen) at 37° C., 5% CO₂. The cells were washed twice with pre-warmed Dulbecco's phosphate buffered saline (DPBS, Life Technologies) when ˜80% confluency was reached. Then a solution of NSa1-AF488 labeled protein variants (2 μM) was diluted in DMEM growth medium supplemented with 10% FBS (without phenol red, Life Technologies) to obtain a final concentration of 40 nM (200 μL) NSa1-labeled proteins per well using a Cellstar 48-well plate (Greiner Bio-one). The cells were incubated for 5 hours at 37° C., 5% CO₂ before washing three times with pre-warmed DPBS containing 20 U/mL heparin. Next, the cells were trypsinized and collected into 1.5 mL microcentrifuge tubes following a brief centrifugation (400×g, 5 min, 22° C.). Lastly, the cells were collected and resuspended in DPBS for flow cytometry analysis. The samples were loaded into a BD Biosciences LSR Fortessa X-20 flow cytometer and analyzed based on GFP-channel fluorescence. The data was plotted and analyzed using FCS Express 7 research edition software.

Confocal imaging of mammalian cells treated with NSa1-AF488 proteins. HeLa cells were maintained in growth medium containing Dulbecco's modified eagle medium (DMEM, Life Technologies) supplemented with 10% (v/v) fetal bovine serum (FBS, Life Technologies) and 10 μg/mL Gentamycin (Gibco) and 2 μg/mL Plasmocin (InvivoGen) at 37° C., 5% CO₂. The cells were washed twice with pre-warmed Dulbecco's phosphate buffered saline (DPBS, Life Technologies) when ˜80% confluency was reached. Then, a solution of NSa1-AF488 labeled protein variants (2 μM) was diluted in DMEM growth medium supplemented with 10% FBS (without phenol red, Life Technologies) to obtain a final concentration of 40 nM (200 μL) NSa1-C1-AF488 labeled proteins per well using an 8-well chambered cover glass plate (Nunc™ Lab-Tek™ II, ThermoFisher). The cells were incubated for desired time points (1, 3, 5, or 18 hours) at 37° C., 5% CO₂ before washing three times with pre-warmed DPBS containing 20 U/mL heparin. The DPBS solution was then switched to Fluorobrite DMEM (Life Technologies) before laser scanning confocal microscopy. The confocal images were acquired using a Zeiss LSM 710 equipped with Plan-Apochromat 20×/0.8 M27 or 40×/1.3 Oil DIC M27 objective with ex. 488/em. 493-598 nm for the GFP channel and ex. 350/em. 461 nm for the DAPI channel. Images were analyzed using Zen 3.2 blue edition (Zeiss) software.

### Example 4

This example provides a description of the preparation, characterization, and use of non-crosslinked proteins and crosslinked proteins of the present disclosure.

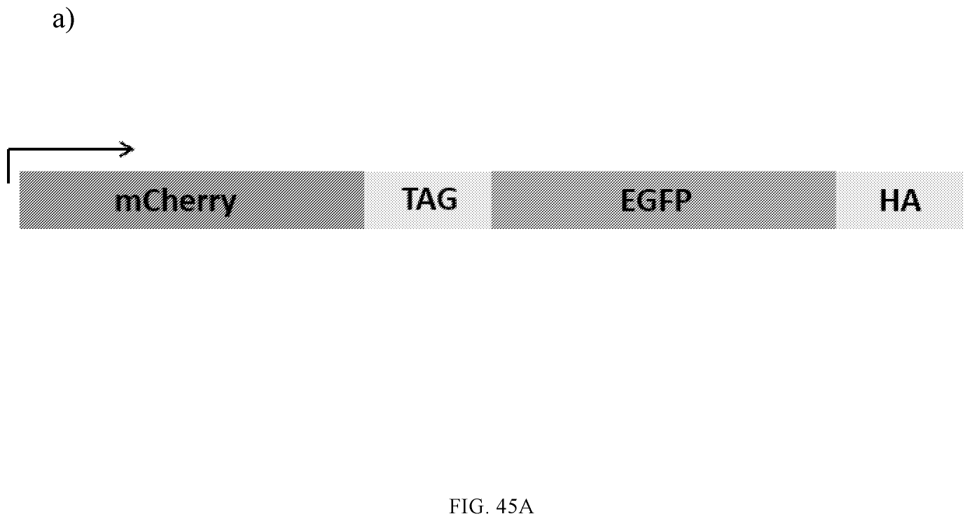

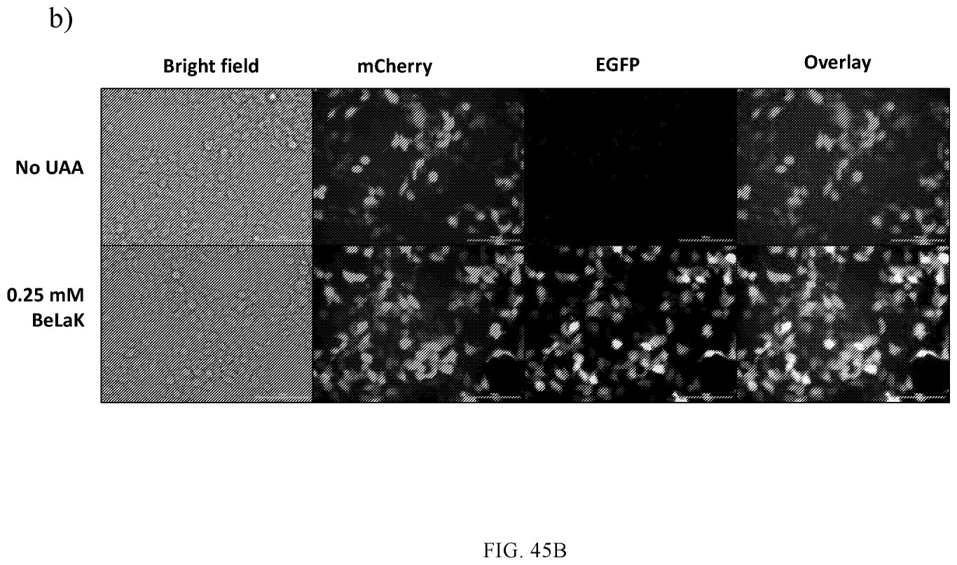

Site-specific incorporation of BeLaK into mCherry-TAG-EGFP in mammalian cells. HEK293T cells were seeded into a 24-well plate and grown in DMEM supplemented with 10% FBS (HyClone™ GE Healthcare Life Sciences) and 10 μg/mL Gentamycin (Gibco) and 2 μg/mL Plasmocin at 37° C., 5% CO₂ until ˜80% confluency. The medium was replaced with DMEM, and cells were transfected with two plasmids, one encoding wtPylRS/tRNAPyl CUA pair and another encoding mCherry-TAG-EGFP-HA, using PEI (Polysciences) in Opti-MEM® (Gibco). Six hours post-transfection, the medium was replaced with fresh DMEM with 10% FBS in the presence or absence of 0.25 mM BeLaK. After 24 hours, live cell images were recorded using Lionheart™ FX automated microscope (BioTek). Results are shown in FIG. 45.

Although the present disclosure has been described with respect to one or more particular examples, it will be understood that other examples of the present disclosure may be made without departing from the scope of the present disclosure.

1. A compound comprising the following structure: 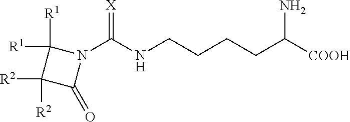
or a structural analog thereof, or a pharmaceutically acceptable salt, a salt, a partial salt, a solvate, a polymorph thereof, or a stereoisomer or a mixture of stereoisomers, an isotopic variant, or a tautomer thereof, wherein
X is O or S or the like,
R¹ and R² are independently at each occurrence chosen from hydrogen group, halide groups, alkyl groups, cycloalkyl groups, alkoxy groups, alkylamino groups, alkylthiol groups, and structural analogs thereof, and
optionally, a R¹ and a R² form a hydrocarbon ring or a heterocyclic ring,
or 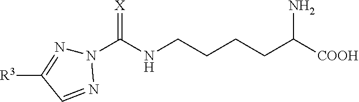
or a structural analog thereof, or a pharmaceutically acceptable salt, a salt, a partial salt, a solvate, a polymorph, or a stereoisomer or a mixture of stereoisomers, an isotopic variant, or a tautomer thereof, wherein
X is O or S or the like, and
R³ is chosen from hydrogen group, alkyl groups, cycloalkyl groups, aromatic groups, heteroaromatic groups, and structural analogs thereof.

2. The compound of claim 1, wherein the R³ group comprises the following structure: 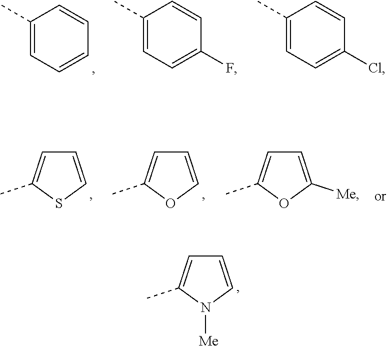
wherein Me is a methyl group, or a structural analog thereof.

3. The compound of claim 1, wherein the compound comprises the following structure: 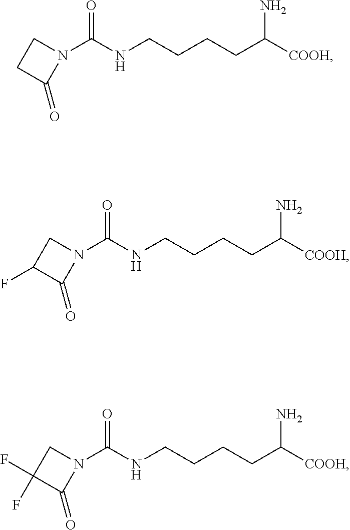 [SMILES: NC(CCCCNC(=O)N1CCC1=O)C(=O)O ; NC(CCCCNC(=O)N1CC(F)C1=O)C(=O)O ; NC(CCCCNC(=O)N1CC(F)(F)C1=O)C(=O)O]
or a structural analog thereof.

4. (canceled)

5. (canceled)

6. A protein comprising:
one or more first amino acid residue(s) comprising a side-chain reactive site, the first amino acid residue(s) comprising the following structure: 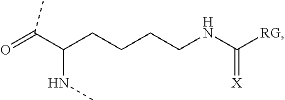
wherein RG is a reactive group independently at each occurrence comprising (or consisting of) the following structure: 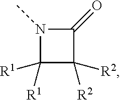
wherein X is O or S, R¹ and R² are independently at each occurrence chosen from hydrogen group, halide groups, alkyl groups, cycloalkyl groups, alkoxy groups, alkylamino groups, alkylthiol groups, and structural analogs thereof, and optionally, a R¹ and a R² form a hydrocarbon ring or a heterocyclic ring,
or 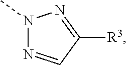
wherein R³ is chosen from hydrogen group, alkyl groups, cycloalkyl groups, aromatic groups, heteroaromatic groups, and structural analogs thereof.

7. The protein of claim 6, wherein RG is independently at each occurrence comprises the following structure: 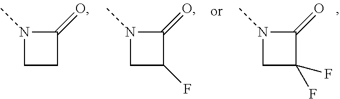
or a structural analog thereof.

8. The protein of claim 6, wherein the R³ group independently at each occurrence comprises: 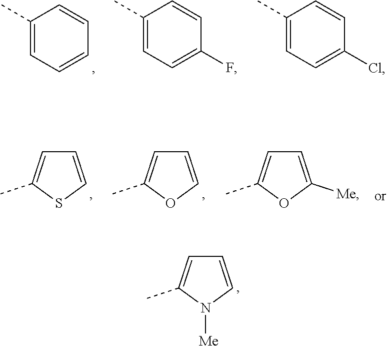
wherein Me is a methyl group, or a structural analog thereof.

9. The protein of claim 6, further comprising one or more second amino acid residue(s), comprising a nucleophilic side-chain reactive site, wherein one or more or all of the first amino acid residue(s) is/are each in proximity to a second amino acid residue, such that the side-chain reactive site of each of the one or more or all first amino acid residue(s) is capable of reacting with the side-chain reactive site of a second amino acid residue in proximity thereto to form one or more intramolecular crosslink(s) and/or one or more intermolecular crosslink(s).

10. (canceled)

11. The protein of claim 6, wherein the second amino acid residue(s) is/are independently at each occurrence chosen from lysine, tyrosine, histidine, cysteine, serine, and threonine.

12. The protein of claim 6, wherein the protein further comprises one or more cysteine disulfide bond(s).

13. The protein of claim 6, wherein the protein is capable of forming the one or more intramolecular and/or one or more intermolecular crosslink(s) without interfering with one or more cysteine disulfide bond(s) and/or one or more other cysteine residue(s) which are not second amino acid residue(s).

14. The protein of claim 6, wherein the protein is a single protein capable of forming one or more inter-strand intramolecular crosslink(s) and/or one or more intra-strand intramolecular crosslink(s) and/or the protein is a complex of a plurality of single proteins, wherein each single protein of the plurality is capable of forming one or more intramolecular crosslink(s) and/or one or more intermolecular crosslink(s) with one or more other single protein(s) of the plurality of single proteins.

15. (canceled)

16. (canceled)

17. (canceled)

18. The protein of claim 6, wherein the protein comprises an overall net surface charge of from about +1 to about +20.

19. The protein of claim 6, wherein the protein is an engineered protein.

20. The protein of claim 6, wherein the protein comprises an antibody or a portion thereof.

21. The protein of claim 20, wherein the antibody is a monoclonal antibody, an antibody fragment, a single-chain variable fragment, a fusion protein, a monobody, a nanobody, an affibody, an aptamer, an affilin, an affimer, an affitin, an alphabody, an anticalin, an avimer, a knottin, an armadillo repeat protein, designed ankyrin repeat proteins (DARPins), fynomers, gastrobodies, clostridal antibody mimetic proteins (nanoCLAMPs), optimers, repebodies, recombinant fibronectins, a centyrin, or an obody.

22. The protein of claim 6, wherein the protein further comprises one or more therapeutic modalit(ies), one or more diagnostic modalit(ies), or any combination thereof.

23. (canceled)

24. A crosslinked protein comprising:
one or more intramolecular crosslink(s) and/or one or more intermolecular crosslink(s), the intramolecular crosslink(s) and/or the intermolecular crosslink(s) independently at each occurrence comprising the following structure: 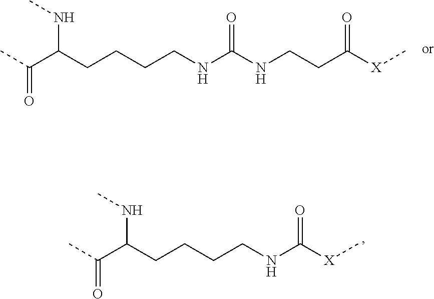
wherein X is independently at each occurrence an O atom, S atom, N atom, or NH group.

25. (canceled)

26. The crosslinked protein of claim 25, wherein a first protein comprises the first amino acid residue(s) and a second protein comprises the second amino acid residue(s), and
wherein the first protein and the second protein are comprised within a single protein and
wherein the crosslink(s) is/are intramolecular crosslink(s) and/or wherein the first protein and the second protein are comprised within separate proteins and wherein the crosslinks(s) is/are intermolecular crosslink(s).

27. (canceled)

28. (canceled)

29. (canceled)

30. (canceled)

31. The crosslinked protein of claim 24, wherein the crosslinked protein comprises an overall net surface charge of from about +1 to about +20.

32. The crosslinked protein of claim 24, wherein the crosslinked protein is a crosslinked engineered protein.

33. The crosslinked protein of claim 24, wherein the crosslinked protein comprises a protein chosen from antibodies, monoclonal antibodies, antibody fragments, single-chain variable fragments, fusion proteins, monobodies, nanobodies, affibodies, aptamers, affilins, affimers, affitins, alphabodies, anticalins, avimers, knottins, armadillo repeat proteins, designed ankyrin repeat proteins (DARPins), fynomers, gastrobodies, clostridal antibody mimetic proteins (nanoCLAMPs), optimers, repebodies, recombinant fibronectins, centyrins, and obodies.

34. The crosslinked protein of claim 33, wherein the crosslinked protein further comprises one or more therapeutic modalit(ies), one or more diagnostic modalit(ies), or any combination thereof.

35. (canceled)

36. (canceled)

37. (canceled)

38. (canceled)

39. (canceled)

40. (canceled)

41. (canceled)

42. A method of forming a crosslinked protein of claim 24 comprising
contacting a first protein with a second protein,
wherein the first protein comprises one or more first amino acid residue(s) comprising a side-chain reactive site, the first amino acid residue(s) comprising the following structure: 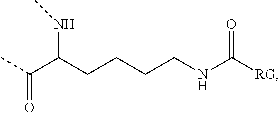
wherein RG is a reactive group independently at each occurrence comprising the following structure: 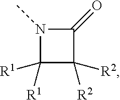
wherein R¹ and R² are independently at each occurrence chosen from hydrogen group, halide groups, alkyl groups, cycloalkyl groups, alkoxy groups, alkylamino groups, alkylthiol groups, and structural analogs thereof, and optionally, a R¹ and a R² form a hydrocarbon ring or a heterocyclic ring,
or 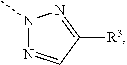
wherein R³ is chosen from hydrogen group, alkyl groups, cycloalkyl groups, aromatic groups, heteroaromatic groups, and structural analogs thereof, and
wherein the second protein comprises one or more second amino acid residue(s) comprising a nucleophilic side-chain reactive site, wherein one or more or all of the first amino acid residue(s) is/are each in proximity to a second amino acid residue, such that the side-chain reactive site of each of the one or more or all first amino acid residue(s) is capable of reacting with the side-chain reactive site of a second amino acid residue in proximity thereto to form one or more intramolecular crosslink(s) and/or one or more intermolecular crosslink(s),
thereby forming the crosslinked protein.

43. The method of claim 42, wherein the first protein and the second protein are comprised within a single protein and wherein the crosslink(s) is/are intramolecular crosslink(s) and/or first protein and the second protein are comprised within separate proteins and wherein the crosslinks(s) is/are intermolecular crosslink(s).

44. (canceled)

45. The method of claim 42, wherein the contacting is performed inside a cell or at the surface of a cell and/or the contacting is performed in solution.

46. (canceled)

47. (canceled)

48. (canceled)

49. A method of covalent binding a protein to a target on a cell, the method comprising contacting the cell with one or more protein(s) of claim 6, wherein the protein(s) is/are independently capable of specifically binding to a target on the surface of the cell, whereby the protein forms one or more intermolecular crosslink(s) with the target.

50. (canceled)

51. (canceled)

52. (canceled)

53. (canceled)

54. (canceled)

55. (canceled)

56. (canceled)

57. (canceled)

58. (canceled)

59. A method of cellular delivery, the method comprising:
contacting one or more crosslinked protein(s) of claim 24 with a cell or a population of cells, wherein the crosslinked protein(s) are delivered into the cell or the population of cells.

60. (canceled)

61. (canceled)

62. An engineered pyrrolysyl-tRNA synthetase comprising one or more amino acid mutation(s) within a substrate-binding site as compared to a wild-type pyrrolysyl-tRNA synthetase, wherein the substrate-binding site comprises amino acid 306, amino acid 309, amino acid 348 of SEQ ID NO: 24 or in corresponding positions thereto in a variant thereof.

63. (canceled)

64. (canceled)

65. (canceled)

66. (canceled)

67. (canceled)

68. (canceled)

69. (canceled)

70. (canceled)

71. A complex comprising the engineered pyrrolysyl-tRNA synthetase of claim 62 and a compound of claim 1.

72. (canceled)

73. A method of producing a protein of claim 6, comprising contacting a nucleic acid with an engineered pyrrolysyl-tRNA synthetase of claim 62, a tRNA^{Pyl}, and a compound of claim 1,
wherein the nucleic acid encodes a protein, and wherein the nucleic acid comprises at least one codon recognized by a tRNA^{Pyl}, thereby producing the protein.

74. (canceled)

75. (canceled)

76. (canceled)
